(19) United States
(12) Patent Application Publication
(10) Pub. No.: US 2023/0226224 A1
(43) Pub. Date: Jul. 20, 2023

(54) POLYNUCLEOTIDE

(71) Applicant: Freeline Therapeutics Limited, Stevenage (GB)

(72) Inventors: Azadeh KIA, Enfield (GB); Jonathan FOLEY, London (GB); Miriam CANAVESE, London (GB); Romuald CORBAU, Chiswell Green (GB); Samantha CORREIA, London (GB)

(21) Appl. No.: 18/011,624

(22) PCT Filed: Jun. 25, 2021
(86) PCT No.: PCT/GB2021/051627

§ 371 Date: Dec. 20, 2022

(30) Foreign Application Priority Data
Jun. 25, 2020 (GB) 2009741.6

(51) Int. Cl. A61K 48/00; C12N 15/86; C07K 14/47

(52) U.S. Cl. CPC A61K 48/0066; A61K 48/0058; C07K 14/472; C12N 15/86; C12N 2750/14143; C12N 2750/14171

(57) ABSTRACT

The present invention relates to a polynucleotide comprising a Complement Factor I (CFI) nucleotide sequence encoding a CFI polypeptide or a fragment thereof. The invention further relates to a viral particle comprising a recombinant genome comprising the polynucleotide of the invention, and a composition comprising the polynucleotide or viral particle of the invention. The invention also relates to methods of using, and uses of, the polynucleotide, viral particle and/or composition of the invention. The invention also relates to methods of using, and uses of, a polynucleotide comprising a CFI nucleotide sequence, a viral particle comprising a recombinant genome comprising the polynucleotide, or a composition comprising the polynucleotide or viral particle.

### FIELD OF THE INVENTION

The present invention relates to a polynucleotide comprising a Complement Factor I (CFI) nucleotide sequence encoding a CFI polypeptide or a fragment thereof. The invention further relates to a viral particle comprising a recombinant genome comprising the polynucleotide of the invention, and a composition comprising the polynucleotide or viral particle of the invention. The invention also relates to methods of using, and uses of, the polynucleotide, viral particle and/or composition of the invention. The invention also relates to methods of using, and uses of, a polynucleotide comprising a CFI nucleotide sequence, a viral particle comprising a recombinant genome comprising the polynucleotide, or a composition comprising the polynucleotide or viral particle.

### BACKGROUND TO THE INVENTION

Complement Factor I (CFI) is a protein involved in the complement system. The complement system is a part of the innate immune system, and forms a first line of defence against infections by triggering inflammatory responses. The complement system comprises a number of proteins that are synthesised primarily by the liver and circulate in the blood as inactive precursors. The stimulation of the complement system, for instance by a foreign entity, triggers proteases in the system to cleave the precursors and release molecules such as cytokines to initiate a further amplifying cascade. The activation of the complement system can stimulate phagocytes, stimulate inflammation and activate the cell-killing membrane attack complex. The complement system can be activated by the classical pathway, lectin pathway and alternative pathway. All three pathways converge at the cleavage and activation of C3 by C3-convertase. C3a and C3b are formed, causing a cascade of further cleavage and activation events.

The alternative pathway is constitutively active at low levels (so called “tick-over”) and can also serve as an amplification loop when C3b is released into the fluid phase or, more typically, becomes opsonized to a foreign entity. Also, the alternative pathway can be triggered by damaged tissues or propagated on tissues lacking essential regulators. Under normal circumstances, the alternative pathway is governed by a balance between two competing cycles, namely a C3b feedback cycle that enhances complement amplification and a C3b breakdown cycle that leads to down regulation.

The alternative pathway is continuously activated at a low level due to spontaneous C3 hydrolysis to form C3b. C3b can be covalently bound to the surface of a cell or pathogen. The surface-bound C3b can combine with Factor B to form a complex, C3bB. C3bB can be cleaved in the presence of Factor D to form C3bBb (alternative pathway C3-convertase) that can convert more C3 into C3b. The complexed C3bBb can bind Factor P (properdin) to form the stabilised C3-convertase C3bBbP, which is able to cleave more C3 to C3b, thereby amplifying the response. When the C3bBbP complex forms on a pathogen or cell surface, it is able to bind an additional C3b to form alternative pathway C5-convertase, consisting of (C3b)₂BbP. C5-convertase cleaves C5 into C5a and C5b. The membrane attack complex can then be formed from C5b binding sequentially to C6, C7, C8 and multiple C9 molecules.

A C3 breakdown cycle exists to prevent excessive C3b generation. In particular, CFI plays a role in the C3 breakdown cycle. Its role is to regulate complement activation by cleaving C3b and iC3b in two steps. In the first step, CFI, in the presence of the co-factor Factor H, catalyses the cleavage of C3b to iC3b. iC3b cannot form part of a C3 or C5 convertase, but is pro-inflammatory. In the second step, CFI, in the presence of the co-factor CR1 (CD35), then further breaks down iC3b yielding a C3 fragment, C3d,g.

The complement system plays a role in many diseases with an immune component, such as lupus nephritis and C3 glomerulopathy. Dysregulation of these pathways therefore has clinical implications. For example, if CFI is not functioning or lacking, or the alternative pathway is over stimulated or inappropriately stimulated, the balance of the complement system can be then disrupted, leading to a build-up of inflammatory factors e.g. in the kidney, which can lead to kidney pathology. Thus, it is desirable to rebalance the cycles of the alternative pathway, particularly by promoting C3b and iC3b breakdown, and thereby reduce inflammation. Thus, it would be therapeutically advantageous to restore or increase the level of CFI.

Accordingly, there is a need to provide an effective therapy vector for complement-mediated disorders which would allow the expression levels of functional CFI to be restored or increased. Restoring or increasing the expression levels of functional CFI could be useful to treat complement-mediated disorders, such as kidney glomerular diseases.

### SUMMARY OF THE INVENTION

The present application demonstrates that administration of a polynucleotide comprising a CFI nucleotide sequence which encodes a CFI polypeptide or a fragment thereof to the liver can be used to provide the CFI polypeptide or the fragment thereof systemically. In order to achieve this, CFI nucleotide sequences which express the CFI polypeptide or the fragment thereof at high levels are advantageous. In particular, such nucleotide sequences can be administered at lower doses, with a lower cost of goods and with a lower toxicity.

The present application demonstrates that an improved expression level of the CFI polypeptide or fragment thereof can be achieved in vitro and/or in vivo. This improved expression level can be achieved by modifying the nucleotide sequence which encodes the CFI polypeptide or fragment thereof. Thus, the present invention provides a polynucleotide comprising a CFI nucleotide sequence, wherein the CFI nucleotide sequence encodes a CFI polypeptide or a fragment thereof and wherein at least a portion of the CFI nucleotide sequence is not wild-type.

In addition, the present application demonstrates that polynucleotides comprising a CFI nucleotide sequence that encodes a CFI polypeptide or fragment thereof can be used to treat complement-mediated disorders using mouse models. Thus, the present invention also provides a polynucleotide comprising a CFI nucleotide sequence, a viral particle comprising a recombinant genome comprising the polynucleotide, or a composition comprising the polynucleotide or viral particle for use in a method of treatment, wherein the method of treatment is a method of treating a complement-mediated disorder, and the CFI nucleotide sequence encodes a CFI polypeptide or a fragment thereof.

Accordingly, in a first aspect of the invention, there is provided a polynucleotide comprising a Complement Factor I (CFI) nucleotide sequence, wherein the CFI nucleotide sequence encodes a CFI polypeptide or a fragment thereof and wherein at least a portion of the CFI nucleotide sequence is not wild-type.

In a second aspect of the invention, there is provided a polynucleotide comprising a CFI nucleotide sequence, wherein the CFI nucleotide sequence encodes a CFI polypeptide or a fragment thereof and the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1-40.

In a third aspect of the invention, there is provided a viral particle comprising a recombinant genome comprising the polynucleotide of the invention.

In a fourth aspect of the invention, there is provided a composition comprising the polynucleotide or viral particle of the invention, and a pharmaceutically acceptable excipient.

In a fifth aspect of the invention, there is provided the polynucleotide, viral particle or composition of the invention for use in a method of treatment.

In a sixth aspect of the invention, there is provided a method of treatment comprising administering an effective amount of the polynucleotide, viral particle or composition of the invention.

In a seventh aspect of the invention, there is provided use of the polynucleotide, viral particle or composition of the invention in the manufacture of a medicament for use in a method of treatment.

In an eighth aspect of the invention, there is provided a polynucleotide comprising a Complement Factor I (CFI) nucleotide sequence, a viral particle comprising a recombinant genome comprising the polynucleotide, or a composition comprising the polynucleotide or viral particle for use in a method of treatment, wherein the method of treatment is a method of treating a complement-mediated disorder, and the CFI nucleotide sequence encodes a CFI polypeptide or a fragment thereof.

In a ninth aspect of the invention, there is provided a method of treatment comprising administering an effective amount of a polynucleotide comprising a CFI nucleotide sequence, a viral particle comprising a recombinant genome comprising the polynucleotide, or a composition comprising the polynucleotide or viral particle, wherein the method of treatment is a method of treating a complement-mediated disorder, and the CFI nucleotide sequence encodes a CFI polypeptide or a fragment thereof.

In a tenth aspect of the invention, there is provided use of a polynucleotide comprising a CFI nucleotide sequence, a viral particle comprising a recombinant genome comprising the polynucleotide, or a composition comprising the polynucleotide or viral particle in the manufacture of a medicament for use in a method of treatment, wherein the method of treatment is a method of treating a complement-mediated disorder, and the CFI nucleotide sequence encodes a CFI polypeptide or a fragment thereof.

The present application demonstrates that using a codon-optimised CFI nucleotide sequence can improve the expression of the encoded CFI polypeptide. Such modified CFI nucleotide sequences may be further modified to provide further improvements in the expression of the encoded CFI polypeptide. Further modifications may include providing further modifications in the CFI nucleotide sequence such as the removal of CpG motifs.

### DESCRIPTION OF THE FIGURES

FIG. 1 shows a Spearman rank of both CFI expression levels and peritoneal neutrophil counts in C57BL/b mice. The mice were administered via intravenous injection with 6×10¹¹, 2×10¹² or 6×10¹³ vg/kg of AAV comprising CFI, or were untreated (“UT”), and their human CFI expression levels in blood (collected from the tail vein) were determined by ELISA. N=3 for untreated; n = 4 for 6×10¹¹ vg/kg dose and for 2×10¹² vg/kg dose; and n=2 for 6×10¹³ vg/kg dose. The AAV comprised the Co01 codon-optimised CFI nucleotide sequence and the CAG promoter. The levels of human CFI (“Factor I”) were measured at 4, 8 and 12 weeks post-administration and the mean values are plotted in FIG. 1A. Error bars indicate one standard deviation. At 16 weeks post-administration of AAV, the mice were treated with Zymosan suspended in PBS via intraperitoneal injection. The number of peritoneal neutrophils were determined 4 hours after Zymosan treatment and the mean values are plotted in FIG. 1B (Y-axis = Neutrophil Count x 10⁶/mL). Error bars indicate one standard deviation. The lane marked “control” was not administered with AAV and was treated with PBS rather than Zymosan. N=3 for the “control”. The CFI expression levels at 12 weeks post-administration and the neutrophil counts at 16 weeks post-administration of AAV were correlated using Spearman’s rank. The results are shown in FIG. 1C. For the Spearman’s rank, r = -0.82 and p < 0.001.

FIG. 2 shows the levels of proteinuria (as measured by ACR) for the different treatment groups in the MRL-lpr mouse model. “Untreated” is treatment group 1. “AAV-CFI” is treatment group 2. “MRL/Mp” is treatment group 3. “Cyclophos.” is treatment group 4. The mean values are plotted. Error bars indicate one standard deviation.

FIG. 3 shows the semi-quantitative grading of the PAS stained sections for the four treatment groups.

FIG. 4 - Sequence listing

FIG. 5 shows the CFI expression levels observed for a wild-type human CFI nucleotide sequence and a variety of codon-optimised human CFI sequences in the culture media of Huh-7 cells following transfection. The results are shown in the order of expression level. The CFI expression levels have been normalised using luciferase expression levels in order to account for transfection efficiency. The CFI expression levels are presented as ng/ml (ng of CFI per ml of media). Each transfection was performed in duplicate and the value obtained for each repeat is shown as a dot. Error bars = mean± SD. The sequences were expressed using the LSP-S promoter and the BgpA polyA sequence. LOQ, limit of quantification.

FIG. 6 shows the results of an in vitro study comparing expression of a wild-type human CFI protein following plasmid transfection in Huh7 cells. The plasmids used either the FRE72 promoter, or the LSP-S or LSP-L promoters (each of which is a liver-specific transcription regulatory element). The level of protein in culture supernatant was measured at day 3 post-transfection using an ELISA. A CMV-luciferase control plasmid was used as co-transfection vector for normalisation of transfection efficiency. FIG. 6A shows the results prior to luciferase correction and FIG. 6B shows the results following luciferase correction. The bars represent median values.

FIG. 7 - Schematic showing the different sequence elements present in FRE72 (SEQ ID NO: 66), HCR-hAAT (SEQ ID NO: 67), NP1 (SEQ ID NO: 68), NP2 (SEQ ID NO: 69), NP3 (SEQ ID NO: 70), NP4 (SEQ ID NO: 71), NP5 (SEQ ID NO: 72), NP6 (SEQ ID NO: 73), and NP7 (SEQ ID NO: 74). The labels in each section of the schematics show the names of the various sequence elements. The sequence element referred to as ApoE-HCR1 is a sequence element of SEQ ID NO: 92. The sequence element referred to as hAAT is a sequence element of SEQ ID NO: 94. The sequence element referred to as AMBP is a sequence element of SEQ ID NO: 97. The sequence element referred to as CRM6 is a sequence element of SEQ ID NO: 98. The sequence element referred to as ALDOB is a sequence element of SEQ ID NO: 102. The sequence element referred to as F2 is a sequence element of SEQ ID NO: 104. The labels to the left of the schematics show the name of the TREs. The numbers to the right of the schematic show the length of the corresponding TREs.

FIGS. 8 - (A) Schematic showing the sequence elements present in NP3 (SEQ ID NO: 70), including the location of the ApoE enhancer within an ApoE HCR-1 sequence element and the High Homology region within a hAAT sequence element. The scale above shows the nucleotide position of each of the different elements. (B) Table summarising the features of a group of TREs based on NP3 (NP3-derived TREs). The table indicates the differences between various NP3-derived TREs and NP3.

FIG. 9 - Graph showing the fold change in expression levels of CFI promoted by NP1, NP2, NP3, NP4, NP5, NP6, and NP7 in Huh7 cells compared to HCR-hAAT. HLP2 and FRE72 are included as controls. Huh7 human hepatocytes were transfected with plasmid DNA comprising TREs operably linked to CFI alongside a control plasmid comprising a luciferase encoding nucleotide sequence operably linked to a CMV promoter. Cells were then grown, harvested and the expression levels of CFI measured by ELISA. In parallel to the ELISA, a luciferase assay was performed to determine luciferase expression levels. CFI expression levels for each sample were normalised against the corresponding luciferase expression levels. Error bars show the mean + 1 SD.

FIG. 10 - Graph showing the fold change in expression levels of CFI promoted by NP12-NP24 in Huh7 cells compared to HCR-hAAT. HLP2, FRE72, and NP3 are included as controls. Huh7 human hepatocytes were transfected with plasmid DNA comprising TREs operably linked to CFI alongside a control plasmid comprising a luciferase encoding nucleotide sequence operably linked to a CMV promoter. Cells were then grown, harvested and the expression levels of CFI measured by ELISA. In parallel to the ELISA, a luciferase assay was performed to determine luciferase expression levels. CFI expression levels for each sample were normalised against the corresponding luciferase expression levels. Error bars show the mean +/- 1 SD.

FIG. 11 - Graph showing the fold change in expression levels of CFI promoted by NP8-NP11 in Huh7 cells compared to HCR-hAAT. HLP2, FRE72, and NP3 are included as controls. Huh7 human hepatocytes were transfected with plasmid DNA comprising TREs operably linked to CFI alongside a control plasmid comprising a luciferase encoding nucleotide sequence operably linked to a CMV promoter. Cells were then grown, harvested and the expression levels of CFI measured by ELISA. In parallel to the ELISA, a luciferase assay was performed to determine luciferase expression levels. CFI expression levels for each sample were normalised against the corresponding luciferase expression levels. Error bars show the mean +/- 1 SD.

FIG. 12 shows an outline of the steps of complement activation. FD, FB, FH, and FI stand for Factors D, B, H, and I respectively.

FIG. 13 shows the feedback loop of the alternative pathway of vertebrate complement. Dashed lines indicate enzymatic reaction. Solid lines indicate conversions of proteins. D, B, H, and I stand for Factors D, B, H, and I respectively.

Description of the Sequences

| SEQ ID NO | Sequence description |
|---|---|
| 1 | Codon-optimised CFI nucleotide sequence without signal peptide portion (Co01) |
| 2 | Codon-optimised CFI nucleotide sequence without signal peptide portion (Co02) |
| 3 | Codon-optimised CFI nucleotide sequence without signal peptide portion (Co03) |
| 4 | Codon-optimised CFI nucleotide sequence without signal peptide portion (Co04) |
| 5 | Codon-optimised CFI nucleotide sequence without signal peptide portion (Co05) |
| 6 | Codon-optimised CFI nucleotide sequence without signal peptide portion (Co06) |
| 7 | Codon-optimised CFI nucleotide sequence without signal peptide portion (Co07) |
| 8 | Codon-optimised CFI nucleotide sequence without signal peptide portion (Co08) |
| 9 | Codon-optimised CFI nucleotide sequence without signal peptide portion (Co09) |
| 10 | Codon-optimised CFI nucleotide sequence without signal peptide portion (Co10) |
| 11 | Codon-optimised CFI nucleotide sequence without signal peptide portion (Co11) |
| 12 | Codon-optimised CFI nucleotide sequence without signal peptide portion (Co12) |
| 13 | Codon-optimised CFI nucleotide sequence without signal peptide portion (Co13) |
| 14 | Codon-optimised CFI nucleotide sequence without signal peptide portion (Co14) |
| 15 | Codon-optimised CFI nucleotide sequence without signal peptide portion (Co15) |
| 16 | Codon-optimised CFI nucleotide sequence without signal peptide portion (Co16) |
| 17 | Codon-optimised CFI nucleotide sequence without signal peptide portion (Co17) |
| 18 | Codon-optimised CFI nucleotide sequence without signal peptide portion (Co18) |
| 19 | Codon-optimised CFI nucleotide sequence without signal peptide portion (Co19) |
| 20 | Codon-optimised CFI nucleotide sequence without signal peptide portion (Co20) |
| 21 | Codon-optimised CFI nucleotide sequence with signal peptide portion (Co01) |
| 22 | Codon-optimised CFI nucleotide sequence with signal peptide portion (Co02) |
| 23 | Codon-optimised CFI nucleotide sequence with signal peptide portion (Co03) |
| 24 | Codon-optimised CFI nucleotide sequence with signal peptide portion (Co04) |
| 25 | Codon-optimised CFI nucleotide sequence with signal peptide portion (Co05) |
| 26 | Codon-optimised CFI nucleotide sequence with signal peptide portion (Co06) |
| 27 | Codon-optimised CFI nucleotide sequence with signal peptide portion (Co07) |
| 28 | Codon-optimised CFI nucleotide sequence with signal peptide portion (Co08) |
| 29 | Codon-optimised CFI nucleotide sequence with signal peptide portion (Co09) |
| 30 | Codon-optimised CFI nucleotide sequence with signal peptide portion (Co10) |
| 31 | Codon-optimised CFI nucleotide sequence with signal peptide portion (Co11) |
| 32 | Codon-optimised CFI nucleotide sequence with signal peptide portion (Co12) |
| 33 | Codon-optimised CFI nucleotide sequence with signal peptide portion (Co13) |
| 34 | Codon-optimised CFI nucleotide sequence with signal peptide portion (Co14) |
| 35 | Codon-optimised CFI nucleotide sequence with signal peptide portion (Co15) |
| 36 | Codon-optimised CFI nucleotide sequence with signal peptide portion (Co16) |
| 37 | Codon-optimised CFI nucleotide sequence with signal peptide portion (Co 17) |
| 38 | Codon-optimised CFI nucleotide sequence with signal peptide portion (Co18) |
| 39 | Codon-optimised CFI nucleotide sequence with signal peptide portion (Co19) |
| 40 | Codon-optimised CFI nucleotide sequence with signal peptide portion (Co20) |
| 41 | Wild type human CFI nucleotide sequence with signal peptide |
| 42 | Wild type human CFI nucleotide sequence without signal peptide |
| 43 | Polypeptide sequence of wild type human CFI with signal peptide (immature) |
| 44 | Polypeptide sequence of wild type human CFI without signal peptide (mature) |
| 45 | Wild type CFI nucleotide sequence corresponding to signal peptide |
| 46 | Wild type CFI polypeptide sequence of signal peptide |
| 47 | A1AT promoter portion of LSP-S |
| 48 | A1AT promoter portion of LSP-L |
| 49 | HCR enhancer portion of LSP-S |
| 50 | HCR enhancer portion of LSP-L |
| 51 | LSP-S transcription regulatory element |
| 52 | LSP-L transcription regulatory element |
| 53 | AAV2 5′ ITR sequence |
| 54 | AAV2 3′ ITR sequence |
| 55 | Nucleotide sequence of bovine growth hormone poly A sequence |
| 56 | Polypeptide sequence of liver-tropic capsid |
| 57 | Polypeptide sequence of liver-tropic capsid |
| 58 | Polypeptide sequence of liver-tropic capsid |
| 59 | Polypeptide sequence of liver-tropic capsid |
| 60 | Nucleotide sequence of SV40 intron |
| 61 | CAG promoter |
| 62 | Woodchuck hepatitis post-transcriptional regulatory element |
| 63 | Woodchuck hepatitis post-transcriptional regulatory element |
| 64 | Woodchuck hepatitis post-transcriptional regulatory element |
| 65 | FRE72 transcription regulatory element |
| 66 | FRE72 |
| 67 | HCR-hAAT |
| 68 | NP1 |
| 69 | NP2 |
| 70 | NP3 |
| 71 | NP4 |
| 72 | NP5 |
| 73 | NP6 |
| 74 | NP7 |
| 75 | NP8 |
| 76 | NP9 |
| 77 | NP10 |
| 78 | NP11 |
| 79 | NP12 |
| 80 | NP13 |
| 81 | NP14 |
| 82 | NP15 |
| 83 | NP16 |
| 84 | NP17 |
| 85 | NP18 |
| 86 | NP19 |
| 87 | NP20 |
| 88 | NP21 |
| 89 | NP22 |
| 90 | NP23 |
| 91 | NP24 |
| 92 | ApoE-HCR1 |
| 93 | ApoE-HCR1 High Homology Region |
| 94 | hAAT |
| 95 | hAAT Core Sequence |
| 96 | hAAT Extended Core Sequence |
| 97 | AMBP |
| 98 | AMBP High Homology Region |
| 99 | CRM6 |
| 100 | CRM6 High Homology Region 1 |
| 101 | CRM6 High Homology Region 2 |
| 102 | ALDOB |
| 103 | ALDOB High Homology Region |
| 104 | F2 |
| 105 | F2 High Homology Region |

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a polynucleotide comprising a CFI nucleotide sequence” should be interpreted to mean that the polynucleotide has a CFI nucleotide sequence, but the polynucleotide may contain additional nucleotides.

In some embodiments of the invention, the word “comprising” can be replaced with the phrase “consisting of”. The term “consisting of” is intended to be limiting. For example, the phrase “a polynucleotide consisting of a CFI nucleotide sequence” should be understood to mean that the polynucleotide has a CFI nucleotide sequence and no additional nucleotides.

In some embodiments of the invention, the word “have” or “has” can be replaced with the word “comprise” or the phrase “consist of”.

As used herein, “between” when referring to two endpoints to define a range of values should be taken to mean “between and including”. Thus, a range defined as “between 5 and 10” includes all values greater than 5 and less than 10, as well as the discrete values 5 and 10 themselves.

The term “around” used in the context of describing the length of nucleotide sequences indicates that a sequence may comprise or consist of a defined number of nucleotides, plus or minus 10%, more particularly plus or minus 5%, more particularly plus or minus 1%, or more particularly plus or minus a single integer. For example, reference to a nucleotide sequence of “around” 1698 nucleotides in length may refer to a nucleotide sequence of 1529-1867 nucleotides, more particularly 1614-1782 nucleotides, more particularly 1682-1714 nucleotides, and more particularly 1697-1699 nucleotides in length.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotides or amino acid residues at each position are then compared. When a position in the first sequence is occupied by the same nucleotide or amino acid residue as the corresponding position in the second sequence, then the nucleotides or amino acids are identical at that position. The percent identity between the two sequences is a function of the number of identical positions shared by the sequences (i.e., % identity = number of identical positions /total number of positions in the reference sequence x 100).

Typically the sequence comparison is carried out over the length of the reference sequence. For example, if the user wished to determine whether a given (“test”) sequence is 95% identical to SEQ ID NO: 5, SEQ ID NO: 5 would be the reference sequence. For example, to assess whether a sequence is at least 95% identical to SEQ ID NO: 5 (an example of a reference sequence), the skilled person would carry out an alignment over the length of SEQ ID NO: 5, and identify how many positions in the test sequence were identical to those of SEQ ID NO: 5. If at least 95% of the positions are identical, the test sequence is at least 95% identical to SEQ ID NO: 5. If the sequence is shorter than SEQ ID NO: 5, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleotide sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

For the purposes of the present invention, the term “fragment” refers to a contiguous portion of a sequence. For example, a fragment of SEQ ID NO: 5 of 50 nucleotides refers to 50 contiguous nucleotides of SEQ ID NO: 5.

The terms “protein” and “polypeptide” are used interchangeably herein, and are intended to refer to a polymeric chain of amino acids of any length.

The terms “Factor I” and “Complement Factor I” are used interchangeably herein.

### A Polynucleotide

The present invention provides a polynucleotide comprising a Complement Factor I (CFI) nucleotide sequence, wherein the CFI nucleotide sequence encodes a CFI polypeptide or a fragment thereof and wherein at least a portion of the CFI nucleotide sequence is not wild type. Optionally, the CFI nucleotide sequence is a human CFI nucleotide sequence. Optionally, the CFI polypeptide is a human CFI polypeptide.

The present invention also provides a polynucleotide comprising a CFI nucleotide sequence, wherein the CFI nucleotide sequence encodes a CFI polypeptide or a fragment thereof and the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1-40. Optionally, the CFI nucleotide sequence is a human CFI nucleotide sequence. Optionally, the CFI polypeptide is a human CFI polypeptide.

The terms “nucleic acid molecule”, “polynucleotide” and “nucleotide sequence” are intended to refer to a polymeric chain of any length of nucleotides, including deoxyribonucleotides, ribonucleotides, or analogs thereof. For example, the nucleic acid molecule, polynucleotide or nucleotide sequence may comprise DNA (deoxyribonucleotides) or RNA (ribonucleotides). The nucleic acid molecule, polynucleotide or nucleotide sequence may consist of DNA. The nucleic acid molecule, polynucleotide or nucleotide sequence may be mRNA. Since the nucleic acid molecule, polynucleotide or nucleotide sequence may comprise RNA or DNA, all references to T (thymine) nucleotides may be replaced with U (uracil).

In some embodiments, the term “nucleotide sequence” can be replaced with the term “nucleic acid molecule”.

In some embodiments, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1-40. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 1-20.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 3-20 and 23-40. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 3-20.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1, 2, 3, 5, 6, 7, 9-12, 14-17, 20, 21, 22, 23, 25, 26, 27, 29-32, 34-37, and 40. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 1, 2, 3, 5, 6, 7, 9-12, 14-17, and 20.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 2, 3, 5, 6, 7, 9-12, 14-17, 20, 22, 23, 25, 26, 27, 29-32, 34-37, and 40. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 2, 3, 5, 6, 7, 9-12, 14-17, and 20.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1, 5, 7, 10-14, 16, 17, 21, 25, 27, 30-34, 36, and 37. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 1, 5, 7, 10-14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1, 5, 7, 11-14, 16, 17, 21, 25, 27, 31-34, 36, and 37. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 1, 5, 7, 11-14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1, 5, 7, 11, 12, 14, 16, 17, 21, 25, 27, 31, 32, 34, 36, and 37. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 1, 5, 7, 11, 12, 14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 5, 7, 12-14, 16, 17, 25, 27, 32-34, 36, and 37. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 5, 7, 12-14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 5, 7, 12, 14, 16, 17, 25, 27, 32, 34, 36, and 37. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 5, 7, 12, 14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 5, 7, 14, 25, 27, and 34. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 5, 7, and 14.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 3, 5-7, 10, 14, 17, 20, 23, 25-27, 30, 34, 37, and 40. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 3, 5-7, 10, 14, 17, and 20.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 5, 10, 17, 25, 30, and 37. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of any one of SEQ ID NOs: 5, 10, and 17.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of SEQ ID NOs: 5 or 25. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of SEQ ID NO: 5.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of SEQ ID NOs: 7 or 27. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of SEQ ID NO: 7.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of SEQ ID NOs: 10 or 30. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of SEQ ID NO: 10.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of SEQ ID NOs: 14 or 34. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of SEQ ID NO: 14.

Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of SEQ ID NOs: 17 or 37. Optionally, the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, between 1200 and 1698, between 1500 and 1698, or around 1698 nucleotides of SEQ ID NO: 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1-40. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1-20. Optionally, the CFI nucleotide sequence comprises a nucleotide sequence of any one of SEQ ID NOs: 1-20.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 3-20 and 23-40. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 3-20. Optionally, the CFI nucleotide sequence comprises a nucleotide sequence of any one of SEQ ID NOs: 3-20.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 2, 3, 5, 6, 7, 9-12, 14-17, 20, 21, 22, 23, 25, 26, 27, 29-32, 34-37, and 40. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 2, 3, 5, 6, 7, 9-12, 14-17, and 20 . Optionally, the CFI nucleotide sequence comprises a nucleotide sequence of any one of SEQ ID NOs: 1, 2, 3, 5, 6, 7, 9-12, 14-17, and 20.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 2, 3, 5, 6, 7, 9-12, 14-17, 20, 22, 23, 25, 26, 27, 29-32, 34-37, and 40. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 2, 3, 5, 6, 7, 9-12, 14-17, and 20. Optionally, the CFI nucleotide sequence comprises a nucleotide sequence of any one of SEQ ID NOs: 2, 3, 5, 6, 7, 9-12, 14-17, and 20.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 10-14, 16, 17, 21, 25, 27, 30-34, 36, and 37. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 10-14, 16, and 17. Optionally, the CFI nucleotide sequence comprises a nucleotide sequence of any one of SEQ ID NOs: 1, 5, 7, 10-14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 11-14, 16, 17, 21, 25, 27, 31-34, 36, and 37. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 11-14, 16, and 17. Optionally, the CFI nucleotide sequence comprises a nucleotide sequence of any one of SEQ ID NOs: 1, 5, 7, 11-14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 11, 12, 14, 16, 17, 21, 25, 27, 31, 32, 34, 36, and 37. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 11, 12, 14, 16, and 17. Optionally, the CFI nucleotide sequence comprises a nucleotide sequence of any one of SEQ ID NOs: 1, 5, 7, 11, 12, 14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, 12-14, 16, 17, 25, 27, 32-34, 36, and 37. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, 12-14, 16, and 17. Optionally, the CFI nucleotide sequence comprises a nucleotide sequence of any one of SEQ ID NOs: 5, 7, 12-14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, 12, 14, 16, 17, 25, 27, 32, 34, 36, and 37. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, 12, 14, 16, and 17. Optionally, the CFI nucleotide sequence comprises a nucleotide sequence of any one of SEQ ID NOs: 5, 7, 12, 14, 16, and 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, 14, 25, 27, and 34. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, and 14. Optionally, the CFI nucleotide sequence comprises a nucleotide sequence of any one of SEQ ID NOs: 5, 7, and 14.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 3, 5-7, 10, 14, 17, 20, 23, 25-27, 30, 34, 37, and 40. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 3, 5-7, 10, 14, 17, and 20. Optionally, the CFI nucleotide sequence comprises a nucleotide sequence of any one of SEQ ID NOs: 3, 5-7, 10, 14, 17, and 20.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 10, 17, 25, 30, and 37. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 10, and 17. Optionally, the CFI nucleotide sequence comprises a nucleotide sequence of any one of SEQ ID NOs: 5, 10, and 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 5 or 25. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 5. Optionally, the CFI nucleotide sequence comprises a nucleotide sequence of SEQ ID NO: 5.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 7 or 27. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 7. Optionally, the CFI nucleotide sequence comprises a nucleotide sequence of SEQ ID NO: 7.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 10 or 30. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 10. Optionally, the CFI nucleotide sequence comprises a nucleotide sequence of SEQ ID NO: 10.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 14 or 34. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 14. Optionally, the CFI nucleotide sequence comprises a nucleotide sequence of SEQ ID NO: 14.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 17 or 37. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 17. Optionally, the CFI nucleotide sequence comprises a nucleotide sequence of SEQ ID NO: 17.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 1 or 21. Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 1. Optionally, the CFI nucleotide sequence comprises a nucleotide sequence of SEQ ID NO: 1.

The polynucleotide of the invention comprises a CFI nucleotide sequence and the CFI nucleotide sequence encodes a CFI polypeptide or a fragment thereof. The term “sequence that encodes” or “sequence encodes” refers to a nucleotide sequence comprising an open reading frame comprising codons that encode the encoded polypeptide. For example, a nucleotide sequence that encodes a CFI polypeptide or a fragment thereof comprises codons that encode the amino acid sequence of the CFI polypeptide or the fragment thereof. An example of a CFI nucleotide sequence that encodes a wild type CFI polypeptide is provided in SEQ ID NO: 41 or 42.

The codons that encode the polypeptide are also referred to as “coding nucleotides”. A CFI nucleotide sequence may be interrupted by non-coding nucleotides (e.g. an intron), but only nucleotides that encode the polypeptide (i.e. the coding nucleotides) should be considered to be part of the CFI nucleotide sequence. For example, a CFI nucleotide sequence that encodes a CFI polypeptide will comprise any codons (i.e. the coding nucleotides) that encode an amino acid forming part of the CFI polypeptide, irrespective of whether those codons are contiguous in sequence or separated by one or more non-coding nucleotides. In other words, a polynucleotide which contains stretches of coding nucleotides interrupted by a stretch of non-coding nucleotides will be considered to comprise a “CFInucleotide sequence” consisting of the non-contiguous stretches of coding nucleotides immediately juxtaposed (i.e. minus the stretches of non-coding nucleotides). However, herein, the nucleotides of the stop codon will be considered coding nucleotides.

The CFI nucleotide sequence encoding a CFI polypeptide or a fragment thereof may also comprise a sequence that encodes a signal peptide. It is well known that some proteins, particularly those which are exported to different tissues, are expressed with a signal peptide. Signal peptides can be at the N-terminus of a protein sequence (such as at the 5′ end of a coding sequence) and many signal peptides are cleaved following cellular processing. Thus, herein, a mature protein or polypeptide (such as a mature CFI protein or polypeptide) will be considered to be the resulting protein or polypeptide after the signal peptide has been processed and removed/cleaved (and thus no longer forms part of the polypeptide sequence).

The following Table describes codons that encode each amino acid:

| Amino Acid | Codon | Amino Acid | Codon | Amino Acid | Codon |
|---|---|---|---|---|---|
| Phenylalanine | TTC | Proline | CCT | Asparagine | AAT |
| | TTT | | CCC | | AAC |
| | | | CCA | | |
| | | | CCG | | |
| Leucine | TTA | Threonine | ACT | Lysine | AAA |
| | TTG | | ACC | | AAG |
| | CTT | | ACA | | |
| | CTC | | ACG | | |
| | CTA | | | | |
| | CTG | | | | |
| Isoleucine | ATT | Alanine | GCT | Aspartic Acid | GAT |
| | ATC | | GCC | | GAC |
| | ATA | | GCA | | |
| | | | GCG | | |
| Methionine | ATG | Tyrosine | TAT | Glutamic Acid | GAA |
| | | | TAC | | GAG |
| Valine | GTT | Histidine | CAT | Cysteine | TGT |
| | GTC | | CAC | | TGC |
| | GTA | | | | |
| | GTG | | | | |
| Serine | TCT | Glutamine | CAA | Tryptophan | TGG |
| | TCC | | CAG | | |
| | TCA | | | | |
| | TCG | | | | |
| | AGT | | | | |
| | AGC | | | | |
| Arginine | CGT | Glycine | GGT | | |
| | CGC | | GGC | | |
| | CGA | | GGA | | |
| | CGG | | GGG | | |
| | AGA | | | | |
| | AGG | | | | |

The corresponding RNA codons will contain Us in place of the Ts in the Table above.

CFI polypeptide is a glycoprotein heterodimer consisting of a heavy chain and light chain. CFI plays a role in the C3 breakdown cycle. CFI can regulate complement activation by inactivating C3b and degrading iC3b. CFI can catalyse the cleavage of C3b to iC3b (also referred to as “C3b-inactivating” activity). CFI can breakdown iC3b to C3d,g (also referred to as “iC3b-degradation” activity). A typical wild type CFI polypeptide is encoded by SEQ ID NO: 41 or 42.

Typically, CFI polypeptide is initially expressed as a precursor “immature” form (e.g. a CFI of SEQ ID NO: 43 encoded by SEQ ID NO: 41), comprising a signal peptide (e.g. amino acid residues 1 to 18 of SEQ ID NO: 43 and codons 1 to 18 of SEQ ID NO: 41), and a mature CFI polypeptide region. After processing, the “mature” form of CFI lacks the signal peptide. The term “mature CFI” or “mature CFI polypeptide” refers to a CFI polypeptide that does not comprise the signal peptide, such as a CFI encoded by SEQ ID NOs: 1-20 and 42. A typical wild type CFI signal peptide may be encoded by a nucleotide sequence of SEQ ID NO: 45 and have the polypeptide sequence of SEQ ID NO: 46.

Preferably, the CFI polypeptide or the fragment thereof is functional. It is within the abilities of the person skilled in the art to determine whether a CFI polypeptide or a fragment thereof encoded by a CFI nucleotide sequence is functional. The skilled person merely needs to express the CFI nucleotide sequence, and test whether the expressed polypeptide is active. For example, the skilled person could prepare a viral particle of the invention comprising a CFI nucleotide sequence linked to an operable promoter, and transduce cells with the viral particle under conditions suitable for expression of the CFI polypeptide or the fragment thereof. If a CFI polypeptide or a fragment thereof has at least 20% of the CFI activity of wild-type CFI, then it is functional. Preferably, the CFI activity comprises C3b-inactivating and/or iC3b-degradation activity.

Whether a polypeptide has C3b-inactivating activity can be determined by a person skilled in the art, e.g. by using a gel-based cleavage assay. For example, the polypeptide to be tested could be mixed with cofactor Factor H. Trace amounts of ¹²³I-labeled C3b could then be added and the sample incubated (e.g. overnight at 37° C.). The reaction could then be terminated by adding reducing (DDT) SDS PAGE sample buffer. The sample could then be denatured (e.g. by heating to 95° C. for 5 mins) and run on an SDS-PAGE gel (e.g. 10-15% gradient). The gel could then be dried and the proteins visualised (e.g. by using a PhosphorImager; Molecular Dynamics, Sunnyvale, CA). The intensity of the α′-chain and the 43 kDa fragment of C3b bands could then be analysed (e.g. using ImageGauge; Fujifilm). The bands could be quantified by densitometry. The ratio of the 43 kDa fragment of C3b (product) to the α′-chain (substrate) can be used to indicate how active the polypeptide is at inactivating C3b. The larger the ratio, the higher the activity.

Whether a polypeptide has iC3b-degradation activity can be determined by a person skilled in the art, e.g. by using an ELISA assay such as a paired ELISA assay. For example, the polypeptide to be tested could be mixed with serum and then an activator of the alternative complement pathway (e.g. Zymosan). The mixture could then be incubated over a time course (e.g. 0, 30, 60, 120, 180, 240 and 480 minutes) to allow the polypeptide being tested to inactivate C3b and subsequently degrade the iC3b. Samples could be collected (e.g. samples could be collected and microfuged to remove Zymosan) at each time point. How the levels of iC3b change over the time course could be used to determine the iC3b-degradation activity of the polypeptide being tested. The levels of iC3b could be determined by performing an ELISA on the samples. The OD level achieved in such an ELISA would be proportional to the amount of iC3b in the sample which has not been degraded. The higher the OD level, the more iC3b present in a sample. Over the time course, the maximum OD achieved in the first 2 hours can be taken as a measure of the extent the alternative pathway was activated. The percentage reduction of the maximum OD to the lowest OD over the time course (e.g. at 8 hours) can be used as a measure of the degradation of iC3b to C3c and C3dg, and thus can be used as a measure of the iC3b-degradation activity. The higher the percentage reduction of the maximum OD to the lowest OD over the time course, the higher the iC3b-degradation activity. An example of an ELISA which could be used is referred to in Lay et al. Clinical and Experimental Immunology, 181: 314-3212. An example of an ELISA which could be used is as follows.

An anti-C3g antibody (such as that disclosed in Lay et al. Clinical and Experimental Immunology, 181: 314-3212) could be bound to a plate (e.g. coated to a microtitre plate). Each sample could be loaded onto the plate and incubated (e.g. for 1 hour at room temperature). Region(s) of C3g are present as a neo-antigen in iC3b and in C3dg, but are not exposed in native C3 or in C3b. Therefore, an anti-C3g antibody (which binds to the neo-antigen) will only capture iC3b or C3dg from each sample (and will not capture C3 or C3b). Captured iC3b could then be detected. The captured iC3b could be detected by washing the plate and incubating with an anti-C3c antibody (e.g. for 1 hour at room temperature). The anti-C3c antibody will only bind iC3b and not C3dg since C3c is contained within iC3b (i.e. the anti-C3c antibody will bind iC3b) and released when iC3b is converted to C3dg (i.e. the anti-C3c antibody will not bind C3dg). Therefore, the antiC3c antibody will only recognise iC3b and not C3dg. The level of anti-C3c antibody present will indicate the amount of iC3b present which has not been degraded. The level of anti-C3c antibody that binds to iC3b on the plate could be measured by measuring the amount of the anti-C3c antibody. For example, the anti-C3c antibody could be a biotinylated antibody so that it could be detected using an avidin-peroxidase (e.g. extravidin-peroxidase, Sigma-Aldrich) which can bind biotin and catalyse the production of a blue reaction product from a substrate such as TMB (3,3′,5,5′-tetramethylbenzidine). For example, the plate could be washed and further incubated with avidin-peroxidase (e.g. for 1 h at room temperature). After further washing, TMB (3,3′,5,5′-tetramethylbenzidine) could be added. The level of the blue product (OD) can be detected by absorbance at 450 nm. The level of the blue product (OD) is proportional to the amount of anti-C3c antibody that remained after the washing step, which is proportional to the amount of iC3b in the sample which has not been degraded. The higher the OD level, the more iC3b is present in a sample.

Optionally in the ELISA assay described above, the cofactor CR1 (or a recombinant CR1 functional protein fragment, such as Mirococept) can be added with the serum and activator of the alternative complement pathway.

Optionally, the CFI polypeptide or the fragment thereof has at least 70%, at least 80%, at least 90%, or at least 100% of the CFI activity of wild-type CFI. Preferably, the CFI activity comprises C3b-inactivating activity and/or iC3b-degradation activity. Optionally, the CFI polypeptide or the fragment thereof has at least 70%, at least 80%, at least 90%, or at least 100% of the C3b-inactivating activity of wild-type CFI. Optionally, the CFI polypeptide or the fragment thereof has at least 70%, at least 80%, at least 90%, or at least 100% of the iC3b-degradation activity of wild-type CFI. Optionally, the CFI polypeptide or the fragment thereof has at least 70%, at least 80%, at least 90%, or at least 100% of the C3b-inactivating activity of wild-type CFI and the CFI polypeptide or the fragment thereof has at least 70%, at least 80%, at least 90%, or at least 100% of the iC3b-degradation activity of wild-type CFI.

Optionally, the CFI polypeptide or the fragment thereof encoded by the CFI nucleotide sequence comprises a sequence: (a) at least 95%, at least 98%, at least 99%, at least 99.5%, at least 99.8%, or 100% identical to SEQ ID NO: 43 or 44; or (b) at least 95%, at least 98%, at least 99%, at least 99.5%, at least 99.8%, or 100% identical to a fragment of SEQ ID NO: 43 or 44 at least 300, at least 350, at least 400, 583 or fewer, 565 or fewer, between 300 and 583, or between 300 and 565 amino acids in length.

In an embodiment, the CFI polypeptide or the fragment thereof is at least 95%, at least 98%, at least 99%, at least 99.5%, at least 99.8%, or 100% identical to SEQ ID NO: 44; or at least 95%, at least 98%, at least 99%, at least 99.5%, at least 99.8%, or 100% identical to a fragment of SEQ ID NO: 44 of about 565 amino acids in length. Optionally, the CFI polypeptide or a fragment thereof does not comprise the signal peptide of SEQ ID NO: 46.

The CFI nucleotide sequence may comprise a sequence of any one of SEQ ID NOs: 1-20, or a variant thereof encoding a CFI polypeptide comprising a sequence at least 95% identical to the wild type CFI amino acid sequence of SEQ ID NO: 44. Optionally, the variant is a variant of any one of SEQ ID NOs: 5, 7, and 14. Optionally, the variant is a variant of any one of SEQ ID NOs: 5, 10, and 17. Optionally, the variant encodes a CFI polypeptide having CFI activity. Preferably, the variant encodes a CFI polypeptide which is functional. Optionally, the variant encodes a CFI polypeptide having at least 70%, at least 80%, at least 90%, or at least 100% of the CFI activity of wild-type CFI. Preferably, the CFI activity comprises C3b-inactivating activity and/or iC3b-degradation activity. Optionally, the variant encodes a CFI polypeptide having at least 70%, at least 80%, at least 90%, or at least 100% of the C3b-inactivating activity of wild-type CFI. Optionally, the variant encodes a CFI polypeptide having at least 70%, at least 80%, at least 90%, or at least 100% of the iC3b-degradation activity of wild-type CFI. Optionally, the variant encodes a CFI polypeptide having at least 70%, at least 80%, at least 90%, or at least 100% of the C3b-inactivating activity of wild-type CFI and the variant encodes a CFI polypeptide having at least 70%, at least 80%, at least 90%, or at least 100% of the iC3b-degradation activity of wild-type CFI.

Optionally, the variant is a variant of any one of SEQ ID NOs: 1, 2, 3, 4, 5, 6, 7, 8, 9, 10, 11, 12, 13, 14, 15, 16, 17, 18, 19, or 20. In some embodiments, the variant of SEQ ID NO: 1, 2, 3, 4, 5, 6, 7, 8, 9, 10, 11, 12, 13, 14, 15, 16, 17, 18, 19, or 20 is identical to SEQ ID NO: 1, 2, 3, 4, 5, 6, 7, 8, 9, 10, 11, 12, 13, 14, 15, 16, 17, 18, 19, or 20, respectively, except that it comprises nucleotide substitutions such that the encoded CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO: 44. In some embodiments, the variant of SEQ ID NO: 1, 2, 3, 4, 5, 6, 7, 8, 9, 10, 11, 12, 13, 14, 15, 16, 17, 18, 19, or 20 has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 1, 2, 3, 4, 5, 6, 7, 8, 9, 10, 11, 12, 13, 14, 15, 16, 17, 18, 19, or 20, respectively. The variant of SEQ ID NO: 1, 2, 3, 4, 5, 6, 7, 8, 9, 10, 11, 12, 13, 14, 15, 16, 17, 18, 19, or 20 may have 1, 2 or fewer, 3 or fewer, 4 or fewer, 5 or fewer, or 6 or fewer nucleotide substitutions relative to the sequence of SEQ ID NO: 1, 2, 3, 4, 5, 6, 7, 8, 9, 10, 11, 12, 13, 14, 15, 16, 17, 18, 19, or 20, respectively. In one embodiment, the variant of SEQ ID NO: 1, 2, 3, 4, 5, 6, 7, 8, 9, 10, 11, 12, 13, 14, 15, 16, 17, 18, 19, or 20 has 4 or fewer nucleotide substitutions relative to the sequence of SEQ ID NO: 1, 2, 3, 4, 5, 6, 7, 8, 9, 10, 11, 12, 13, 14, 15, 16, 17, 18, 19, or 20, respectively, and/or encodes a CFI polypeptide having 3 or fewer amino acid substitutions relative to the wild type CFI amino acid sequence of SEQ ID NO: 44. In one embodiment, the variant of SEQ ID NO: 1, 2, 3, 4, 5, 6, 7, 8, 9, 10, 11, 12, 13, 14, 15, 16, 17, 18, 19, or 20 has 3 or fewer nucleotide substitutions relative to the sequence of SEQ ID NO: 1, 2, 3, 4, 5, 6, 7, 8, 9, 10, 11, 12, 13, 14, 15, 16, 17, 18, 19, or 20, respectively, and/or encodes a CFI polypeptide having 2 or fewer amino acid substitutions relative to the wild type CFI amino acid sequence of SEQ ID NO: 44. In one embodiment, the variant of SEQ ID NO: 1, 2, 3, 4, 5, 6, 7, 8, 9, 10, 11, 12, 13, 14, 15, 16, 17, 18, 19, or 20 has 1 nucleotide substitution relative to the sequence of SEQ ID NO: 1, 2, 3, 4, 5, 6, 7, 8, 9, 10, 11, 12, 13, 14, 15, 16, 17, 18, 19, or 20, respectively, and/or encodes a CFI polypeptide having 1 amino acid substitutions relative to the wild type CFI amino acid sequence of SEQ ID NO: 44. In one embodiment, the variant of SEQ ID NO: 1, 2, 3, 4, 5, 6, 7, 8, 9, 10, 11, 12, 13, 14, 15, 16, 17, 18, 19, or 20 encodes a CFI polypeptide having 3 or fewer amino acid substitutions relative to the wild type CFI amino acid sequence of SEQ ID NO: 44. In one embodiment, the variant of SEQ ID NO: 1, 2, 3, 4, 5, 6, 7, 8, 9, 10, 11, 12, 13, 14, 15, 16, 17, 18, 19, or 20 encodes a CFI polypeptide having 2 or fewer amino acid substitutions relative to the wild type CFI amino acid sequence of SEQ ID NO: 44. In an embodiment, the variant of SEQ ID NO: 1, 2, 3, 4, 5, 6, 7, 8, 9, 10, 11, 12, 13, 14, 15, 16, 17, 18, 19, or 20 encodes a CFI polypeptide having 1 amino acid substitutions relative to the wild type CFI amino acid sequence of SEQ ID NO: 44.

Optionally, the variant is a variant of SEQ ID NO: 5. In some embodiments, the variant of SEQ ID NO: 5 is identical to SEQ ID NO: 5 except that it comprises nucleotide substitutions such that the CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO: 44. In some embodiments, the variant of SEQ ID NO: 5 has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 5. The variant of SEQ ID NO: 5 may have 1, 2 or fewer, 3 or fewer, 4 or fewer, 5 or fewer, or 6 or fewer nucleotide substitutions relative to the sequence of SEQ ID NO: 5. In one embodiment, the variant of SEQ ID NO: 5 has 4 or fewer nucleotide substitutions relative to the sequence of SEQ ID NO: 5 and/or encodes a CFI polypeptide having 3 or fewer amino acid substitutions relative to the wild type amino acid CFI sequence of SEQ ID NO: 44. In one embodiment, the variant of SEQ ID NO: 5 has 3 or fewer nucleotide substitutions relative to the sequence of SEQ ID NO: 5 and/or encodes a CFI polypeptide having 2 or fewer amino acid substitutions relative to the wild type CFI amino acid sequence of SEQ ID NO: 44. In one embodiment, the variant of SEQ ID NO: 5 has 1 nucleotide substitution relative to the sequence of SEQ ID NO: 5 and/or encodes a CFI polypeptide having 1 amino acid substitution relative to the wild type CFI amino acid sequence of SEQ ID NO: 44. In one embodiment, the variant of SEQ ID NO: 5 encodes a CFI polypeptide having 3 or fewer amino acid substitutions relative to the wild type CFI amino acid sequence of SEQ ID NO: 44. In one embodiment, the variant of SEQ ID NO: 5 encodes a CFI polypeptide having 2 or fewer amino acid substitutions relative to the wild type CFI amino acid sequence of SEQ ID NO: 44. In an embodiment, the variant of SEQ ID NO: 5 encodes a CFI polypeptide having 1 amino acid substitution relative to the wild type CFI amino acid sequence of SEQ ID NO: 44.

Optionally, the variant is a variant of SEQ ID NO: 7. In some embodiments, the variant of SEQ ID NO: 7 is identical to SEQ ID NO: 7 except that it comprises nucleotide substitutions such that the CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO: 44. In some embodiments, the variant of SEQ ID NO: 7 has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 7. The variant of SEQ ID NO: 7 may have 1, 2 or fewer, 3 or fewer, 4 or fewer, 5 or fewer, or 6 or fewer nucleotide substitutions relative to the sequence of SEQ ID NO: 7. In one embodiment, the variant of SEQ ID NO: 7 has 4 or fewer nucleotide substitutions relative to the sequence of SEQ ID NO: 7 and/or encodes a CFI polypeptide having 3 or fewer amino acid substitutions relative to the wild type amino acid CFI sequence of SEQ ID NO: 44. In one embodiment, the variant of SEQ ID NO: 7 has 3 or fewer nucleotide substitutions relative to the sequence of SEQ ID NO: 7 and/or encodes a CFI polypeptide having 2 or fewer amino acid substitutions relative to the wild type CFI amino acid sequence of SEQ ID NO: 44. In one embodiment, the variant of SEQ ID NO: 7 has 1 nucleotide substitution relative to the sequence of SEQ ID NO: 7 and/or encodes a CFI polypeptide having 1 amino acid substitution relative to the wild type CFI amino acid sequence of SEQ ID NO: 44. In one embodiment, the variant of SEQ ID NO: 7 encodes a CFI polypeptide having 3 or fewer amino acid substitutions relative to the wild type CFI amino acid sequence of SEQ ID NO: 44. In one embodiment, the variant of SEQ ID NO: 7 encodes a CFI polypeptide having 2 or fewer amino acid substitutions relative to the wild type CFI amino acid sequence of SEQ ID NO: 44. In one embodiment, the variant of SEQ ID NO: 7 encodes a CFI polypeptide having 1 amino acid substitution relative to the wild type CFI amino acid sequence of SEQ ID NO: 44.

Optionally, the variant is a variant of SEQ ID NO: 10. In some embodiments, the variant of SEQ ID NO: 10 is identical to SEQ ID NO: 10 except that it comprises nucleotide substitutions such that the CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO: 44. In some embodiments, the variant of SEQ ID NO: 10 has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 10. The variant of SEQ ID NO: 10 may have 1, 2 or fewer, 3 or fewer, 4 or fewer, 5 or fewer, or 6 or fewer nucleotide substitutions relative to the sequence of SEQ ID NO: 10. In one embodiment, the variant of SEQ ID NO: 10 has 4 or fewer nucleotide substitutions relative to the sequence of SEQ ID NO: 10 and/or encodes a CFI polypeptide having 3 or fewer amino acid substitutions relative to the wild type amino acid CFI sequence of SEQ ID NO: 44. In one embodiment, the variant of SEQ ID NO: 10 has 3 or fewer nucleotide substitutions relative to the sequence of SEQ ID NO: 10 and/or encodes a CFI polypeptide having 2 or fewer amino acid substitutions relative to the wild type CFI amino acid sequence of SEQ ID NO: 44. In one embodiment, the variant of SEQ ID NO: 10 has 1 nucleotide substitution relative to the sequence of SEQ ID NO: 10 and/or encodes a CFI polypeptide having 1 amino acid substitution relative to the wild type CFI amino acid sequence of SEQ ID NO: 44. In one embodiment, the variant of SEQ ID NO: 10 encodes a CFI polypeptide having 3 or fewer amino acid substitutions relative to the wild type CFI amino acid sequence of SEQ ID NO: 44. In one embodiment, the variant of SEQ ID NO: 10 encodes a CFI polypeptide having 2 or fewer amino acid substitutions relative to the wild type CFI amino acid sequence of SEQ ID NO: 44. In an embodiment, the variant of SEQ ID NO: 10 encodes a CFI polypeptide having 1 amino acid substitution relative to the wild type CFI amino acid sequence of SEQ ID NO: 44.

Optionally, the variant is a variant of SEQ ID NO: 14. In some embodiments, the variant of SEQ ID NO: 14 is identical to SEQ ID NO: 14 except that it comprises nucleotide substitutions such that the CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO: 44. In some embodiments, the variant of SEQ ID NO: 14 has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 14. The variant of SEQ ID NO: 14 may have 1, 2 or fewer, 3 or fewer, 4 or fewer, 5 or fewer, or 6 or fewer nucleotide substitutions relative to the sequence of SEQ ID NO: 14. In one embodiment, the variant of SEQ ID NO: 14 has 4 or fewer nucleotide substitutions relative to the sequence of SEQ ID NO: 14 and/or encodes a CFI polypeptide having 3 or fewer amino acid substitutions relative to the wild type amino acid CFI sequence of SEQ ID NO: 44. In one embodiment, the variant of SEQ ID NO: 14 has 3 or fewer nucleotide substitutions relative to the sequence of SEQ ID NO: 14 and/or encodes a CFI polypeptide having 2 or fewer amino acid substitutions relative to the wild type CFI amino acid sequence of SEQ ID NO: 44. In one embodiment, the variant of SEQ ID NO: 14 has 1 nucleotide substitution relative to the sequence of SEQ ID NO: 14 and/or encodes a CFI polypeptide having 1 amino acid substitution relative to the wild type CFI amino acid sequence of SEQ ID NO: 44. In one embodiment, the variant of SEQ ID NO: 14 encodes a CFI polypeptide having 3 or fewer amino acid substitutions relative to the wild type CFI amino acid sequence of SEQ ID NO: 44. In one embodiment, the variant of SEQ ID NO: 14 encodes a CFI polypeptide having 2 or fewer amino acid substitutions relative to the wild type CFI amino acid sequence of SEQ ID NO: 44. In one embodiment, the variant of SEQ ID NO: 14 encodes a CFI polypeptide having 1 amino acid substitution relative to the wild type CFI amino acid sequence of SEQ ID NO: 44.

Optionally, the variant is a variant of SEQ ID NO: 17. In some embodiments, the variant of SEQ ID NO: 17 is identical to SEQ ID NO: 17 except that it comprises nucleotide substitutions such that the CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO: 44. In some embodiments, the variant of SEQ ID NO: 17 has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 17. The variant of SEQ ID NO: 17 may have 1, 2 or fewer, 3 or fewer, 4 or fewer, 5 or fewer, or 6 or fewer nucleotide substitutions relative to the sequence of SEQ ID NO: 17. In one embodiment, the variant of SEQ ID NO: 17 has 4 or fewer nucleotide substitutions relative to the sequence of SEQ ID NO: 17 and/or encodes a CFI polypeptide having 3 or fewer amino acid substitutions relative to the wild type amino acid CFI sequence of SEQ ID NO: 44. In one embodiment, the variant of SEQ ID NO: 17 has 3 or fewer nucleotide substitutions relative to the sequence of SEQ ID NO: 17 and/or encodes a CFI polypeptide having 2 or fewer amino acid substitutions relative to the wild type CFI amino acid sequence of SEQ ID NO: 44. In one embodiment, the variant of SEQ ID NO: 17 has 1 nucleotide substitution relative to the sequence of SEQ ID NO: 17 and/or encodes a CFI polypeptide having 1 amino acid substitution relative to the wild type CFI amino acid sequence of SEQ ID NO: 44. In one embodiment, the variant of SEQ ID NO: 17 encodes a CFI polypeptide having 3 or fewer amino acid substitutions relative to the wild type CFI amino acid sequence of SEQ ID NO: 44. In one embodiment, the variant of SEQ ID NO: 17 encodes a CFI polypeptide having 2 or fewer amino acid substitutions relative to the wild type CFI amino acid sequence of SEQ ID NO: 44. In an embodiment, the variant of SEQ ID NO: 17 encodes a CFI polypeptide having 1 amino acid substitution relative to the wild type CFI amino acid sequence of SEQ ID NO: 44.

The CFI nucleotide sequence may encode a CFI polypeptide having 1, 2 or fewer, 3 or fewer, 4 or fewer, or 5 or fewer amino acid substitutions relative to the wild type CFI amino acid sequence of SEQ ID NO: 44. The CFI nucleotide sequence may encode a CFI polypeptide having 3 or fewer amino acid substitutions relative to the wild type CFI amino acid sequence of SEQ ID NO: 44. The CFI nucleotide sequence may encode a CFI polypeptide having 2 or fewer amino acid substitutions relative to the wild type CFI amino acid sequence of SEQ ID NO: 44. The CFI nucleotide sequence may encode a CFI polypeptide having 1 amino acid substitution relative to the wild type CFI amino acid sequence of SEQ ID NO: 44.

The term “nucleotide substitution” is intended to mean the substitution of one nucleotide in a nucleotide sequence with a different nucleotide. The term “amino acid substitution” is intended to mean the substitution of one amino acid in an amino acid sequence with a different amino acid.

In one embodiment, the CFI nucleotide sequence comprises a sequence that is at least 98% identical to a fragment of at least 1500 nucleotides of any one of SEQ ID NOs: 1-40. In one embodiment, the CFI nucleotide sequence comprises a sequence that is at least 99% identical to a fragment of at least 1500 nucleotides of any one of SEQ ID NOs: 1-40. In one embodiment, the CFI nucleotide sequence comprises a sequence that is at least 98% identical to a nucleotide sequence of any one of SEQ ID NOs: 1-40. In one embodiment, the CFI nucleotide sequence comprises a sequence that is at least 99% identical to a nucleotide sequence of any one of SEQ ID NOs: 1-40. In one embodiment, the CFI nucleotide sequence comprises a nucleotide sequence of any one of SEQ ID NOs: 1-40.

In one embodiment, the CFI nucleotide sequence comprises a sequence that is at least 98% identical to a fragment of at least 1500 nucleotides of any one of SEQ ID NOs: 3-20 and 23-40. In one embodiment, the CFI nucleotide sequence comprises a sequence that is at least 99% identical to a fragment of at least 1500 nucleotides of any one of SEQ ID NOs: 3-20 and 23-40. In one embodiment, the CFI nucleotide sequence comprises a sequence that is at least 98% identical to a nucleotide sequence of any one of SEQ ID NOs: 3-20 and 23-40. In one embodiment, the CFI nucleotide sequence comprises a sequence that is at least 99% identical to a nucleotide sequence of any one of SEQ ID NOs: 3-20 and 23-40. In one embodiment, the CFI nucleotide sequence comprises a sequence of any one of SEQ ID NOs: 3-20 and 23-40.

In one embodiment, the CFI nucleotide sequence comprises a sequence that is at least 98% identical to a fragment of at least 1500 nucleotides of any one of SEQ ID NOs: 1, 2, 3, 5, 6, 7, 9-12, 14-17, 20, 21, 22, 23, 25, 26, 27, 29-32, 34-37, and 40. In one embodiment, the CFI nucleotide sequence comprises a sequence that is at least 99% identical to a fragment of at least 1500 nucleotides of any one of SEQ ID NOs: 1, 2, 3, 5, 6, 7, 9-12, 14-17, 20, 21, 22, 23, 25, 26, 27, 29-32, 34-37, and 40. In one embodiment, the CFI nucleotide sequence comprises a sequence that is at least 98% identical to a nucleotide sequence of any one of SEQ ID NOs: 1, 2, 3, 5, 6, 7, 9-12, 14-17, 20, 21, 22, 23, 25, 26, 27, 29-32, 34-37, and 40. In one embodiment, the CFI nucleotide sequence comprises a sequence that is at least 99% identical to a nucleotide sequence of any one of SEQ ID NOs: 1, 2, 3, 5, 6, 7, 9-12, 14-17, 20, 21, 22, 23, 25, 26, 27, 29-32, 34-37, and 40. In one embodiment, the CFI nucleotide sequence comprises a sequence of any one of SEQ ID NOs: 1, 2, 3, 5, 6, 7, 9-12, 14-17, 20, 21, 22, 23, 25, 26, 27, 29-32, 34-37, and 40.

In one embodiment, the CFI nucleotide sequence comprises a sequence that is at least 98% identical to a fragment of at least 1500 nucleotides of any one of SEQ ID NOs: 2, 3, 5, 6, 7, 9-12, 14-17, 20, 22, 23, 25, 26, 27, 29-32, 34-37, and 40. In one embodiment, the CFI nucleotide sequence comprises a sequence that is at least 99% identical to a fragment of at least 1500 nucleotides of any one of SEQ ID NOs: 2, 3, 5, 6, 7, 9-12, 14-17, 20, 22, 23, 25, 26, 27, 29-32, 34-37, and 40. In one embodiment, the CFI nucleotide sequence comprises a sequence that is at least 98% identical to a nucleotide sequence of any one of SEQ ID NOs: 2, 3, 5, 6, 7, 9-12, 14-17, 20, 22, 23, 25, 26, 27, 29-32, 34-37, and 40. In one embodiment, the CFI nucleotide sequence comprises a sequence that is at least 99% identical to a nucleotide sequence of any one of SEQ ID NOs: 2, 3, 5, 6, 7, 9-12, 14-17, 20, 22, 23, 25, 26, 27, 29-32, 34-37, and 40. In one embodiment, the CFI nucleotide sequence comprises a sequence of any one of SEQ ID NOs: 2, 3, 5, 6, 7, 9-12, 14-17, 20, 22, 23, 25, 26, 27, 29-32, 34-37, and 40.

In one embodiment, the CFI nucleotide sequence comprises a sequence that is at least 98% identical to a fragment of at least 1500 nucleotides of any one of SEQ ID NOs: 1, 5, 7, 10-14, 16, 17, 21, 25, 27, 30-34, 36, and 37. In one embodiment, the CFI nucleotide sequence comprises a sequence that is at least 99% identical to a fragment of at least 1500 nucleotides of any one of SEQ ID NOs: 1, 5, 7, 10-14, 16, 17, 21, 25, 27, 30-34, 36, and 37. In one embodiment, the CFI nucleotide sequence comprises a sequence that is at least 98% identical to a nucleotide sequence of any one of SEQ ID NOs: 1, 5, 7, 10-14, 16, 17, 21, 25, 27, 30-34, 36, and 37. In one embodiment, the CFI nucleotide sequence comprises a sequence that is at least 99% identical to a nucleotide sequence of any one of SEQ ID NOs: 1, 5, 7, 10-14, 16, 17, 21, 25, 27, 30-34, 36, and 37. In one embodiment, the CFI nucleotide sequence comprises a sequence of any one of SEQ ID NOs: 1, 5, 7, 10-14, 16, 17, 21, 25, 27, 30-34, 36, and 37.

In one embodiment, the CFI nucleotide sequence comprises a sequence that is at least 98% identical to a fragment of at least 1500 nucleotides of any one of SEQ ID NOs: 1, 5, 7, 11-14, 16, 17, 21, 25, 27, 31-34, 36, and 37. In one embodiment, the CFI nucleotide sequence comprises a sequence that is at least 99% identical to a fragment of at least 1500 nucleotides of any one of SEQ ID NOs: 1, 5, 7, 11-14, 16, 17, 21, 25, 27, 31-34, 36, and 37. In one embodiment, the CFI nucleotide sequence comprises a sequence that is at least 98% identical to a nucleotide sequence of any one of SEQ ID NOs: 1, 5, 7, 11-14, 16, 17, 21, 25, 27, 31-34, 36, and 37. In one embodiment, the CFI nucleotide sequence comprises a sequence that is at least 99% identical to a nucleotide sequence of any one of SEQ ID NOs: 1, 5, 7, 11-14, 16, 17, 21, 25, 27, 31-34, 36, and 37. In one embodiment, the CFI nucleotide sequence comprises a sequence of any one of SEQ ID NOs: 1, 5, 7, 11-14, 16, 17, 21, 25, 27, 31-34, 36, and 37.

In one embodiment, the CFI nucleotide sequence comprises a sequence that is at least 98% identical to a fragment of at least 1500 nucleotides of any one of SEQ ID NOs: 1, 5, 7, 11, 12, 14, 16, 17, 21, 25, 27, 31, 32, 34, 36, and 37. In one embodiment, the CFI nucleotide sequence comprises a sequence that is at least 99% identical to a fragment of at least 1500 nucleotides of any one of SEQ ID NOs: 1, 5, 7, 11, 12, 14, 16, 17, 21, 25, 27, 31, 32, 34, 36, and 37. In one embodiment, the CFI nucleotide sequence comprises a sequence that is at least 98% identical to a nucleotide sequence of any one of SEQ ID NOs: 1, 5, 7, 11, 12, 14, 16, 17, 21, 25, 27, 31, 32, 34, 36, and 37. In one embodiment, the CFI nucleotide sequence comprises a sequence that is at least 99% identical to a nucleotide sequence of any one of SEQ ID NOs: 1, 5, 7, 11, 12, 14, 16, 17, 21, 25, 27, 31, 32, 34, 36, and 37. In one embodiment, the CFI nucleotide sequence comprises a sequence of any one of SEQ ID NOs: 1, 5, 7, 11, 12, 14, 16, 17, 21, 25, 27, 31, 32, 34, 36, and 37.

In one embodiment, the CFI nucleotide sequence comprises a sequence that is at least 98% identical to a fragment of at least 1500 nucleotides of any one of SEQ ID NOs: 5, 7, 12-14, 16, 17, 25, 27, 32-34, 36, and 37. In one embodiment, the CFI nucleotide sequence comprises a sequence that is at least 99% identical to a fragment of at least 1500 nucleotides of any one of SEQ ID NOs: 5, 7, 12-14, 16, 17, 25, 27, 32-34, 36, and 37. In one embodiment, the CFI nucleotide sequence comprises a sequence that is at least 98% identical to a nucleotide sequence of any one of SEQ ID NOs: 5, 7, 12-14, 16, 17, 25, 27, 32-34, 36, and 37. In one embodiment, the CFI nucleotide sequence comprises a sequence that is at least 99% identical to a nucleotide sequence of any one of SEQ ID NOs: 5, 7, 12-14, 16, 17, 25, 27, 32-34, 36, and 37. In one embodiment, the CFI nucleotide sequence comprises a sequence of any one of SEQ ID NOs: 5, 7, 12-14, 16, 17, 25, 27, 32-34, 36, and 37.

In one embodiment, the CFI nucleotide sequence comprises a sequence that is at least 98% identical to a fragment of at least 1500 nucleotides of any one of SEQ ID NOs: 5, 7, 12, 14, 16, 17, 25, 27, 32, 34, 36, and 37. In one embodiment, the CFI nucleotide sequence comprises a sequence that is at least 99% identical to a fragment of at least 1500 nucleotides of any one of SEQ ID NOs: 5, 7, 12, 14, 16, 17, 25, 27, 32, 34, 36, and 37. In one embodiment, the CFI nucleotide sequence comprises a sequence that is at least 98% identical to a nucleotide sequence of any one of SEQ ID NOs: 5, 7, 12, 14, 16, 17, 25, 27, 32, 34, 36, and 37. In one embodiment, the CFI nucleotide sequence comprises a sequence that is at least 99% identical to a nucleotide sequence of any one of SEQ ID NOs: 5, 7, 12, 14, 16, 17, 25, 27, 32, 34, 36, and 37. In one embodiment, the CFI nucleotide sequence comprises a sequence of any one of SEQ ID NOs: 5, 7, 12, 14, 16, 17, 25, 27, 32, 34, 36, and 37.

In one embodiment, the CFI nucleotide sequence comprises a sequence of any one of SEQ ID NOs: 5, 7, 12, 14, 16, 17, 25, 27, 32-34, 36, and 37.

In one embodiment, the CFI nucleotide sequence comprises a sequence that is at least 98% identical to a fragment of at least 1500 nucleotides of any one of SEQ ID NOs: 5, 7, 14, 25, 27, and 34. In one embodiment, the CFI nucleotide sequence comprises a sequence that is at least 99% identical to a fragment of at least 1500 nucleotides of any one of SEQ ID NOs: 5, 7, 14, 25, 27, and 34. In one embodiment, the CFI nucleotide sequence comprises a sequence that is at least 98% identical to a nucleotide sequence of any one of SEQ ID NOs: 5, 7, 14, 25, 27, and 34. In one embodiment, the CFI nucleotide sequence comprises a sequence that is at least 99% identical to a nucleotide sequence of any one of SEQ ID NOs: 5, 7, 14, 25, 27, and 34. In one embodiment, the CFI nucleotide sequence comprises a sequence of any one of SEQ ID NOs: 5, 7, 14, 25, 27, and 34.

In one embodiment, the CFI nucleotide sequence comprises a sequence that is at least 98% identical to a fragment of at least 1500 nucleotides of any one of SEQ ID NOs: 3, 5-7, 10, 14, 17, 20, 23, 25-27, 30, 34, 37, and 40. In one embodiment, the CFI nucleotide sequence comprises a sequence that is at least 99% identical to a fragment of at least 1500 nucleotides of any one of SEQ ID NOs: 3, 5-7, 10, 14, 17, 20, 23, 25-27, 30, 34, 37, and 40. In one embodiment, the CFI nucleotide sequence comprises a sequence that is at least 98% identical to a nucleotide sequence of any one of SEQ ID NOs: 3, 5-7, 10, 14, 17, 20, 23, 25-27, 30, 34, 37, and 40. In one embodiment, the CFI nucleotide sequence comprises a sequence that is at least 99% identical to a nucleotide sequence of any one of SEQ ID NOs: 3, 5-7, 10, 14, 17, 20, 23, 25-27, 30, 34, 37, and 40. In one embodiment, the CFI nucleotide sequence comprises a sequence of any one of SEQ ID NOs: 3, 5-7, 10, 14, 17, 20, 23, 25-27, 30, 34, 37, and 40.

In one embodiment, the CFI nucleotide sequence comprises a sequence that is at least 98% identical to a fragment of at least 1500 nucleotides of any one of SEQ ID NOs: 5, 10, 17, 25, 30, and 37. In one embodiment, the CFI nucleotide sequence comprises a sequence that is at least 99% identical to a fragment of at least 1500 nucleotides of any one of SEQ ID NOs: 5, 10, 17, 25, 30, and 37. In one embodiment, the CFI nucleotide sequence comprises a sequence that is at least 98% identical to a nucleotide sequence of any one of SEQ ID NOs: 5, 10, 17, 25, 30, and 37. In one embodiment, the CFI nucleotide sequence comprises a sequence that is at least 99% identical to a nucleotide sequence of any one of SEQ ID NOs: 5, 10, 17, 25, 30, and 37. In one embodiment, the CFI nucleotide sequence comprises a sequence of any one of SEQ ID NOs: 5, 10, 17, 25, 30, and 37.

In one embodiment, the CFI nucleotide sequence comprises a sequence that is at least 98% identical to a fragment of at least 1500 nucleotides of SEQ ID NO: 5 or 25. In one embodiment, the CFI nucleotide sequence comprises a sequence that is at least 99% identical to a fragment of at least 1500 nucleotides of SEQ ID NO: 5 or 25. In one embodiment, the CFI nucleotide sequence comprises a sequence that is at least 98% identical to a nucleotide sequence of SEQ ID NO: 5 or 25. In one embodiment, the CFI nucleotide sequence comprises a sequence that is at least 99% identical to a nucleotide sequence of SEQ ID NO: 5 or 25. In one embodiment, the CFI nucleotide sequence comprises a sequence of SEQ ID NO: 5 or 25.

In one embodiment, the CFI nucleotide sequence comprises a sequence that is at least 98% identical to a fragment of at least 1500 nucleotides of SEQ ID NO: 7 or 27. In one embodiment, the CFI nucleotide sequence comprises a sequence that is at least 99% identical to a fragment of at least 1500 nucleotides of SEQ ID NO: 7 or 27. In one embodiment, the CFI nucleotide sequence comprises a sequence that is at least 98% identical to a nucleotide sequence of SEQ ID NO: 7 or 27. In one embodiment, the CFI nucleotide sequence comprises a sequence that is at least 99% identical to a nucleotide sequence of SEQ ID NO: 7 or 27. In one embodiment, the CFI nucleotide sequence comprises a sequence of SEQ ID NO: 7 or 27.

In one embodiment, the CFI nucleotide sequence comprises a sequence that is at least 98% identical to a fragment of at least 1500 nucleotides of SEQ ID NO: 10 or 30. In one embodiment, the CFI nucleotide sequence comprises a sequence that is at least 99% identical to a fragment of at least 1500 nucleotides of SEQ ID NO: 10 or 30. In one embodiment, the CFI nucleotide sequence comprises a sequence that is at least 98% identical to a nucleotide sequence of SEQ ID NO: 10 or 30. In one embodiment, the CFI nucleotide sequence comprises a sequence that is at least 99% identical to a nucleotide sequence of SEQ ID NO: 10 or 30. In one embodiment, the CFI nucleotide sequence comprises a sequence of SEQ ID NO: 10 or 30.

In one embodiment, the CFI nucleotide sequence comprises a sequence that is at least 98% identical to a fragment of at least 1500 nucleotides of SEQ ID NO: 14 or 34. In one embodiment, the CFI nucleotide sequence comprises a sequence that is at least 99% identical to a fragment of at least 1500 nucleotides of SEQ ID NO: 14 or 34. In one embodiment, the CFI nucleotide sequence comprises a sequence that is at least 98% identical to a nucleotide sequence of SEQ ID NO: 14 or 34. In one embodiment, the CFI nucleotide sequence comprises a sequence that is at least 99% identical to a nucleotide sequence of SEQ ID NO: 14 or 34. In one embodiment, the CFI nucleotide sequence comprises a sequence of SEQ ID NO: 14 or 34.

In one embodiment, the CFI nucleotide sequence comprises a sequence that is at least 98% identical to a fragment of at least 1500 nucleotides of SEQ ID NO: 17 or 37. In one embodiment, the CFI nucleotide sequence comprises a sequence that is at least 99% identical to a fragment of at least 1500 nucleotides of SEQ ID NO: 17 or 37. In one embodiment, the CFI nucleotide sequence comprises a sequence that is at least 98% identical to a nucleotide sequence of SEQ ID NO: 17 or 37. In one embodiment, the CFI nucleotide sequence comprises a sequence that is at least 99% identical to a nucleotide sequence of SEQ ID NO: 17 or 37. In one embodiment, the CFI nucleotide sequence comprises a sequence of SEQ ID NO: 17 or 37.

Optionally, at least a portion of the CFI nucleotide sequence, for example the coding sequence that encodes CFI polypeptide or a fragment thereof, is not wild type. A wild type CFI-encoding CFI nucleotide sequence is represented by SEQ ID NO: 41 or 42, and a CFI nucleotide sequence that comprises a portion differing in sequence from that of SEQ ID NO: 41 or 42 comprises a portion that is not wild type.

The CFI nucleotide sequence, or the portion of CFI nucleotide sequence that is not wild type, may be codon-optimised. The polynucleotide of the invention may comprise a portion that is not codon-optimised. For example, the CFI nucleotide sequence may comprise a portion that is not codon-optimised.

In an embodiment, the portion of the CFI nucleotide sequence that is not wild type is codon-optimised. Optionally, all or a portion of the CFI nucleotide sequence is codon-optimised. Codon-optimisation can improve expression of the nucleotide sequence, for example a CFI nucleotide sequence, in a particular tissue and/or in a particular organism. For example, if a nucleotide sequence is codon-optimised for expression in the human liver, the nucleotide sequence may be modified to increase the number of codons that may be favoured (in the sense that such codons correspond to tRNA species which are more abundant than other tRNA species specific for the same amino acid) in the human liver. As a further example, if a nucleotide sequence is codon-optimised for expression in the human liver cells, the nucleotide sequence may be modified to increase the number of codons that may be favoured (in the sense that such codons correspond to tRNA species which are more abundant than other tRNA species specific for the same amino acid) in human liver cells. The skilled person would appreciate that codon-optimising a sequence may not entail changing every codon, not least because a “favoured codon” may already be present at some positions.

Such codon-optimisation may be subject to other factors. For example, favoured codons may not be introduced at positions where doing so introduces CpGs into the sequence; this will still be considered to be codon-optimisation. In an embodiment, a favoured codon that ends with a C nucleotide will not be included in the portion of the coding sequence that is codon-optimised, where the next codon in the sequence begins with a G. For example, codon GCC encodes alanine. Optionally, where GCC is a favoured codon, it should not be used for encoding alanine where the next codon in the sequence begins with a G, such as codon GAC (or alternatively, the next codon — where possible — could be selected to avoid a G at the first position).

It is straightforward to determine the frequency of each codon used in a portion of a nucleotide sequence. The skilled person merely needs to enter the sequence of that portion into one of the readily-available algorithms that looks at codon usage and review the results. Alternatively, the user could simply count them.

In an embodiment, the favoured codons may be selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC. In some embodiments, at least one amino acid does not have a favoured codon.

In one embodiment, in the CFI nucleotide sequence or the portion of the CFI nucleotide sequence that is codon-optimised, at least 70%, at least 75%, at least 76%, at least 77%, at least 78%, at least 79%, at least 80%, or at least 81%, at least 82%, at least 83%, or at least 84% of the codons may be selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC. In one embodiment, in the CFI nucleotide sequence or the portion of the CFI nucleotide sequence that is codon-optimised, at least 80%, at least 81%, at least 82%, at least 83%, or at least 84% of the codons may be selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC. In one embodiment, in the CFI nucleotide sequence or the portion of the CFI nucleotide sequence that is codon-optimised, at least 83%, or at least 84% of the codons may be selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC.

In one embodiment, in the CFI nucleotide sequence or the portion of the CFI nucleotide sequence that is codon-optimised, at least 75%, at least 76%, at least 77%, at least 78%, at least 79%, at least 80%, or at least 81% of the codons may be selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC. In one embodiment, in the CFI nucleotide sequence or the portion of the CFI nucleotide sequence that is codon-optimised, at least 76%, at least 77%, at least 78%, at least 79%, at least 80%, or at least 81% of the codons may be selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC. In one embodiment, in the CFI nucleotide sequence or the portion of the CFI nucleotide sequence that is codon-optimised, at least 80%, or at least 81% of the codons may be selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC.

Optionally, the portion of the CFI nucleotide sequence that is codon-optimised is codon-optimised for expression in human liver cells. Optionally, the portion of the CFI nucleotide sequence that is codon-optimised is a contiguous portion. Optionally, the CFI nucleotide sequence is codon-optimised for expression in human liver cells. Optionally, the CFI nucleotide sequence or the portion thereof is codon optimised for expression in human liver cells if the CFI nucleotide sequence expresses the CFI polypeptide or the fragment thereof in human liver cells at a higher level compared to expression of a CFI polypeptide encoded by a reference CFI nucleotide sequence. Optionally, the CFI nucleotide sequence or the portion thereof is codon optimised for expression in human liver cells if the CFI nucleotide sequence expresses the CFI polypeptide or the fragment thereof in human liver cells at a higher level on transfection of the polynucleotide comprising the CFI nucleotide sequence compared to expression of the CFI polypeptide encoded by the reference CFI nucleotide sequence on transfection with an equivalent polynucleotide comprising the reference CFI nucleotide sequence. In such embodiments, the reference CFI nucleotide sequence may be a wild-type CFI nucleotide sequence. The wild type CFI nucleotide may be the sequence of SEQ ID NO: 41 or 42. The “equivalent” polynucleotide comprising the reference CFI nucleotide is a polynucleotide which is identical (i.e. comprises the same transcription regulatory elements etc.) to the polynucleotide of the invention except that the CFI nucleotide sequences are different. For example, the different CFI nucleotide sequences being compared are operably linked to the same promoter sequence. Optionally, the human liver cell is a Huh-7 cell.

The portion that is codon-optimised can correspond to a sequence encoding part of, or the entirety of, the CFI polypeptide or the fragment thereof. Optionally, the portion of the CFI nucleotide sequence that is codon-optimised is at least 1000, at least 1200, at least 1300, at least 1400, at least 1500, at least 1600, at least 1700, 1752 or fewer, between 1300 and 1752, between 1500 and 1752, between 1600 and 1752, or around 1752 nucleotides in length. Optionally, the portion of the CFI nucleotide sequence that is codon-optimised is at least 1000, at least 1200, at least 1300, at least 1400, at least 1500, 1698 or fewer, between 1300 and 1698, between 1500 and 1698, or around 1698 nucleotides in length. For example, the coding sequence could encode a full-length CFI polypeptide (such as SEQ ID NO: 43), including a signal peptide which is not part of the mature CFI polypeptide, and the entire coding sequence could be codon-optimised. Optionally, the portion of the CFI nucleotide sequence that is codon-optimised encodes a mature CFI polypeptide. In some embodiments, the portion of the CFI nucleotide sequence that is codon-optimised does not encode all or a portion of a signal peptide. For example, the CFI nucleotide sequence may encode an immature CFI polypeptide (i.e. including a signal peptide), and the portion of the CFI nucleotide sequence that is codon-optimised encodes the mature CFI protein and does not encode all or a portion of the signal peptide. In some embodiments, the portion of the CFI nucleotide sequence that is codon-optimised encodes all or a portion of a signal peptide. For example, the CFI nucleotide sequence may encode an immature CFI polypeptide (i.e. including a signal peptide), and the portion of the CFI nucleotide sequence that is codon-optimised encodes the mature CFI protein and all or a portion of the signal peptide. In some embodiments, the CFI nucleotide sequence encoding the entire mature CFI polypeptide sequence is codon optimised.

The presence of CpGs (i.e. CG dinucleotides) in a gene therapy vector may have an adverse effect on expression of a therapeutic transgene (e.g. the durability of said expression). This is because CpGs may be methylated, and their methylation may lead to gene silencing thereby reducing expression. Also, it is possible that high CpG content could trigger a TLR response, increasing the risk of an anti-AAV immune response. The CFI nucleotide sequence or portion of the CFI nucleotide sequence that is codon-optimised may comprise a reduced number of CpGs compared to a corresponding portion of a reference CFI nucleotide sequence. In such embodiments, the reference CFI nucleotide sequence may be a wild-type CFI nucleotide sequence or the nucleotide sequence of SEQ ID NO: 1 or 21. The reference CFI nucleotide sequence may be a wild-type CFI nucleotide sequence. The wild type CFI nucleotide may be the sequence of SEQ ID NO: 41 or 42. The reference CFI nucleotide sequence may be the nucleotide sequence of SEQ ID NO: 1 or 21. The phrase “a corresponding portion of a reference CFI nucleotide sequence” is intended to refer to the portion of the reference CFI nucleotide sequence which “corresponds to” the CFI nucleotide sequence or portion of the CFI nucleotide sequence that is codon-optimised. It is within the capabilities of the person skilled in the art to determine the portion of the reference CFI nucleotide sequence which “corresponds to” the CFI nucleotide sequence or portion of the CFI nucleotide sequence that is codon-optimised. For example, the person skilled in the art merely needs to perform a sequence alignment of the reference CFI nucleotide sequence with the CFI nucleotide sequence of the invention using a suitable alignment algorithm such as that of Needleman and Wunsch described above, and determine which region of the reference CFI nucleotide sequence aligns with the CFI nucleotide sequence or portion of the CFI nucleotide sequence that is codon-optimised.

In an embodiment, the CFI nucleotide sequence or portion of the CFI nucleotide sequence that is codon-optimised (which may be all of the CFI nucleotide sequence) comprises 40 or fewer, 20 or fewer, 15 or fewer, 10 or fewer, or 5 or fewer CpGs. In an embodiment, the CFI nucleotide sequence or the portion of the CFI nucleotide sequence that is codon-optimised (which may be all of the CFI nucleotide sequence) comprises 5 or fewer, 4 or fewer, 3 or fewer, or 2 or fewer CpGs per 100 nucleotides. In some embodiments, the CFI nucleotide sequence or the portion of the CFI nucleotide sequence that is codon-optimised is CpG-free, i.e. contains no (0) CG dinucleotides.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1-20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1, 2, 3, 5, 6, 7, 9-12, 14-17, and 20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 2, 3, 5, 6, 7, 9-12, 14-17, and 20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1, 5, 7, 10-14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1, 5, 7, 11-14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1, 5, 7, 11, 12, 14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 5, 7, 12-14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 5, 7, 12, 14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 5, 7, and 14.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 3, 5-7, 10, 14, 17, and 20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 5, 10, and 17.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to SEQ ID NOs: 1-20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 1, 2, 3, 5, 6, 7, 9-12, 14-17, and 20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 2, 3, 5, 6, 7, 9-12, 14-17, and 20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 1, 5, 7, 10-14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 1, 5, 7, 11-14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 1, 5, 7, 11, 12, 14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 5, 7, 12-14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 5, 7, 12, 14, 16, and 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 5, 7, and 14.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 3, 5-7, 10, 14, 17, and 20. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 5, 10, and 17.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of SEQ ID NO: 5. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to SEQ ID NO: 5. In an embodiment, the portion of the CFI nucleotide sequence that is codon-optimised is at least 99.5%, at least 99.8%, or 100% identical to a fragment of at least 1500 nucleotides of SEQ ID NO: 5. In an embodiment, the portion of the CFI nucleotide sequence that is codon-optimised is at least 99%, at least 99.5%, at least 99.8%, or 100% identical to SEQ ID NO: 5.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of SEQ ID NO: 7. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to SEQ ID NO: 7. In an embodiment, the portion of the CFI nucleotide sequence that is codon-optimised is at least 99.5%, at least 99.8%, or 100% identical to a fragment of at least 1500 nucleotides of SEQ ID NO: 7. In an embodiment, the portion of the CFI nucleotide sequence that is codon-optimised is at least 99%, at least 99.5%, at least 99.8%, or 100% identical to SEQ ID NO: 7.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of SEQ ID NO: 10. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to SEQ ID NO: 10. In an embodiment, the portion of the CFI nucleotide sequence that is codon-optimised is at least 99.5%, at least 99.8%, or 100% identical to a fragment of at least 1500 nucleotides of SEQ ID NO: 10. In an embodiment, the portion of the CFI nucleotide sequence that is codon-optimised is at least 99%, at least 99.5%, at least 99.8%, or 100% identical to SEQ ID NO: 10.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of SEQ ID NO: 14. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to SEQ ID NO: 14. In an embodiment, the portion of the CFI nucleotide sequence that is codon-optimised is at least 99.5%, at least 99.8%, or 100% identical to a fragment of at least 1500 nucleotides of SEQ ID NO: 14. In an embodiment, the portion of the CFI nucleotide sequence that is codon-optimised is at least 99%, at least 99.5%, at least 99.8%, or 100% identical to SEQ ID NO: 14.

In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of SEQ ID NO: 17. In an embodiment, the portion of the CFI nucleotide sequence that is codon-optimised is at least 80%, at least 85%, at least 90%, at least 95%, at least 98%, at least 99%, at least 99.5%, at least 99.8%, or 100% identical to SEQ ID NO: 17. In an embodiment, the portion of the CFI nucleotide sequence that is codon-optimised is at least 99.5%, at least 99.8%, or 100% identical to a fragment of at least 1500 nucleotides of SEQ ID NO: 17. In an embodiment, the portion of the CFI nucleotide sequence that is codon-optimised is at least 99%, at least 99.5%, at least 99.8%, or 100% identical to SEQ ID NO: 17.

The CFI polypeptide or fragment thereof encoded by the CFI nucleotide sequence may be expressed at the same or a higher level in a human liver cell compared to expression of a CFI polypeptide encoded by a reference CFI nucleotide sequence. Optionally, the CFI polypeptide or the fragment thereof encoded by the CFI nucleotide sequence is expressed at the same or a higher level on transfection of the polynucleotide comprising the CFI nucleotide sequence into a human liver cell compared to expression of the CFI polypeptide encoded by the reference CFI nucleotide sequence on transfection with an equivalent polynucleotide comprising the reference CFI nucleotide sequence. Optionally, the human liver cell is a Huh-7 cell. The “equivalent” polynucleotide comprising the reference CFI nucleotide is a polynucleotide which is identical (i.e. comprises the same transcription regulatory elements etc.) to the polynucleotide of the invention except that the CFI nucleotide sequences are different. For example, the different CFI nucleotide sequences being compared are operably linked to the same promoter sequence. In embodiments where the expression of the CFI polypeptide or the fragment thereof encoded by the CFI nucleotide sequence encoded by the CFI nucleotide sequence is at the same level compared to the expression of the CFI polypeptide encoded by the reference CFI nucleotide sequence, the reference CFI nucleotide sequence may be the nucleotide sequence of any one of SEQ ID NOs: 1, 21, 5 or 25. The reference CFI nucleotide sequence may be the nucleotide sequence of SEQ ID NO: 1 or 21. The reference CFI nucleotide sequence may be the nucleotide sequence of SEQ ID NO: 5 or 25. Optionally, the expression of the CFI polypeptide or the fragment thereof encoded by the CFI nucleotide sequence is at a higher level compared to the expression of the CFI polypeptide encoded by the reference CFI nucleotide sequence. Optionally, the expression of the CFI polypeptide or fragment thereof encoded by the CFI nucleotide sequence may be at least 1.5x, at least 2x, at least 3x, at least 5x, at least 8x, at least 10x, at least 20x, at least 30x, at least 40x or at least 50x higher compared to the CFI polypeptide encoded by the reference CFI nucleotide sequence. Optionally, the expression of the CFI polypeptide or fragment thereof encoded by the CFI nucleotide sequence may be between 1.5x and 50x, between 5x and 50x, between 10x and 50x, or between 30x and 50x higher compared to the CFI polypeptide encoded by the reference CFI nucleotide sequence.

Optionally, the expression of the CFI polypeptide or fragment thereof encoded by the CFI nucleotide sequence may be at least 80x, at least 100x, at least 200x, at least 250x, at least 300x, at least 350x, or at least 400x higher compared to the CFI polypeptide encoded by the reference CFI nucleotide sequence. Optionally, the expression of the CFI polypeptide or fragment thereof encoded by the CFI nucleotide sequence may be at least 400x higher compared to the CFI polypeptide encoded by the reference CFI nucleotide sequence. Optionally, the expression of the CFI polypeptide or fragment thereof encoded by the CFI nucleotide sequence may be between 1.5x and 500x, between 10x and 500x, between 100x and 500x, or between 20x and 500x higher compared to the CFI polypeptide encoded by the reference CFI nucleotide sequence. Optionally, the expression of the CFI polypeptide or fragment thereof encoded by the CFI nucleotide sequence is at least one standard deviation higher than the expression of the CFI polypeptide or fragment thereof encoded by at least one SEQ ID NOs: 1-4, 6-24, and 26-40. Optionally, the expression of the CFI polypeptide or fragment thereof encoded by the CFI nucleotide sequence is at least one standard deviation higher than the expression of the CFI polypeptide or fragment thereof encoded by at least one SEQ ID NOs: 17 or 37.

In embodiments where the expression of the CFI polypeptide or the fragment thereof encoded by the CFI nucleotide sequence encoded by the CFI nucleotide sequence is at a higher level compared to the expression of the CFI polypeptide encoded by the reference CFI nucleotide sequence, the reference CFI nucleotide sequence may be a wild-type CFI nucleotide sequence or the nucleotide sequence of any one of SEQ ID NOs: 1, 21, 5 or 25. The reference CFI nucleotide sequence may be a wild-type CFI nucleotide sequence. The wild type CFI nucleotide may be the sequence of SEQ ID NO: 41 or 42. The reference CFI nucleotide sequence may be the nucleotide sequence of SEQ ID NO: 1 or 21. The reference CFI nucleotide sequence may be the nucleotide sequence of SEQ ID NO: 5 or 25. Optionally, the expression of the CFI polypeptide or the fragment thereof encoded by the CFI nucleotide sequence of the invention is at least 50%, at least 60%, at least 70%, at least 80%, at least 90%, at least 95%, or at least 100% of the expression of the CFI polypeptide encoded by the sequence of SEQ ID NO: 5 or 25.

The level of expression of the CFI polypeptide or the fragment thereof may typically be determined by measuring the level of the CFI polypeptide or the fragment thereof in a sample. Optionally, the level of expression of a CFI polypeptide or the fragment thereof is determined by using an ELISA. The level of expression of a CFI polypeptide or the fragment thereof may be determined using the ELISA assay as described in Example 1. Optionally, the expression level is normalised for transfection efficiency. Optionally, the expression level is not normalised for transfection efficiency. One way to normalise for transfection efficiency uses a luciferase assay.

The ELISA to determine the level of expression of a CFI polypeptide or the fragment thereof may be performed using a sandwich ELISA using two antibodies that bind to CFI polypeptide or a fragment thereof (for example, using the CFI ELISA kit (e.g. Hycult cat# HK355-01) according to the manufacturer’s instructions). For example, a first antibody that binds to CFI or a fragment thereof may be immobilised to a plate, and a sample comprising the CFI polypeptide or fragment thereof may be applied to the plate with the first antibody immobilised on it. The plate may be washed, and then a second antibody that binds to CFI polypeptide or fragment thereof and is conjugated to a label may be applied to the plate. The second antibody may be conjugated to biotin, which binds to streptavidin-peroxidase and in this case the streptavidin-peroxidase acts as the label. The plate may be washed again, and the amount of CFI polypeptide or fragment thereof bound to the plate measured by assessing the amount of the label present. For example, if the label is streptavidin-peroxidase, the substrate tetramethylbenzidine (TMB) may be added, which the streptavidin-peroxidase reacts with. The enzyme reaction may be stopped by adding oxalic acid. For each of the samples and standards, the absorbance at 450 nm may be measured using a spectrophotometer. A standard curve may be obtained by plotting the absorbance (linear) against the corresponding concentrations of human CFI standards (log). The concentration of human CFI in the samples may then be determined from the standard curve.

Optionally, the level of expression of the CFI polypeptide or the fragment thereof is determined following the transfection of a human liver cell (e.g. Huh-7 cell) with a polynucleotide comprising the CFI nucleotide sequence encoding the CFI polypeptide or the fragment thereof. Optionally, detecting the level of expression of the CFI polypeptide or the fragment thereof comprises harvesting medium in which the transfected human liver cells were cultured and detecting the level of expression in the medium. As mentioned above, the expression level of a CFI polypeptide or the fragment thereof may be normalised for transfection efficiency using a luciferase assay. Optionally, the transfection may comprise co-transfecting a polynucleotide comprising a transgene encoding luciferase. This allows the users to normalise the measured expression to reflect the number of cells that have been transfected (thereby accounting for differences in transfection efficiency), as the proportion of cells that are transfected by the polynucleotide comprising the CFI nucleotide sequence encoding the CFI polypeptide or the fragment thereof will be proportionate to the fluorescent signal produced by the luciferase expressed from the polynucleotide comprising the luciferase transgene. Optionally, the polynucleotide comprising the luciferase transgene further comprises a CMV or TK promoter, and the luciferase transgene is operably linked to the CMV or TK promoter. Optionally, the luciferase expression may be measured in the cells (or a portion of the cells). For example, the cells (or a portion of the cells) may be harvested and lysed, and the luciferase expression measured in the lysate. Optionally, the luciferase expression may be measured in the medium in which the transfected human liver cells were cultured (or a portion of the medium). Optionally, the luciferase expression may be measured by performing a luciferase assay. Optionally, the luminescence may be measured by using a luminometer. Luciferase expression may be measured by measuring luminescence on a Molecular Devices SpectraMax i3x plate reader.

The CFI nucleotide sequence may encode a signal peptide. Optionally, the amino acid sequence of the signal peptide is an amino acid sequence of a wild type CFI signal peptide. The amino acid sequence of a wild type CFI signal peptide may be the amino acid sequence of SEQ ID NO: 46. The signal peptide may be encoded by a nucleotide sequence of SEQ ID NO: 45. The signal peptide may not be encoded by a wild-type CFI signal peptide-encoding nucleotide sequence. The signal peptide may be encoded by nucleotides 1 to 54 of any one of SEQ ID NOs: 21 to 40. Optionally, the amino acid sequence of the signal peptide is an amino acid sequence of a signal peptide which is not a wild type CFI signal peptide. For example, the signal peptide may not be a wild type CFI signal peptide. The signal peptide may be the signal peptide from another polypeptide. Optionally, the CFI nucleotide sequence encodes a signal peptide. Optionally, the CFI nucleotide sequence does not encode a signal peptide. Optionally, the CFI polypeptide or fragment thereof comprises a signal peptide. Optionally, the CFI polypeptide or fragment thereof does not comprise a signal peptide.

The polynucleotide of the invention may comprise a transcription regulatory element.

Any appropriate transcription regulatory element may be used, such as HLP2, HLP1, LP1, HCR-hAAT, ApoE-hAAT, or LSP, which are all liver-specific transcription regulatory elements. These transcription regulatory elements are described in more detail in the following references: HLP1: McIntosh J. et al., Blood 2013 Apr 25, 121(17):3335-44; LP1: Nathwani et al., Blood. 2006 April 1, 107(7): 2653-2661; HCR-hAAT: Miao et al., Mol Ther. 2000; 1: 522-532; ApoE-hAAT: Okuyama et al., Human Gene Therapy, 7, 637-645 (1996); and LSP: Wang et al., Proc Natl Acad Sci USA. 1999 March 30, 96(7): 3906-3910. The transcription regulatory element may comprise a liver-specific promoter.

The transcription regulatory element may comprise a promoter and/or an enhancer, such as the promoter element and/or enhancer element from HLP2, HLP1, LP1, HCR-hAAT, ApoE-hAAT, or LSP. Each of these transcription regulatory elements comprises a promoter, an enhancer, and optionally other nucleotides. The transcription regulatory element may be operably linked to the CFI nucleotide sequence of the invention. The promoter and/or enhanced may operably linked to the CFI nucleotide sequence of the invention.

In some embodiments, the transcription regulatory element comprises a promoter which is a human alpha-1 anti-trypsin promoter (A1AT; Miao et al (2000), Molecular Therapy 1(6):522), or a fragment thereof. In an embodiment, the fragment of an A1AT promoter is at least 100, at least 120, at least 150, at least 180, 255 or fewer, between 100 and 255, between 150 and 225, between 150 and 300, or between 180 and 255 nucleotides in length. Optionally, the fragment of an A1AT promoter is between 150 and 300 nucleotides in length. Optionally, the fragment of an A1AT promoter is between 180 and 255 nucleotides in length. In an embodiment, the fragment of an A1AT promoter is at least 200, at least 250, at least 300, 500 or fewer, between 200 and 500, between 250 and 500, or between 350 and 450, or around 418 nucleotides in length. Optionally, the fragment of an A1AT promoter is between 350 and 450 nucleotides in length.

Suitable A1AT promoter fragments are described in SEQ ID NOs: 47 and 48. Optionally, the transcription regulatory element comprises a promoter that is at least 100, at least 120, at least 150, at least 180, less than 255, between 100 and 255, between 150 and 300, or between 180 and 255 nucleotides in length and the promoter comprises a polynucleotide sequence that is at least 80%, at least 85%, at least 90%, at least 95%, at least 98%, at least 99%, at least 99.5%, at least 99.8%, or 100% identical to SEQ ID NO: 47. Optionally, the transcription regulatory element comprises a promoter that is between 180 and 255 nucleotides in length and the promoter comprises a polynucleotide sequence that is at least 98%, at least 99%, at least 99.5%, at least 99.8%, or 100% identical to SEQ ID NO: 47. Optionally, the polynucleotide comprises a promoter that is at least 80%, at least 85%, at least 90%, at least 95%, at least 98%, at least 99%, at least 99.5%, at least 99.8%, or 100% identical to a fragment of at least 100, at least 120, or at least 150 nucleotides of SEQ ID NO: 47. Optionally, the polynucleotide comprises a promoter that is at least 80%, at least 85%, at least 90%, at least 95%, at least 98%, at least 99%, at least 99.5%, at least 99.8%, or 100% identical to SEQ ID NO: 47. Optionally, the polynucleotide comprises a promoter that is at least 98%, at least 99%, at least 99.5%, at least 99.8%, or 100% identical to SEQ ID NO: 47. Optionally, the polynucleotide comprises a promoter of SEQ ID NO: 47.

The transcription regulatory element may comprise a promoter that is at least 200, at least 250, at least 300, 500 or fewer, between 200 and 500, between 250 and 500, between 350 and 450, or around 418 nucleotides in length and the promoter comprises a polynucleotide sequence that is at least 80%, at least 85%, at least 90%, at least 95%, at least 98%, at least 99%, at least 99.5%, at least 99.8%, or 100% identical to SEQ ID NO: 48. Optionally, the transcription regulatory element comprises a promoter that is between 350 and 450 nucleotides in length and the promoter comprises a polynucleotide sequence that is at least 98%, at least 99%, at least 99.5%, at least 99.8%, or 100% identical to SEQ ID NO: 48. Optionally, the polynucleotide comprises a promoter that is at least 80%, at least 85%, at least 90%, at least 95%, at least 98%, at least 99%, at least 99.5%, at least 99.8%, or 100% identical to a fragment of at least 350 nucleotides of SEQ ID NO: 48. Optionally, the polynucleotide comprises a promoter that is at least 80%, at least 85%, at least 90%, at least 95%, at least 98%, at least 99%, at least 99.5%, at least 99.8%, or 100% identical to SEQ ID NO: 48. Optionally, the polynucleotide comprises a promoter that is at least 98%, at least 99%, at least 99.5%, at least 99.8%, or 100% identical to SEQ ID NO: 48. Optionally, the polynucleotide comprises a promoter of SEQ ID NO: 48.

Optionally, the transcription regulatory element comprises a fragment of an A1AT promoter that is equal to or fewer than 418 nucleotides, equal to or fewer than 255 nucleotides, or equal to or fewer than 185 nucleotides in length and comprises SEQ ID NO: 47.

If the polynucleotide is intended for expression in the liver, the promoter may be a liver-specific promoter. Optionally, the promoter is a human liver-specific promoter.

The transcription regulatory element may comprise an enhancer. In some embodiments, the transcription regulatory element comprises an enhancer which is the human apolipoprotein E (ApoE) hepatic locus control region (HCR; Miao et al (2000), Molecular Therapy 1(6):522), or a fragment thereof. In an embodiment, the transcription regulatory element comprises a fragment of the HCR enhancer which is a fragment of at least 80, at least 90, at least 100, 192 or fewer, between 80 and 192, between 90 and 192, between 100 and 250, or between 117 and 192 nucleotides in length. Optionally, the fragment of the HCR enhancer is between 100 and 250 nucleotides in length. Optionally, the fragment of the HCR enhancer is between 117 and 192 nucleotides in length. In an embodiment, the HCR enhancer or the fragment of an HCR enhancer is a fragment of at least 150, at least 190, at least 230, fewer than 400, between 150 and 400, between 190 and 370, between 230 and 340, between 250 and 340, or around 321 nucleotides in length. Optionally, the HCR enhancer or the fragment of the HCR enhancer is between 250 and 340 nucleotides in length.

Suitable HCR enhancer element fragments are defined in SEQ ID NOs: 49 and 50. Optionally, the transcription regulatory element comprises an enhancer that is at least 80, at least 90, at least 100, less than 192, between 80 and 192, between 90 and 192, between 100 and 250, or between 117 and 192 nucleotides in length and the enhancer comprises a polynucleotide sequence that is at least 80%, at least 85%, at least 90%, at least 95%, at least 98%, at least 99%, at least 99.5%, at least 99.8%, or 100% identical to SEQ ID NO: 49. Optionally, the transcription regulatory element comprises an enhancer that is between 117 and 192 nucleotides in length and the enhancer comprises a polynucleotide sequence that is at least 98%, at least 99%, at least 99.5%, at least 99.8%, or 100% identical to SEQ ID NO: 49. Optionally, the transcription regulatory element comprises an enhancer that is at least 80%, at least 85%, at least 90%, at least 95%, at least 98%, at least 99%, at least 99.5%, at least 99.8%, or 100% identical to a fragment of at least 90, at least 100, or at least 110 nucleotides of SEQ ID NO: 49. Optionally, the polynucleotide comprises an enhancer that is at least 80%, at least 85%, at least 90%, at least 95% at least 98%, at least 99%, at least 99.5%, at least 99.8%, or 100% identical to SEQ ID NO: 49. Optionally, the polynucleotide comprises an enhancer that is at least 98%, at least 99%, at least 99.5%, at least 99.8%, or 100% identical to SEQ ID NO: 49. Optionally, the polynucleotide comprises an enhancer of SEQ ID NO: 49.

In some embodiments, the transcription regulatory element comprises an enhancer or a fragment of an enhancer that is at least 150, at least 190, at least 230, fewer than 400, between 150 and 400, between 190 and 370, between 230 and 340, between 250 and 340, or around 321 nucleotides in length and the enhancer comprises a polynucleotide sequence that is at least 80%, at least 85%, at least 90%, at least 95%, at least 98%, at least 99%, at least 99.5%, at least 99.8%, or 100% identical to SEQ ID NO: 50. Optionally, the transcription regulatory element comprises an enhancer that is between 250 and 340 nucleotides in length and the enhancer comprises a polynucleotide sequence that is at least 98%, at least 99%, at least 99.5%, at least 99.8%, or 100% identical to SEQ ID NO: 50. Optionally, the transcription regulatory element comprises an enhancer that is at least 80%, at least 85%, at least 90%, at least 95%, at least 98%, at least 99%, at least 99.5%, at least 99.8%, or 100% identical to a fragment of at least 250 nucleotides of SEQ ID NO: 50. Optionally, the polynucleotide comprises an enhancer that is at least 80%, at least 85%, at least 90%, at least 95% at least 98%, at least 99%, at least 99.5%, at least 99.8%, or 100% identical to SEQ ID NO: 50. Optionally, the polynucleotide comprises an enhancer that is at least 98%, at least 99%, at least 99.5%, at least 99.8%, or 100% identical to SEQ ID NO: 50. Optionally, the polynucleotide comprises an enhancer of SEQ ID NO: 50.

Optionally, the transcription regulatory element comprises a fragment of an HCR enhancer that is equal to or fewer than 321 nucleotides, equal to or fewer than 192 nucleotides, or equal to or fewer than 117 nucleotides in length and comprises SEQ ID NO: 49.

In an embodiment, the transcription regulatory element is at least 80%, at least 85%, at least 90%, at least 95%, at least 98%, at least 99%, at least 99.5%, at least 99.8%, or 100% identical to SEQ ID NO: 51. In an embodiment, the polynucleotide comprises a transcription regulatory element that is at least 98%, at least 99%, at least 99.5%, at least 99.8%, or 100% identical to SEQ ID NO: 51. Optionally, the polynucleotide comprises a transcription regulatory element that is at least 98% identical to SEQ ID NO: 51. Optionally, the polynucleotide comprises a transcription regulatory element that has a sequence of SEQ ID NO: 51. Optionally, the polynucleotide comprises a transcription regulatory element of SEQ ID NO: 51. Optionally, the polynucleotide comprises a transcription regulatory element consisting of SEQ ID NO: 51.

In an embodiment, the transcription regulatory element is at least 80%, at least 85%, at least 90%, at least 95%, at least 98%, at least 99%, at least 99.5%, at least 99.8%, or 100% identical to SEQ ID NO: 52. In an embodiment, the polynucleotide comprises a transcription regulatory element that is at least 98%, at least 99%, at least 99.5%, at least 99.8%, or 100% identical to SEQ ID NO: 52. Optionally, the polynucleotide comprises a transcription regulatory element that is at least 98% identical to SEQ ID NO: 52. Optionally, the polynucleotide comprises a transcription regulatory element that has a sequence of SEQ ID NO: 52. Optionally, the polynucleotide comprises a transcription regulatory element of SEQ ID NO: 52. Optionally, the polynucleotide comprises a transcription regulatory element consisting of SEQ ID NO: 52.

In an embodiment, the CFI nucleotide sequence comprises a sequence that is at least 95%, at least 98%, at least 99%, at least 99.5%, at least 99.8%, or 100% identical to SEQ ID NO: 5 or 25; and the polynucleotide comprises a promoter that is at least 98%, at least 99%, at least 99.5%, at least 99.8%, or 100% identical to SEQ ID NO: 47 and/or an enhancer element that is at least 98%, at least 99%, at least 99.5%, at least 99.8% or 100% identical to SEQ ID NO: 49. In another embodiment, the CFI nucleotide sequence comprises a sequence that is at least 95%, at least 98%, at least 99%, at least 99.5%, at least 99.8%, or 100% identical to SEQ ID NO: 5 or 25; and the polynucleotide comprises a transcription regulatory element that is at least 98%, at least 99%, at least 99.5%, at least 99.8%, or 100% identical to SEQ ID NO: 51.

In an embodiment, the CFI nucleotide sequence comprises a sequence that is at least 95%, at least 98%, at least 99%, at least 99.5%, at least 99.8%, or 100% identical to SEQ ID NO: 7 or 27; and the polynucleotide comprises a promoter that is at least 98%, at least 99%, at least 99.5%, at least 99.8%, or 100% identical to SEQ ID NO: 47 and/or an enhancer element that is at least 98%, at least 99%, at least 99.5%, at least 99.8% or 100% identical to SEQ ID NO: 49. In another embodiment, the CFI nucleotide sequence comprises a sequence that is at least 95%, at least 98%, at least 99%, at least 99.5%, at least 99.8%, or 100% identical to SEQ ID NO: 7 or 27; and the polynucleotide comprises a transcription regulatory element that is at least 98%, at least 99%, at least 99.5%, at least 99.8%, or 100% identical to SEQ ID NO: 51.

In an embodiment, the CFI nucleotide sequence comprises a sequence that is at least 95%, at least 98%, at least 99%, at least 99.5%, at least 99.8%, or 100% identical to SEQ ID NO: 10 or 30; and the polynucleotide comprises a promoter that is at least 98%, at least 99%, at least 99.5%, at least 99.8%, or 100% identical to SEQ ID NO: 47 and/or an enhancer element that is at least 98%, at least 99%, at least 99.5%, at least 99.8% or 100% identical to SEQ ID NO: 49. In another embodiment, the CFI nucleotide sequence comprises a sequence that is at least 95%, at least 98%, at least 99%, at least 99.5%, at least 99.8%, or 100% identical to SEQ ID NO: 10 or 30; and the polynucleotide comprises a transcription regulatory element that is at least 98%, at least 99%, at least 99.5%, at least 99.8%, or 100% identical to SEQ ID NO: 51.

In an embodiment, the CFI nucleotide sequence comprises a sequence that is at least 95%, at least 98%, at least 99%, at least 99.5%, at least 99.8%, or 100% identical to SEQ ID NO: 14 or 34; and the polynucleotide comprises a promoter that is at least 98%, at least 99%, at least 99.5%, at least 99.8%, or 100% identical to SEQ ID NO: 47 and/or an enhancer element that is at least 98%, at least 99%, at least 99.5%, at least 99.8% or 100% identical to SEQ ID NO: 49. In another embodiment, the CFI nucleotide sequence comprises a sequence that is at least 95%, at least 98%, at least 99%, at least 99.5%, at least 99.8%, or 100% identical to SEQ ID NO: 14 or 34; and the polynucleotide comprises a transcription regulatory element that is at least 98%, at least 99%, at least 99.5%, at least 99.8%, or 100% identical to SEQ ID NO: 51.

In an embodiment, the CFI nucleotide sequence comprises a sequence that is at least 95%, at least 98%, at least 99%, at least 99.5%, at least 99.8%, or 100% identical to SEQ ID NO: 17 or 37; and the polynucleotide comprises a promoter that is at least 98%, at least 99%, at least 99.5%, at least 99.8%, or 100% identical to SEQ ID NO: 47 and/or an enhancer element that is at least 98%, at least 99%, at least 99.5%, at least 99.8% or 100% identical to SEQ ID NO: 49. In another embodiment, the CFI nucleotide sequence comprises a sequence that is at least 95%, at least 98%, at least 99%, at least 99.5%, at least 99.8%, or 100% identical to SEQ ID NO: 17 or 37; and the polynucleotide comprises a transcription regulatory element that is at least 98%, at least 99%, at least 99.5%, at least 99.8%, or 100% identical to SEQ ID NO: 51.

In an embodiment, the CFI nucleotide sequence comprises a sequence that is at least 95%, at least 98%, at least 99%, at least 99.5%, at least 99.8%, or 100% identical to SEQ ID NO: 5 or 25; and the polynucleotide comprises a promoter that is at least 98%, at least 99%, at least 99.5%, at least 99.8%, or 100% identical to SEQ ID NO: 48 and/or an enhancer element that is at least 98%, at least 99%, at least 99.5%, at least 99.8% or 100% identical to SEQ ID NO: 50. In another embodiment, the CFI nucleotide sequence comprises a sequence that is at least 95%, at least 98%, at least 99%, at least 99.5%, at least 99.8%, or 100% identical to SEQ ID NOs: 5 or 25; and the polynucleotide comprises a transcription regulatory element that is at least 98%, at least 99%, at least 99.5%, at least 99.8%, or 100% identical to SEQ ID NO: 52.

In an embodiment, the CFI nucleotide sequence comprises a sequence that is at least 95%, at least 98%, at least 99%, at least 99.5%, at least 99.8%, or 100% identical to SEQ ID NO: 7 or 27; and the polynucleotide comprises a promoter that is at least 98%, at least 99%, at least 99.5%, at least 99.8%, or 100% identical to SEQ ID NO: 48 and/or an enhancer element that is at least 98%, at least 99%, at least 99.5%, at least 99.8% or 100% identical to SEQ ID NO: 50. In another embodiment, the CFI nucleotide sequence comprises a sequence that is at least 95%, at least 98%, at least 99%, at least 99.5%, at least 99.8%, or 100% identical to SEQ ID NOs: 7 or 27; and the polynucleotide comprises a transcription regulatory element that is at least 98%, at least 99%, at least 99.5%, at least 99.8%, or 100% identical to SEQ ID NO: 52.

In an embodiment, the CFI nucleotide sequence comprises a sequence that is at least 95%, at least 98%, at least 99%, at least 99.5%, at least 99.8%, or 100% identical to SEQ ID NO: 10 or 30; and the polynucleotide comprises a promoter that is at least 98%, at least 99%, at least 99.5%, at least 99.8%, or 100% identical to SEQ ID NO: 48 and/or an enhancer element that is at least 98%, at least 99%, at least 99.5%, at least 99.8% or 100% identical to SEQ ID NO: 50. In another embodiment, the CFI nucleotide sequence comprises a sequence that is at least 95%, at least 98%, at least 99%, at least 99.5%, at least 99.8%, or 100% identical to SEQ ID NOs: 10 or 30; and the polynucleotide comprises a transcription regulatory element that is at least 98%, at least 99%, at least 99.5%, at least 99.8%, or 100% identical to SEQ ID NO: 52.

In an embodiment, the CFI nucleotide sequence comprises a sequence that is at least 95%, at least 98%, at least 99%, at least 99.5%, at least 99.8%, or 100% identical to SEQ ID NO: 14 or 34; and the polynucleotide comprises a promoter that is at least 98%, at least 99%, at least 99.5%, at least 99.8%, or 100% identical to SEQ ID NO: 48 and/or an enhancer element that is at least 98%, at least 99%, at least 99.5%, at least 99.8% or 100% identical to SEQ ID NO: 50. In another embodiment, the CFI nucleotide sequence comprises a sequence that is at least 95%, at least 98%, at least 99%, at least 99.5%, at least 99.8%, or 100% identical to SEQ ID NOs: 14 or 34; and the polynucleotide comprises a transcription regulatory element that is at least 98%, at least 99%, at least 99.5%, at least 99.8%, or 100% identical to SEQ ID NO: 52.

In an embodiment, the CFI nucleotide sequence comprises a sequence that is at least 95%, at least 98%, at least 99%, at least 99.5%, at least 99.8%, or 100% identical to SEQ ID NO: 17 or 37; and the polynucleotide comprises a promoter that is at least 98%, at least 99%, at least 99.5%, at least 99.8%, or 100% identical to SEQ ID NO: 48 and/or an enhancer element that is at least 98%, at least 99%, at least 99.5%, at least 99.8% or 100% identical to SEQ ID NO: 50. In another embodiment, the CFI nucleotide sequence comprises a sequence that is at least 95%, at least 98%, at least 99%, at least 99.5%, at least 99.8%, or 100% identical to SEQ ID NOs: 17 or 37; and the polynucleotide comprises a transcription regulatory element that is at least 98%, at least 99%, at least 99.5%, at least 99.8%, or 100% identical to SEQ ID NO: 52.

In some embodiments, the transcription regulatory element comprises a ubiquitously expressing promoter. Optionally, the transcription regulatory element comprises a CAG promoter. Optionally, the transcription regulatory element comprises a sequence that is at least 95%, at least 96%, at least 97%, at least 98%, at least 99%, at least 99.5%, at least 99.8%, or 100% identical to the nucleotide sequence of a CAG promoter (e.g. SEQ ID NO: 61). Optionally, the polynucleotide of the invention comprises a sequence that is at least 95%, at least 96%, at least 97%, at least 98%, at least 99%, at least 99.5%, at least 99.8%, or 100% identical to the nucleotide sequence of a CAG promoter and the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 1 or 21. Optionally, the polynucleotide of the invention comprises a CAG promoter and the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 1 or 21. Optionally, the polynucleotide of the invention comprises a CAG promoter and the CFI nucleotide sequence comprises a sequence of SEQ ID NO: 1 or 21.

In an embodiment, the polynucleotide of the invention comprises a woodchuck hepatitis post-transcriptional regulatory element (WPRE). For example, the polynucleotide of the invention comprises the mutated WPRE sequence described in Zanta-Boussif et al (2009), Gene Therapy, 16:605-619. In another embodiment, the polynucleotide of the invention does not comprise a woodchuck hepatitis post-transcriptional regulatory element (WPRE).

In an embodiment, the polynucleotide of the invention comprises a woodchuck hepatitis post-transcriptional regulatory element or a variant thereof. Optionally, the woodchuck hepatitis post-transcriptional regulatory element is at least 75%, at least 80%, at least 85%, at least 90%, at least 95%, at least 96%, at least 97%, at least 98%, at least 99%, at least 99.5%, at least 99.8%, or 100% identical to any one of SEQ ID NOs: 62 to 64. Optionally, the polynucleotide comprises a woodchuck hepatitis post-transcriptional regulatory element that is at least 98%, at least 99%, at least 99.5%, at least 99.8%, or 100% identical to any one of SEQ ID NOs 62 to 64. Optionally, the polynucleotide comprises a woodchuck hepatitis post-transcriptional regulatory element that is at least 98% identical to any one of SEQ ID NOs: 62 to 64. Optionally, the polynucleotide comprises a woodchuck hepatitis post-transcriptional regulatory element that has a sequence of any one of SEQ ID NOs: 62 to 64. Optionally, the polynucleotide comprises a woodchuck hepatitis post-transcriptional regulatory element of any one of SEQ ID NOs: 62 to 64. Optionally, the polynucleotide comprises a woodchuck hepatitis post-transcriptional regulatory element consisting of any one of SEQ ID NOs: 62 to 64.

Optionally, the transcription regulatory element comprises two fragments of an A1AT promoter. Optionally, the first fragment of an A1AT promoter is between 75 and 100 nucleotides in length and the second fragment of an A1AT promoter is between 25 and 50 nucleotides in length. Optionally, the polynucleotide comprises a promoter that is at least 80%, at least 85%, at least 90%, at least 95%, at least 98%, at least 99%, at least 99.5%, or 100% identical to SEQ ID NO: 66.

The transcription regulatory element may be a transcription regulatory element that:
(a) promotes greater expression than HCR-hAAT;
(b) promotes greater expression than FRE72;
(c) comprises a hAAT sequence element; and/or
(d) comprises an AMBP sequence element.

A TRE may comprise one or more promoter and/or enhancer elements, such as a promoter and/or enhancer element selected from the group consisting of a hAAT sequence element, an AMBP sequence element, an ApoE-HCR1 sequence element, an ALDOB sequence element, a CRM6 sequence element, and an F2 sequence element. For example, a polynucleotide which comprises a hAAT sequence element is a TRE.

In one aspect, the TRE is greater than 500, greater than 600, greater than 700, greater than 800, greater than 900, greater than 1000, greater than 1100, greater than 1200, greater than 1300, greater than 1400, greater than 1500, or greater than 1600 nucleotides in length. In an aspect, the TRE is less than 2000, less than 1800, less than 1600, less than 1500, less than 1400, less than 1300, less than 1200, less than 1100, less than 1000, less than 900, less than 800, less than 700, or less than 600 nucleotides in length. In an aspect, the TRE is between 400 and 2000, between 500 and 1800, between 600 and 1400, between 800 and 1600, between 500 and 600, between 600 and 700, between 700 and 800, between 800 and 900, between 900 and 1000, between 1000 and 1100, between 1100 and 1200, between 1200 and 1300, between 1300 and 1400, between 1400 and 1500, or between 1500 and 1600, or between 1600 and 1700 nucleotides in length.

In one embodiment, the TRE is between 563 and 1628 nucleotides in length. In one embodiment the TRE is between 563 and 583 nucleotides in length. In one embodiment, the TRE is between 566 and 586 nucleotides in length. In one embodiment, the TRE is between 888 and 908 nucleotides in length. In one embodiment, the TRE is between 893 and 913 nucleotides in length. In one embodiment, the TRE is between 897 and 917 nucleotides in length. In one embodiment, the TRE is between 899 and 919 nucleotides in length. In one embodiment, the TRE is between 903 and 923 nucleotides in length. In one embodiment, the TRE is between 905 and 925 nucleotides in length. In one embodiment, the TRE is between 909 and 929 nucleotides in length. In one embodiment, the TRE is between 913 and 933 nucleotides in length. In one embodiment, the TRE is between 923 and 943 nucleotides in length. In one embodiment, the TRE is between 933 and 953 nucleotides in length. In one embodiment, the TRE is between 1353 and 1373 nucleotides in length. In one embodiment, the TRE is between 1608 and 1628 nucleotides in length. In one embodiment, the TRE is between 1474 and 1494 nucleotides in length.

In one embodiment, the TRE is longer than FRE72. In one embodiment, the TRE is at least 450, at least 770, at least 1240, at least 1340, or at least 1390 nucleotides longer than FRE72. In one embodiment, the TRE is shorter than HCR-hAAT. In one embodiment, the TRE is at least 160 nucleotides shorter than HCR-hAAT. In one embodiment, the TRE is longer than HCR-hAAT. In one embodiment, the TRE is at least 150, at least 620, at least 720, or at least 770 nucleotides longer than HCR-hAAT. In one embodiment, the TRE is longer than FRE72 and shorter than HCR-hAAT. In one embodiment, the TRE is longer than 898 nucleotides in length. In an embodiment, the TRE is longer than 1363 nucleotides in length.

When determining the length of a TRE, the skilled person would understand that all nucleotides that are considered to be part of a TRE should be included (for example all nucleotide corresponding to a hAAT sequence element, an AMBP sequence element, an ApoE-HCR1, an ALDOB sequence element, a CRM6 sequence element, or an F2 sequence element should be included, and so should nucleotides corresponding to any other known transcription regulatory elements). For example, if a construct comprises two copies of the same TRE, then both copies should be included when the length is calculated.

In particular, when determining the length of a TRE, the nucleotides of any contiguous portion of a polynucleotide must be counted towards the length of the TRE if the contiguous portion comprises a nucleotide sequence having:
(i) at least 95% sequence identity to any of SEQ ID NO: 66-105;
(ii) at least 95% sequence identity to any known promoter or enhancer element or fragment of any known promoter or enhancer element; and/or
(iii)at least 95% sequence identity to any known transcription factor binding site.

In one aspect, the TRE comprises or consists of:
(a) a nucleotide sequence having at least 80%, at least 85%, at least 90%, at least 95%, at least 98%, or at least 99% sequence identity to any one of SEQ ID NO: 68-91; or
(b) a nucleotide sequence of any one of SEQ ID NO: 68-91.

In one aspect, the TRE comprises or consists of:
(a) a nucleotide sequence having at least 80%, at least 85%, at least 90%, at least 95%, at least 98%, or at least 99% sequence identity to SEQ ID NO: 70; or
(b) a nucleotide sequence of SEQ ID NO: 70.

In one aspect, the TRE comprises or consists of:
(a) a nucleotide sequence having at least 80%, at least 85%, at least 90%, at least 95%, at least 98%, or at least 99% sequence identity to SEQ ID NO: 74; or
(b) a nucleotide sequence of SEQ ID NO: 74.

In one embodiment, the TRE comprises or consists of a nucleotide sequence having at least 95% sequence identity to SEQ ID NO: 68-91, In one embodiment, the TRE comprises or consists of a nucleotide sequence having at least 95% sequence identity to SEQ ID NO: 70. In one embodiment, the TRE comprises or consists of a nucleotide sequence having at least 95% sequence identity to SEQ ID NO: 74.

In one embodiment, the TRE comprises or consists of a nucleotide sequence having at least 98% sequence identity to SEQ ID NO: 68-91, In one embodiment, the TRE comprises or consists of a nucleotide sequence having at least 98% sequence identity to SEQ ID NO: 70. In one embodiment, the TRE comprises or consists of a nucleotide sequence having at least 98% sequence identity to SEQ ID NO: 74.

HCR-hAAT is a hybrid TRE derived from the human alpha-1-antitrypsin promoter and the enhancer/hepatic locus control region (HCR) of the ApoE gene. FRE72 is a TRE that comprises only the core region of the hAAT promoter. HCR-hAAT and FRE72 have been shown to promote high levels of expression of transgenes in human hepatocytes which have been transduced with a viral vector comprising an expression cassette comprising HCR-hAAT or FRE72 operably linked to said transgene.

In one aspect, the TRE promotes greater expression than HCR-hAAT. HCR-hAAT is a TRE that is described in more detail in Miao et al., Mol Ther. 2000;1(6): 522-532, and has a sequence of SEQ ID NO: 67. The TRE may promote expression that is at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3 fold higher compared to HCR-hAAT. In one embodiment, the TRE promotes expression at least 1.8 fold higher compared to HCR-hAAT.

In an aspect, the TRE promotes expression that is between 1.2 fold and 3 fold, between 1.5 fold and 3 fold, between 1.8 fold and 3 fold, between 2 fold and 3 fold, between 2.5 fold and 3 fold, between 1.2 fold and 5 fold, between 1.5 fold and 5 fold, between 1.8 fold and 5 fold, between 2 fold and 5 fold, between 2.5 fold and 5 fold, or between 3 fold and 5 fold higher compared to HCR-hAAT. In one embodiment, the TRE promotes expression between 2 and 3 fold higher compared to HCR-hAAT.

In an aspect, the TRE promotes greater expression than FRE72. FRE72 is a promoter that is described in more detail in WO2021/084277 and has a sequence of SEQ ID NO: 66. The TRE may promote expression that is at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3 fold higher compared to FRE72. In one embodiment, the TRE promotes expression at least 1.8 fold higher compared to FRE72.

In an aspect, the TRE which promotes expression that is between 1.2 fold and 3 fold, between 1.5 fold and 3 fold, between 1.8 fold and 3 fold, between 2 fold and 3 fold, between 2.5 fold and 3 fold, between 1.2 fold and 5 fold, between 1.5 fold and 5 fold, between 1.8 fold and 5 fold, between 2 fold and 5 fold, between 2.5 fold and 5 fold, or between 3 fold and 5 fold higher compared to FRE72. In one embodiment, the TRE promotes expression between 2 and 3 fold higher compared to FRE72.

The skilled person will understand that there are many suitable methods for determining the level of expression promoted by a TRE. A suitable expression assay is described in the Example and below.

Optionally, expression is measured by an expression assay comprising:
(i) transfecting human hepatocytes with a plasmid comprising an expression cassette comprising a transgene operably linked to the TRE;
(ii) incubating the transfected cells under conditions suitable for transgene expression to occur; and
(iii) measuring the level of the transgene by ELISA using an antibody specific for a protein encoded by the transgene.

Optionally, the transgene encodes a wild-type human CFI polypeptide. Optionally, transfection step (i) comprises mixing the plasmid with a transfection reagent (such as FUGENE®HD (Promega, cat no.E2311) to form a transfection mixture, and delivering the transfection mixture to the hepatocytes. Optionally, the hepatocytes are Huh7 cells. The step of transfecting human hepatocytes may be a step of incubating the human hepatocytes with the transfection mixture overnight. The step of incubating the transfected cells may be a step of growing the transfected cells in suitable growth conditions for 5 days. The step of measuring the level of the transgene may comprise harvesting cells and media produced by the step of incubating the transfected cells after the step of incubating the transfected cells.

Optionally, transfection step (i) comprises co-transfecting the human hepatocytes with a plasmid comprising an expression cassette comprising a transgene encoding luciferase operably linked to a promoter and measuring total luciferase expression.

The step of measuring the level of the transgene by ELISA may be performed using a sandwich ELISA using two antibodies that bind to CFI (for example, using the CFI ELISA kit (e.g. Hycult cat# HK355-01) according to the manufacturer’s instructions). For example, a first antibody that binds to CFI may be immobilised to a plate, and a portion of the cells and media that were optionally harvested as part of the step of measuring the level of the transgene may be applied to the plate with the first antibody immobilised on it. The plate may be washed, and then a second antibody that binds to CFI and is conjugated to a label may be applied to the plate. The second antibody may be conjugated to biotin, which binds to streptavidin-peroxidase, and in this case the streptavidin-peroxidase acts as the label. The plate may be washed again, and the amount of CFI bound to the plate measured by assessing the amount of the label present. For example, if the label is streptavidin-peroxidase, the substrate tetramethylbenzidine (TMB) may be added, which the streptavidin-peroxidase reacts with. The enzyme reaction may be stopped by adding oxalic acid. For each of the samples and standards, the absorbance at 450 nm may be measured using a spectrophotometer. A standard curve may be obtained by plotting the absorbance (linear) against the corresponding concentrations of human CFI standards. The concentration of human CFI in the samples may then be determined from the standard curve.

The measured expression may be compared to the expression measured when a different (reference) TRE is used, for example HCR-hAAT or FRE72. In such cases the above steps are repeated using the reference TRE in place of the TRE in a polynucleotide of the invention (the ‘test” TRE) (i.e. the user repeats the same steps, but inserts the reference TRE into the plasmid used in transfection step (i) in place of the test TRE).

As mentioned above, transfection step (i) may comprise co-transfecting the human hepatocytes with a plasmid comprising an expression cassette comprising a transgene encoding luciferase operably linked to a promoter and measuring the total luciferase expression. The plasmid comprising an expression cassette comprising a transgene encoding luciferase may be identical to the plasmid comprising an expression cassette comprising a transgene operably linked to the TRE (except that the transgenes are different and the TREs may be different).This allows the users to normalise the measured expression to reflect the number of cells that have been transfected (thereby accounting for differences in transfection efficiency), as the proportion of cells that are transfected by the vector (i.e. the transfection efficiency) will be proportionate to the total luciferase expression. In such cases, the expression assay further comprises a step of detecting the total luciferase expression. Optionally, the luciferase is operably linked to a CMV promoter.

The total luciferase expression may be measured by performing a luciferase assay on a portion of the cells that were optionally harvested as part of the step of measuring the level of transgene. The total luciferase expression may be measured by measuring luminescence using a luminometer. For example, the cells may be washed with phosphate buffered saline (PBS) twice and then treated with 100µl of luciferase lysis buffer from the Luciferase assay kit (Promega cat# E1501/4530). Luciferase expression may be measured by measuring luminescence on a Molecular Devices SpextraMax i3x plate reader.

Optionally, the expression assay comprises calculating a normalised value for expression by dividing the level of the transgene measured by ELISA by the total luciferase expression from the corresponding cells.

Optionally, the TRE is liver-specific. A TRE is “liver-specific” if it drives a higher level of expression in liver cells compared to other cells in general. For example, the skilled person can determine whether a TRE is a liver-specific TRE by comparing expression of the polynucleotide in liver cells (such as Huh 7 cells) with expression of the polynucleotide in cells from other tissues (such as kidney cells, for example HEK293T cells). If the level of expression is higher in the liver cells, compared to the cells from other tissues, the TRE is a liver-specific TRE. Optionally, a liver-specific TRE does not drive an appreciable level of expression in non-liver cells.

In one aspect, the TRE comprises a hAAT sequence element. A hAAT sequence element is a polynucleotide derived from the human alpha-1-antitrypsin promoter (hAAT promoter). An exemplary sequence of the hAAT promoter is given as SEQ ID NO: 94.

In one embodiment, the TRE comprises a hAAT sequence element wherein the hAAT sequence element comprises a nucleotide sequence of SEQ ID NO: 95 or a variant of SEQ ID NO: 95 that differs by 1, 2, or 3 nucleotides. In one embodiment, the TRE comprises a hAAT sequence element wherein the hAAT sequence element comprises a nucleotide sequence of SEQ ID NO: 95. In one embodiment, the TRE comprises a hAAT sequence element wherein the hAAT sequence element comprises a nucleotide sequence of SEQ ID NO: 96 or a variant of SEQ ID NO: 96 that differs by 1, 2, or 3 nucleotides. In one embodiment, the TRE comprises a hAAT sequence element wherein the hAAT sequence element comprises a nucleotide sequence of SEQ ID NO: 96.

In one embodiment, the TRE comprises a hAAT sequence element wherein the hAAT sequence element comprises a nucleotide sequence at least 80%, at least 85%, at least 90%, at least 95%, at least 98%, or at least 99% identical to SEQ ID NO: 94 or a fragment of SEQ ID NO: 94 that is at least 200, at least 300, or at least 400 nucleotides long. In one embodiment, the TRE comprises a hAAT sequence element wherein the hAAT sequence element comprises a nucleotide sequence at least 98% identical to SEQ ID NO: 94. In one embodiment, the TRE comprises a hAAT sequence element wherein the hAAT sequence element comprises a nucleotide sequence of SEQ ID NO: 94. In one embodiment, the TRE comprises a second hAAT sequence element.

In one embodiment, the TRE comprises a first and a second hAAT sequence element, wherein the second hAAT sequence element is 5′ of the first hAAT sequence element and 5′ of the AMBP sequence element. In one embodiment, the second hAAT sequence element comprises a nucleotide sequence of SEQ ID NO: 95 or a variant of SEQ ID NO: 95 that differs by 1, 2, or 3 nucleotides. In one embodiment, the second hAAT sequence element comprises a nucleotide sequence of SEQ ID NO: 95. In embodiment, the first hAAT sequence element comprises SEQ ID NO: 94 and the second hAAT sequence element comprises SEQ ID NO: 95.

In one aspect, the TRE comprises an AMBP sequence element. An AMBP sequence element is a polynucleotide derived from the human alpha1-microglobulin/bikunin enhancer (AMBP enhancer). The AMBP enhancer is further described in Rouet et al., Journal of Biological Chemistry, 1992, 267; 20765-20773. An exemplary sequence for the AMBP enhancer is given as SEQ ID NO: 097.

In one embodiment, the TRE comprises an AMBP sequence element wherein the AMBP sequence element comprises a nucleotide sequence of SEQ ID NO: 98 or a variant of SEQ ID NO: 98 that differs by 1, 2, or 3 nucleotides. In one embodiment, the TRE comprises an AMBP sequence element wherein the AMBP sequence element comprises a nucleotide sequence of SEQ ID NO: 98. In one embodiment, the TRE comprises an AMBP sequence element wherein the AMBP sequence element comprises a nucleotide sequence of SEQ ID NO: 97 or a variant of SEQ ID NO: 97 that differs by 1, 2, or 3 nucleotides. In one embodiment, the TRE comprises an AMBP sequence element wherein the AMBP sequence element comprises a nucleotide sequence of SEQ ID NO: 97.

An AMBP sequence element may comprise binding sites for one or more transcription factors. Accordingly, in one aspect, the AMBP sequence element comprises at least one binding site selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises at least two binding sites selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises at least three binding sites selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises at least four binding sites selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises an HNF1-1 binding site, an HNF4 binding site, an HNF3a binding site, an HNF1-2 binding site, and an HNF3-2 binding site.

In one aspect, the AMBP sequence element is 5′ of a hAAT sequence element. Optionally, the AMBP sequence element is less than 10, less than 8, or less than 5 nucleotides 5′ of the hAAT sequence element. Optionally, the AMBP sequence element is immediately 5′ of the hAAT sequence element.

In one aspect, a TRE comprising the AMBP sequence element promotes equivalent or greater expression than a corresponding TRE lacking an AMBP sequence element. The skilled person can determine whether a TRE comprising an AMBP sequence element (a test TRE) promotes equivalent or greater expression than a second TRE not comprising an AMBP sequence element (a reference TRE) by determining the level of expression promoted by the test TRE comprising an AMBP sequence element and the reference TRE using the techniques described under the heading “expression assay”. Optionally, the user determines whether a TRE comprising an AMBP sequence element (the test AMBP sequence element) promotes equivalent or greater expression than a reference TRE not comprising an AMBP sequence element by creating a test TRE that comprises the test AMBP sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an AMBP sequence element.

In one aspect, the TRE comprises an ApoE-HCR1 sequence element. An ApoE-HCR1 sequence element is a polynucleotide derived from the hepatic locus control region (HCR) of the ApoE gene. The ApoE-HCR1 is described further in Miao et al (2000), Molecular Therapy 1(6):522. An exemplary sequence for the ApoE-HCR1 is given as SEQ ID NO: 92.

In one embodiment, the TRE comprises an ApoE-HCR1 sequence element wherein the ApoE-HCR1 sequence element comprises a nucleotide sequence of SEQ ID NO: 93 or a variant of SEQ ID NO: 93 that differs by 1, 2, or 3 nucleotides. In one embodiment, the TRE comprises an ApoE-HCR1 sequence element wherein the ApoE-HCR1 sequence element comprises a nucleotide sequence of SEQ ID NO: 93. In one embodiment, the TRE comprises an ApoE-HCR1 sequence element wherein the ApoE-HCR1 sequence element comprises a nucleotide sequence of SEQ ID NO: 92 or a variant of SEQ ID NO: 92 that differs by 1, 2, or 3 nucleotides. In one embodiment, the TRE comprises an ApoE-HCR1 sequence element wherein the ApoE-HCR1 sequence element comprises a nucleotide sequence of SEQ ID NO: 92.

In one aspect, the ApoE-HCR1 sequence element is 5′ of a hAAT sequence element. Optionally, the ApoE-HCR1 sequence element is less than 10, less than 8, or less than 5 nucleotides 5′ of the hAAT sequence element. Optionally, the ApoE-HCR1 sequence element is immediately 5′ of the hAAT sequence element. In one aspect, the ApoE-HCR1 sequence element is 5′ of an AMBP sequence element. Optionally, the ApoE-HCR1 sequence element is less than 10, less than 8, or less than 5 nucleotides 5′ of the AMBP sequence element. Optionally, the ApoE-HCR1 sequence element is immediately 5′ of the AMBP sequence element. In an aspect, the ApoE-HCR1 sequence element is 3′ of an AMBP sequence element. Optionally, the ApoE-HCR1 sequence element is less than 10, less than 8, or less than 5 nucleotides 5′ of the AMBP sequence element. Optionally, the ApoE-HCR1 sequence element is immediately 5′ of the AMBP sequence element. In an aspect, the ApoE-HCR1 sequence element is 5′ of both an hAAT sequence element and an AMBP sequence element. In an aspect, the ApoE-HCR1 sequence element is 5′ of a hAAT sequence element and 3′ of an AMBP sequence element.

In one aspect, a TRE comprising the ApoE-HCR1 sequence element promotes equivalent or greater expression than a corresponding TRE lacking an ApoE-HCR1 sequence element. The skilled person can determine whether a TRE comprising an ApoE-HCR1 sequence element (a test TRE) promotes equivalent or greater expression than a second TRE not comprising an ApoE-HCR1 sequence element (a reference TRE) by determining the level of expression promoted by the test TRE comprising an ApoE-HCR1 and the reference TRE using the techniques described under the heading “expression assay”. Optionally, the user determines whether a TRE comprising an ApoE-HCR1 sequence element (the test ApoE-HCR1 sequence element) promotes equivalent or greater expression than a reference TRE not comprising an ApoE-HCR1 sequence element by creating a test TRE that comprises the test ApoE-HCR1 sequence element 5′ of hAAT and comparing the level of expression of the test TRE with that of hAAT (the reference TRE). If the level of expression of the test TRE is equivalent or higher than the level of expression of hAAT, then the TRE promotes equivalent or greater expression than a corresponding TRE not comprising an ApoE-HCR1 sequence element.

In one aspect, the TRE comprises a CRM6 sequence element. A CRM6 sequence element is a polynucleotide derived from CRM6. CRM6 is described in further detail in Chuah et al., Molecular Therapy, 2014, 22; 1605-1613. An exemplary sequence for CRM6 is given as SEQ ID NO: 99.

In one embodiment, the TRE comprises a CRM6 sequence element wherein the CRM6 sequence element comprises a nucleotide sequence of SEQ ID NO: 100 or a variant of SEQ ID NO: 100 that differs by 1, 2, or 3 nucleotides. In one embodiment, the TRE comprises a CRM6 sequence element wherein the CRM6 sequence element comprises a nucleotide sequence of SEQ ID NO: 100. In one embodiment, the TRE comprises a CRM6 sequence element wherein the CRM6 sequence element comprises a nucleotide sequence of SEQ ID NO: 101 or a variant of SEQ ID NO: 101 that differs by 1, 2, or 3 nucleotides. In one embodiment, the TRE comprises a CRM6 sequence element wherein the CRM6 sequence element comprises a nucleotide sequence of SEQ ID NO: 101. In one embodiment, the TRE comprises a CRM6 sequence element wherein the CRM6 sequence element comprises a nucleotide sequence of SEQ ID NO: 99 or a variant of SEQ ID NO: 99 that differs by 1, 2, or 3 nucleotides. In one embodiment, the TRE comprises a CRM6 sequence element wherein the CRM6 sequence element comprises a nucleotide sequence of SEQ ID NO: 99.

A CRM6 sequence element may comprise binding sites for one or more transcription factors. Accordingly, in one aspect, the CRM6 sequence element comprises at least one binding site selected from the group consisting of an Sp1 binding site, an Sp2 binding site, an HNF3A binding site, and an HNF-1 binding site. In an aspect, the CRM6 sequence element comprises at least two binding sites selected from the group consisting of an Sp1 binding site, an Sp2 binding site, an HNF3A binding site, and an HNF-1 binding site. In an aspect the CRM6 sequence element comprises at least three binding sites selected from the group consisting of an Sp1 binding site, an Sp2 binding site, an HNF3A binding site, and an HNF-1 binding site. In an aspect, the TRE comprises a CRM6 sequence element, wherein the CRM6 sequence element comprises an Sp1 binding site, an Sp2 binding site, an HNF3A binding site, and an HNF-1 binding site.

In one aspect, the CRM6 sequence element is 5′ of a hAAT sequence element. Optionally, the CRM6 sequence element is less than 10, less than 8, or less than 5 nucleotides 5′ of the hAAT sequence element. Optionally, the CRM6 sequence element is immediately 5′ of the hAAT sequence element. In one aspect, the CRM6 sequence element is 5′ of an AMBP sequence element. Optionally, the CRM6 sequence element is less than 10, less than 8, or less than 5 nucleotides 5′ of the AMBP sequence element. Optionally, the CRM6 sequence element is immediately 5′ of the AMBP sequence element. In an aspect, the CRM6 sequence element is 3′ of an AMBP sequence element. Optionally, the CRM6 sequence element is less than 10, less than 8, or less than 5 nucleotides 3′ of the AMBP sequence element. Optionally, the CRM6 sequence element is immediately 3′ of the AMBP sequence element. In an aspect, the CRM6 sequence element is 5′ of both an hAAT sequence element and an AMBP sequence element. In an aspect, the CRM6 sequence element is 5′ of a hAAT sequence element and 3′ of an AMBP sequence element.

In one aspect, a TRE comprising the CRM6 sequence element promotes equivalent or greater expression than a corresponding TRE lacking an CRM6 sequence element. The skilled person can determine whether a TRE comprising an CRM6 sequence element (a test TRE) promotes equivalent or greater expression than a second TRE not comprising an CRM6 sequence element (a reference TRE) by determining the level of expression promoted by the test TRE comprising a CRM6 sequence element and the reference TRE using the techniques described under the heading “expression assay”. Optionally, the user determines whether a TRE comprising an CRM6 sequence element (the test CRM6 sequence element) promotes equivalent or greater expression than a reference TRE not comprising an CRM6 sequence element by creating a test TRE that comprises the test CRM6 sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an CRM6 sequence element.

In one aspect, the TRE comprises an ALDOB sequence element. An ALDOB sequence element is a polynucleotide derived from the enhancer of the aldolase B gene. The ALDOB enhancer is described in further detail in Gregori 2002 and Patwardhan et al. 2012. An exemplary sequence for ALDOB is given as SEQ ID NO: 102.

In one embodiment, the TRE comprises an ALDOB sequence element wherein the ALDOB sequence element comprises a nucleotide sequence of SEQ ID NO: 103 or a variant of SEQ ID NO: 103 that differs by 1, 2, or 3 nucleotides. In one embodiment, the TRE comprises an ALDOB sequence element wherein the ALDOB sequence element comprises a nucleotide sequence of SEQ ID NO: 103. In one embodiment, the TRE comprises an ALDOB sequence element wherein the ALDOB sequence element comprises a nucleotide sequence of SEQ ID NO: 102 or a variant of SEQ ID NO: 102 that differs by 1, 2, or 3 nucleotides. In one embodiment, the TRE comprises an ALDOB sequence element wherein the ALDOB sequence element comprises a nucleotide sequence of SEQ ID NO: 102.

An ALDOB sequence element may comprise binding sites for one or more transcription factors. Accordingly, in one aspect, the ALDOB sequence element comprises at least one binding site selected from the group consisting of an SP1 binding site, an HNF1-a binding site, a C/EBP binding site, a GATA2 binding site, a USF1 binding site, and a USF2 binding site. In an aspect, the ALDOB sequence element comprises at least two binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least three binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least four binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least five binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least one SP1 binding site, at least one HNF1-a binding site, at least one C/EBP binding site, at least one GATA2 binding site, at least one USF1 binding site, and at least one USF2 binding site.

In one aspect, the ALDOB sequence element is 5′ of a hAAT sequence element. Optionally, the ALDOB sequence element is less than 10, less than 8, or less than 5 nucleotides 5′ of the hAAT sequence element. Optionally, the ALDOB sequence element is directly 5′ of the hAAT sequence element. In one aspect, the ALDOB sequence element is 5′ of an AMBP sequence element. Optionally, the ALDOB sequence element is less than 10, less than 8, or less than 5 nucleotides 5′ of the AMBP sequence element. Optionally, the ALDOB sequence element is immediately 5′ of the AMBP sequence element. In an aspect, the ALDOB sequence element is 3′ of an AMBP sequence element. Optionally, the ALDOB sequence element is less than 10, less than 8, or less than 5 nucleotides 3′ of the AMBP sequence element. Optionally, the ALDOB sequence element is immediately 3′ of the AMBP sequence element. In an aspect, the ALDOB sequence element is 5′ of both an hAAT sequence element and an AMBP sequence element. In an aspect, the ALDOB sequence element is 5′ of a hAAT sequence element and 3′ of an AMBP sequence element.

In one aspect, a TRE comprising the ALDOB sequence element promotes equivalent or greater expression than a corresponding TRE lacking an ALDOB sequence element. The skilled person can determine whether a TRE comprising an ALDOB sequence element (a test TRE) promotes equivalent or greater expression than a second TRE not comprising an ALDOB sequence element (a reference TRE) by determining the level of expression promoted by the test TRE comprising an AMBP sequence and the reference TRE using the techniques described under the heading “expression assay”. Optionally, the user determines whether a TRE comprising an ALDOB sequence element (the test ALDOB sequence element) promotes equivalent or greater expression than a reference TRE not comprising an ALDOB sequence element by creating a test TRE that comprises the test ALDOB sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an ALDOB sequence element.

In one aspect, the TRE comprises an F2 sequence element. An F2 sequence element is a polynucleotide derived from the enhancer of the prothrombin gene. An exemplary sequence for the prothrombin enhancer is given as SEQ ID NO: 104.

In one embodiment, the TRE comprises an F2 sequence element wherein the F2 sequence element comprises a nucleotide sequence of SEQ ID NO: 105 or a variant of SEQ ID NO: 105 that differs by 1, 2, or 3 nucleotides. In one embodiment, the TRE comprises an F2 sequence element wherein the F2 sequence element comprises a nucleotide sequence of SEQ ID NO: 105. In one embodiment, the TRE comprises an F2 sequence element wherein the F2 sequence element comprises a nucleotide sequence of SEQ ID NO: 104 or a variant of SEQ ID NO: 104 that differs by 1, 2, or 3 nucleotides. In one embodiment, the TRE comprises an F2 sequence element wherein the F2 sequence element comprises a nucleotide sequence of SEQ ID NO: 104.

An F2 sequence element may comprise binding sites for one or more transcription factors. Accordingly, in one aspect, the F2 sequence element comprises at least one binding site selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least two binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least three binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least four binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least five binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least six binding sites selected from the group consisting of HNF4a binding site, an HNF3a binding site, an HNF1a binding site, an HNF3b binding site, an STAT3 binding site, an RBPJ binding site, and an SP3 binding site. In an aspect, the TRE comprises an F2 sequence element, wherein the F2 sequence element comprises an HNF4a binding site, an HNF3a binding site, an HNF1a binding site, an HNF3b binding site, an STAT3 binding site, an RBPJ binding site, and an SP3 binding site.

In one aspect, the F2 sequence element is 5′ of a hAAT sequence element. Optionally, the F2 sequence element is less than 10, less than 8, or less than 5 nucleotides 5′ of the hAAT sequence element. Optionally, the F2 sequence element is immediately 5′ of the hAAT sequence element. In one aspect, the F2 sequence element is 5′ of an AMBP sequence element. Optionally, the F2 sequence element is less than 10, less than 8, or less than 5 nucleotides 5′ of the AMBP sequence element. Optionally, the F2 sequence element is immediately 5′ of the AMBP sequence element. In an aspect, the F2 sequence element is 3′ of an AMBP sequence element. Optionally, the F2 sequence element is less than 10, less than 8, or less than 5 nucleotides 3′ of the AMBP sequence element. Optionally, the F2 sequence element is immediately 5′ of the AMBP sequence element. In an aspect, the F2 sequence element is 5′ of both an hAAT sequence element and an AMBP sequence element. In an aspect, the F2 sequence element is 5′ of a hAAT sequence element and 3′ of an AMBP sequence element.

In one aspect, a TRE comprising the F2 sequence element promotes equivalent or greater expression than a corresponding TRE lacking an F2 sequence element. The skilled person can determine whether a TRE comprising an F2 sequence element (a test TRE) promotes equivalent or greater expression than a second TRE not comprising an F2 sequence element (a reference TRE) by determining the level of expression promoted by the test TRE comprising an F2 sequence element and the reference TRE using the techniques described under the heading “expression assay. Optionally, the user determines whether a TRE comprising an F2 sequence element (the test F2 sequence element) promotes equivalent or greater expression than a reference TRE not comprising an F2 sequence element by creating a test TRE that comprises the test F2 sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an F2 sequence element.

In one embodiment, the TRE (such as NP1) comprises:
(i) an AMBP sequence element; and
(ii) a hAAT sequence element, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the AMBP sequence element is 5′ of the hAAT sequence element; and
   (iv) the TRE is between 550 and 600 nucleotides in length.

In one embodiment, the TRE (such as NP2) comprises:
(i) an AMBP sequence element;
(ii) a hAAT sequence element; and
(iii) an ApoE-HCR1 sequence element, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of both the AMBP sequence element and the hAAT sequence element; and
   (vi) the TRE is between 875 and 925 nucleotides in length.

In one embodiment, the TRE (such as NP3) comprises:
(i) an AMBP sequence element;
(ii) a hAAT sequence element; and
(iii) an ApoE-HCR1 sequence element, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element; and
   (vi) the TRE is between 875 and 925 nucleotides in length.

In one embodiment, the TRE (such as NP4) comprises:
(i) an AMBP sequence element;
(ii) a hAAT sequence element; and
(iii) an F2 sequence element, wherein:
   (i) the AMBP sequence element comprises a nucleotide sequence of SEQ ID NO: 97;
   (ii) the hAAT sequence element comprises a fragment of SEQ ID NO: 94 that is between 300 and 350 nucleotides in length;
   (iii) the hAAT sequence element comprises a nucleotide sequence of SEQ ID NO: 95;
   (iv) the F2 sequence element comprises a nucleotide sequence of SEQ ID NO: 104;
   (v) the AMBP sequence element is 5′ of the hAAT sequence element;
   (vi) the F2 sequence element is 5′ of both the AMBP sequence element and the hAAT sequence element; and
   (vii) the TRE is between 550 and 600 nucleotides in length.

In one embodiment, the TRE (such as NP5) comprises:
(i) an AMBP sequence element;
(ii) a hAAT sequence element;
(iii) an ApoE-HCR1 sequence element;
(iv) a CRM6 sequence element; and
(v) an ALDOB sequence element, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the CRM6 sequence element comprises a nucleotide sequence of SEQ ID NO: 99;
   (v) the ALDOB sequence element comprises a nucleotide sequence of SEQ ID NO: 102;
   (vi) the AMBP sequence element is 5′ of the hAAT sequence element;
   (vii) the ApoE-HCR1 sequence element is 5′ of both the AMBP sequence element and the hAAT sequence element;
   (viii) the CRM6 sequence element is 5′ of the AMBP sequence element, the hAAT sequence element, and the ApoE-HCR1 sequence element;
   (ix) the ALDOB sequence element is 5′ of the hAAT sequence element and 3′ of the AMBP sequence element; and
   (x) the TRE is between 1600 and 1650 nucleotides in length.

In one embodiment, the TRE (such as NP6) comprises:
(i) an AMBP sequence element;
(ii) a first hAAT sequence element;
(iii) a second hAAT sequence element;
(iv) an F2 sequence element; and
(v) an ALDOB sequence element, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the second hAAT sequence element comprises a nucleotide sequence of SEQ ID NO: 95;
   (iv) the F2 sequence element comprises a nucleotide sequence of SEQ ID NO: 104;
   (v) the ALDOB sequence element comprises a nucleotide sequence of SEQ ID NO: 102;
   (vi) the AMBP sequence element is 5′ of the hAAT sequence element;
   (vii) the second hAAT sequence element is 5′ of both the AMBP sequence element and the hAAT sequence element;
   (viii) the F2 sequence element is 5′ of the AMBP sequence element, the hAAT sequence element, and the second hAAT sequence element;
   (ix) the ALDOB sequence element is 5′ of the hAAT sequence element, the AMBP sequence element, the second hAAT sequence element, and the F2 sequence element; and
   (x) the TRE is between 1350 and 1400 nucleotides in length.

In one embodiment, the TRE (such as NP7) comprises:
(i) an AMBP sequence element;
(ii) a hAAT sequence element;
(iii) an ApoE-HCR1 sequence element; and
(iv) an ALDOB sequence element, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the ALDOB sequence element comprises a nucleotide sequence of SEQ ID NO: 102;
   (v) the AMBP sequence element is 5′ of the hAAT sequence element;
   (vi) the ApoE-HCR1 sequence element is 5′ of both the AMBP sequence element and the hAAT sequence element;
   (vii) the ALDOB sequence element is 5′ of the hAAT sequence element, the AMBP sequence element, and the ApoE-HCR1 sequence element; and
   (viii) the TRE is between 1450 and 1500 nucleotides in length.

A transcription factor binding site is a nucleotide sequence to which a transcription factor can bind. Binding of a transcription factor to a transcription factor binding site may result in changes to the expression level of the polynucleotide comprises said binding site. Table 1 provides exemplary sequences for several transcription factor binding sites, but the skilled person will understand that this list is non-exhaustive and transcription factors may bind to alternative sites.

| TF Binding Site | Sequence | SEQ ID NO |
|---|---|---|
| Alb | TAGACTAATATTTG | 106 |
| C/EBP | CTTGCAAAA | 107 |
| | TATGTACATT | 108 |
| | CCCACAACAT | 109 |
| | TTGAGAAAT | 110 |
| | GATTCATCTC | 111 |
| | TTGGTCTAAC | 112 |
| | CTTTCTACCTTG | 113 |
| | CTGTTTGCTC | 114 |
| C/EBP alpha | CTTGCAAAA | 115 |
| | CTGTTTGCTC | 116 |
| CTCT | CCCAGGTGT | 117 |
| CUX2 | TATTGATT | 118 |
| DUX4 | AAAAGCAGTCA | 119 |
| E2F6 | AGGCAGGAGAA | 120 |
| | GAGAAGAAATC | 121 |
| ELK4 | CAACATCCTGG | 122 |
| | ACTTATCCTCT | 123 |
| | CGTGTTCCTGC | 124 |
| FOX | GCAAACAGG | 125 |
| | TCATGTT | 126 |
| FOXD1 | ATCAACAT | 127 |
| GATA1 | ATAATCTCAGGAGCACAA | 128 |
| GATA2 | TTATCT | 129 |
| | TGATAG | 130 |
| | AGATAG | 131 |
| | GACTTATCCTC | 132 |
| HNF1 | TGTTTATTCAACACCTCT | 133 |
| | GTTAATAATC | 134 |
| HNF1a | GACTAATATTTGC | 135 |
| | CTTGCAAAATTAT | 136 |
| | GCTGCTAATTTTT | 137 |
| | ACAAATAATTAAC | 138 |
| HNF1-1 | GTTAATTTTTAAA | 139 |
| HNF1-2 | GGTTAATAATC | 140 |
| HNF1b | TGGTTAATATTC | 141 |
| HNF3 | TGTTTGCTCT | 142 |
| | AGCACAAACA | 143 |
| | TGTTTGCTC | 144 |
| HNF3a | TGTTTGC | 145 |
| | TGTTTGCT | 146 |
| | AGAAATCAACA | 147 |
| | CTAATATTTGCCTTG | 148 |
| HNF3b | GACTAATATTTGC | 149 |
| | TCTGTTTGCTCT | 150 |
| HNF4a | TTCCTGCTCTTTGTCCC | 151 |
| | CAGAGTTT | 152 |
| | CCCTGTTTGCTC | 153 |
| | TGGCCCTTGC | 154 |
| HNF4g | AGCAGTCAAAAGTCC | 155 |
| HNF6 | TTATTGATATTC | 156 |
| | TATTGATT | 157 |
| IRF | TTTTTTTTCCAATAT | 158 |
| | ATTTACTCTCTCTGTTTGCTC | 159 |
| | TTACTCTCTCTGTTTGCTCTG | 160 |
| JUN | TTAATAATCTCAG | 161 |
| LF-B1 | TGGTTAATATTCACCAG | 162 |
| KLF4 | CAACATCCTGG | 163 |
| MXL1 | AGTCCAAGTGGCC | 164 |
| MYC | GTCCAAGTGGCC | 165 |
| MZF1 | CTCTGGTTAA | 166 |
| | AAATGGGGGA | 167 |
| NF-kB | TCAAAAGTCC | 168 |
| NHF1 | TAGACTAATATTTG | 169 |
| NR2C2 | CAGGAGAAGAAATCA | 170 |
| Oct-1 | TGTTAATTAAAAGC | 171 |
| POU6F1 | GTTAATTTTT | 172 |
| PRDM1 | TTACTCTCTC | 173 |
| RBPJ | CTTGGGTACT | 174 |
| Serpina | GCAAACAGG | 175 |
| SHOX | TTAATTTT | 176 |
| SP1 | GGGATTCA | 177 |
| | GGTACTGT | 178 |
| | TTAATCCC | 179 |
| | GGTATTGA | 180 |
| | GGCTCTAA | 181 |
| | GGCATGAT | 182 |
| | TTCAAACC | 183 |
| | ATAGAACC | 184 |
| | TGGAGGCAGG | 185 |
| SPIB | ACAGGAA | 186 |
| SPL1/SP3 | ATGGGGGAGGGAC | 187 |
| SRY | GCAGTCAAA | 188 |
| | GAGCACAAA | 189 |
| STAT1 | ATTCCTGGAGG | 190 |
| STAT3 | CAAACAGGAAA | 191 |
| TATA Box | TATAAA | 192 |
| | TATATAAA | 193 |
| TEAD2 | AAACATTCCTGGA | 194 |
| USF1 | ACGTGTG | 195 |
| | CAGGTGTCA | 196 |
| | CAGATGTCA | 197 |
| | TCCAAGTGGCC | 198 |
| USF1/2 | AGCATGTGATA | 199 |

In an aspect, the TRE comprises one or more transcription factor binding sites. In an aspect, the TRE comprises
a) a TATA box;
b) an HNF6 binding site;
c) an HNF4a binding site;
d) an HNF1 binding site; and/or
e) an HNF3 binding site.

In an aspect, the TRE comprises a TATA box, optionally a canonical TATA box, optionally wherein the TRE comprises a hAAT sequence element and the TATA box is inserted into the hAAT sequence element.

A canonical TATA box is an AT-rich nucleotide sequence, capable of binding TATA-binding protein (TBP), which comprises the nucleotide sequence TATA, for example TATAAAA (SEQ ID NO: 200) is a canonical TATA box. A non-canonical TATA box is an AT-rich nucleotide sequence, capable of binding TATA-binding protein (TBP), which does not comprise the nucleotide sequence TATA, for example TTAAATA (SEQ ID NO: 201). A hAAT sequence element may comprise a non-canonical TATA box. The TRE may comprise a hAAT sequence element, wherein the hAAT sequence element comprises a non-canonical TATA box.

The hAAT sequence element may comprise a nucleotide sequence of SEQ ID NO: 95 or a variant of SEQ ID NO: 95 that differs by 1, 2, or 3 nucleotides and the TATA box may be 3′ of the nucleotide sequence of SEQ ID NO: 95 or variant of SEQ ID NO: 95. The hAAT sequence element may comprise a nucleotide sequence of SEQ ID NO: 95 or a variant of SEQ ID NO: 95 that differs by 1, 2, or 3 nucleotides and the TATA box may be 5′ of the nucleotide sequence of SEQ ID NO: 95 or variant of SEQ ID NO: 95.

The TRE may promote equivalent or greater expression than a corresponding TRE not comprising the TATA box.

In one aspect, a TRE comprising the TATA box promotes equivalent or greater expression than a corresponding TRE lacking a TATA box. The skilled person can determine whether a TRE comprising a TATA box (a test TRE) promotes equivalent or greater expression than a second TRE not comprising a TATA box (a reference TRE) by determining the level of expression promoted by the test TRE comprising a TATA box and the reference TRE using the techniques described under the heading “expression assay”. Optionally, the user determines whether a TRE comprising a TATA box (the test TATA box) promotes equivalent or greater expression than a reference TRE not comprising a TATA box by creating a test TRE that comprises the test TATA box 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising a TATA box.

In an aspect, the TRE comprises an HNF6 binding site. Optionally, the HNF6 binding site has a nucleotide sequence of SEQ ID NO: 156 or 157.

In an aspect, the TRE comprises an HNF6 binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF6 binding site is 5′ of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 96 or a variant of SEQ ID NO: 96 that differs by 1, 2, or 3 nucleotides and the HNF6 binding site is 5′ of the hAAT sequence element that comprises a nucleotide sequence of SEQ ID NO: 96 or a variant of SEQ ID NO: 96 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF6 binding site is 5′ of the hAAT sequence element that comprises a nucleotide sequence of SEQ ID NO: 94 or a variant of SEQ ID NO: 94 that differs by 1, 2, or 3 nucleotides; and/or
d. the TRE comprises a hAAT sequence element that comprises a nucleotide sequence of SEQ ID NO: 95 or a variant of SEQ ID NO: 95 that differs by 1, 2, or 3 nucleotides and the HNF6 binding site is 3′ of the hAAT sequence element that comprises a nucleotide sequence of SEQ ID NO: 95 or a variant of SEQ ID NO: 95 that differs by 1, 2, or 3 nucleotides; and/or
e. the TRE comprises a hAAT sequence element that comprises a nucleotide sequence of SEQ ID NO: 96 or a variant of SEQ ID NO: 96 that differs by 1, 2, or 3 nucleotides and the HNF6 binding site is 3′ of the hAAT sequence element that comprises a nucleotide sequence of SEQ ID NO: 96 or a variant of SEQ ID NO: 96 that differs by 1, 2, or 3 nucleotides; and/or
f. the TRE comprises a hAAT sequence element that comprises a nucleotide sequence of SEQ ID NO: 94 or a variant of SEQ ID NO: 94 that differs by 1, 2, or 3 nucleotides and the HNF6 binding site is 3′ of the hAAT sequence element that comprises a nucleotide sequence of SEQ ID NO: 94 or a variant of SEQ ID NO: 94 that differs by 1, 2, or 3 nucleotides; and/or
g. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF6 binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
h. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF6 binding site is 3′ of the ApoE-HCR1 sequence element that comprises a nucleotide sequence of SEQ ID NO: 92 or a variant of SEQ ID NO: 92 that differs by 1, 2, or 3 nucleotides; and/or
i. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF6 binding site is 5′ of the ApoE-HCR1 sequence element that comprises a nucleotide sequence of SEQ ID NO: 93 or a variant of SEQ ID NO: 93 that differs by 1, 2, or 3 nucleotides; and/or
j. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF6 binding site is 5′ of the ApoE-HCR1 sequence element that comprises a nucleotide sequence of SEQ ID NO: 92 or a variant of SEQ ID NO: 92 that differs by 1, 2, or 3 nucleotides; and/or
k. the TRE comprises an AMBP sequence element that comprises a nucleotide sequence of SEQ ID NO: 98 or a variant of SEQ ID NO: 98 that differs by 1, 2, or 3 nucleotides and the HNF6 binding site is 3′ of the AMBP sequence element that comprises a nucleotide sequence of SEQ ID NO: 98 or a variant of SEQ ID NO: 98 that differs by 1, 2, or 3 nucleotides; and/or
l. the TRE comprises an AMBP sequence element that comprises a nucleotide sequence of SEQ ID NO: 97 or a variant of SEQ ID NO: 97 that differs by 1, 2, or 3 nucleotides and the HNF6 binding site is 3′ of the AMBP sequence element that comprises a nucleotide sequence of SEQ ID NO: 97 or a variant of SEQ ID NO: 97 that differs by 1, 2, or 3 nucleotides; and/or
m. the TRE comprises an AMBP sequence element that comprises a nucleotide sequence of SEQ ID NO: 98 or a variant of SEQ ID NO: 98 that differs by 1, 2, or 3 nucleotides and the HNF6 binding site is 5′ of the AMBP sequence element that comprises a nucleotide sequence of SEQ ID NO: 98 or a variant of SEQ ID NO: 98 that differs by 1, 2, or 3 nucleotides; and/or
n. the TRE comprises an AMBP sequence element that comprises a nucleotide sequence of SEQ ID NO: 97 or a variant of SEQ ID NO: 97 that differs by 1, 2, or 3 nucleotides and the HNF6 binding site is 5′ of the AMBP sequence element that comprises a nucleotide sequence of SEQ ID NO: 97 or a variant of SEQ ID NO: 97 that differs by 1, 2, or 3 nucleotides.

The TRE may promote equivalent or greater expression than a corresponding TRE not comprising the HNF6 binding site.

In one aspect, a TRE comprising the HNF6 binding site promotes equivalent or greater expression than a corresponding TRE lacking an HNF6 binding site. The skilled person can determine whether a TRE comprising an HNF6 binding site (a test TRE) promotes equivalent or greater expression than a second TRE not comprising an HNF6 binding site (a reference TRE) by determining the level of expression promoted by the test TRE comprising an HNF6 binding site and the reference TRE using the techniques described under the heading “expression assay”. Optionally, the user determines whether a TRE comprising an HNF6 binding site (the test HNF6 binding site) promotes equivalent or greater expression than a reference TRE not comprising an HNF6 binding site by creating a test TRE that comprises the test HNF6 binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF6 binding site.

A TRE comprising a HNF6 binding site and a TATA box may promote equivalent or greater expression than a corresponding TRE not comprising the HNF6 binding site and the TATA box.

In an aspect, the TRE further comprises an HNF4a binding site. Optionally, the HNF4a binding site has a nucleotide sequence of any one of SEQ ID NO: 151-154.

In an aspect, the TRE comprises an HNF4a binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF4a binding site is 5′ of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 96 or a variant of SEQ ID NO: 96 that differs by 1, 2, or 3 nucleotides and the HNF4a binding site is 5′ of the hAAT sequence element that comprises a nucleotide sequence of SEQ ID NO: 96 or a variant of SEQ ID NO: 96 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF4a binding site is 5′ of the hAAT sequence element that comprises a nucleotide sequence of SEQ ID NO: 94 or a variant of SEQ ID NO: 94 that differs by 1, 2, or 3 nucleotides; and/or
d. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF4a binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
e. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF4a binding site is 5′ of the ApoE-HCR1 sequence element that comprises a nucleotide sequence of SEQ ID NO: 93 or a variant of SEQ ID NO: 93 that differs by 1, 2, or 3 nucleotides; and/or
f. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF4a binding site is 5′ of the ApoE-HCR1 sequence element that comprises a nucleotide sequence of SEQ ID NO: 92 or a variant of SEQ ID NO: 92 that differs by 1, 2, or 3 nucleotides; and/or
g. the TRE comprises an AMBP sequence element that comprises a nucleotide sequence of SEQ ID NO: 98 or a variant of SEQ ID NO: 98 that differs by 1, 2, or 3 nucleotides and the HNF4a binding site is 3′ of the AMBP sequence element that comprises a nucleotide sequence of SEQ ID NO: 98 or a variant of SEQ ID NO: 98 that differs by 1, 2, or 3 nucleotides; and/or
h. the TRE comprises an AMBP sequence element that comprises a nucleotide sequence of SEQ ID NO: 97 or a variant of SEQ ID NO: 97 that differs by 1, 2, or 3 nucleotides and the HNF4a binding site is 3′ of the AMBP sequence element that comprises a nucleotide sequence of SEQ ID NO: 97 or a variant of SEQ ID NO: 97 that differs by 1, 2, or 3 nucleotides; and/or
i. the TRE comprises an AMBP sequence element that comprises a nucleotide sequence of SEQ ID NO: 98 or a variant of SEQ ID NO: 98 that differs by 1, 2, or 3 nucleotides and the HNF4a binding site is 5′ of the AMBP sequence element that comprises a nucleotide sequence of SEQ ID NO: 98 or a variant of SEQ ID NO: 98 that differs by 1, 2, or 3 nucleotides; and/or
j. the TRE comprises an AMBP sequence element that comprises a nucleotide sequence of SEQ ID NO: 97 or a variant of SEQ ID NO: 97 that differs by 1, 2, or 3 nucleotides and the HNF4a binding site is 5′ of the AMBP sequence element that comprises a nucleotide sequence of SEQ ID NO: 97 or a variant of SEQ ID NO: 97 that differs by 1, 2, or 3 nucleotides.

The TRE may promote equivalent or greater expression than a corresponding TRE not comprising the HNF4a binding site.

In one aspect, a TRE comprising the HNF4a binding site promotes equivalent or greater expression than a corresponding TRE lacking an HNF4a binding site. The skilled person can determine whether a TRE comprising an HNF4a binding site (a test TRE) promotes equivalent or greater expression than a second TRE not comprising an HNF4a binding site (a reference TRE) by determining the level of expression promoted by the test TRE comprising an HNF4a binding site and the reference TRE using the techniques described under the heading “expression assay”. Optionally, the user determines whether a TRE comprising an HNF4a binding site (the test HNF4a binding site) promotes equivalent or greater expression than a reference TRE not comprising an HNF4a binding site by creating a test TRE that comprises the test HNF4a binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF4a binding site.

A TRE comprising a HNF6 binding site and an HNF4a binding site may promote equivalent or greater expression than a corresponding TRE not comprising the HNF6 binding site and the HNF4a binding site.

In an aspect, the TRE further comprises an HNF1 binding site. Optionally, the HNF1 binding site has a nucleotide sequence of SEQ ID NO: 133 or 134.

In an aspect, the TRE comprises an HNF1 binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF1 binding site is 5′of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 96 or a variant of SEQ ID NO: 96 that differs by 1, 2, or 3 nucleotides and the HNF1 binding site is 5′of the hAAT sequence element that comprises a nucleotide sequence of SEQ ID NO: 96 or a variant of SEQ ID NO: 96 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF1 binding site is 5′of the hAAT sequence element that comprises a nucleotide sequence of SEQ ID NO: 94 or a variant of SEQ ID NO: 94 that differs by 1, 2, or 3 nucleotides; and/or
d. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF1 binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
e. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF1 binding site is 3′ of the ApoE-HCR1 sequence element that comprises a nucleotide sequence of SEQ ID NO: 92 or a variant of SEQ ID NO: 92 that differs by 1, 2, or 3 nucleotides; and/or
f. the TRE comprises an AMBP sequence element that comprises a nucleotide sequence of SEQ ID NO: 98 or a variant of SEQ ID NO: 98 that differs by 1, 2, or 3 nucleotides and the HNF1 binding site is 3′ of the AMBP sequence element that comprises a nucleotide sequence of SEQ ID NO: 98 or a variant of SEQ ID NO: 98 that differs by 1, 2, or 3 nucleotides; and/or
g. the TRE comprises an AMBP sequence element that comprises a nucleotide sequence of SEQ ID NO: 97 or a variant of SEQ ID NO: 97 that differs by 1, 2, or 3 nucleotides and the HNF1 binding site is 3′ of the AMBP sequence element that comprises a nucleotide sequence of SEQ ID NO: 97 or a variant of SEQ ID NO: 97 that differs by 1, 2, or 3 nucleotides.

The TRE may promote equivalent or greater expression than a corresponding TRE not comprising the HNF1 binding site.

In one aspect, a TRE comprising the HNF1 binding site promotes equivalent or greater expression than a corresponding TRE lacking an HNF1 binding site. The skilled person can determine whether a TRE comprising an HNF1 binding site (a test TRE) promotes equivalent or greater expression than a second TRE not comprising an HNF1 binding site (a reference TRE) by determining the level of expression promoted by the test TRE comprising an HNF1 binding site and the second TRE using the techniques described under the heading “expression assay”. Optionally, the user determines whether a TRE comprising an HNF1 binding site (the test HNF1 binding site) promotes equivalent or greater expression than a reference TRE not comprising an HNF1 binding site by creating a test TRE that comprises the test HNF1 binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF1 binding site.

A TRE comprising an HNF6 binding site, an HNF4a binding site, and a TATA box may promote equivalent or greater expression than a corresponding TRE not comprising the HNF6 binding site, the HNF4a binding site, and the TATA box.

In an aspect, the TRE further comprises an HNF3 binding site. Optionally, the HNF3 binding site has a nucleotide sequence of any one of SEQ ID NO: 142-144.

In an aspect, the TRE comprises an HNF3 binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF3 binding site is 5′of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 96 or a variant of SEQ ID NO: 96 that differs by 1, 2, or 3 nucleotides and the HNF3 binding site is 5′of the hAAT sequence element that comprises a nucleotide sequence of SEQ ID NO: 96 or a variant of SEQ ID NO: 96 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF3 binding site is 5′of the hAAT sequence element that comprises a nucleotide sequence of SEQ ID NO: 94 or a variant of SEQ ID NO: 94 that differs by 1, 2, or 3 nucleotides; and/or
d. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF3 binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
e. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF3 binding site is 3′ of the ApoE-HCR1 sequence element that comprises a nucleotide sequence of SEQ ID NO: 92 or a variant of SEQ ID NO: 92 that differs by 1, 2, or 3 nucleotides; and/or
f. the TRE comprises an AMBP sequence element that comprises a nucleotide sequence of SEQ ID NO: 98 or a variant of SEQ ID NO: 98 that differs by 1, 2, or 3 nucleotides and the HNF3 binding site is 3′ of the AMBP sequence element that comprises a nucleotide sequence of SEQ ID NO: 98 or a variant of SEQ ID NO: 98 that differs by 1, 2, or 3 nucleotides; and/or
g. the TRE comprises an AMBP sequence element that comprises a nucleotide sequence of SEQ ID NO: 97 or a variant of SEQ ID NO: 97 that differs by 1, 2, or 3 nucleotides and the HNF3 binding site is 3′ of the AMBP sequence element that comprises a nucleotide sequence of SEQ ID NO: 97 or a variant of SEQ ID NO: 97 that differs by 1, 2, or 3 nucleotides.

The TRE may promote equivalent or greater expression than a corresponding TRE not comprising the HNF3 binding site.

In one aspect, a TRE comprising the HNF3 binding site promotes equivalent or greater expression than a corresponding TRE lacking an HNF3 binding site. The skilled person can determine whether a TRE comprising an HNF3 binding site (a test TRE) promotes equivalent or greater expression than a second TRE not comprising an HNF3 binding site (a reference TRE) by determining the level of expression promoted by the test TRE and the reference TRE using the techniques described under the heading “expression assay”. Optionally, the user determines whether a TRE comprising an HNF3 binding site (the test HNF3 binding site) promotes equivalent or greater expression than a reference TRE not comprising an HNF3 binding site by creating a test TRE that comprises the test HNF3 binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF3 binding site.

A TRE comprising an HNF3 binding site and a TATA box and the TRE may promote equivalent or greater expression than a corresponding TRE not comprising the HNF3 binding site and the TATA box.

In one embodiment, the TRE comprises:
(i) an AMBP sequence element;
(ii) a hAAT sequence element;
(iii) an ApoE-HCR1 sequence element;
(iv) a Transcription Start Site (TSS); and
(v) a TATA box, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the TATA box is at position -37 from the TSS; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site;
(v) an HNF4a binding site;
(vi) a TATA box; and
(vii) a second HNF6 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is 3′ of the AMBP sequence element and 5′ of the ApoE-HCR1 sequence element;
   (vii) the HNF4a binding site is 3′ of the ApoE-HCR-1 sequence element and 5′ of the hAAT sequence element;
   (viii) the TATA box is immediately 5′ of a nucleotide sequence corresponding to the nucleotide sequence of SEQ ID NO: 95;
   (ix) the second HNF6 binding site is immediately 3′ of a nucleotide sequence corresponding to the nucleotide sequence of SEQ ID NO: 95; and
   (x) the TRE is between 900 and 950 nucleotides in length.

In one embodiment, the TRE comprises:
(i) an AMBP sequence element;
(ii) a hAAT sequence element;
(iii) an ApoE-HCR1 sequence element;
(iv) an HNF6 binding site;
(v) an HNF1 binding site;
(vi) an HNF4a binding site;
(vii) a TATA box; and
(viii) a second HNF6 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is 3′ of the AMBP sequence element and 5′ of the ApoE-HCR1 sequence element;
   (vii) the HNF1 binding site is immediately 3′ of the HNF4a binding site;
   (viii) HNF4a binding site is 3′ of the ApoE-HCR-1 sequence element and 5′ of the hAAT sequence element;
   (ix) the TATA box is immediately 5′ of a nucleotide sequence corresponding to the nucleotide sequence of SEQ ID NO: 95;
   (x) the second HNF6 binding site is immediately 3′ of a nucleotide sequence corresponding to the nucleotide sequence of SEQ ID NO: 95; and
   (xi) the TRE is between 900 and 950 nucleotides in length.

In one embodiment, the TRE comprises:
(i) an AMBP sequence element;
(ii) a hAAT sequence element;
(iii) an ApoE-HCR1 sequence element;
(iv) an HNF3 binding site; and
(v) a TATA box, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF3 binding site is 3′ of the ApoE-HCR1 sequence element and 5′ of the hAAT sequence element;
   (vii) the TATA box is immediately 5′ of a nucleotide sequence corresponding to the nucleotide sequence of SEQ ID NO: 95; and
   (viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) a TATA box, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is 3′ of the AMBP sequence element and 5′ of the ApoE-HCR1 sequence element;
   (vii) the TATA box is immediately 5′ of a nucleotide sequence corresponding to the nucleotide sequence of SEQ ID NO: 95; and
   (viii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF6 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is 3′ of the AMBP sequence element and 5′ of the ApoE-HCR1 sequence element; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF4 binding site is 3′ of the AMBP sequence element and 5′ of the ApoE-HCR1 sequence element; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF6 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is 3′ of the ApoE-HCR1 sequence element and 5′ of the hAAT sequence element; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF4 binding site is 3′ of the ApoE-HCR1 sequence element and 5′ of the hAAT sequence element; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF4 binding site is immediately 3′ of a nucleotide sequence corresponding to a nucleotide sequence of SEQ ID NO: 93; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) a TATA box, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is immediately 3′ of a nucleotide sequence corresponding to the nucleotide sequence of SEQ ID NO: 95;
   (vii) the TATA box is immediately 5′ of the HNF6 binding site; and
   (viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF4 binding site; and
(v) an HNF6 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF4 binding site is 3′ of the ApoE-HCR1 sequence element and 5′ of the hAAT sequence element;
   (vii) the HNF6 binding site is 3′ of the AMBP sequence element and 5′ of the ApoE-HCR1 sequence element; and
   (viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) an HNF4 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is 3′ of the ApoE-HCR1 sequence element and 5′ of the hAAT sequence element;
   (vii) the HNF4 binding site is 3′ of the AMBP sequence element and 5′ of the ApoE-HCR1 sequence element; and
   (viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) a TATA box, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is immediately 3′ of a nucleotide sequence corresponding to the nucleotide sequence of SEQ ID NO: 95;
   (vii) the TATA box is immediately 5′ of a nucleotide sequence corresponding to the nucleotide sequence of SEQ ID NO: 95; and
   (viii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF6 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is 5′ of the AMBP sequence element; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF4 binding site is 5′ of the AMBP sequence element; and
   (vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF6 binding site, wherein:
   (i) the AMBP sequence element comprises a nucleotide sequence of SEQ ID NO: 97;
   (ii) the hAAT sequence element comprises a nucleotide sequence of SEQ ID NO: 94;
   (iii) the ApoE-HCR1 sequence element comprises a nucleotide sequence of SEQ ID NO: 92;
   (iv) the AMBP sequence element is 5′ of the hAAT sequence element;
   (v) the ApoE-HCR1 sequence element is 5′ of the hAAT sequence element and 3′ of the AMBP sequence element;
   (vi) the HNF6 binding site is immediately 3′ of a nucleotide sequence corresponding to the nucleotide sequence of SEQ ID NO: 95; and
   (vii) the TRE is between 875 and 925 nucleotides in length.

The polynucleotide of the invention may further comprise one or two ITRs. In one embodiment, the nucleotide sequence of the or each ITR is fewer than 157, fewer than 154, or around 145 nucleotides in length. Optionally, the or each ITR is a wild-type ITR. Optionally, the or each ITR is an AAV2 ITR. In some embodiments, the nucleotide sequence of the or each ITR comprises a nucleotide sequence of SEQ ID NO: 53 or SEQ ID NO: 54.

The polynucleotide of the invention may further comprise a polyA nucleotide sequence. The poly A sequence may be a bovine growth hormone poly A sequence (bGHpA - SEQ ID NO: 55). In some embodiments, the polyA nucleotide sequence comprises the nucleotide sequence of SEQ ID NO: 55. The poly A sequence may be between 200 to 220, 205 to 215, or around 208 nucleotides in length. The poly A sequence may be positioned downstream of the CFI nucleotide sequence of the invention.

The polynucleotide of the invention may further comprise an intron sequence, such as a viral intron sequence, optionally an SV40 intron sequence (SEQ ID NO: 60).

### A Viral Particle Comprising the Polynucleotide

The invention further provides a viral particle comprising a recombinant genome comprising the polynucleotide of the invention. For the purposes of the present invention, the term “viral particle” refers to all or part of a virion. For example, the viral particle comprises a recombinant genome and may further comprise a capsid. The viral particle may be a gene therapy vector. Herein, the terms “viral particle” and “vector” are used interchangeably. For the purpose of the present application, a “gene therapy” vector is a viral particle that can be used in gene therapy, i.e. a viral particle that comprises all the required functional elements to express a transgene, such as a CFI nucleotide sequence, in a host cell after administration.

Suitable viral particles include a parvovirus, a retrovirus, a lentivirus or a herpes simplex virus. The parvovirus may be an adeno-associated virus (AAV). Optionally, the viral particle is an AAV, adenoviral or lentiviral particle. The viral particle is preferably a recombinant AAV vector or a lentiviral vector. More preferably, the viral particle is an AAV viral particle. The terms AAV and rAAV are used interchangeably herein, unless context obviously suggests otherwise.

The genomic organization of all known AAV serotypes is very similar. The genome of AAV is a linear, single-stranded DNA molecule that is less than about 5,000 nucleotides in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal ~145 nt (ITRs) are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

As described above, the polynucleotide of the invention may comprise one or two ITR(s). Thus, the recombinant viral genome may comprise one or two ITR(s) as described above. It is possible for an AAV vector of the invention to function with only one ITR. Thus, the viral genome typically comprises at least one ITR, but, more typically, two ITRs (generally with one either end of the viral genome, i.e. one at the 5′ end and one at the 3′ end). There may be intervening sequences between the CFI nucleotide sequence of the invention and one or both of the ITRs. The CFI nucleotide sequence may be incorporated into a viral particle located between two regular ITRs or located on either side of an ITR engineered with two D regions.

AAV sequences that may be used in the present invention for the production of AAV vectors can be derived from the genome of any AAV serotype. Generally, the AAV serotypes have genomic sequences of significant homology at the amino acid and the nucleotide levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chiorini et al, 1997; Srivastava et al, 1983; Chiorini et al, 1999; Rutledge et al, 1998; and Wu et al, 2000. AAV serotype 1, 2, 3, 3B, 4, 5, 6, 7, 8, 9, 10, 11 or 12 may be used in the present invention. The sequences from the AAV serotypes may be mutated or engineered when being used in the production of gene therapy vectors.

Optionally, an AAV vector comprises ITR sequences which are derived from AAV1, AAV2, AAV4 and/or AAV6. Preferably the ITR sequences are AAV2 ITR sequences. Herein, the term AAVx/y refers to a viral particle that comprises genomic components such as at least ITRs from AAVx (wherein x is a AAV serotype number) and has the capsid from AAVy (wherein y is the number of the same or different serotype). For example, an AAV2/8 vector may comprise a portion of a viral genome, including the ITRs, from an AAV2 strain, and a capsid from an AAV8 strain.

In an embodiment, the viral particle comprises a capsid. In an embodiment, the viral particle is an AAV viral particle comprising a capsid. AAV capsids are generally formed from three proteins, VP1, VP2 and VP3. The amino acid sequence of VP1 comprises the sequence of VP2. The portion of VP1 which does not form part of VP2 is referred to as VP1unique or VP1U. The amino acid sequence of VP2 comprises the sequence of VP3. The portion of VP2 which does not form part of VP3 is referred to as VP2unique or VP2U. Optionally, the viral particle comprises a liver-tropic capsid. Whether a viral particle (capsid) is tropic for a particular tissue can be evaluated for example by administering such a particle expressing a marker gene such as luciferase and imaging in vivo at multiple time points (for example as described in Zincarelli et al (2008), Molecular Therapy, 16:1073-1080). A particle driving strong marker expression in liver tissues, especially if in contrast to lesser expression in other tissues, would be considered liver-tropic.

In some embodiments, a liver-tropic capsid can be an AAV3-, AAV3B-, AAV5, or AAV8-derived capsid. Optionally, the liver-tropic capsid can be an AAV3-, AAV3B-, or AAV8-derived capsid. Optionally, the liver-tropic capsid comprises a sequence at least 98%, at least 99%, or at least 99.5% identical to a fragment of at least 600, at least 650, at least 700, between 600 and 734, between 600 and 736, between 650 and 734, between 650 and 736, between 700 and 734, between 700 and 736, around 734, or around 736 amino acids of any one of SEQ ID NOs: 56-59. Optionally, the liver-tropic capsid comprises a sequence at least 99% identical to any one of SEQ ID NOs: 56-59. Optionally, the liver-tropic capsid comprises a sequence at least 99% identical to SEQ ID NO: 57 or 58. Optionally, the liver-tropic capsid comprises a sequence at least 99% identical to SEQ ID NO: 57.

In some embodiments, the capsid is not an AAV2-derived capsid. In some embodiments, the capsid is not an AAV8-derived capsid.

A viral particle of the invention may be a “hybrid” particle in which the viral ITRs and viral capsid are from different parvoviruses, such as different AAV serotypes. Preferably, the viral ITRs and capsid are from different serotypes of AAV, in which case such viral particles are known as transcapsidated or pseudotyped. Likewise, the parvovirus may have a “chimeric” capsid (e. g., containing sequences from different parvoviruses, preferably different AAV serotypes) or a “targeted” capsid (e. g., a directed tropism).

In some embodiments, the recombinant AAV genome comprises intact ITRs, comprising functional terminal resolution sites (TRS). Such an AAV genome may contain one or two resolvable ITRs, i.e. ITRs containing a functional TRS at which site-specific nicking can take place to create a free 3′ hydroxyl group which can serve as a substrate for DNA polymerase to unwind and copy the ITR.

Preferably, the recombinant genome is single-stranded (i.e., it is packaged into the viral particle in a single-stranded form). Optionally, the recombinant genome is not packaged in self-complementary configuration, i.e. the genome does not comprise a single covalently-linked polynucleotide strand with substantial self-complementary portions that anneal in the viral particle. Alternatively, the recombinant genome may be packaged in “monomeric duplex” form. “Monomeric duplexes” are described in WO 2011/122950. The genome may be packaged as two substantially complementary but non-covalently linked polynucleotides which anneal in the viral particle.

As described above, the polynucleotide of the invention may comprise polyA nucleotide sequence. Thus, the viral particle may comprise the poly A sequence as described above.

As described above, the polynucleotide of the invention may comprise an intron sequence. Thus, the polynucleotide of the invention may comprise the intron sequence as described above.

In an embodiment, the viral particle comprises a polynucleotide sequence comprising a transcription regulatory element (comprising e.g. a promoter and/or enhancer), a CFI nucleotide sequence, and a poly A sequence, such as the bGHpA sequence. In such embodiments, the poly A sequence, such as the bGHpA sequence, may be located downstream of the CFI nucleotide sequence. An intron sequence, such as the SV40 intron sequence, may be located between the transcription regulatory element and the CFI nucleotide sequence.

In an embodiment, the CFI polypeptide or the fragment thereof encoded by the CFI nucleotide sequence is expressed at the same or a higher level on administration of the viral particle compared to expression of a CFI polypeptide encoded by a reference CFI nucleotide sequence on administration of an equivalent viral particle comprising the reference CFI nucleotide sequence. The reference CFI nucleotide sequence may be the nucleotide sequence of SEQ ID NO: 1 or 21. The reference CFI nucleotide sequence may be the nucleotide sequence of SEQ ID NO: 5 or 25. The “equivalent” viral particle comprising the reference CFI nucleotide is a viral particle which is identical (i.e. the viral particle comprises the same capsid and the recombinant genome comprises the same transcription regulatory elements etc.) to the viral particle of the invention except that the CFI nucleotide sequences are different. For example, the different CFI nucleotide sequences being compared are operably linked to the same promoter sequence. In one embodiment, the expression of the CFI polypeptide or the fragment thereof encoded by the CFI nucleotide sequence is at a higher level compared to the expression of the CFI polypeptide encoded by a reference CFI nucleotide sequence. In one embodiment, the expression of the CFI polypeptide or the fragment thereof encoded by the CFI nucleotide sequence is at least 1.5x, at least 2x, at least 3x, at least 5x, at least 8x, or at least 10x higher compared to the CFI polypeptide encoded by the reference CFI nucleotide sequence. Optionally, the expression of the CFI polypeptide or the fragment thereof encoded by the CFI nucleotide sequence may be between 1.5x and 10x, between 2x and 10x, between 5x and 10x, or between 8x and 10x higher compared to the CFI polypeptide encoded by the reference CFI nucleotide sequence. The reference CFI nucleotide sequence may be a wild-type CFI nucleotide sequence or the nucleotide sequence of any one of SEQ ID NOs: 1, 21, 5 or 25. The reference CFI nucleotide sequence may be a wild-type CFI nucleotide sequence. The wild type CFI nucleotide may be the sequence of SEQ ID NO: 41 or 42. The reference CFI nucleotide sequence may be the nucleotide sequence of SEQ ID NO: 1 or 21. The reference CFI nucleotide sequence may be the nucleotide sequence of SEQ ID NO: 5 or 25.

In an embodiment, the expression of the CFI polypeptide or the fragment thereof encoded by the CFI nucleotide sequence is at least 50%, at least 60%, at least 70%, at least 80%, at least 90%, at least 95%, or at least 100% of the expression level of the CFI polypeptide encoded by the sequence of SEQ ID NO: 5 or 25.

The level of expression of the CFI polypeptide or the fragment thereof may typically be determined by measuring the level of the CFI polypeptide or the fragment thereof in a sample. Optionally, the level of expression of a CFI polypeptide or the fragment thereof is determined by using an ELISA. The level of expression of a CFI polypeptide or the fragment thereof may be determined using the ELISA assay as described in Example 2. Optionally, the expression level is normalised by vector genome copy number. Optionally, the expression level is not normalised by vector genome copy number.

Optionally, the level of expression of the CFI polypeptide or the fragment thereof is determined by using an ELISA as described above.

Optionally, the administration of the viral particle described above is administration to a mouse (e.g. C57/B1/6 mouse). As mentioned above, the expression level of a CFI polypeptide or the fragment thereof may be normalised by vector genome copy number. Vector genome copy number may be determined by performing qPCR. The vector genome copy number in the murine liver (or a portion of) may be determined.

Optionally, the viral particle comprises a liver-tropic capsid (e.g. a liver-tropic capsid comprising a sequence at least 99% identical to SEQ ID NO: 57) and the polynucleotide comprises a liver-specific transcription regulatory element (e.g. at least 99% identical to SEQ ID NO: 74). Optionally, the viral particle comprises a liver-tropic capsid (e.g. a liver-tropic capsid comprising a sequence at least 99% identical to SEQ ID NO: 57), the polynucleotide comprises a liver-specific transcription regulatory element (e.g. at least 99% identical to SEQ ID NO: 74), and the viral particle is not administered intraocularly.

### Compositions, Methods and Uses

In a further aspect of the invention, there is provided a composition comprising the polynucleotide or vector/viral particle of the invention and a pharmaceutically acceptable excipient.

The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

The invention further provides a CFI polynucleotide, vector/viral particle or composition of the invention for use in a method of treatment. Optionally the method of treatment comprises administering an effective amount of the CFI polynucleotide, vector/viral particle, or composition of the invention to a patient.

The invention further provides a method of treatment comprising administering an effective amount of the CFI polynucleotide, vector/viral particle, or composition of the invention. Optionally the method of treatment comprises administering an effective amount of the polynucleotide or vector/viral particle of the invention to a patient.

The invention further provides use of the CFI polynucleotide, vector/viral particle or composition of the invention in the manufacture of a medicament for use in a method of treatment. Optionally the method of treatment comprises administering an effective amount of the polynucleotide or vector/viral particle of the invention to a patient.

Optionally, the method of treatment is a gene therapy. A “gene therapy” involves administering a vector/viral particle of the invention that is capable of expressing a transgene (such as CFI nucleotide sequence) in the host (e.g. patient) to which it is administered.

The invention further provides a polynucleotide comprising a CFI nucleotide sequence, a vector/viral particle comprising a recombinant genome comprising the polynucleotide, or a composition comprising the polynucleotide or vector/viral particle for use in a method of treatment, wherein the method of treatment is a method of treating a complement-mediated disorder, and the CFI nucleotide sequence encodes a CFI polypeptide or a fragment thereof.

The invention further provides a method of treatment comprising administering an effective amount of a polynucleotide comprising a CFI nucleotide sequence, a vector/viral particle comprising a recombinant genome comprising the polynucleotide, or a composition comprising the polynucleotide or vector/viral particle, wherein the method of treatment is a method of treating a complement-mediated disorder, and the CFI nucleotide sequence encodes a CFI polypeptide or a fragment thereof.

The invention further provides use of a polynucleotide comprising a CFI nucleotide sequence, a vector/viral particle comprising a recombinant genome comprising the polynucleotide, or a composition comprising the polynucleotide or vector/viral particle in the manufacture of a medicament for use in a method of treatment, wherein the method of treatment is a method of treating a complement-mediated disorder, and the CFI nucleotide sequence encodes a CFI polypeptide or a fragment thereof.

Optionally, the method of treatment comprises administering an effective amount of the polynucleotide, vector/viral particle, or composition to a patient.

Optionally, the patient has a level of CFI protein that is lower than normal. Optionally, the patient has a mutation in the CFI gene or in one or more of the transcription regulatory elements for the CFI gene. Optionally, the patient is deficient in CFI. Optionally, the patient has a normal level of CFI protein. Optionally, the administration of the polynucleotide, viral particle, or composition increases the level of CFI protein. Optionally, the administration of the polynucleotide, viral particle, or composition increases the level of CFI protein above a normal level.

Optionally, the polynucleotide is the polynucleotide of the invention as described herein. Optionally, the viral particle is the viral particle of the invention as described herein. Optionally, the composition is the composition of the invention as described herein.

Optionally, the method of treatment is a method of treating a complement-mediated disorder. Optionally, the complement-mediated disorder is a C3-mediated disorder. Optionally, the complement-mediated disorder is a kidney disorder.

Optionally, the complement-mediated disorder is a disorder which can be treated by increasing the level of CFI protein in the patient. Optionally, the complement-mediated disorder is a disorder associated with increased and/or chronic inflammation. Optionally, the complement-mediated disorder is a disorder where the patient would benefit from reducing inflammation. Optionally, the complement-mediated disorder is not caused by a deficient level of functional CFI protein and/or from a defect in the complement system, but nevertheless may be treated by increasing the level of CFI protein. Optionally, the complement-mediated disorder is systemic lupus erythematosus.

Optionally, the complement-mediated disorder is associated with over-activity of the complement C3b feedback cycle. Optionally, the complement-mediated disorder is selected from C3 glomerulopathy, IgA nephropathy, lupus nephritis, systemic lupus erythematosus, membranous nephropathy, membranoproliferative glomerulonephritis, paroxysmal nocturnal hemoglobinuria, atypical hemolytic uremic syndrome, autoimmune haemolytic anemia, ANCA-associated vasculitis, Gaucher disease, peritonitis, age-related macular degeneration, diabetic retinopathy, dense deposit disease, age-related inflammatory or autoinflammatory diseases, autoimmune arthritis such as rheumatoid arthritis, atherosclerosis, chronic cardiovascular disease, Alzheimer’s disease, systemic vasculitis, Guillain-Barre syndrome, and Henoch-Schonlein purpura.

Optionally, the complement-mediated disorder is an age-related inflammatory or autoinflammatory disease. Optionally, the complement-mediated disorder is a chronic inflammatory disease.

Optionally, the complement-mediated disorder is selected from C3 glomerulopathy, C3 glomerulonephritis, and dense deposit disease.

Optionally, the complement-mediated disorder is atypical hemolytic uremic syndrome. Optionally, the complement-mediated disorder is atypical hemolytic uremic syndrome with monoallelic CFH mutation. In other words, the complement-mediated disorder may be atypical hemolytic uremic syndrome, and the group of patients to be treated is a subset of atypical haemolytic uremic syndrome patients that have a monoallelic CFH mutation.

Optionally, the complement-mediated disorder is a kidney glomerular or tubular disorder. Optionally, the disorder is selected from C3 glomerulopathy, IgA nephropathy, lupus nephritis and membranous nephropathy. Optionally, the disorder is lupus nephritis. Optionally, the disorder is systemic lupus erythematosus.

When a disease or disorder is “treated” as discussed herein (for example in the methods or uses of the invention), this means that one or more symptoms of the disease or disorder are ameliorated. It does not mean that the symptoms of the disease or disorder are completely remedied so that they are no longer present in the patient, although in some methods, this may be the case. Thus, in all instances the term “treatment” or “treating” can be replaced with the term “amelioration” or “ameliorating”, respectively. The methods or uses of the invention (such as the methods of treatment or treating) may result in one or more of the symptoms of the disease or disorder being less severe than before treatment. Optionally, relative to the situation pre-administration, the methods or uses of the invention (such as the method of treatment or treating) results in an increase in the amount/concentration of circulating CFI in the blood of the patient detectable within a given volume of blood.

In addition, the methods or uses of the invention may “prevent” disorders. In some embodiments, the term “treatment” or “treating” can be replaced with the term “prevention” or “preventing”, respectively.

A “effective amount” refers to an amount effective, at dosages and for periods of time necessary, to achieve the desired therapeutic result, such as raising the level of functional CFI in a subject (e.g. so as to lead to functional CFI production at a level sufficient to ameliorate the symptoms of the disease or disorder).

Optionally, the vector/viral particle is administered at a dose of less than 1 × 10¹¹, less than 1 × 10¹², less than 5 × 10¹², less than 2 × 10¹², less than 1.5 × 10¹², less than 3 × 10¹², less than 1 × 10¹³, less than 2 × 10¹³, or less than 3 × 10¹³vector genomes per kg of weight of patient (vg/kg). Optionally, the dose of vector/viral particle that is administered is selected such that the subject expresses CFI polypeptide at a level of 10%-90%, 20%-80%, 30%-70%, 25%-50%, 20%-150%, 30%-140%, 40%-130%, 50%-120%, 60%-110% or 70%-100% of the level of a healthy subject who does not suffer from the disorder.

Optionally, the administration of the polynucleotide, viral particle, or composition reduces the inflammation associated with the disorder in a subject. Optionally, administration of the polynucleotide, viral particle or composition reduces the inflammation to a level of less than or around 60%, less than or around 55%, less than or around 50%, less than or around 45%, less than or around 40%, less than or around 35%, less than or around 30%, less than or around 25%, or less than or around 20% of the level of inflammation present before the polynucleotide, viral particle or composition was administered. Optionally, administration of the polynucleotide, viral particle or composition reduces the inflammation to a level of less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of inflammation present before the polynucleotide, viral particle or composition was administered.

Optionally, the method of treatment is a method for reducing inflammation in a patient. Optionally, the method of treatment is a method for reducing inflammation in a patient, thereby treating the complement-mediated disorder. Optionally, the method of treatment is a method for treating a disease by reducing inflammation in a patient. Optionally, reducing inflammation is reducing inflammation to a level of less than or around 60%, less than or around 55%, less than or around 50%, less than or around 45%, less than or around 40%, less than or around 35%, less than or around 30%, less than or around 25%, or less than or around 20% of the level of inflammation present before the polynucleotide, viral particle or composition was administered. Optionally, administration of the polynucleotide, viral particle or composition reduces the inflammation to a level of less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of inflammation present before the polynucleotide, viral particle or composition was administered.

Optionally, the method of treatment comprises administering an effective amount of the polynucleotide, viral particle or composition to a patient, and the effective amount of the CFI polynucleotide, vector/viral particle, or composition is an amount sufficient to reduce inflammation in the patient. Optionally, an amount sufficient to reduce inflammation in the patient is an amount sufficient to reduce the inflammation to a less than or around 60%, less than or around 55%, less than or around 50%, less than or around 45%, less than or around 40%, less than or around 35%, less than or around 30%, less than or around 25%, or less than or around 20% of the level of inflammation present before the polynucleotide, viral particle or composition was administered. Optionally, administration of the polynucleotide, viral particle or composition reduces the inflammation to a level of less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of inflammation present before the polynucleotide, viral particle or composition was administered.

Optionally, the administration of the polynucleotide, viral particle, or composition reduces proteinuria in a subject. Optionally, proteinuria is scored by measuring the albumin-to-creatinine ratio (ACR) in urine. Optionally, administration of the polynucleotide, viral particle or composition reduces proteinuria to a level of less than or around 70%, less than or around 65%, less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of proteinuria present before the polynucleotide, viral particle or composition was administered. Optionally, administration of the polynucleotide, viral particle or composition reduces proteinuria to a level of less than or around 40%, less than or around 35%, less than or around 30%, less than or around 25%, less than or around 20%, less than or around 15%, or less than or around 10% of the level of proteinuria present before the polynucleotide, viral particle or composition was administered.

Optionally, the method of treatment is a method for reducing proteinuria in a patient. Optionally, the method of treatment is a method for reducing proteinuria in a patient, thereby treating the complement-mediated disorder. Optionally, the method of treatment is a method for treating a disease by reducing proteinuria in a patient. Optionally, reducing proteinuria is reducing proteinuria to a level of less than or around 70%, less than or around 65%, less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of proteinuria present before the polynucleotide, viral particle or composition was administered. Optionally, administration of the polynucleotide, viral particle or composition reduces proteinuria to a level of less than or around 40%, less than or around 35%, less than or around 30%, less than or around 25%, less than or around 20%, less than or around 15%, or less than or around 10% of the level of proteinuria present before the polynucleotide, viral particle or composition was administered.

Optionally, the method of treatment comprises administering an effective amount of the CFI polynucleotide, vector/viral particle, or composition to a patient, and the effective amount is an amount sufficient to reduce proteinuria in the patient. Optionally, an amount sufficient to reduce proteinuria in the patient is an amount sufficient to reduce the proteinuria to a level of less than or around 70%, less than or around 65%, less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of proteinuria present before the polynucleotide, viral particle or composition was administered. Optionally, an amount sufficient to reduce proteinuria in the patient is an amount sufficient to reduce the proteinuria to a level of less than or around 40%, less than or around 35%, less than or around 30%, less than or around 25%, less than or around 20%, less than or around 15%, or less than or around 10% of the level of proteinuria present before the polynucleotide, viral particle or composition was administered.

Optionally, the administration of the polynucleotide, viral particle, or composition reduces kidney glomeruli pathology and/or kidney tubule pathology in a subject. Optionally, administration of the polynucleotide, viral particle or composition reduces kidney glomeruli pathology and/or kidney tubule pathology as seen by a reduced level of pathology on a PAS stain.

Optionally, the method of treatment is a method for reducing kidney glomeruli pathology and/or kidney tubule pathology in a patient. Optionally, the method of treatment is a method for reducing kidney glomeruli pathology and/or kidney tubule pathology in a patient, thereby treating the complement-mediated disorder. Optionally, the method of treatment is a method for treating a disease by reducing kidney glomeruli pathology and/or kidney tubule pathology in a patient. Optionally, reducing kidney glomeruli pathology and/or kidney tubule pathology is reducing kidney glomeruli pathology and/or kidney tubule pathology as seen by a reduced level of pathology on a PAS stain. Optionally, the method of treatment comprises administering an effective amount of the polynucleotide, viral particle or composition to a patient, and the effective amount is an amount sufficient to reduce kidney glomeruli pathology and/or kidney tubule pathology in the patient. Optionally, an amount sufficient to reduce kidney glomeruli pathology and/or kidney tubule pathology in the patient is an amount sufficient to reduce the kidney glomeruli pathology and/or kidney tubule pathology as seen by a reduced level of pathology on a PAS stain.

Optionally, the disorder is not an ocular disorder. Optionally, the polynucleotide, viral particle, or composition is not administered intraocularly.

Optionally, the polynucleotide, viral particle, or composition is not administered by subretinal injection. Optionally, the polynucleotide, viral particle, or composition is not administered by direct retinal injection. Optionally, the polynucleotide, viral particle, or composition is not administered by suprachoroidal injection. Optionally, the polynucleotide, viral particle, or composition is not administered by intravitreal injection.

Optionally, the CFI polynucleotide, vector/viral particle and/or composition is administered intravenously. Optionally, the CFI polynucleotide, vector/viral particle and/or composition is administered systemically. Optionally, the CFI polynucleotide, vector/viral particle and/or composition is administered to the liver via peripheral vein infusion. Optionally, the CFI polynucleotide, vector/viral particle and/or composition is administered to the liver via hepatic vessels such as hepatic vein infusion or hepatic artery infusion. Optionally, the CFI polynucleotide, vector/viral particle and/or composition is administered via intraparenchymal administration direct to the liver. Optionally, the CFI polynucleotide, vector/viral particle and/or composition is administered by injection into the renal artery. Optionally, the CFI polynucleotide, vector/viral particle and/or composition is administered by retrograde administration, e.g. via the ureters using a urinary catheter.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 1 or 21.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 5 or 25.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 7 or 27.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 10 or 30.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 14 or 34.

Optionally, the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 17 or 37.

Optionally, the administration of the polynucleotide, viral particle, or composition increases the level of C3b-inactivating and/or iC3b-degradation activity in the patient. Optionally, administration of the polynucleotide, viral particle or composition increases the level of C3b-inactivating and/or iC3b-degradation activity above a normal level. Optionally, administration of the polynucleotide, viral particle or composition increases the level of C3b-inactivating and/or iC3b-degradation activity to a level 1.2-fold, 1.5-fold, 1.8-fold or 2-fold higher than the normal level. Optionally, a normal level is equivalent to that provided by 30-40 µg/ml of CFI polypeptide in the serum.

Optionally, the method of treatment is a method for increasing the level of C3b-inactivating and/or iC3b-degradation in a patient. Optionally, the method of treatment is a method for increasing the level of C3b-inactivating and/or iC3b-degradation in a patient, thereby treating the complement-mediated disorder. Optionally, the method of treatment is a method for treating a disease by increasing the level of C3b-inactivating and/or iC3b-degradation in a patient. Optionally, increasing the level of C3b-inactivating and/or iC3b-degradation in a patient is increasing the level of C3b-inactivating and/or iC3b-degradation activity above a normal level. Optionally, increasing the level of C3b-inactivating and/or iC3b-degradation in a patient is increasing the level of C3b-inactivating and/or iC3b-degradation activity to a level 1.2-fold, 1.5-fold, 1.8-fold or 2-fold higher than the normal level. Optionally, a normal level is equivalent to that provided by 30-40 µg/ml of CFI polypeptide in the serum.

Optionally, the viral particle comprises a liver-tropic capsid (e.g. a liver-tropic capsid comprising a sequence at least 99% identical to SEQ ID NO: 57) and the polynucleotide comprises a liver-specific transcription regulatory element (e.g. at least 99% identical to SEQ ID NO: 74). Optionally, the viral particle comprises a liver-tropic capsid (e.g. a liver-tropic capsid comprising a sequence at least 99% identical to SEQ ID NO: 57), the polynucleotide comprises a liver-specific transcription regulatory element (e.g. at least 99% identical to SEQ ID NO: 74), and the viral particle is not administered intraocularly. Optionally, the CFI nucleotide sequence is the nucleotide sequence of any one of SEQ ID NOs: 1, 2, 3 or 5 from WO2020/086735, or SEQ ID NO: 8 from WO2017/072515. Optionally, the CFI nucleotide sequence is the nucleotide sequence of any one of SEQ ID NOs: 18 to 26 from WO2020/128516.

Optionally, the CFI nucleotide sequence is the nucleotide sequence of any one of SEQ ID NOs: 1, 2, 3 or 5 from WO2020/086735, or a variant thereof encoding a CFI polypeptide comprising a sequence at least 95% identical to the polypeptide encoded by SEQ ID NO: 1, 2, 3 or 5 from WO2020/086735, respectively. Optionally, the CFI nucleotide sequence is the nucleotide sequence of SEQ ID NO: 8 from WO2017/072515, or a variant thereof encoding a CFI polypeptide comprising a sequence at least 95% identical to SEQ ID NO: 8 from WO2017/072515. Optionally, the CFI nucleotide sequence is the nucleotide sequence of any one of SEQ ID NOs: 18 to 26 from WO2020/128516, or a variant thereof encoding a CFI polypeptide comprising a sequence at least 95% identical to the polypeptide encoded by SEQ ID NO: 18, 19, 20, 21, 22, 23, 24, 25, or 26 from WO2020/128516, respectively.

Optionally, the variant is a variant of any one of SEQ ID NOs: 1, 2, 3 or 5 from WO2020/086735, SEQ ID NO: 8 from WO2017/072515, or any one of SEQ ID NOs: 18 to 26 from WO2020/128516. In some embodiments, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, is identical to SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively, except that it comprises nucleotide substitutions such that the encoded CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the CFI polypeptide sequence encoded by SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. In some embodiments, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. The variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, may have 1, 2 or fewer, 3 or fewer, 4 or fewer, 5 or fewer, or 6 or fewer nucleotide substitutions relative to the sequence of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. In one embodiment, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, has 4 or fewer nucleotide substitutions relative to the sequence of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively, and/or encodes a CFI polypeptide having 3 or fewer amino acid substitutions relative to the CFI polypeptide sequence encoded by SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. In one embodiment, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, has 3 or fewer nucleotide substitutions relative to the sequence of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516 respectively, and/or encodes a CFI polypeptide having 2 or fewer amino acid substitutions relative to the CFI polypeptide sequence encoded by SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. In one embodiment, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, has 1 nucleotide substitution relative to the sequence of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively, and/or encodes a CFI polypeptide having 1 amino acid substitution relative to the CFI polypeptide sequence encoded by SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. In one embodiment, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, encodes a CFI polypeptide having 3 or fewer amino acid substitutions relative to the CFI polypeptide sequence encoded by SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. In one embodiment, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, encodes a CFI polypeptide having 2 or fewer amino acid substitutions relative to the CFI polypeptide sequence encoded by SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively. In an embodiment, the variant of SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, encodes a CFI polypeptide having 1 amino acid substitution relative to the CFI polypeptide sequence encoded by SEQ ID NO: 1, 2, 3 or 5 from WO2020/086735, SEQ ID NO: 8 from WO2017/072515, or SEQ ID NO: 18, 19, 20, 21, 22, 23, 24, 25, or 26 from WO2020/128516, respectively.

The invention will now be described with reference to the following examples, which are merely illustrative and should not in any way be construed as limiting the scope of the present invention.

### EXAMPLES

### Example 1 - Huh7 In Vitro Transfection Study of Codon Optimised Sequences

Unique codon optimised human CFI sequences were screened and ranked based on the amount (w/v) of protein produced after transient transfection of Huh7 cells. Each of the codon optimised sequences encoded a wild-type human CFI polypeptide.

### Methods

Expression cassettes were generated which comprise one of the codon optimised CFI sequences or a wild type CFI sequence for control purposes. The promoter used was either “LSP-S” (SEQ ID NO: 51) or “LSP-L” (SEQ ID NO: 52), each of which is a liver-specific transcription regulatory element.

The transient transfection entailed mixing, for each expression cassette comprising one of the unique codon-optimised CFI genes, a plasmid carrying such expression cassette with a transfection reagent (FuGENE®HD (Promega, cat no. E2311)) and delivering the mixture to cells so that the gene can be expressed and protein subsequently quantified by ELISA. Where applicable, a luciferase reporter gene was included in the transfection mixture as a way of evaluating transfection efficiency.

Huh7 cell culture and Transfection: Huh7 cells (JCRB cell bank, no. JCRB0403) were seeded in a 96 well plate (30,000 cells per well) in DMEM low glucose, 10% FBS + Glutamax (D10 media) and cultured at 37° C. and 5% CO₂ (day 1). The next day (approx. 24 hours after cell seeding; day 2), the plasmid DNA-transfection reagent mixture was prepared and transfected into Huh7 cells. Briefly, 0.225 µg of test plasmid DNA and 0.025 µg CMV-Luciferase control plasmid were mixed with FuGENE at a ratio of 4 µl FuGENE per µg of DNA (or 1 µl FuGENE per 0.25 µg of plasmid DNA). For 96-well transfection experiments, 1µl of the FuGENE mix was added per well. The plasmid DNA-transfection reagent mixture was incubated on the cells overnight at 37° C. and 5% CO₂. The next morning (day 3), approximately 18 hours after transfection, the media was replaced with fresh D10 media and cells incubated overnight at 37° C. and 5% CO₂. The next morning (24h later; day 4), media was replaced by fresh DMEM low glucose + Glutamax + Insulin-Transferrin-Selenium supplement (D0/ITS media). Cells and media (supernatant) were harvested the following day (on day 5).

CFI expression in culture media (supernatant) was assessed using a CFI ELISA kit (e.g. Hycult cat# HK355-01) according to manufacturer’s instructions. In brief, culture media samples (diluted with the sample diluent from the kit as necessary) and human CFI standards from the kit were incubated in microtiter wells coated with anti-human CFI antibodies (to capture the human CFI). A biotinylated tracer antibody was added (to bind to the captured human CFI) followed by a streptavidin-peroxidase conjugate (to bind to the biotinylated tracer antibody). The substrate tetramethylbenzidine (TMB) was then added, which the streptavidin-peroxidase conjugate reacts with. The enzyme reaction was stopped by adding oxalic acid. For each of the samples and standards, the absorbance at 450 nm was measured with a spectrophotometer. A standard curve was obtained by plotting the absorbance (linear) against the corresponding concentrations of the human CFI standards (log). The concentration of human CFI in the samples was then determined from the standard curve.

Where applicable, in parallel to the ELISA, Huh7 cells were washed with phosphate buffered saline (PBS) twice and cells treated with 100µl of luciferase lysis buffer from the Luciferase assay kit (Promega cat# E1501/E4530). Cell lysates were stored at -80° C. On the day of luciferase assays, cell lysates were thawed and 20µl of the sample was used to measure the luciferase expression by luminescence on a Molecular Devices SpectraMax i3x plate reader. The detailed protocol is published in the Promega Technical Bullitin #TB281. Where applicable, luciferase expression was used as internal control to normalise the CFI expression levels. Analyses were performed using the software Graphpad Prism v7.

### Results

The CFI expression levels in the culture media following transfection into Huh-7 cells were measured for a wild-type human CFI nucleotide sequence and 38 codon-optimised human CFI sequences. The CFI expression levels were normalised using luciferase expression as described above. Each expression cassette comprised the LSP-L promoter and the BgpA polyA sequence. One of the codon-optimised CFI sequences, “Co01” (SEQ ID NO: 21), expressed more CFI polypeptide than the wild type CFI nucleotide sequence. The codon-optimised CFI sequence Co01 also expressed more CFI polypeptide than the other 37 codon-optimised CFI sequences. Therefore Co01 was ranked the highest expresser.

The CFI expression levels in the culture media following transfection into Huh-7 cells were measured for codon-optimised CFI sequence “Co01” and 19 further codon-optimised CFI sequences, Co02 to Co20 (SEQ ID NOs: 22 to 40, respectively). Each expression cassette comprised the LSP-S promoter and the BgpA polyA sequence. Each codon-optimised CFI sequence (Co02 to Co20) expressed CFI polypeptide at a similar or higher level than Co01. In particular, the codon-optimised CFI sequences Co02, Co03, Co05, Co06, Co07, Co09, Co10, Co11, Co12, Co14, Co15, Co16, Co17 and Co20 were ranked as being higher expressers of CFI polypeptide than Co01.

### Example 2 - In Vivo CFI Expression of Various Codon-Optimised Sequences

The codon optimised CFI sequences Co01, Co05, Co07, Co11, Co12, Co13, Co14, Co16 and Co17 (SEQ ID NOs: 21, 25, 27, 31, 32, 33, 34, 36, and 37, respectively) were further evaluated in vivo by vectorising the expression cassettes into AAV2/8 pseudo-typed capsids and transducing mice by systemic tail vein injection.

### Methods

The unique codon optimised CFI sequences were cloned into inverted terminal repeat-containing plasmids in the orientation of ITR-promoter-[codon optimised CFI]-polyA-ITR. AAV2/8-CFI vectors comprising the LSP-S promoter, the BgpA polyA sequence, and one of the CFI codon-optimised sequences (Co01, Co05, Co07, Co11, Co12, Co13, Co14, Co16 or Co17) were produced by the triple transfection method using polyethylenimine as a transection reagent (Grieger et al. Nature Protocols 2006; 1(3): 1412-1428 or similar), purified by column chromatography and titered using a qPCR assay. For each experimental set of codon optimised CFI vectors, the vectors were diluted to equivalent concentrations and injected into the tail vein of C57B1/6 mice. Doses were 2×10¹² vg/kg and studies were terminated 4-weeks post injection. At the end of the study, blood was collected via cardiac puncture into an anticoagulant (sodium citrate), processed to plasma by centrifugation and stored at -80° C. until CFI levels could be characterised by ELISA. Livers were also collected, snap frozen with liquid nitrogen and stored for future vector genome quantification by qPCR.

CFI expression in murine plasma was assessed using a CFI ELISA kit (e.g. Hycult cat# HK355-01) according to manufacturer’s instructions. In brief, murine plasma samples (diluted with the sample diluent from the kit as necessary) and human CFI standards from the kit were incubated in microtiter wells coated with anti-human CFI antibodies (to capture the human CFI). A biotinylated tracer antibody was added (to bind to the captured human CFI) followed by a streptavidin-peroxidase conjugate (to bind to the biotinylated tracer antibody). The substrate tetramethylbenzidine (TMB) was then added, which the streptavidin-peroxidase conjugate reacts with. The enzyme reaction was stopped by adding oxalic acid. For each of the samples and standards, the absorbance at 450 nm was measured with a spectrophotometer. A standard curve was obtained by plotting the absorbance (linear) against the corresponding concentrations of the human CFI standards (log). The concentration of human CFI in the samples was then determined from the standard curve.

Before vector genome quantification could be conducted by qPCR assay, livers were homogenised and DNA extracted and purified using the QIAGEN DNeasy Blood and Tissue kit (Cat. #69506). RNA was digested as needed by treatment of the sample with RNase A. Isolated DNA was subjected to qPCR reactions: the first to quantify the number of target (i.e. codon optimised CFI) vector genomes in murine livers and the second to quantify copies of a housekeeping gene (e.g. GAPDH). The number of copies of codon optimised vector genomes was divided by the number of copies of the housekeeping gene to determine the number of copies of codon optimised vector genomes per cell. qPCR assays were performed using standard methods. For codon optimised CFI sequences, primer pairs that bind common sequences of the AAV packaged DNA (e.g. sequences within the promoter) were utilised to amplify the DNA. DNA amplification was monitored using PowerUp Sybr green (Life Technologies, Cat. # A25743) and quantified by interpolation from a standard curve.

### Results

Each codon-optimised CFI sequence (Co05, Co07, Co11, Co12, Co13, Co14, Co16 and Co17) expressed CFI polypeptide in mouse plasma at a similar or higher level than Co01. In particular, the codon optimised CFI sequences Co05, Co07, Co12, Co14, Co16 and Co17 were ranked as being higher expressers of CFI protein than Co01. Co05, Co07 and Co14 were ranked as the top three highest expressers. Co05 was ranked as the highest expresser.

### Example 3 - Administration of AAV-CFI to a Zymosan-Induced Peritonitis Model

### Inflammation in Zymosan-Induced Peritonitis Model Dampened by AAV-CFI Administration

Experiments were performed to determine whether treatment with AAV expressing CFI can reduce inflammation in a Zymosan-induced peritonitis model. It has been previously demonstrated that peritonitis induced by Zymosan is at least in part mediated by C3 (Mullaly and Kubes. Eur. J. Immunol. 2007. 37: 224-234). To briefly summarise, C57BL/6 mice and mice homozygous negative for C3 (C3-/-) were intraperitoneally injected with Zymosan (5 mg/kg) or saline and the number of peritoneal neutrophils was determined 4 hours after Zymosan treatment. The number of neutrophils recruited into the peritoneal cavity significantly dropped in the absence of C3.

In order to determine whether AAV expressing CFI can reduce inflammation, C57BL/b mice were administered with AAV8 comprising CFI (made by triple plasmid transient transfection of HEK293T cells) prior to Zymosan treatment. The mice were administered with a 6×10¹¹, 2×10¹² or 6×10¹³ vg/kg dose of AAV. The AAV comprised the Co01 codon-optimised CFI nucleotide sequence (SEQ ID NO: 21) and the CAG promoter (SEQ ID NO: 61). Zymosan was administered at a dose of 5 mg/kg of body weight 16-weeks post-administration of the AAV8 comprising CFI. The number of peritoneal neutrophils were determined 4 hours after Zymosan treatment. The peritoneal neutrophils were collected by lavaging the peritoneal cavity and the cells were stained for FACS analysis and counted in accordance with the method section 3 in Cash et al. (2009), Methods in Enzymology, Volume 461, p.379-396. Human CFI expression levels in blood (collected from the tail vein) were determined by ELISA.

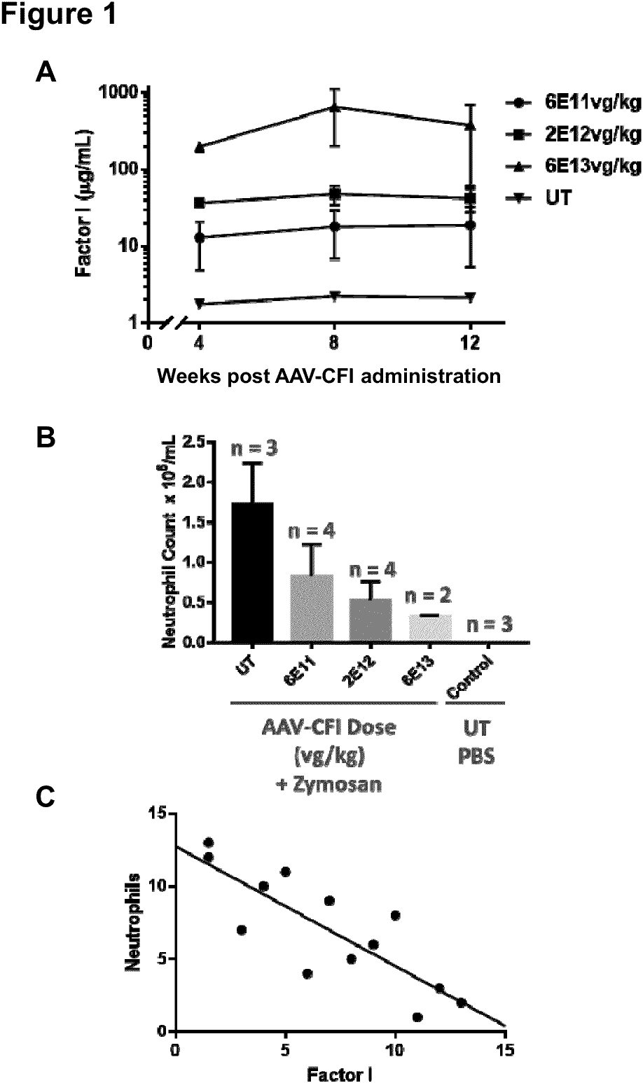

FIG. 1A shows a dose-dependent expression of CFI in blood. CFI expression resulted in a dose-dependent decrease in peritoneal neutrophil infiltration following Zymosan treatment (see FIG. 1B). FIG. 1C shows a negative correlation between CFI expression and neutrophil count.

### Example 4 - Administration of AAV-CFI to a Lupus Nephritis Model

Mice of the MRL-lpr mouse model (lupus nephritis mouse model) were treated with AAV8 comprising CFI (made by triple plasmid transient transfection of HEK293T cells). There were four treatment groups. The mice were treated according to Table 1 below starting at 6 weeks of age. Treatment continued for 19 weeks and mice were euthanized at 25 weeks of age. 26 MRL/MpJ-Fas^{lpr}/J (JAX stock# 000485) female mice were assigned to 3 of the treatment groups (treatment groups 1, 2 and 4). Treatment group 1 contained 10 mice which were treated with vehicle only. Treatment group 2 contained 10 mice which were treated with AAV comprising CFI at a dose of 6x10¹³ vg/kg. The AAV comprised the Co01 codon-optimised CFI nucleotide sequence and the CAG promoter. Treatment group 4 contained 6 mice which were treated with cyclophosphamide at 25 mg/kg. Treatment group 3 was a control group containing 5 MRL/MpJ (JAX stock# 000486) mice which were treated with vehicle only.

MRL/MpJ-Fas^{lpr}/J (commonly known as MRL-lpr or lpr mutant) are homozygous for the lymphoproliferation spontaneous mutation (Fas^{lpr}). (https://www.jax.org/strain/000485). MRL/MpJ are the parent and control strain for MRL/MpJ-Faslpr. MRL/MpJ mice carry the normal Fas gene. (https://www.jax.org/strain/000486).

TABLE 1

| Group | # mice | Strain | Compound | Dosing Route | Dosing Frequency |
|---|---|---|---|---|---|
| 1 | 10 | MRL/Mpl-Fas^{I}^{b}^{r}/J | Vehicle | IV | Single Injection |
| 2 | 10 | MRL/Mpl-Fas^{lbr}/J | AAV-CFl | IV | Single Injection |
| 3 | 5 | MRL/Mpl | Vehicle | IV | Single Injection |
| 4 | 6 | MRL/Mpl-Fas^{Ibr}/J | Cyclophosphamide | IP | Twice weekly |

IV = intravenous; IP = intraperitoneal

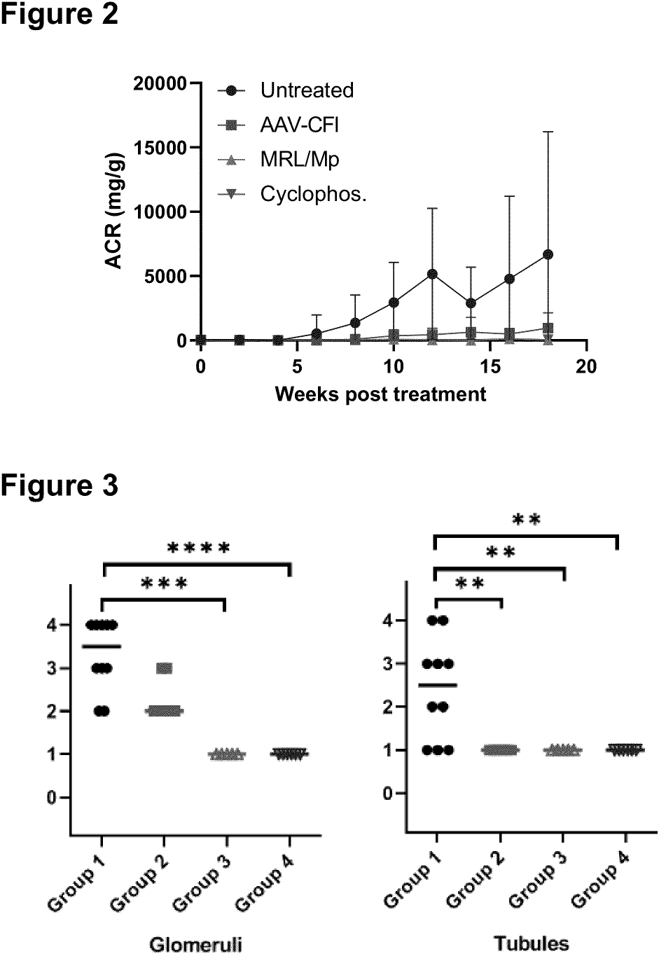

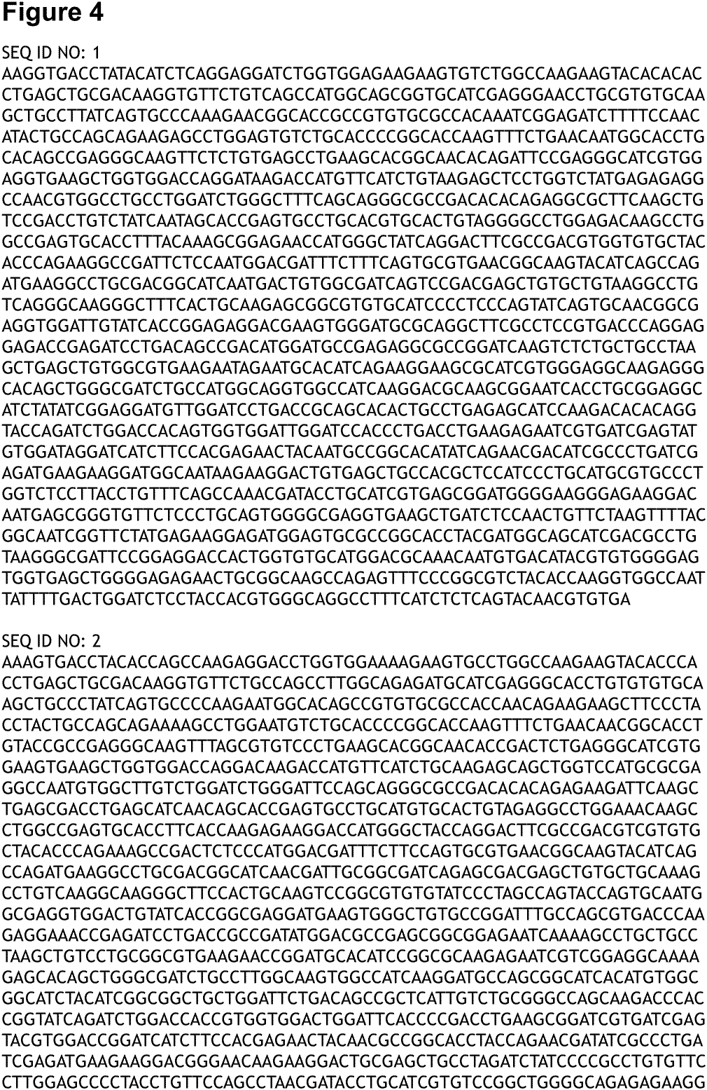

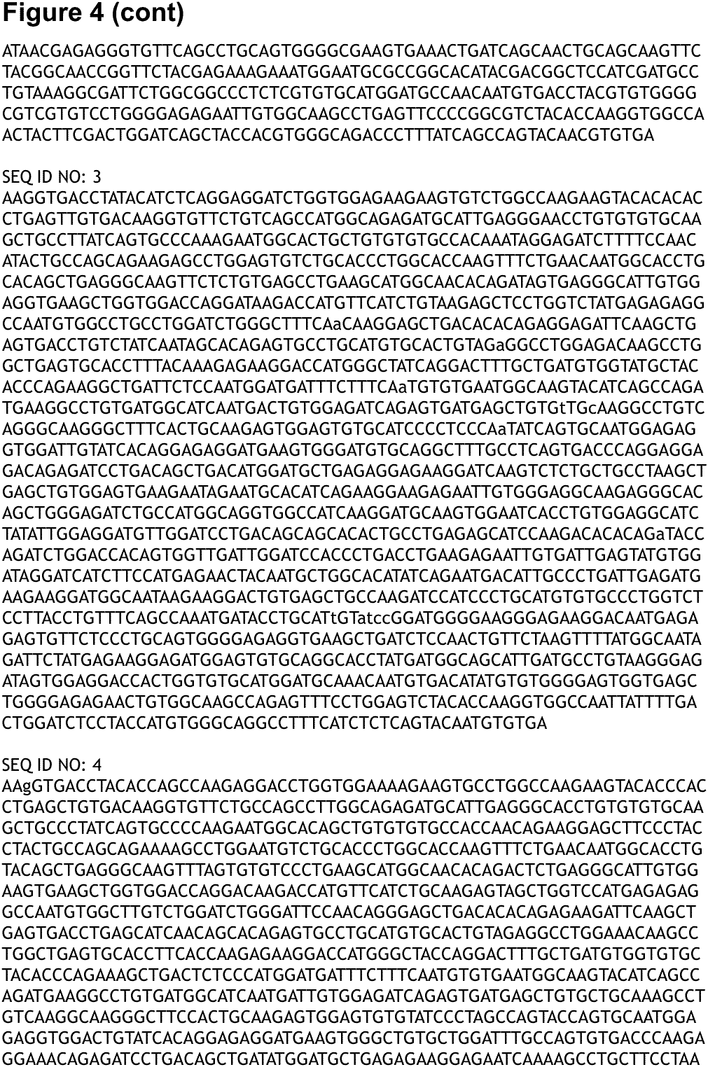

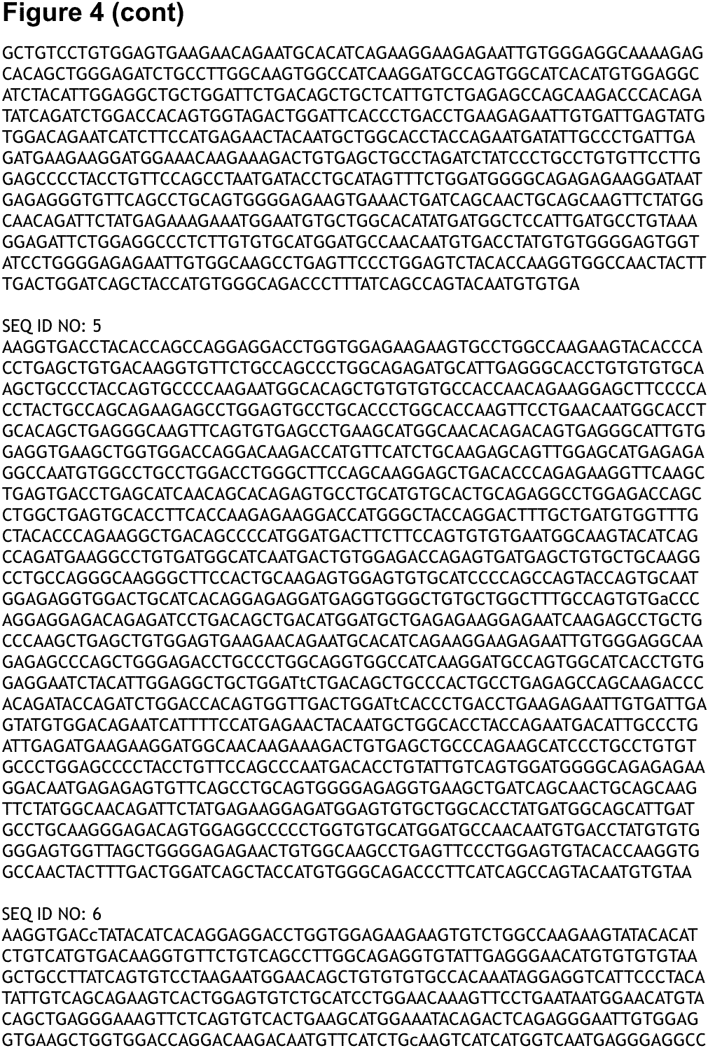

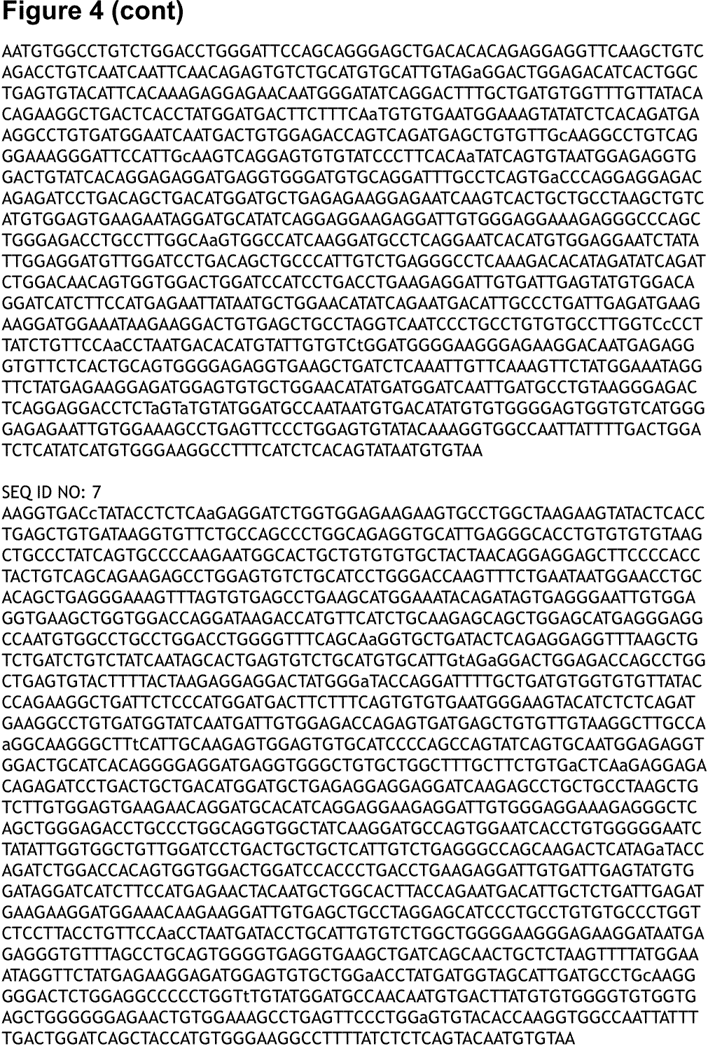

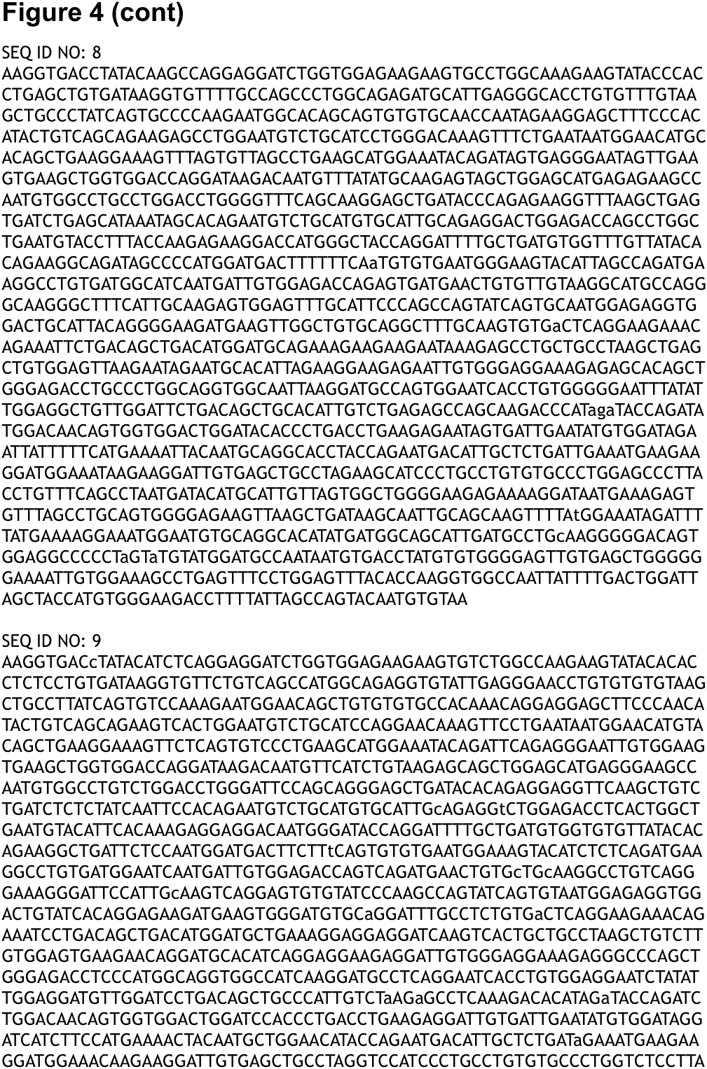

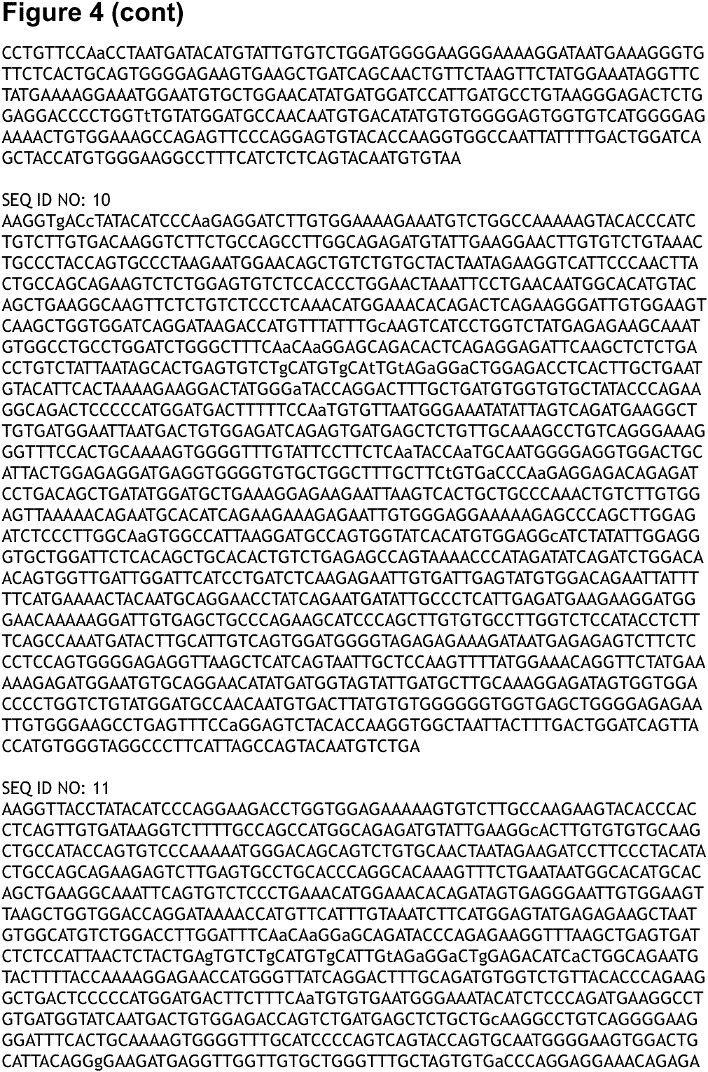

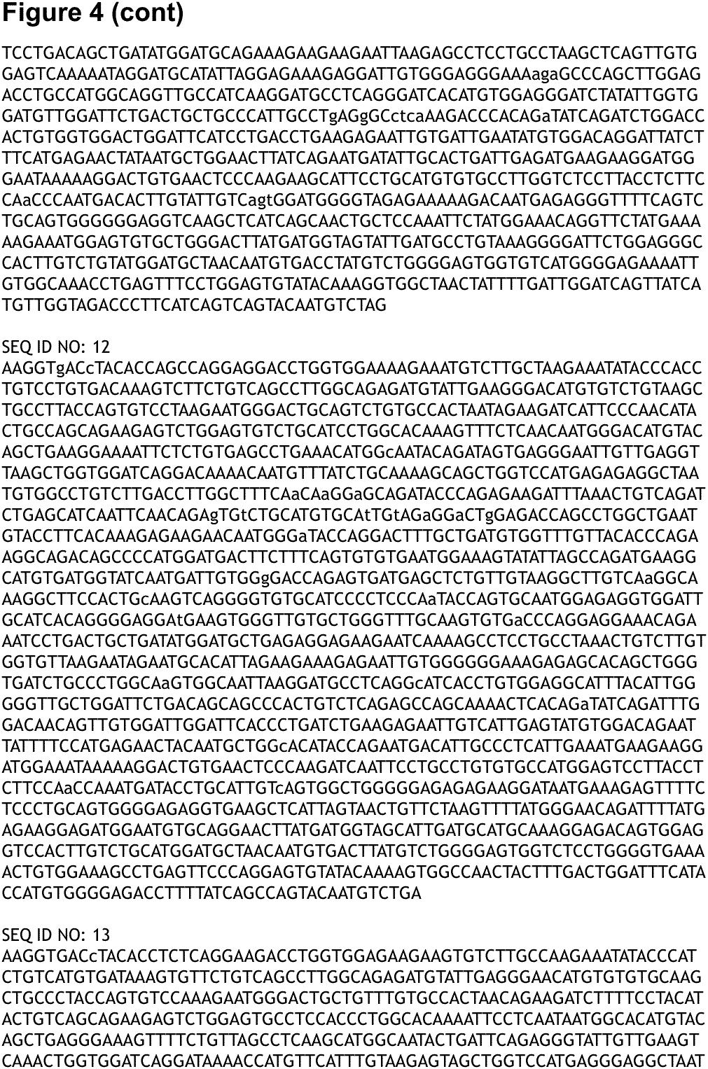

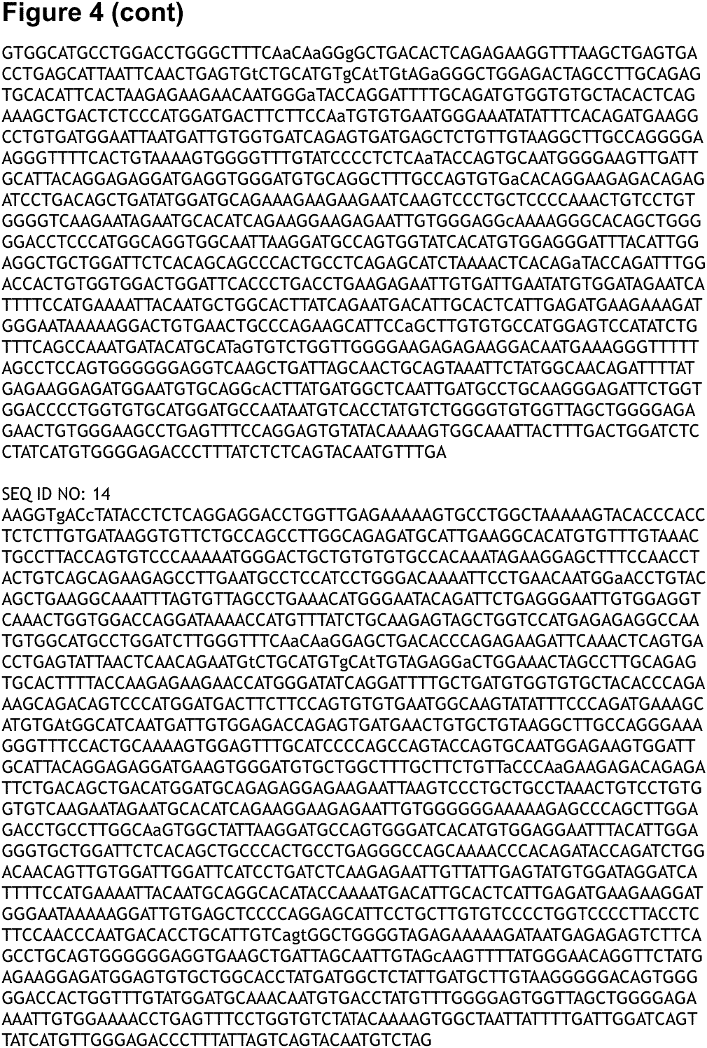

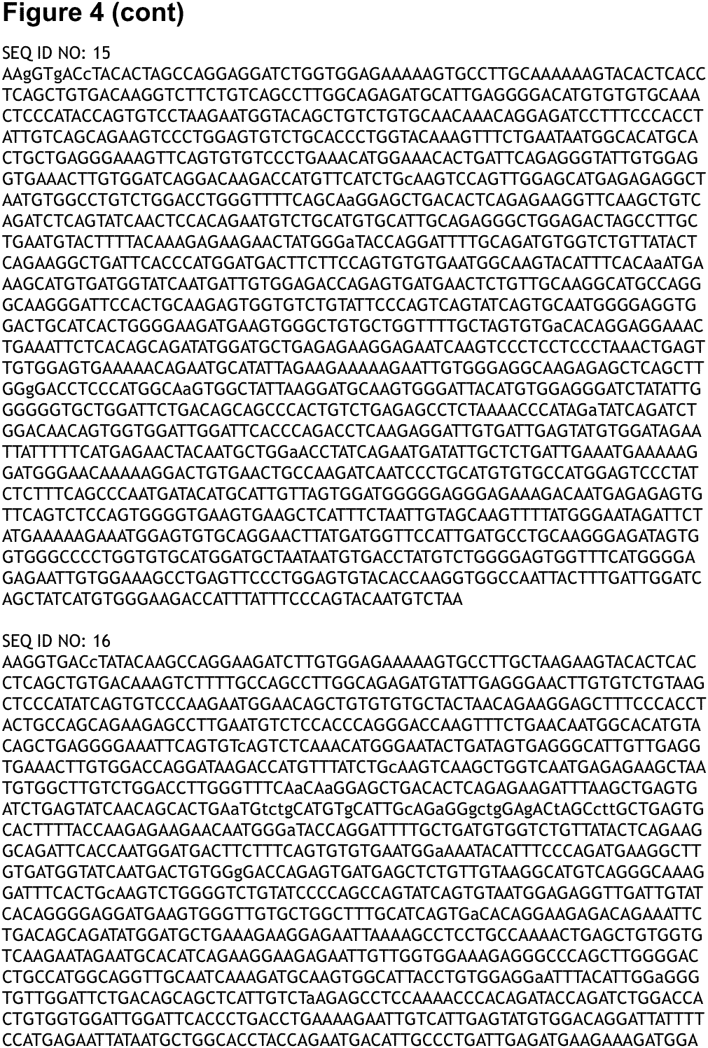

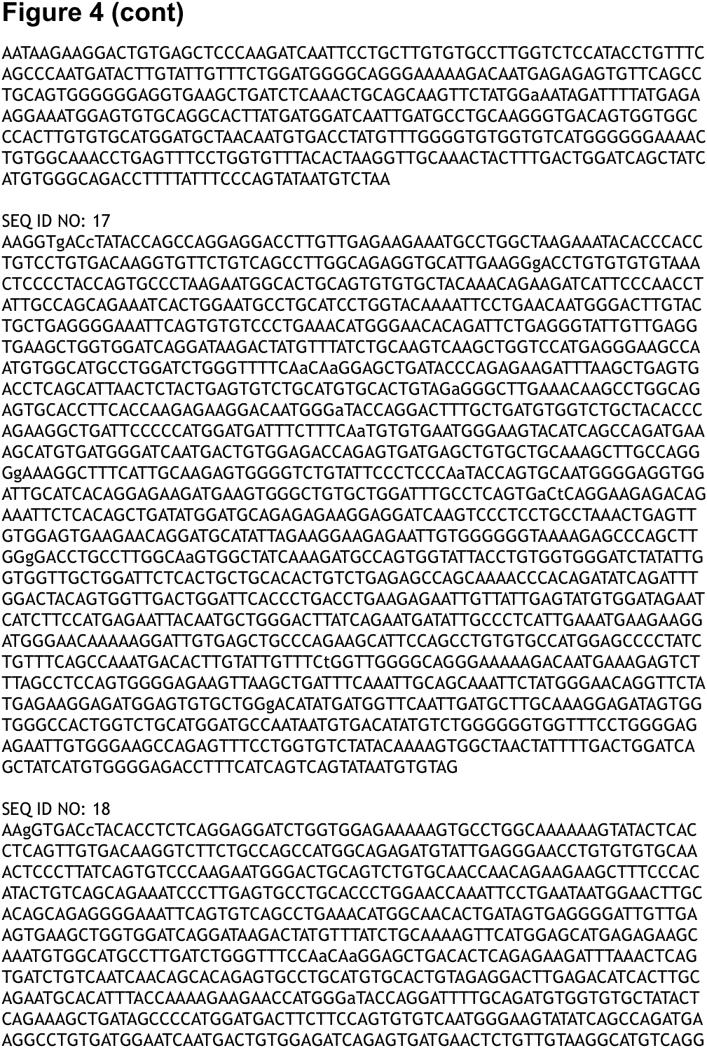

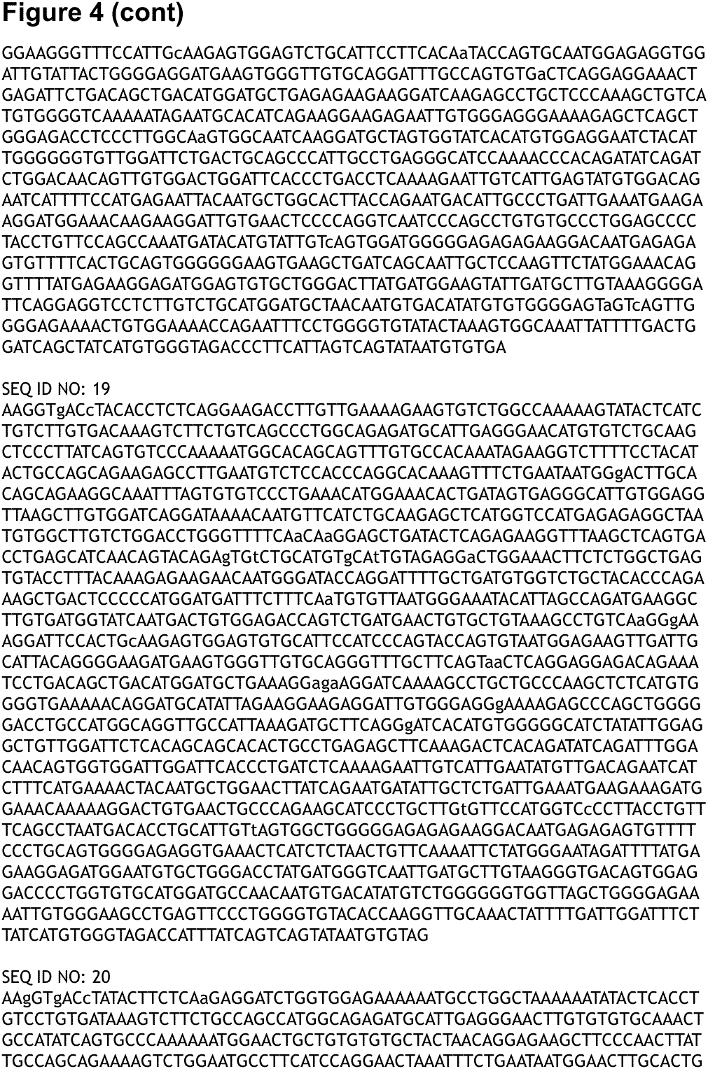

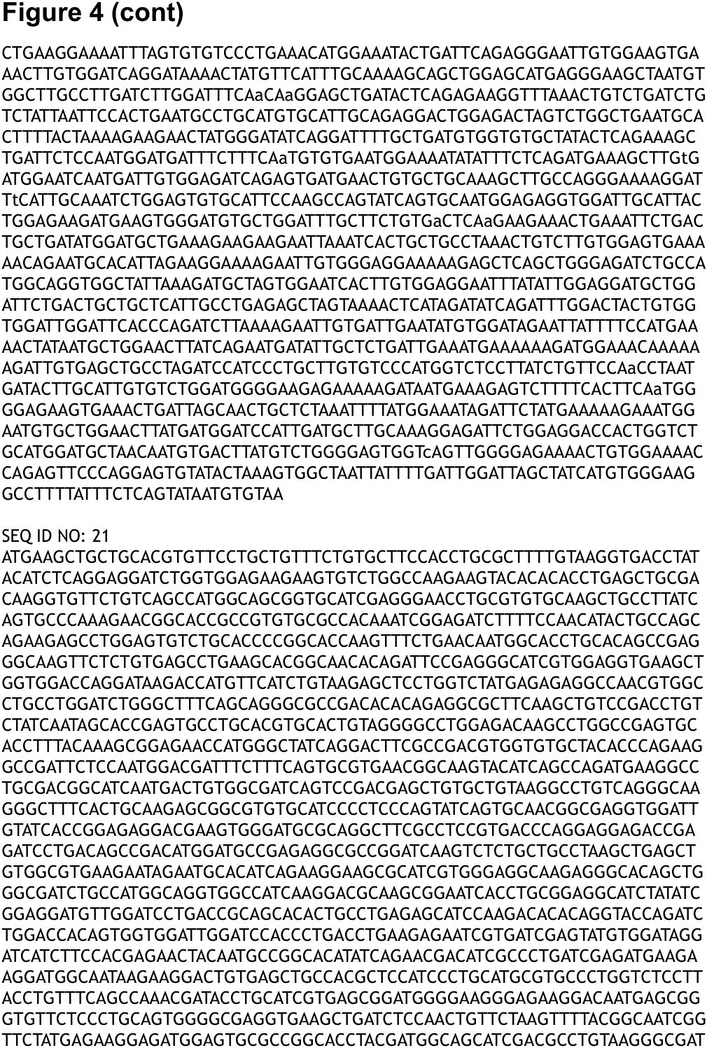

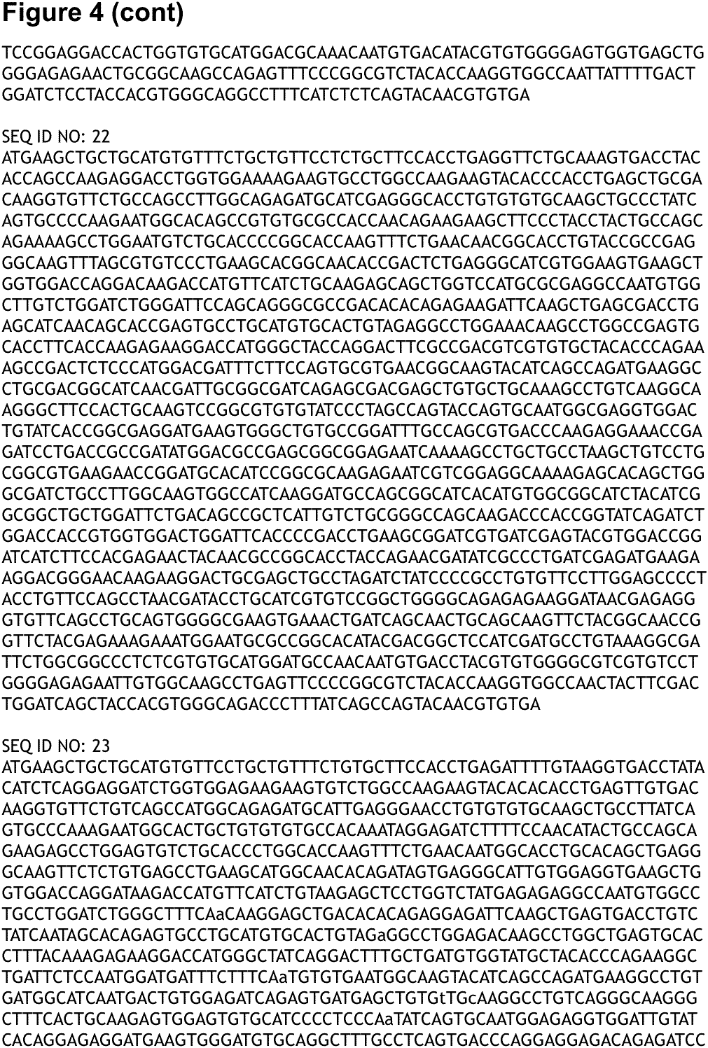

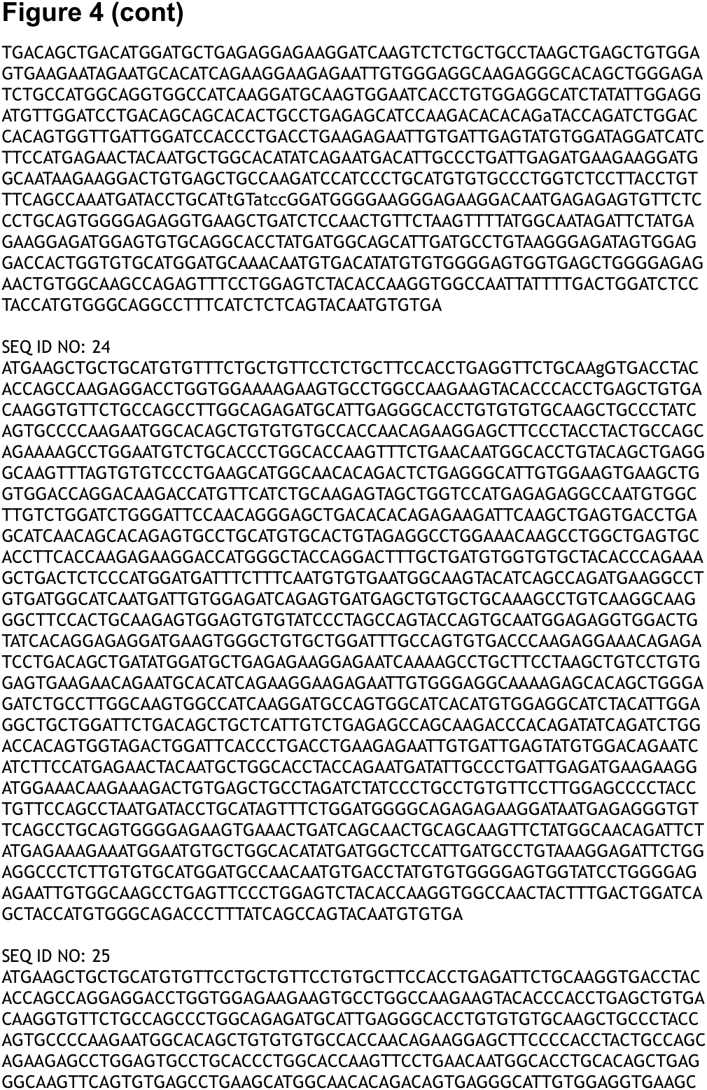

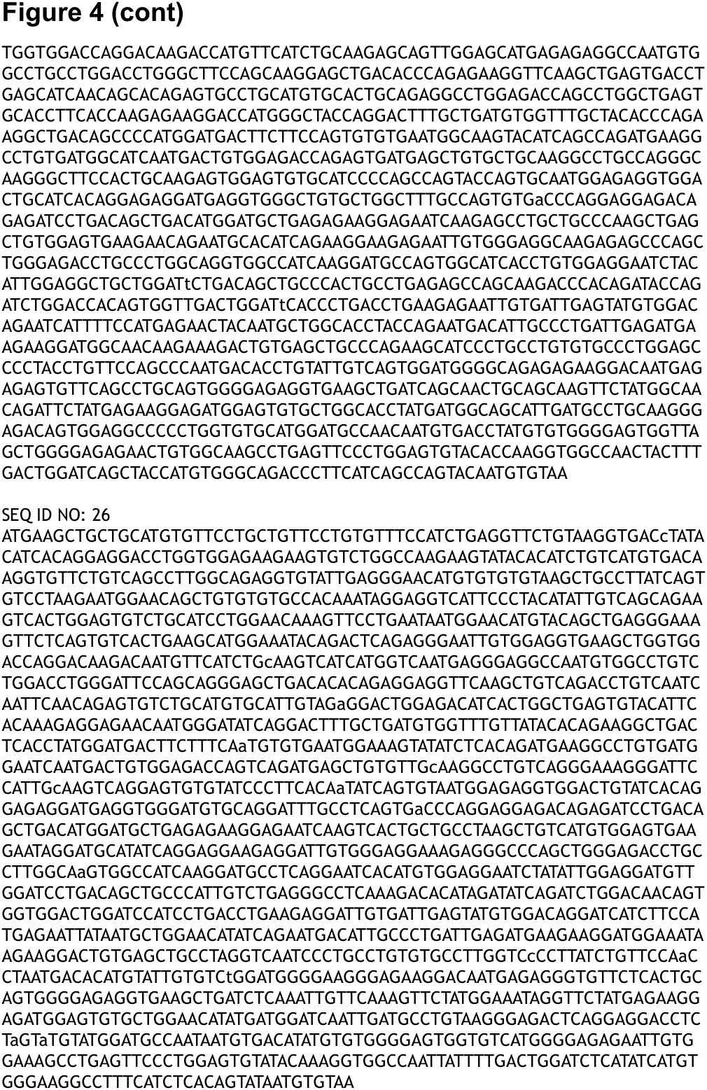

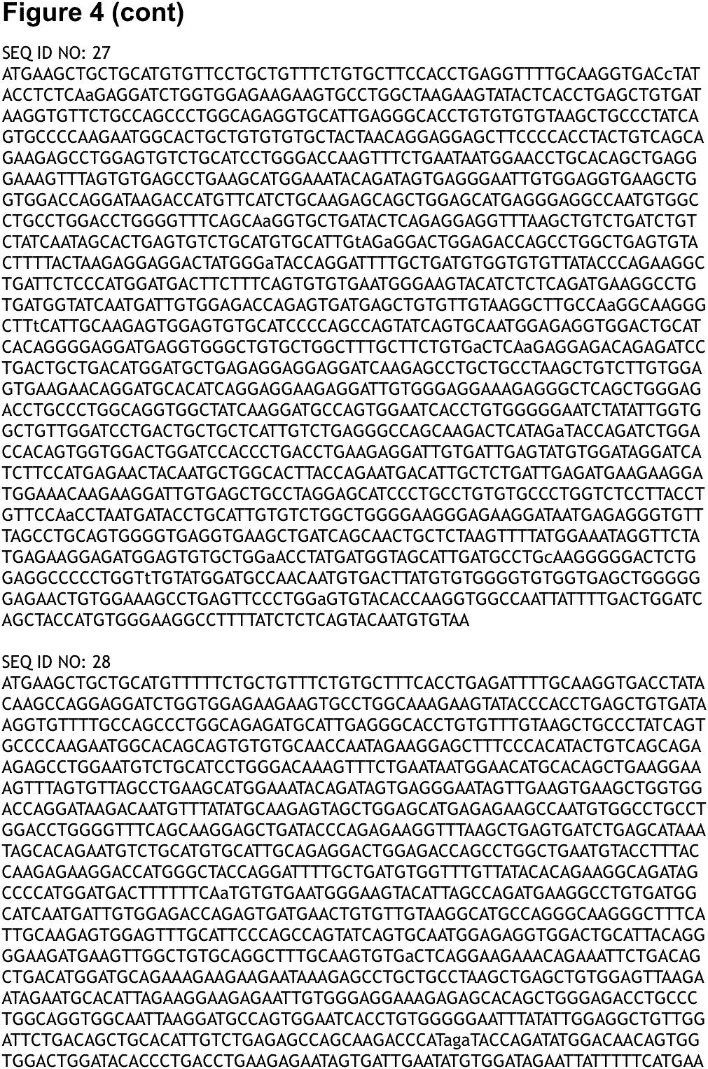

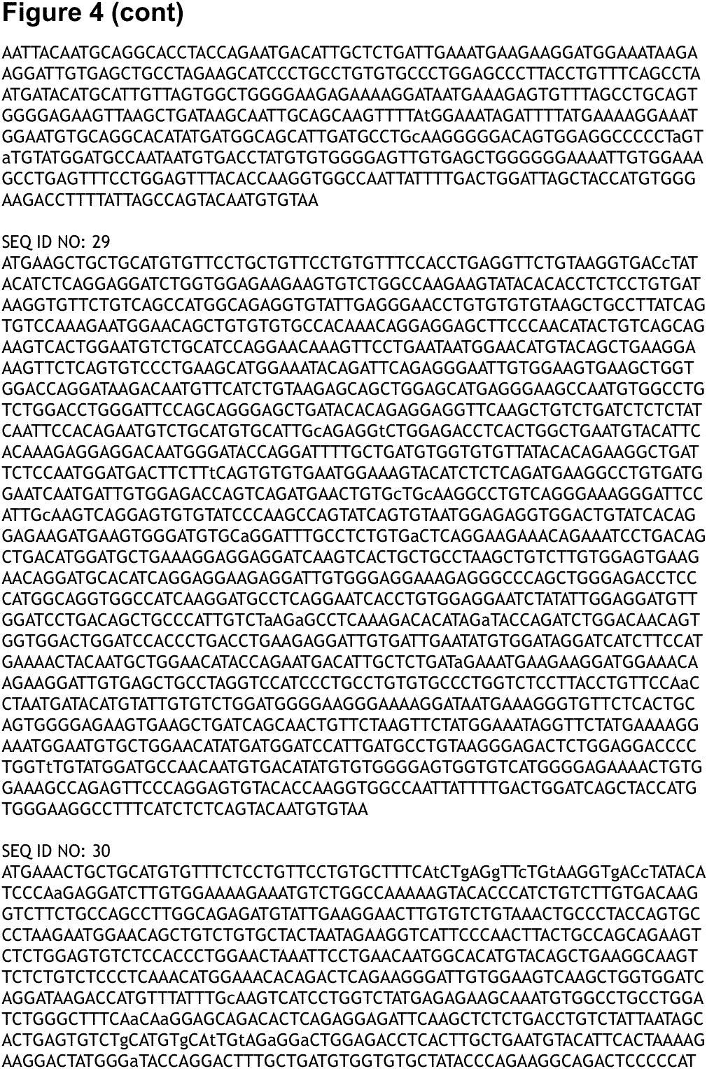

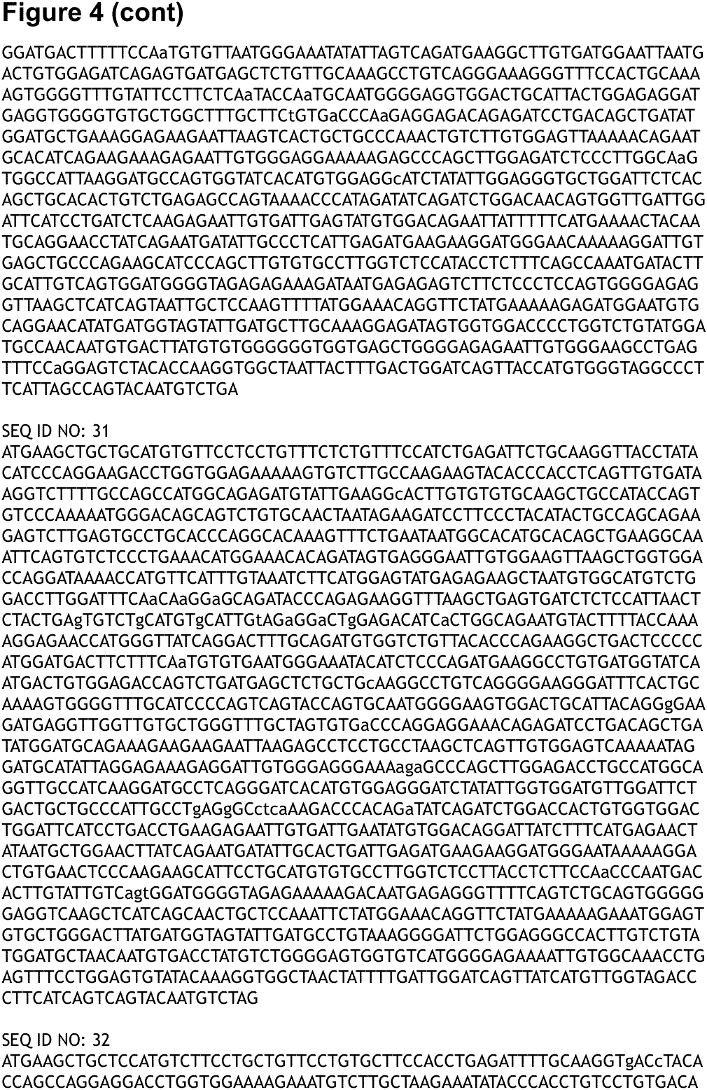

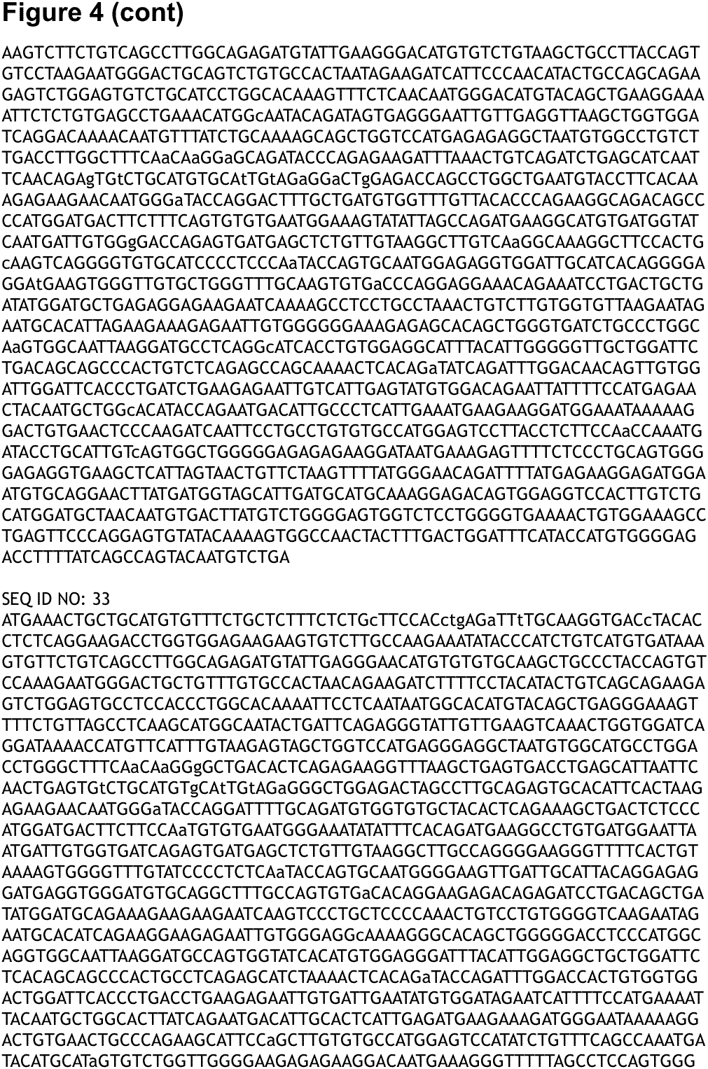

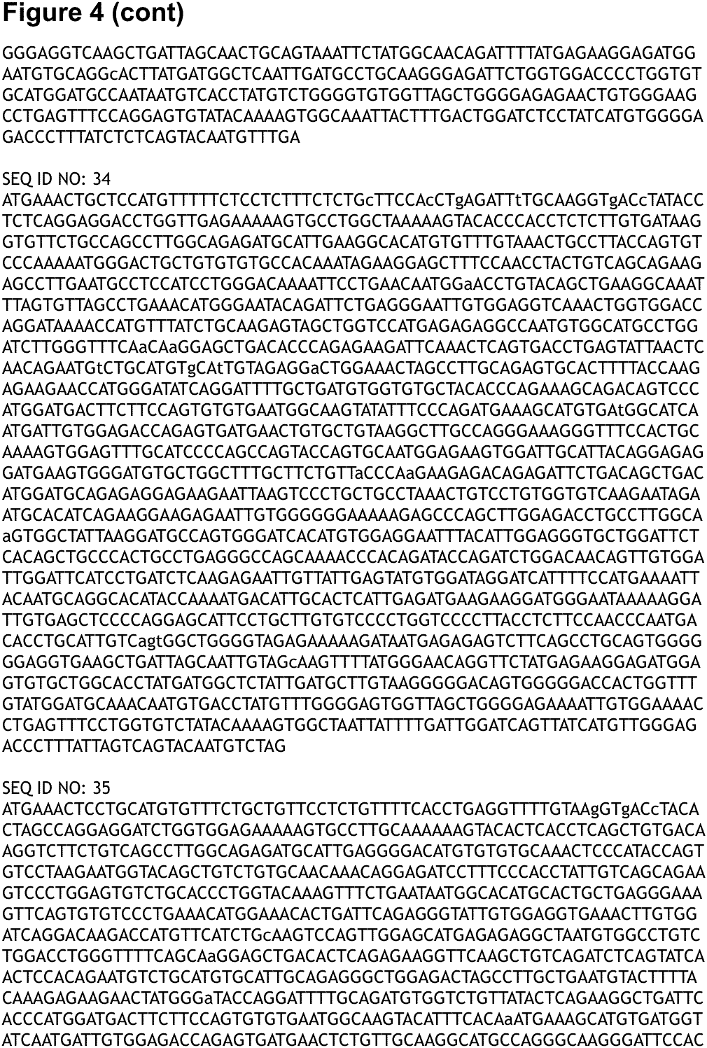

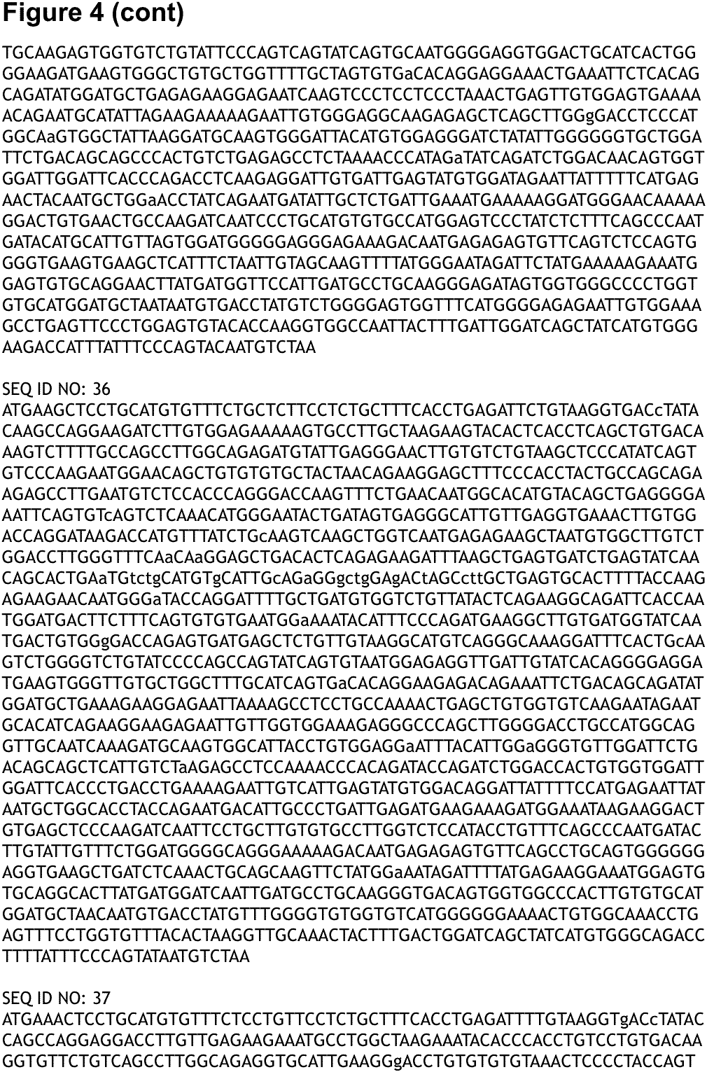

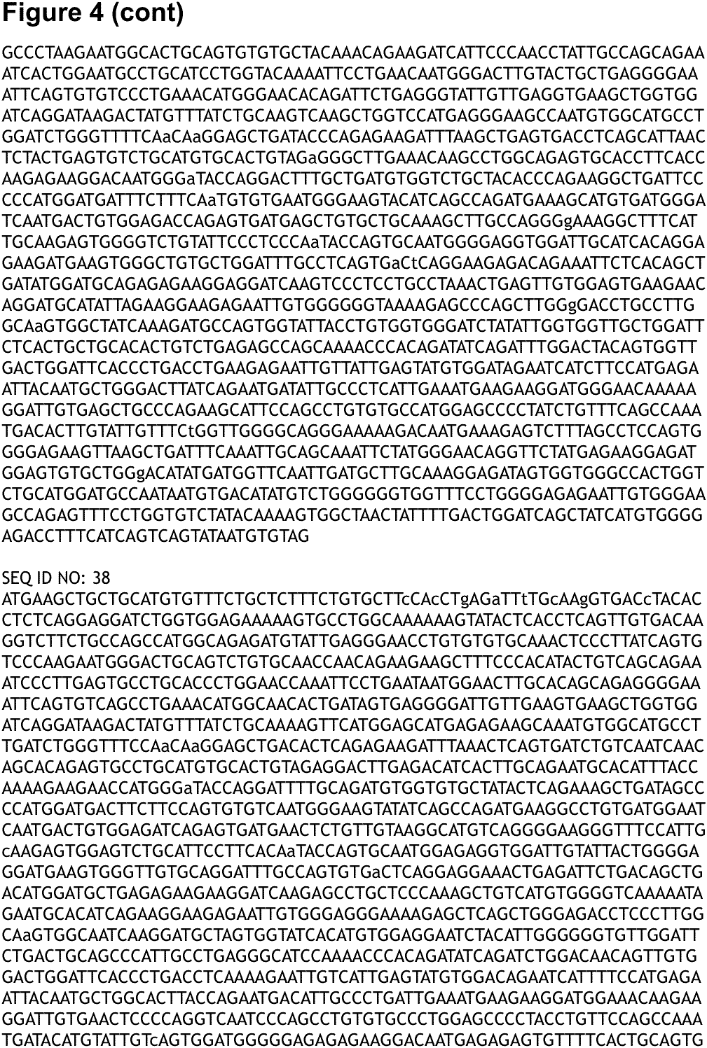

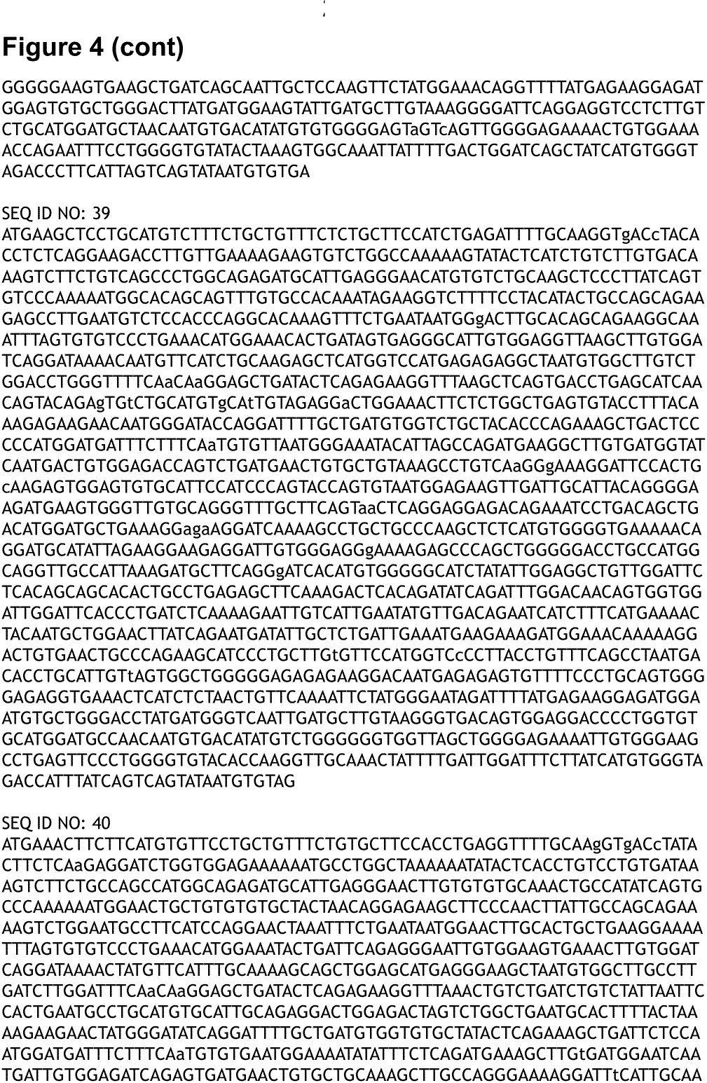

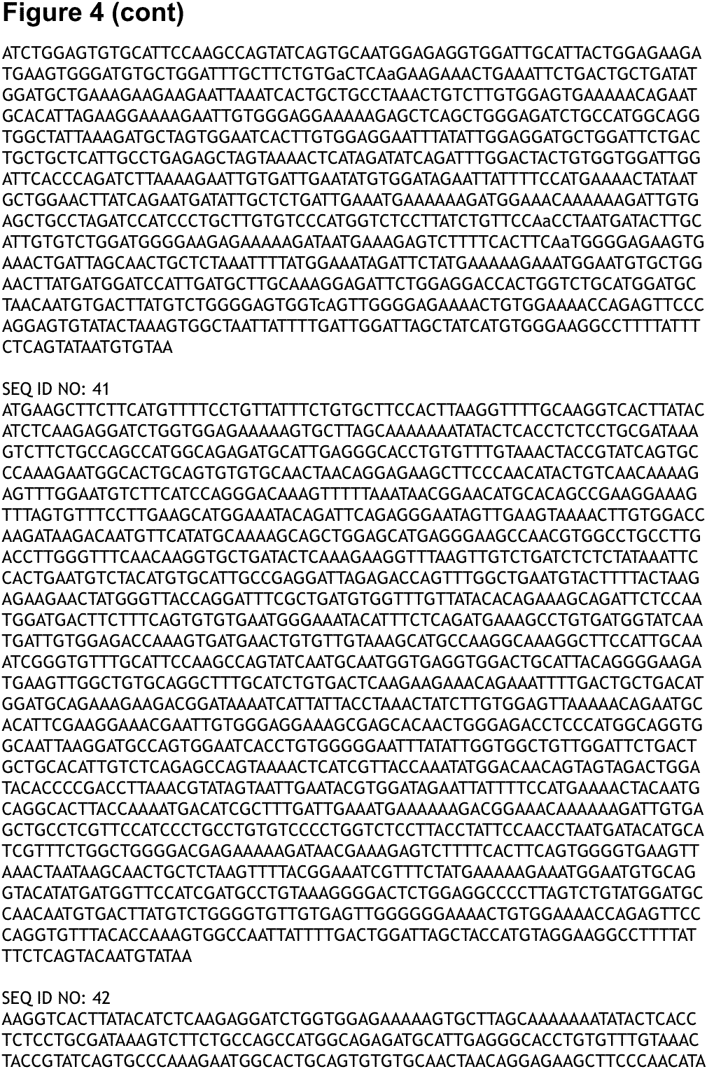

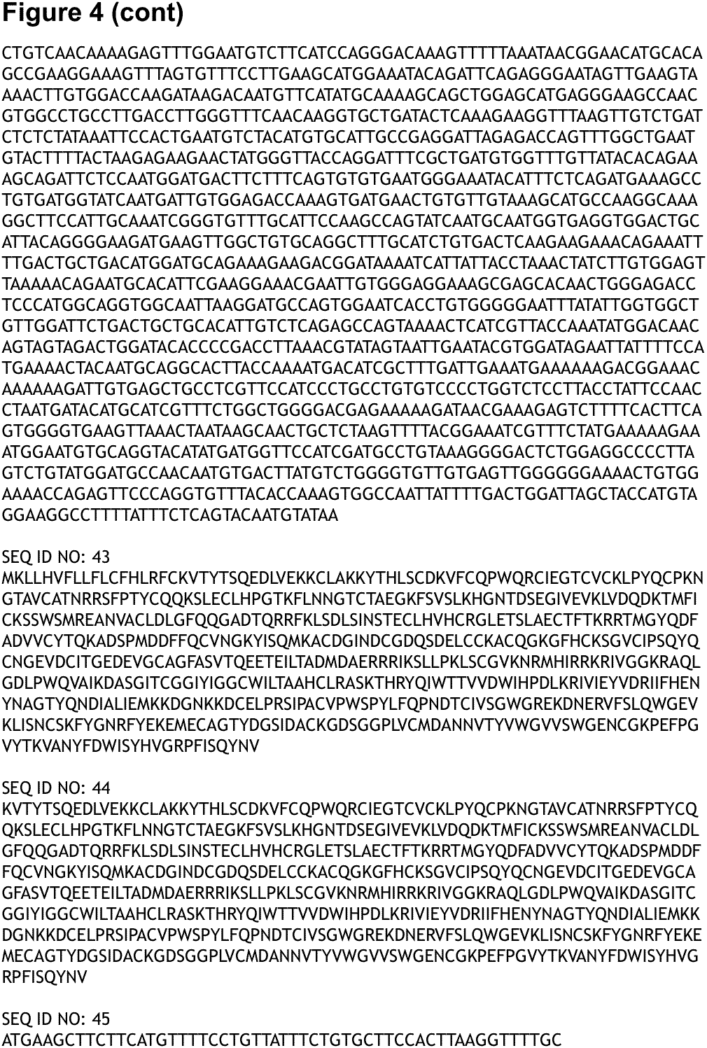

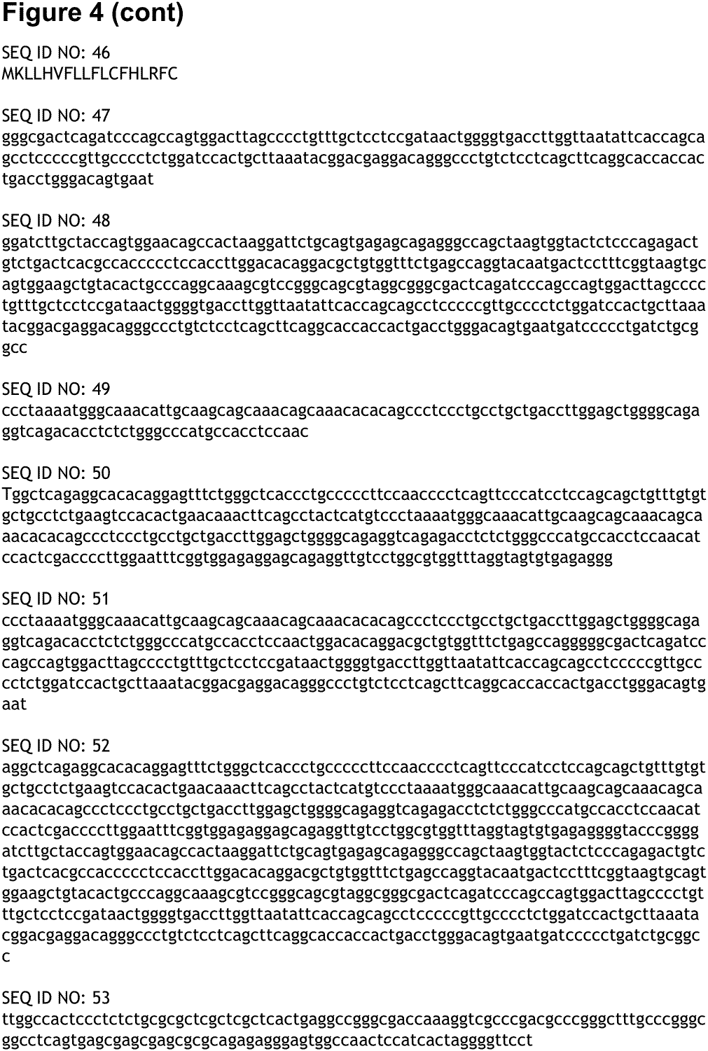

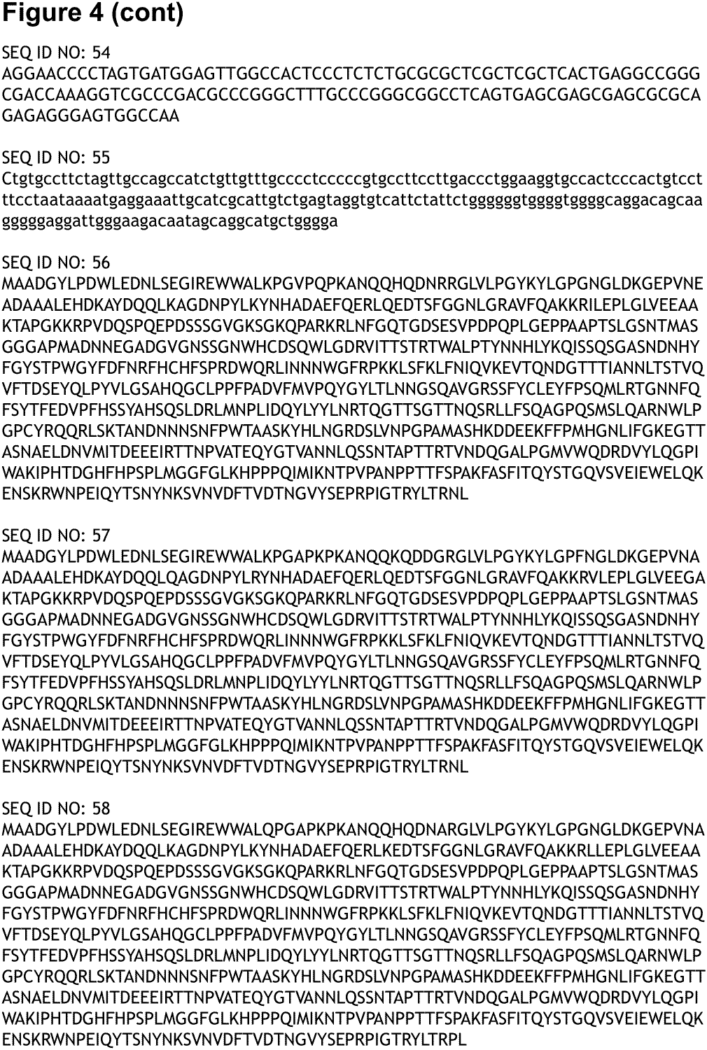

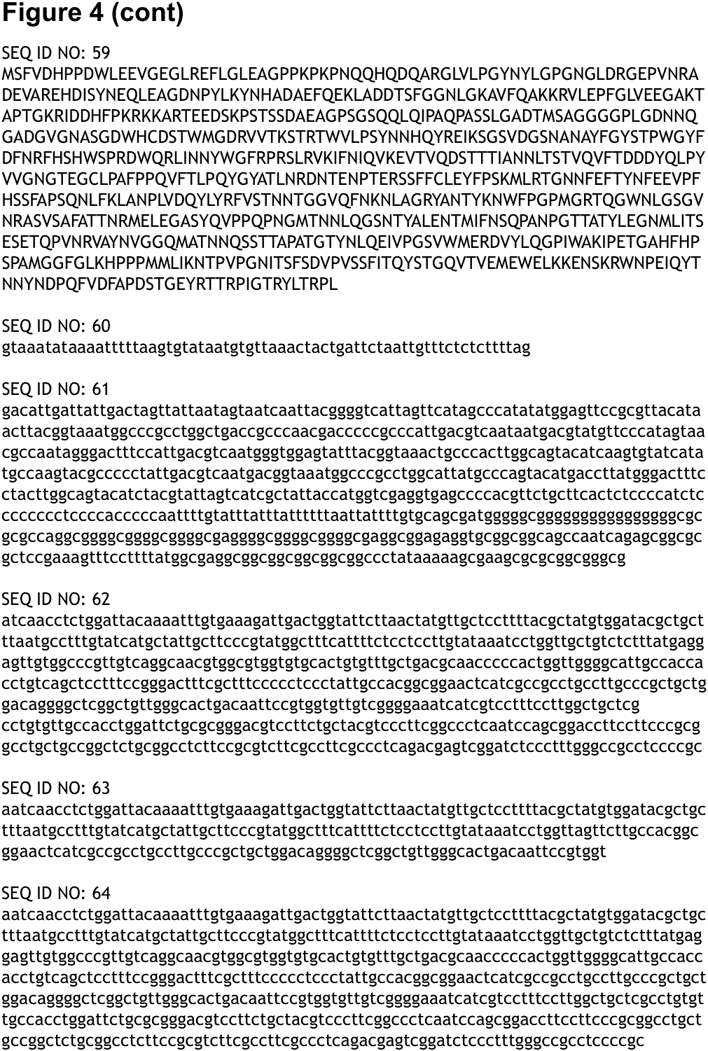

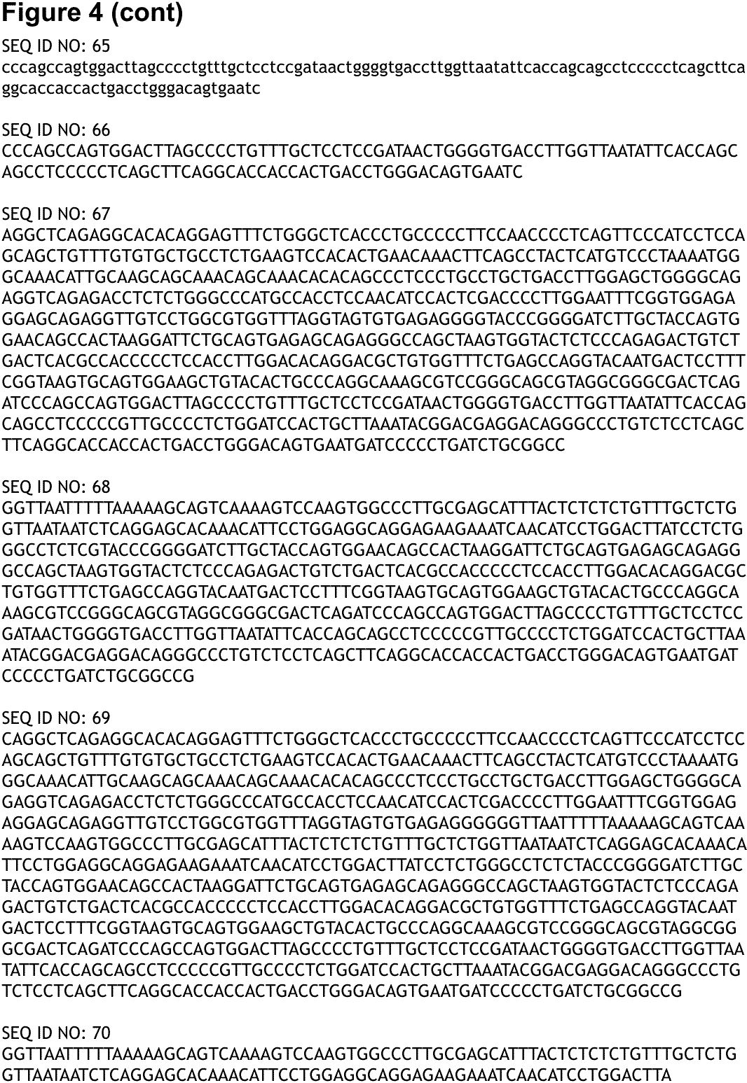

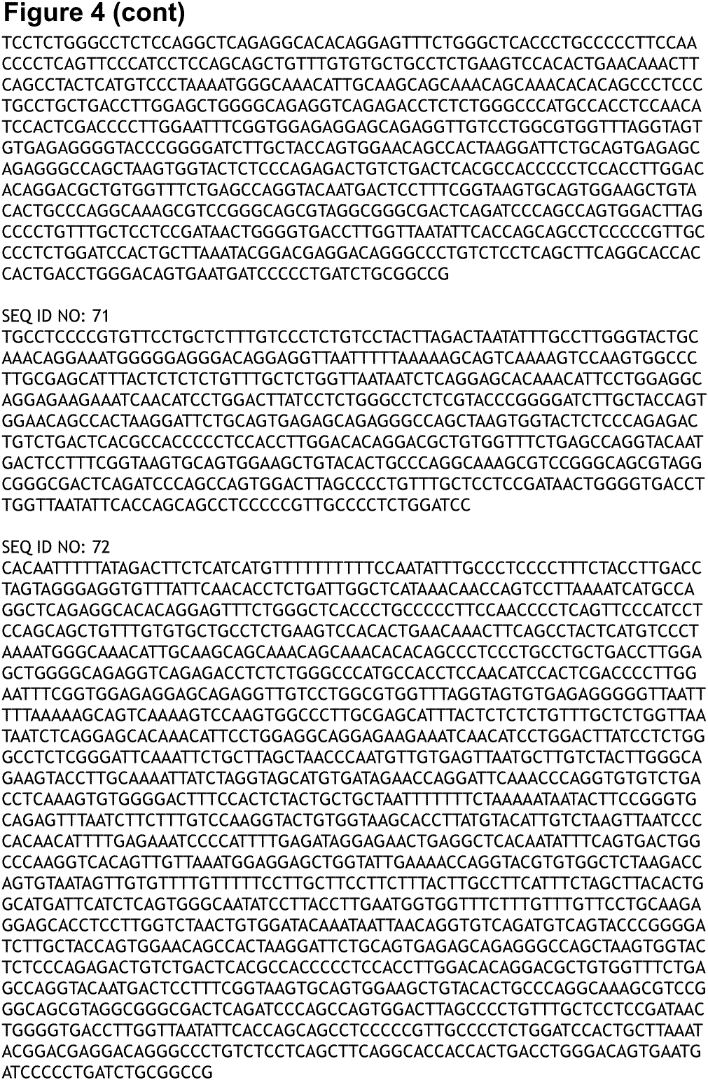

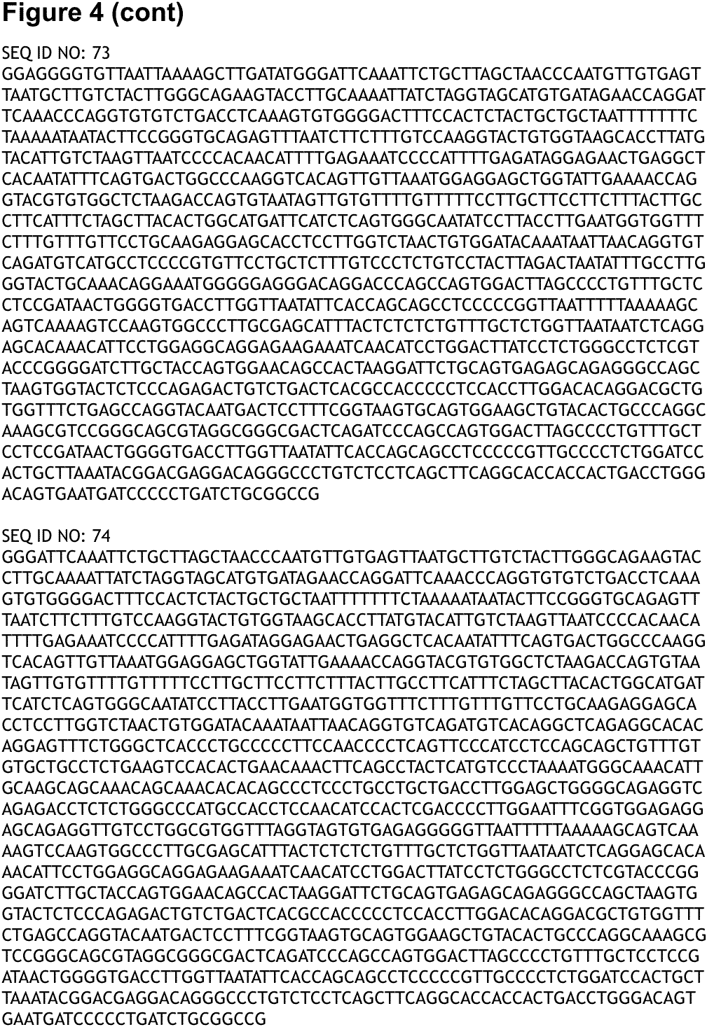

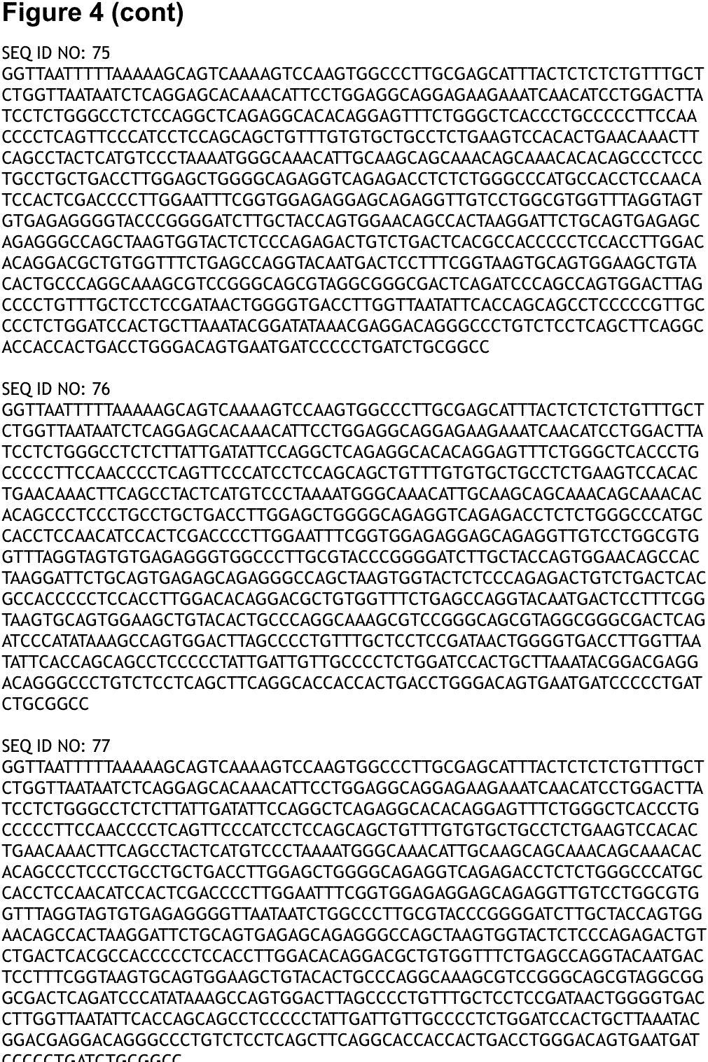

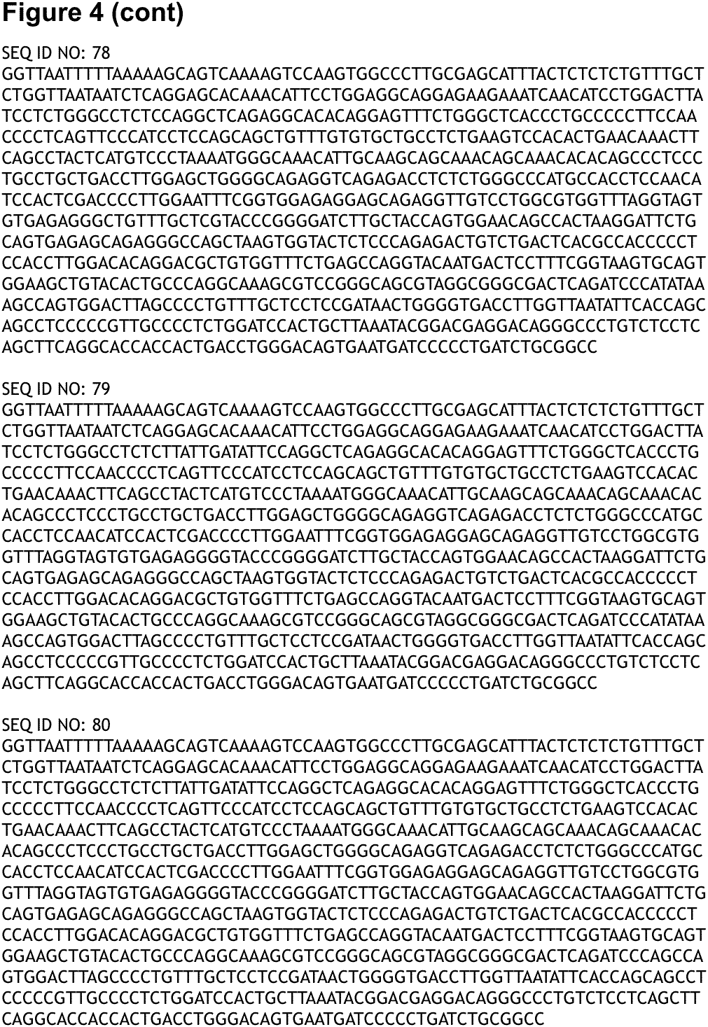

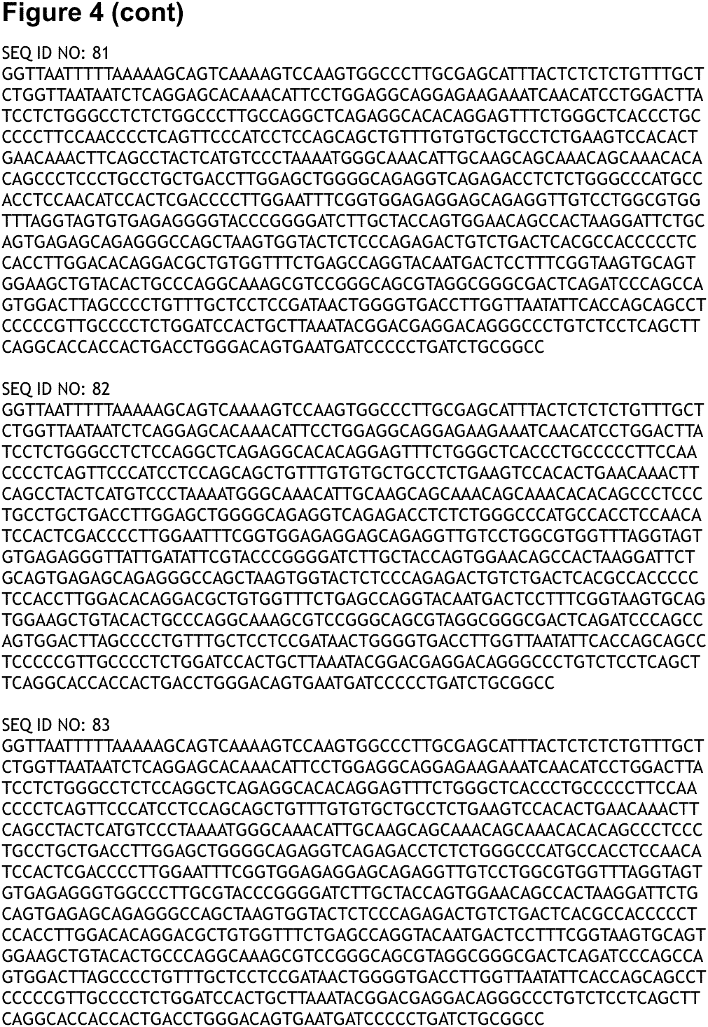

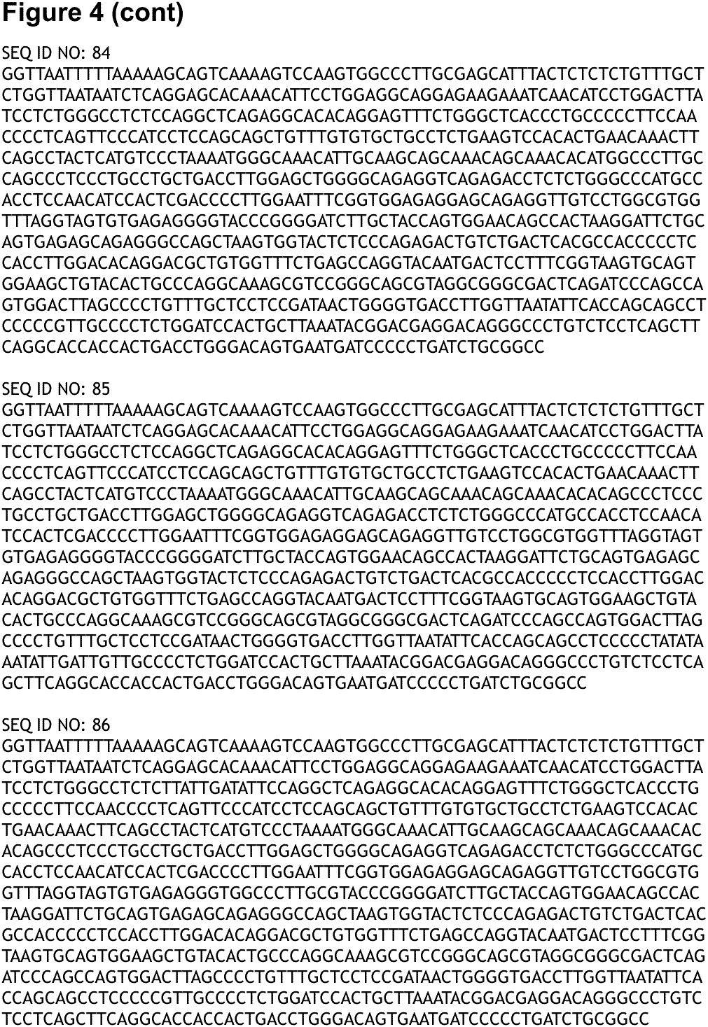

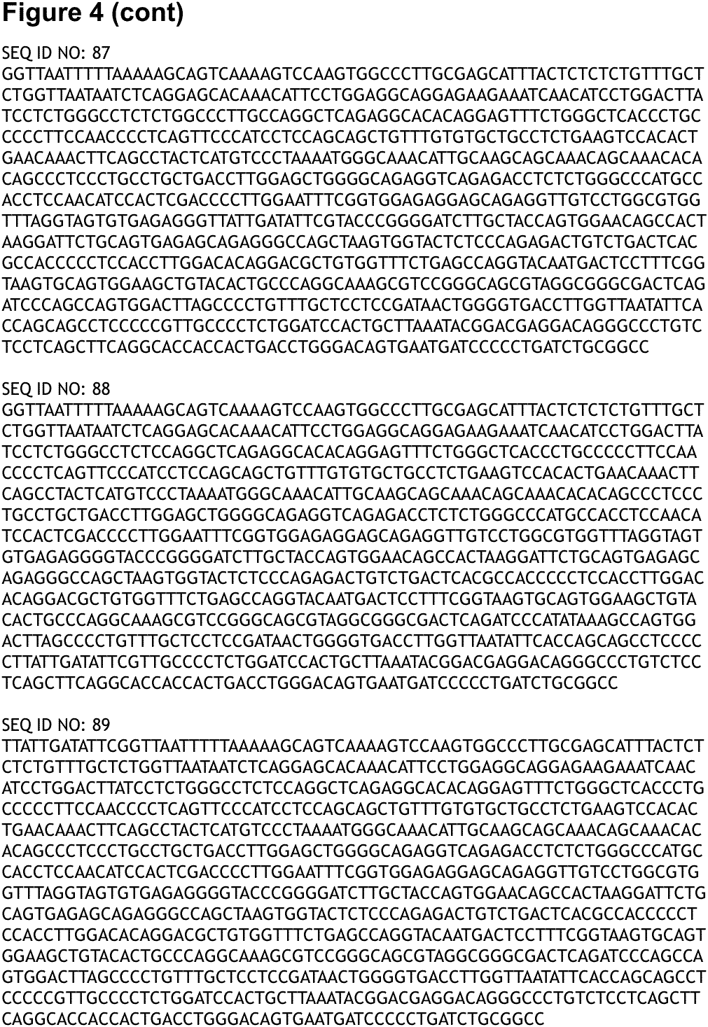

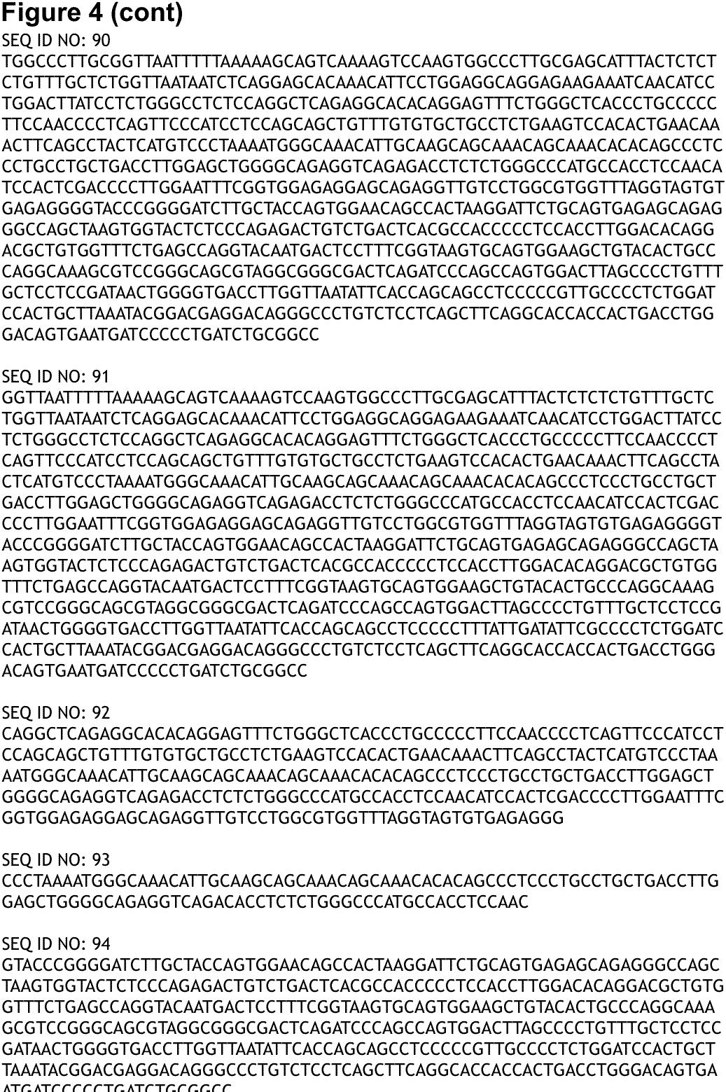

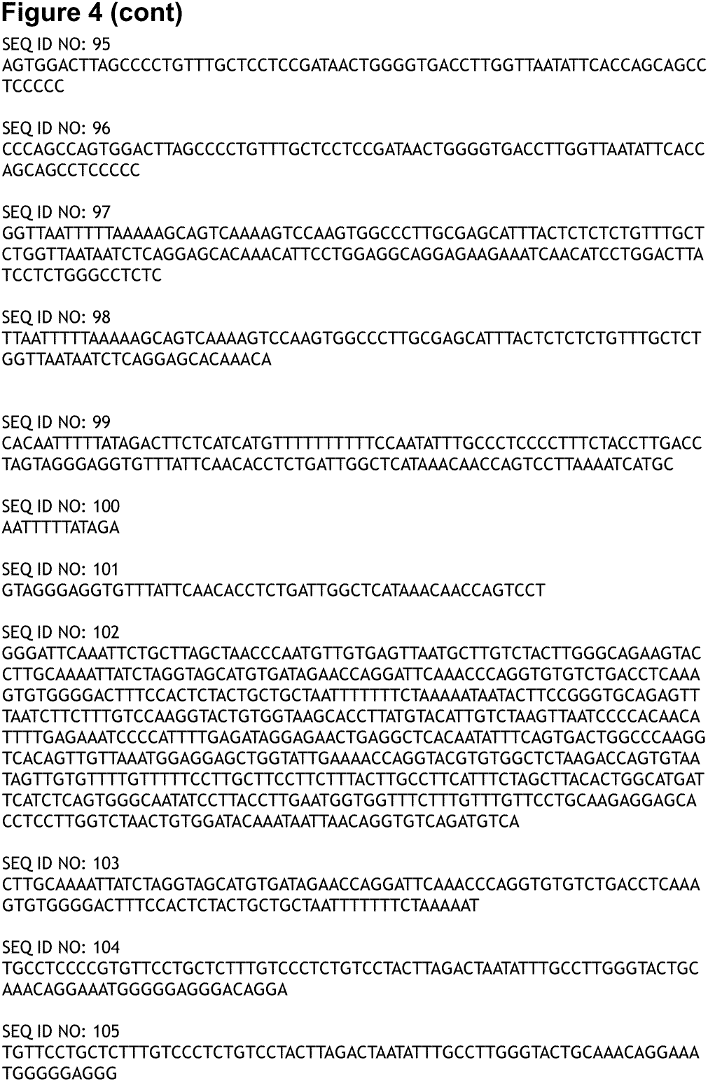

Proteinuria was monitored every two weeks by measuring the ACR (albumin creatinine ratio) in urine using Albustix® and a Beckman AU680 chemical analyzer, and the results are shown in FIG. 2. 3 out of the 10 mice from treatment group 2 died at 9 weeks of age, 15 weeks of age, and 22 weeks of age respectively. Proteinuria data was collected up until death. Treatment with AAV comprising CFI resulted in a reduction of proteinuria.

After euthanasia, the kidneys were collected and one from each mouse was fixed in 10% neutral buffered formalin. The tissues were then processed and embedded in paraffin. The kidneys were stained with Periodic Acid Schiff (PAS) stain. The kidneys were assessed microscopically. Microscopic examination was performed blinded to the treatment group. Pathological changes were graded semi-quantitatively on PAS stained sections on an ascending scale of 0-4 for increased severity, with 0 indicating no pathology, 1 indicating mild pathology, 2 indicating moderate pathology, 3 indicating marked pathology, and 4 indicating severe pathology.

The semi-quantitative scores were compared statistically as nonparametric data using GraphPad Prism version 8.1.1. Mann-Whitney/Kruskal-Wallis tests were used to compare all groups. Treatment effect was analyzed by comparing the treatment group to the vehicle treated group. Statistical significance was assigned to p values <0.05 (* <0.05, ** <0.01, *** <0.001, **** <0.0001). Seven out of the ten mice from treatment group 2 underwent the histopathology.

FIG. 3 shows a reduction in the pathology of the glomeruli and tubules when treating with AAV-CFI (treatment group 2) in comparison to the vehicle-only control (treatment group 1).

### Example 5 - Huh7 in vitro Transfection Study of Codon Optimised Sequences

Unique codon optimised human CFI sequences were screened and ranked based on the amount (w/v) of protein produced after transient reverse transfection of Huh7 cells. Each of the codon optimised sequences encoded a wild-type human CFI polypeptide.

### Methods

Expression cassettes were generated which comprise one of the codon optimised CFI sequences or a wild type CFI sequence for control purposes. The promoter used was “LSP-S” (SEQ ID NO: 51), which is a liver-specific transcription regulatory element.

The transient transfection entails mixing, for each expression cassette comprising one of the unique codon-optimised CFI genes, a plasmid carrying such expression cassette with a transfection reagent (FuGENE®HD (Promega, cat no. E2311)) and delivering the mixture to cells so that the gene can be expressed and protein subsequently quantified by ELISA. Where applicable, a luciferase reporter gene was included in the transfection mixture as a way of evaluating transfection efficiency.

Huh7 cell culture and Transfection: Briefly, 0.8 µg of test plasmid DNA and 0.08 µg TK-Luciferase control plasmid were mixed with FuGENE at a ratio of 3 µL FuGENE per µg of DNA (or 1 µL FuGENE per 0.33 µg of plasmid DNA). 50 µL of the transfection mixture was transferred to a 24-well plate. 300,000 Huh7 cells (JCRB cell bank, no. JCRB0403) were then seeded per well in 450 µL DMEM low glucose, 10% FBS + Glutamax (D10 medium). The plasmid DNA-transfection reagent mixture was cultured with the cells overnight at 37° C. and 5% CO₂. Approximately 24 hours after transfection, the media was replaced with 500 µL DMEM low glucose + Glutamax + Insulin-Transferrin-Selenium supplement (D0/ITS media). 24 hours later the media (supernatant) were harvested.

CFI expression in culture media (supernatant) was assessed using a CFI ELISA kit (Hycult cat# HK355-02), performed in accordance with the manufacturer’s instructions. In brief, undiluted culture media samples and human CFI standards from the kit were incubated in microtiter wells coated with anti-human CFI antibodies (to capture the human CFI). A biotinylated tracer antibody was added (to bind to the captured human CFI) followed by a streptavidin-peroxidase conjugate (to bind to the biotinylated tracer antibody). The substrate tetramethylbenzidine (TMB) was then added, which the streptavidin-peroxidase conjugate reacts with. The enzyme reaction was stopped by adding oxalic acid. For each of the samples and standards, the absorbance at 450 nm was measured with a Molecular Devices SpectraMax i3x plate reader. A standard curve was obtained by plotting the absorbance (linear) against the corresponding concentrations of the human CFI standards (log). The concentration of human CFI in the samples was then determined from the standard curve.

Luciferase expression of the supernatants was measured using a Nano-Glo® Luciferase Assay System (Promega cat# N1120). 20 µL of supernatant was diluted in media to a final volume of 100 µL, and mixed with an equal volume of Nano-Glo® Luciferase Assay Reagent. Following a 3 minute incubation at room temperature, luciferase expression was measured by luminescence on a Molecular Devices SpectraMax i3x plate reader. The detailed protocol is published in the Promega Nano-Glo Luciferase Assay System Technical Manual #TM369.

Luciferase expression was used as an internal control to normalise the CFI expression levels. Analyses were performed using the software Graphpad Prism v9.0.1.

### Results

The CFI expression levels in the culture media following transfection into Huh-7 cells were measured for a wild-type human CFI nucleotide sequence and 20 codon-optimised human CFI sequences (Co01 to Co20, SEQ ID NOs: 21 to 40, respectively). The CFI expression levels were normalised using luciferase expression as described above. Each expression cassette comprised the LSP-S promoter and the BgpA polyA sequence.

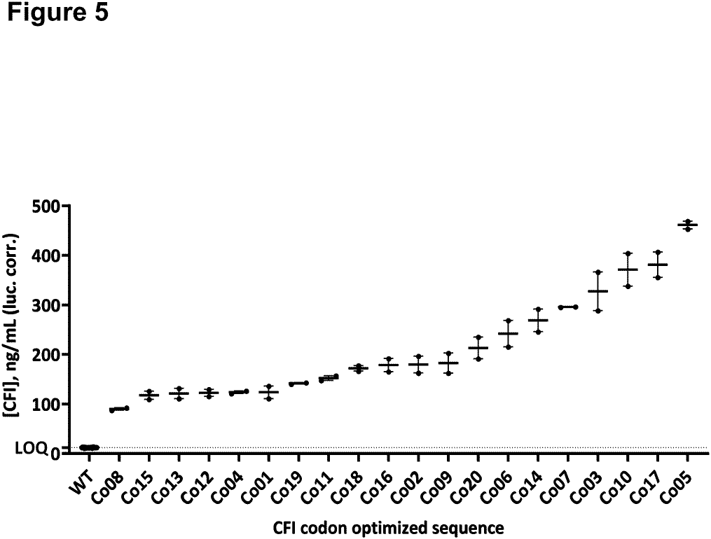

FIG. 5 shows that all of the tested codon optimised CFI sequences (Co01 to Co20) expressed more highly than the wild type CFI sequence.

### Example 6 - Comparison of CFI Expression in Huh7 Cells In Vitro When the Transgene is Under the Control of Different Promoters

Three separate test plasmid DNA constructs were prepared which incorporated a codon-optimised transgene sequence (Co05, SEQ ID NO: 25) encoding a wild-type human CFI protein under the control of either the FRE72 promoter (SEQ ID NO: 65) or the “LSP-S” (SEQ ID NO: 51) or “LSP-L” (SEQ ID NO: 52) promoters, each of which is a liver-specific transcription regulatory element.

To compare expression levels for each of the promoters, Huh7 cells (JCRB cell bank, no. JCRB0403) were seeded in a 96 well plate (30,000 cells per well) in DMEM low glucose, 10% FBS + Glutamax (D10 media) and cultured at 37° C. and 5% CO₂ (day 1). The next day (approx. 24 hours after cell seeding; day 2), the plasmid DNA-transfection reagent mixture was prepared and transfected into Huh7 cells. 0.225 µg of test plasmid DNA and 0.025 µg CMV-Luciferase control plasmid were mixed with FuGENE at a ratio of 4 µl FuGENE per µg of DNA (or 1 µl FuGENE per 0.25 µg of plasmid DNA). For 96-well transfection experiments, 1µl of the FuGENE mix was added per well. The plasmid DNA-transfection reagent mixture was incubated on the cells overnight at 37° C. and 5% CO₂.

The next morning (day 3), approximately 18 hours after transfection, the media was replaced with fresh D10 media and cells incubated overnight at 37° C. and 5% CO₂. The next morning (24 h later; day 4), media was replaced by fresh DMEM low glucose + Glutamax + Insulin-Transferrin-Selenium supplement (D0/ITS media). Cells and media were harvested the following day (on day 5).

Protein expression in culture media was assessed using an ELISA kit.

In parallel to the ELISA, Huh7 cells were washed with Phosphate buffered saline (PBS) twice and cells treated with 100µl of luciferase lysis buffer of the Luciferase assay kit

(Promega cat# E1501/E4530). Cell lysates were stored at -80° C. On the day of luciferase assays, cell lysates were thawed and 20 µl of the sample was used to measure the luciferase expression by luminescence on a Molecular Devices SpectraMax i3x plate reader. The detailed protocol is published in the Promega Technical Bulletin #TB281. Luciferase expression was used as internal control to normalise the protein levels. Analyses were performed using the software Graphpad Prism v7.

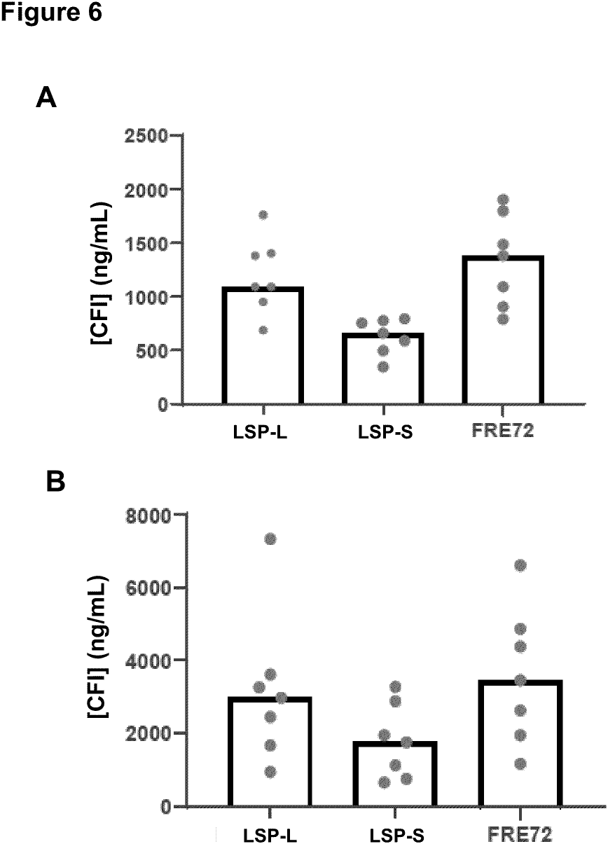

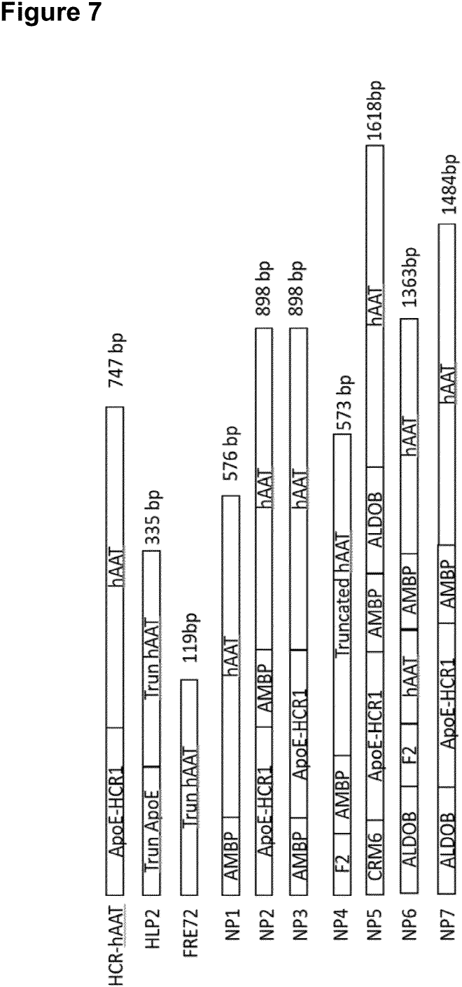

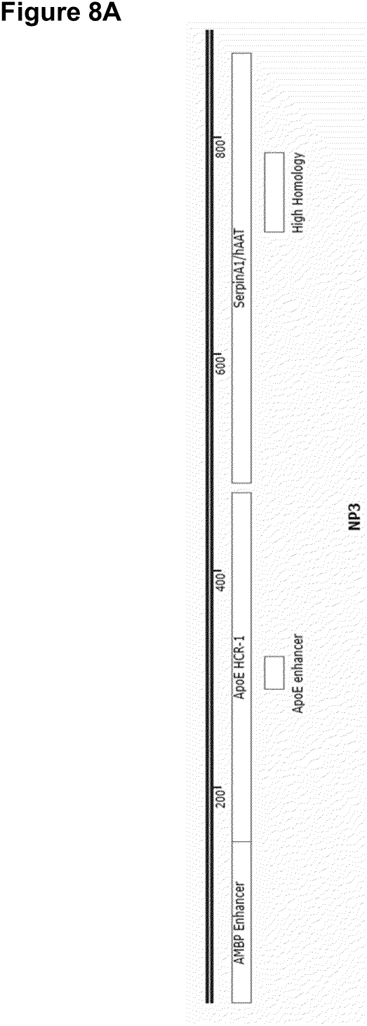

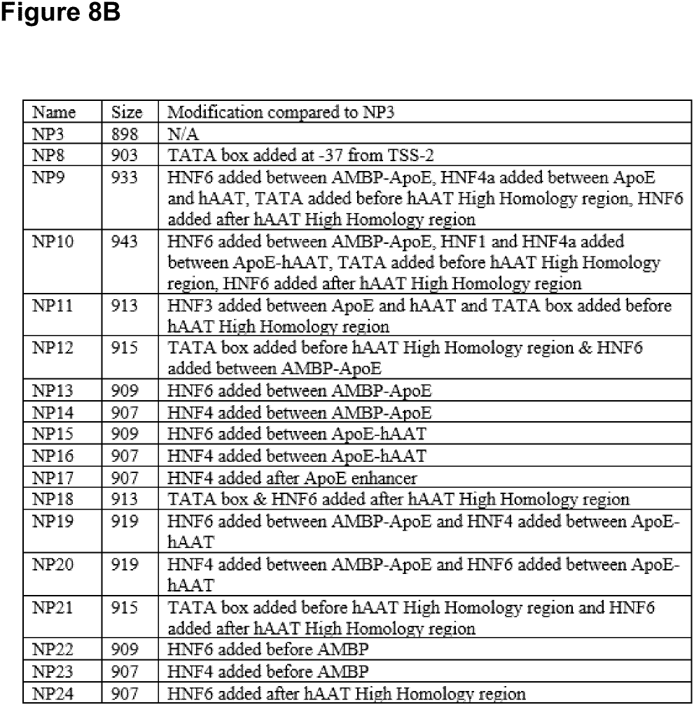

The results are shown in FIG. 6A (before luciferase correction) and FIG. 6B (after luciferase correction).

### Example 7 - Comparison of CFI Expression in Huh7 Cells In Vitro When the Transgene is Under the Control of Different Promoters

Novel hAAT-based TREs were screened and ranked based on the amount (w/v) of protein produced after transient transfection of Huh7 cells. Each of the novel hAAT-based TREs was conjugated to a wild-type human CFI polypeptide (the wild-type human CFI polypeptide having the sequence of SEQ ID NO: 25).

### Methods

Expression cassettes were generated which comprise one of the novel hAAT-based TREs (each TRE having a sequence of one of SEQ ID NO: 68-91) or HCR-hAAT (SEQ ID NO: 67), HLP2 (SEQ ID NO: 51) or FRE72 (SEQ ID NO: 66) for control purposes.

The transient transfection entailed mixing, for each expression cassette comprising one of the TREs, a plasmid carrying such expression cassette with a transfection reagent (FuGENE®HD (Promega, cat no. E2311)) and delivering the mixture to cells so that the gene can be expressed and protein subsequently quantified by ELISA. In each case, a luciferase reporter gene was included in the transfection mixture as a way of evaluating transfection efficiency.

Huh7 cell culture and Transfection: Huh7 cells (JCRB cell bank, no. JCRB0403) were seeded in a 96 well plate (30,000 cells per well) in DMEM low glucose, 10% FBS + Glutamax (D10 media) and cultured at 37° C. and 5% CO₂ (day 1). The next day (approx. 24 hours after cell seeding; day 2), the plasmid DNA-transfection reagent mixture was prepared and transfected into Huh7 cells. Briefly, 0.225 µg of test plasmid DNA and 0.025 µg CMV-Luciferase control plasmid (FLJ-PL282) were mixed with FuGENE at a ratio of 4 µl FuGENE per µg of DNA (or 1 µl FuGENE per 0.25 µg of plasmid DNA). In addition, 0.25 µg of CMV-Luciferase control plasmid (FLJ-PL282) was mixed with FuGENE at a ratio of 4 µl FuGENE per µg of DNA to be used as a positive control for the luciferase assay. For 96-well transfection experiments, 10µl of the plasmid DNA-FuGENE mix was added per well. The plasmid DNA-transfection reagent mixture was incubated on the cells overnight at 37° C. and 5% CO₂. The next morning (day 3), approximately 18 hours after transfection, the media was replaced with fresh D10 media and cells incubated overnight at 37° C. and 5% CO₂. The next morning (24 h later; day 4), media was replaced by fresh DMEM low glucose + Glutamax + Insulin-Transferrin-Selenium supplement (D0/ITS media). Cells and media were harvested the following day (on day 5).

CFI expression in culture media was assessed using a CFI ELISA kit (e.g. Hycult cat# HK355-01) according to manufacturer’s instructions. In brief, culture media samples (diluted with the sample diluent from the kit as necessary) and human CFI standards from the kit were incubated in microtiter wells coated with anti-human CFI antibodies (to capture the human CFI). A biotinylated tracer antibody was added (to bind to the captured human CFI) followed by a streptavidin-peroxidase conjugate (to bind to the biotinylated tracer antibody). The substrate tetramethylbenzidine (TMB) was then added, which the streptavidin-peroxidase conjugate reacts with. The enzyme reaction was stopped by adding oxalic acid. For each of the samples and standards, the absorbance at 450 nm was measured with a spectrophotometer. A standard curve was obtained by plotting the absorbance (linear) against the corresponding concentrations of the human CFI standards (log). The concentration of human CFI in the samples was then determined from the standard curve.

In parallel to the ELISA, Huh7 cells were washed with phosphate buffered saline (PBS) twice and cells treated with 100µl of luciferase lysis buffer from the Luciferase assay kit (Promega cat# E1501/E4530). Cell lysates were stored at -80° C. On the day of luciferase assays, cell lysates were thawed and 20µl of the sample was used to measure the luciferase expression by luminescence on a Molecular Devices SpectraMax i3x plate reader. The detailed protocol is published in the Promega Technical Bullitin #TB281. Where applicable, luciferase expression was used as internal control to normalise the CFI expression levels. Analyses were performed using the software Graphpad Prism v7.

### Results

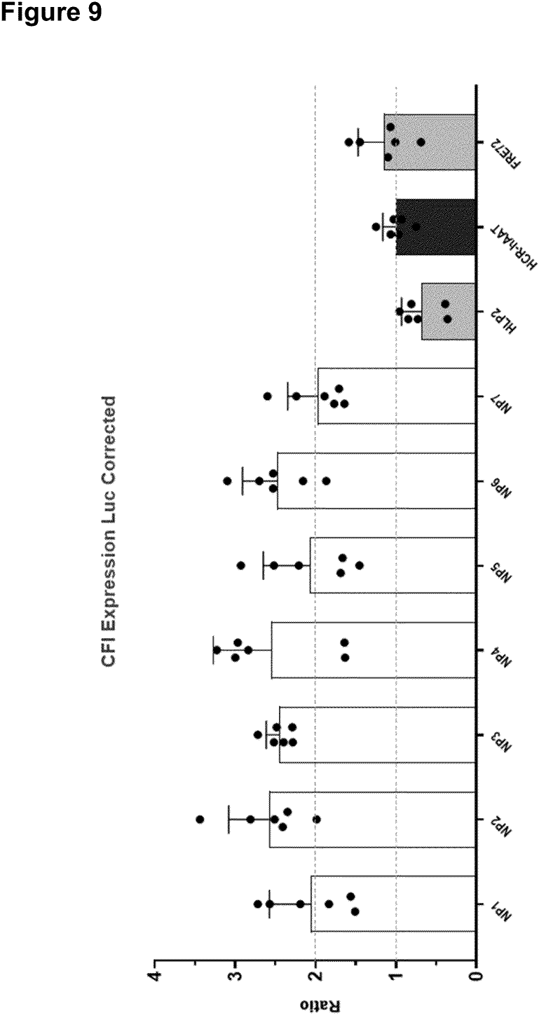

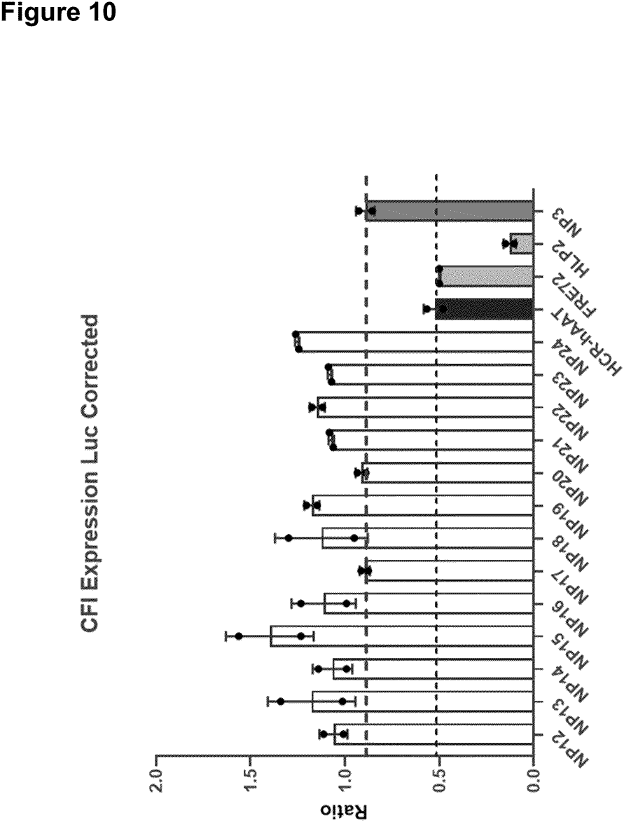

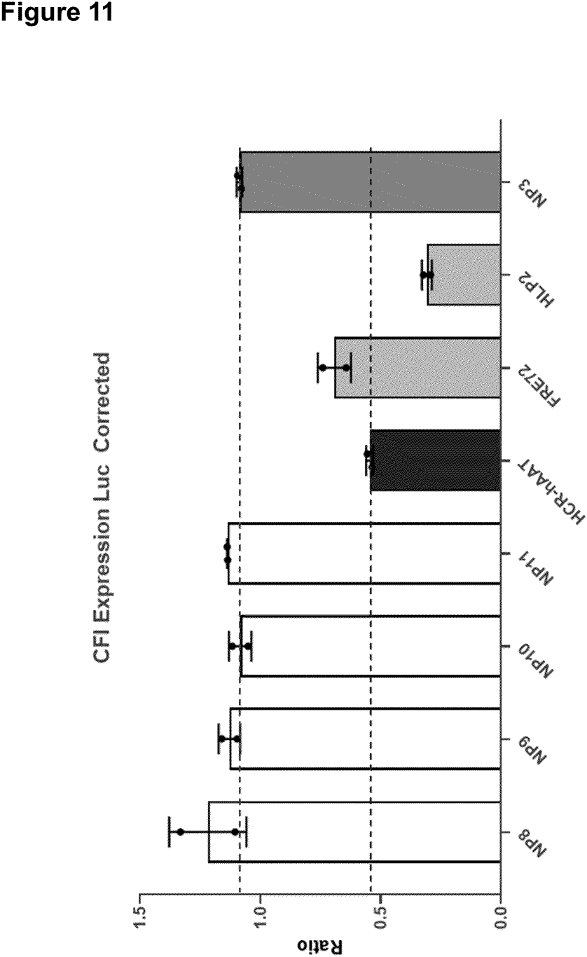

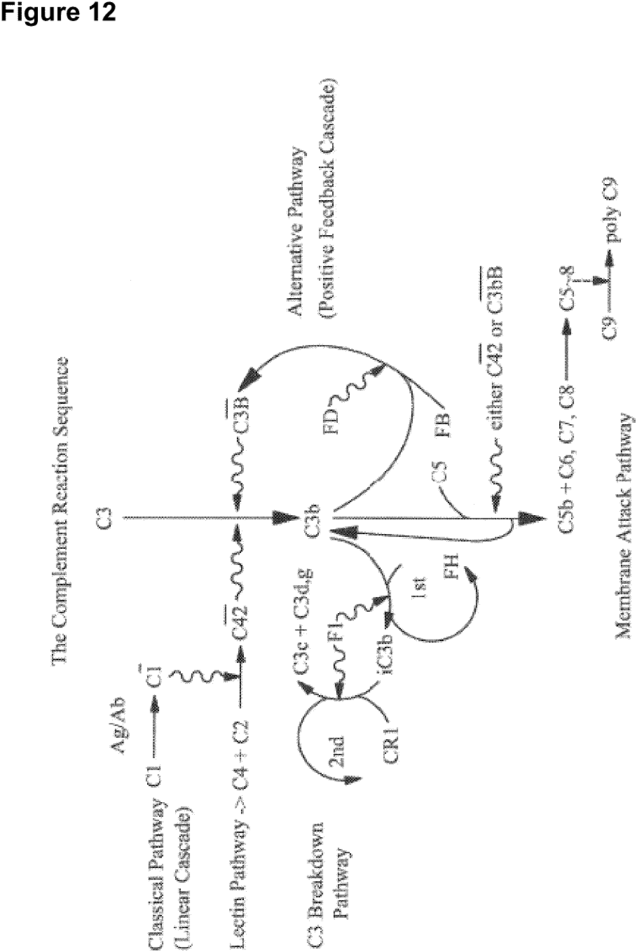

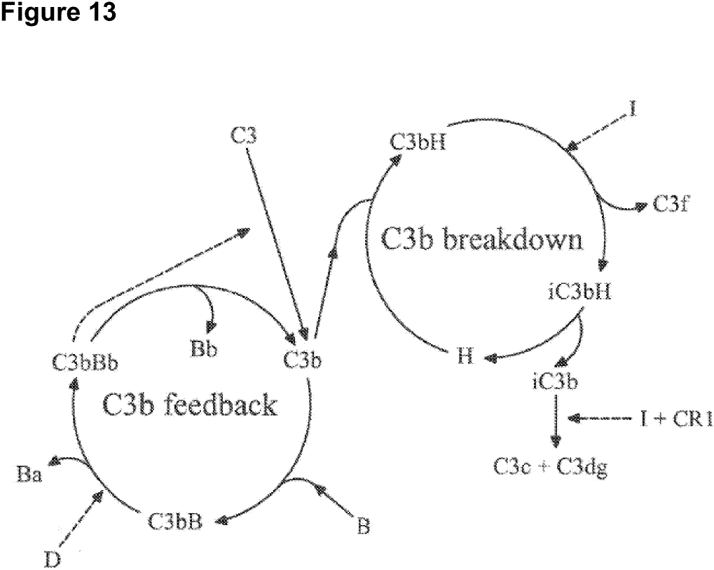

The CFI expression levels in the culture media following transfection into Huh-7 cells were measured for HCR-hAAT, HLP2, FRE72, and twenty-four other hAAT-based TREs. The CFI expression levels were normalised using luciferase expression as described above. Each expression cassette comprised wild-type human CFI and the BgpA polyA sequence. That data is presented in FIGS. 9-11. All of the novel hAAT-based TREs promoted greater expression than all of HCR-hAAT, HLP2, and FRE72.

### Numbered Aspects of the Invention

1. A polynucleotide comprising a Complement Factor I (CFI) nucleotide sequence, wherein the CFI nucleotide sequence encodes a CFI polypeptide or a fragment thereof and wherein at least a portion of the CFI nucleotide sequence is not wild-type.

2. The polynucleotide of aspect 1, wherein the portion of the CFI nucleotide sequence that is not wild type is codon-optimised.

3. The polynucleotide of aspect 1 or 2, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1-40.

4. A polynucleotide comprising a CFI nucleotide sequence, wherein the CFI nucleotide sequence encodes a CFI polypeptide or a fragment thereof and the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1-40.

5. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 2, 3, 5, 6, 7, 9-12, 14-17, 20, 22, 23, 25, 26, 27, 29-32, 34-37, and 40.

6. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1, 5, 7, 11-14, 16, 17, 21, 25, 27, 31-34, 36, and 37.

7. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1, 5, 7, 11, 12, 14, 16, 17, 21, 25, 27, 31, 32, 34, 36, and 37.

8. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 5, 7, 12, 14, 16, 17, 25, 27, 32, 34, 36, and 37.

9. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 5, 7, 14, 25, 27, and 34.

10. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 3, 5-7, 10, 14, 17, 20, 23, 25-27, 30, 34, 37, and 40.

11. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 5, 10, 17, 25, 30, and 37.

12. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NO: 5 or 25.

13. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1-40.

14. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 2, 3, 5, 6, 7, 9-12, 14-17, 20, 22, 23, 25, 26, 27, 29-32, 34-37, and 40.

15. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 11-14, 16, 17, 21, 25, 27, 31-34, 36, and 37.

16. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 1, 5, 7, 11, 12, 14, 16, 17, 21, 25, 27, 31, 32, 34, 36, and 37.

17. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, 12, 14, 16, 17, 25, 27, 32, 34, 36, and 37.

18. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 7, 14, 25, 27, and 34.

19. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 3, 5-7, 10, 14, 17, 20, 23, 25-27, 30, 34, 37, and 40.

20. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of any one of SEQ ID NOs: 5, 10, 17, 25, 30, and 37

21. The polynucleotide of any one of the preceding aspects, wherein the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 5 or 25.

22. The polynucleotide of any one of the preceding aspects, wherein the CFI nucleotide sequence comprises a sequence of any one of SEQ ID NOs: 1-20, or a variant thereof encoding a CFI polypeptide comprising a sequence at least 95% identical to the wild type CFI amino acid sequence of SEQ ID NO: 44.

23. The polynucleotide of aspect 22, wherein the variant is a variant of any one of SEQ ID NOs: 5, 7, and 14.

24. The polynucleotide of aspect 22, wherein the variant is a variant of any one of SEQ ID NOs: 5, 10, and 17.

25. The polynucleotide of aspect 23 or 24, wherein the variant is a variant of SEQ ID NO: 5.

26. The polynucleotide of any one of aspects 22 to 25, wherein the variant encodes a CFI polypeptide having at least 70%, at least 80%, at least 90%, or at least 100% of the CFI activity of wild-type CFI.

27. The polynucleotide of aspect 26 wherein the CFI activity comprises C3b-inactivating and/or iC3b-degradation activity.

28. The polynucleotide of any one of aspects 22 to 27, wherein the variant of SEQ ID NO: 5 is identical to SEQ ID NO: 5 except that it comprises nucleotide substitutions such that the CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO:44.

29. The polynucleotide of any one of aspects 22 to 28, wherein the variant of SEQ ID NO: 5 has 1, 2 or fewer, 3 or fewer, 4 or fewer, 5 or fewer, 6 or fewer, 7 or fewer, 8 or fewer, 9 or fewer, 10 or fewer, 20 or fewer, or 30 or fewer nucleotide substitutions relative to the sequence of SEQ ID NO: 5.

30. The polynucleotide of any one of aspects 22 to 29, wherein the variant of SEQ ID NO: 5 has 1, 2 or fewer, 3 or fewer, 4 or fewer, 5 or fewer, or 6 or fewer nucleotide substitutions relative to the sequence of SEQ ID NO: 5.

31. The polynucleotide of any one of aspects 22 to 30, wherein the variant of SEQ ID NO: 5 has 4 or fewer nucleotide substitutions relative to the sequence of SEQ ID NO: 5 and/or encodes a CFI polypeptide having 3 or fewer amino acid substitutions relative to the wild type CFI amino acid sequence of SEQ ID NO: 44.

32. The polynucleotide of any one of aspects 22 to 31, wherein the variant of SEQ ID NO: 5 has 3 or fewer nucleotide substitutions relative to the sequence of SEQ ID NO: 5 and/or encodes a CFI polypeptide having 2 or fewer amino acid substitutions relative to the wild type CFI amino acid sequence of SEQ ID NO: 44.

33. The polynucleotide of any one of aspects 22 to 32, wherein the variant of SEQ ID NO: 5 has 1 nucleotide substitution relative to the sequence of SEQ ID NO: 5 and/or encodes a CFI polypeptide having 1 amino acid substitution relative to the wild type CFI amino acid sequence of SEQ ID NO: 44.

34. The polynucleotide of any one of the preceding aspects, wherein the CFI nucleotide sequence encodes a CFI polypeptide having 1, 2 or fewer, 3 or fewer, 4 or fewer, or 5 or fewer amino acid substitutions relative to the wild type CFI amino acid sequence of SEQ ID NO: 44.

35. The polynucleotide of any one of the preceding aspects, wherein the CFI nucleotide sequence encodes a CFI polypeptide having 3 or fewer amino acid substitutions relative to the wild type CFI amino acid sequence of SEQ ID NO: 44.

36. The polynucleotide of any one of the preceding aspects, wherein the CFI nucleotide sequence encodes a CFI polypeptide having 2 or fewer amino acid substitutions relative to the wild type CFI amino acid sequence of SEQ ID NO: 44.

37. The polynucleotide of any one of the preceding aspects, wherein the CFI nucleotide sequence encodes a CFI polypeptide having 1 amino acid substitution relative to the wild type CFI amino acid sequence of SEQ ID NO: 44.

38. The polynucleotide of any one of the preceding aspects, wherein the CFI nucleotide sequence comprises a sequence that is at least 98% identical to a fragment of at least 1500 nucleotides of any one of SEQ ID NOs: 1-40.

39. The polynucleotide of any one of the preceding aspects, wherein the CFI nucleotide sequence comprises a sequence that is at least 99% identical to a fragment of at least 1500 nucleotides of any one of SEQ ID NOs: 1-40.

40. The polynucleotide of any one of the preceding aspects, wherein the CFI nucleotide sequence comprises a sequence that is at least 98% identical to a nucleotide sequence of any one of SEQ ID NOs: 1-40.

41. The polynucleotide of any one of the preceding aspects, wherein the CFI nucleotide sequence comprises a sequence that is at least 99% identical to a nucleotide sequence of any one of SEQ ID NOs: 1-40.

42. The polynucleotide of any one of the preceding aspects, wherein the CFI nucleotide sequence comprises a sequence that is at least 98% identical to a fragment of at least 1500 nucleotides of SEQ ID NO: 5 or 25.

43. The polynucleotide of any one of the preceding aspects, wherein the CFI nucleotide sequence comprises a sequence that is at least 99% identical to a fragment of at least 1500 nucleotides of SEQ ID NO: 5 or 25.

44. The polynucleotide of any one of the preceding aspects, wherein the CFI nucleotide sequence comprises a sequence that is at least 98% identical SEQ ID NO: 5 or 25.

45. The polynucleotide of any one of the preceding aspects, wherein the CFI nucleotide sequence comprises a sequence that is at least 99% identical SEQ ID NO: 5 or 25.

46. The polynucleotide of any one of the preceding aspects, wherein the CFI nucleotide sequence comprises a sequence of SEQ ID NO: 5 or 25.

47. The polynucleotide of any one of the aspects 3 to 46, wherein at least a portion of the CFI nucleotide sequence is codon-optimised.

48. The polynucleotide of aspect 2 or 47, wherein the portion of the CFI nucleotide sequence that is codon-optimised is codon-optimised for expression in human liver cells.

49. The polynucleotide of any one of the preceding aspects, wherein the CFI nucleotide sequence is codon-optimised for expression in human liver cells.

50. The polynucleotide of any one of aspects 2, 47 or 48, wherein the portion of the CFI nucleotide sequence that is codon-optimised is a contiguous portion.

51. The polynucleotide of any one of aspects 2, 47, 48 or 50, wherein the portion of the CFI nucleotide sequence that is codon-optimised is at least 1000, at least 1200, at least 1300, at least 1400, at least 1500, at least 1600, at least 1700, 1752 or fewer, between 1300 and 1752, between 1500 and 1752, between 1600 and 1752, or around 1752 nucleotides in length.

52. The polynucleotide of any one of aspects 2, 47, 48, 50 or 51, wherein the portion of the CFI nucleotide sequence that is codon-optimised is at least 1000, at least 1200, at least 1300, at least 1400, at least 1500, 1698 or fewer, between 1300 and 1698, between 1500 and 1698, or around 1698 nucleotides in length.

53. The polynucleotide of any one of aspects 2, 47, 48, or 50 to 52, wherein the portion of the CFI nucleotide sequence that is codon-optimised encodes a mature CFI polypeptide.

54. The polynucleotide of any one of aspects 2, 47, 48, or 50 to 53, wherein the portion of the CFI nucleotide sequence that is codon-optimised encodes all or a portion of a signal peptide.

55. The polynucleotide of any one of aspects 2, 47, 48, or 50 to 54, wherein the portion of the CFI nucleotide sequence that is codon-optimised does not encode all or a portion of a signal peptide.

56. The polynucleotide of any one of aspects 2, 47 to 55, wherein, in the CFI nucleotide sequence or the portion of the CFI nucleotide sequence that is codon-optimised, at least 70%, at least 75%, at least 76%, at least 77%, at least 78%, at least 79%, at least 80%, or at least 81%, at least 82%, at least 83%, or at least 84% of the codons are selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC.

57. The polynucleotide of aspect 56, wherein, in the CFI nucleotide sequence or the portion of the CFI nucleotide sequence that is codon-optimised, at least 83%, or at least 84% of the codons are selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC.

58. The polynucleotide of any one of the preceding aspects, wherein the CFI nucleotide sequence or the portion of the CFI nucleotide sequence that is codon-optimised comprises a reduced number of CpGs compared to a corresponding portion of a reference CFI nucleotide sequence.

59. The polynucleotide of aspect 58, wherein the CFI nucleotide sequence or the portion of the CFI nucleotide sequence that is codon-optimised comprises 40 or fewer, 20 or fewer, 15 or fewer, 10 or fewer, or 5 or fewer CpGs.

60. The polynucleotide of aspect 59, wherein the CFI nucleotide sequence or the portion of the CFI nucleotide sequence that is codon-optimised comprises 5 or fewer, 4 or fewer, 3 or fewer, or 2 or fewer CpGs per 100 nucleotides.

61. The polynucleotide of any one of aspects 58 to 60, wherein the CFI nucleotide sequence or the portion of the CFI nucleotide sequence that is codon-optimised is CpG-free.

62. The polynucleotide of any one of aspects 58 to 61, wherein the reference CFI nucleotide sequence is a wild-type CFI nucleotide sequence.

63. The polynucleotide of aspect 62, wherein the wild-type CFI nucleotide sequence is the nucleotide sequence of SEQ ID NO: 41 or 42.

64. The polynucleotide of any one of aspects 2, 47, 48, or 50 to 63, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1-20.

65. The polynucleotide of any one of aspects 2, 47, 48, or 50 to 64, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 2, 3, 5, 6, 7, 9-12, 14-17, and 20.

66. The polynucleotide of any one of aspects 2, 47, 48, or 50 to 65, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1, 5, 7, 11-14, 16, and 17.

67. The polynucleotide of any one of aspects 2, 47, 48, or 50 to 66, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 1, 5, 7, 11, 12, 14, 16, and 17.

68. The polynucleotide of any one of aspects 2, 47, 48, or 50 to 67, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 5, 7, 12, 14, 16, and 17.

69. The polynucleotide of any one of aspects 2, 47, 48, or 50 to 68, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 5, 7, and 14.

70. The polynucleotide of any one of aspects 2, 47, 48, or 50 to 69, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 3, 5-7, 10, 14, 17, and 20.

71. The polynucleotide of any one of aspects 2, 47, 48, or 50 to 70, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of any one of SEQ ID NOs: 5, 10, and 17.

72. The polynucleotide of any one of aspects 2, 47, 48, or 50 to 71, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of SEQ ID NO: 5.

73. The polynucleotide of any one of aspects 2, 47, 48, or 50 to 72, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 1-20.

74. The polynucleotide of any one of aspects 2, 47, 48, or 50 to 73, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 2, 3, 5, 6, 7, 9-12, 14-17, and 20.

75. The polynucleotide of any one of aspects 2, 47, 48, or 50 to 74, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 1, 5, 7, 11-14, 16, and 17.

76. The polynucleotide of any one of aspects 2, 47, 48, or 50 to 75, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 1, 5, 7, 11, 12, 14, 16, and 17.

77. The polynucleotide of any one of aspects 2, 47, 48, or 50 to 76, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 5, 7, 12, 14, 16, and 17.

78. The polynucleotide of any one of aspects 2, 47, 48, or 50 to 77, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 5, 7, and 14.

79. The polynucleotide of any one of aspects 2, 47, 48, or 50 to 78, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 3, 5-7, 10, 14, 17, and 20.

80. The polynucleotide of any one of aspects 2, 47, 48, or 50 to 79, wherein the portion of the CFI nucleotide sequence that is codon-optimised is at least 80%, at least 85%, at least 90%, at least 95%, at least 98%, at least 99%, at least 99.5%, at least 99.8%, or 100% identical to any one of SEQ ID NOs: 5, 10, and 17.

81. The polynucleotide of any one of aspects 2, 47, 48, or 50 to 80, wherein the portion of the CFI nucleotide sequence that is codon-optimised is at least 80%, at least 85%, at least 90%, at least 95%, at least 98%, at least 99%, at least 99.5%, at least 99.8%, or 100% identical to a fragment of at least 1000, at least 1200, at least 1300, at least 1500, 1698 or fewer, between 1200 and 1698, between 1500 and 1698, or around 1698 nucleotides of SEQ ID NO: 5.

82. The polynucleotide of any one of aspects 2, 47, 48, or 50 to 81, wherein the portion of the CFI nucleotide sequence that is codon-optimised is at least 80%, at least 85%, at least 90%, at least 95%, at least 98%, at least 99%, at least 99.5%, at least 99.8%, or 100% identical to SEQ ID NO: 5.

83. The polynucleotide of any one of aspects 2, 47, 48, or 50 to 82, wherein the portion of the CFI nucleotide sequence that is codon-optimised is at least 99.5%, at least 99.8%, or 100% identical to a fragment of at least 1500 nucleotides of SEQ ID NO: 5.

84. The polynucleotide of any one of aspects 2, 47, 48, or 50 to 83, wherein the portion of the CFI nucleotide sequence that is codon-optimised is at least 99%, at least 99.5%, at least 99.8%, or 100% identical to SEQ ID NO: 5.

85. The polynucleotide of any one of the preceding aspects, wherein the CFI polypeptide or the fragment thereof encoded by the CFI nucleotide sequence is expressed at the same or a higher level in a human liver cell compared to expression of a CFI polypeptide encoded by a reference CFI nucleotide sequence.

86. The polynucleotide of aspect 85, wherein the CFI polypeptide or the fragment thereof encoded by the CFI nucleotide sequence is expressed at the same or a higher level on transfection of the polynucleotide comprising the CFI nucleotide sequence into a human liver cell compared to expression of the CFI polypeptide encoded by the reference CFI nucleotide sequence on transfection with an equivalent polynucleotide comprising the reference CFI nucleotide sequence.

87. The polynucleotide of aspect 85 or 86, wherein the human liver cell is a Huh-7 cell.

88. The polynucleotide of any one of aspects 85 to 87, wherein the expression of the CFI polypeptide or the fragment thereof encoded by the CFI nucleotide sequence is at a higher level compared to the expression of the CFI polypeptide encoded by the reference CFI nucleotide sequence.

89. The polynucleotide of any one of aspects 85 to 88, wherein the expression of the CFI polypeptide or the fragment thereof encoded by the CFI nucleotide sequence is at least 1.5x, at least 2x, at least 3x, at least 5x, at least 8x, at least 10x, at least 20x, at least 30x, at least 40x, or at least 50x higher compared to the CFI polypeptide encoded by the reference CFI nucleotide sequence.

90. The polynucleotide of aspect 88 or 89, wherein the reference CFI nucleotide sequence is a wild type CFI nucleotide sequence.

91. The polynucleotide of aspect 90, wherein the wild type CFI nucleotide sequence is the sequence of SEQ ID NO: 41 or 42.

92. The polynucleotide of any one of aspects 85 to 89, wherein the reference CFI nucleotide sequence is the sequence of SEQ ID NO: 1 or 21.

93. The polynucleotide of any one of aspects 85 to 89, wherein the reference CFI nucleotide sequence is the sequence of SEQ ID NO: 5 or 25.

94. The polynucleotide of any one of the preceding aspects, wherein the expression of the CFI polypeptide or the fragment thereof encoded by the CFI nucleotide sequence is at least 50%, at least 60%, at least 70%, at least 80%, at least 90%, at least 95%, or at least 100% of the expression of the CFI polypeptide encoded by the sequence of SEQ ID NO: 5 or 25.

95. The polynucleotide of any one of the preceding aspects, wherein the CFI nucleotide sequence encodes a signal peptide.

96. The polynucleotide of aspect 95, wherein the amino acid sequence of the signal peptide is an amino acid sequence of a wild type CFI signal peptide.

97. The polynucleotide of aspect 95, wherein the amino acid sequence of the signal peptide is an amino acid sequence of a signal peptide which is not a wild type CFI signal peptide.

98. The polynucleotide of any one of the preceding aspects, wherein the polynucleotide further comprises a transcription regulatory element.

99. The polynucleotide of aspect 98, wherein the transcription regulatory element comprises a liver-specific promoter.

100. The polynucleotide of aspect 98 or 99, wherein the transcription regulatory element comprises an A1AT promoter or a fragment of an A1AT promoter.

101. The polynucleotide of aspect 100, wherein the transcription regulatory element comprises two fragments of an A1AT promoter.

102. The polynucleotide of aspect 101, wherein the first fragment of an A1AT promoter is between 75 and 100 nucleotides in length and the second fragment of an A1AT promoter is between 25 and 50 nucleotides in length.

103. The polynucleotide of any one of aspects 100-102, wherein the polynucleotide comprises a promoter that is at least 80%, at least 85%, at least 90%, at least 95%, at least 98%, at least 99%, at least 99.5%, or 100% identical to SEQ ID NO: 66.

104. The polynucleotide of aspect 100, wherein the fragment of an A1AT promoter is at least 100, at least 120, at least 150, at least 180, 255 or fewer, between 100 and 255, between 150 and 225, between 150 and 300, or between 180 and 255 nucleotides in length.

105. The polynucleotide of aspect 104, wherein the fragment of an A1AT promoter is between 180 and 255 nucleotides in length.

106. The polynucleotide of any one of the aspects 1 to 101, 104 or 105, wherein the polynucleotide comprises a promoter that is at least 80%, at least 85%, at least 90%, at least 95%, at least 98%, at least 99%, at least 99.5%, at least 99.8%, or 100% identical to SEQ ID NO: 47 or SEQ ID NO: 48.

107. The polynucleotide of aspect 106, wherein the polynucleotide comprises a promoter that is at least 98%, at least 99%, at least 99.5%, at least 99.8%, or 100% identical to SEQ ID NO: 47 or SEQ ID NO: 48.

108. The polynucleotide of aspect 107, wherein the polynucleotide comprises a promoter of SEQ ID NO: 47 or SEQ ID NO: 48.

109. The polynucleotide of any one of aspects 98 to 101 or 104 to 108, wherein the transcription regulatory element comprises a fragment of an A1AT promoter that is equal to or fewer than 418 nucleotides, equal to or fewer than 255 nucleotides, or equal to or fewer than 185 nucleotides in length and comprises SEQ ID NO: 47.

110. The polynucleotide of any one of aspects 98 to 101 or 104 to 109, wherein the transcription regulatory element comprises an enhancer.

111. The polynucleotide of aspect 110, wherein the enhancer is an HCR enhancer or a fragment of an HCR enhancer.

112. The polynucleotide of aspect 111, wherein the fragment of an HCR enhancer is a fragment of at least 80, at least 90, at least 100, 192 or fewer, between 80 and 192, between 90 and 192, between 100 and 250, or between 117 and 192 nucleotides in length.

113. The polynucleotide of aspect 112, wherein the fragment of an HCR enhancer is between 117 and 192 nucleotides in length.

114. The polynucleotide of any one of 1 to 101 or 104 to 113, wherein the polynucleotide comprises an enhancer that is at least 80%, at least 85%, at least 90%, at least 95% at least 98%, at least 99%, at least 99.5%, at least 99.8%, or 100% identical to SEQ ID NO: 49 or SEQ ID NO: 50.

115. The polynucleotide of aspect 114, wherein the polynucleotide comprises an enhancer that is at least 98%, at least 99%, at least 99.5%, at least 99.8%, or 100% identical to SEQ ID NO: 49 or SEQ ID NO: 50.

116. The polynucleotide of aspect 115, wherein the polynucleotide comprises an enhancer of SEQ ID NO: 49 or SEQ ID NO: 50.

117. The polynucleotide of any one of aspects 98 to 101 or 104 to 116, wherein the transcription regulatory element comprises a fragment of an HCR enhancer that is equal to or fewer than 321 nucleotides, equal to or fewer than 192 nucleotides or equal to or fewer than 117 nucleotides in length and comprises SEQ ID NO: 49.

118. The polynucleotide of any one of aspects 98 to 101 or 104 to 117, wherein the transcription regulatory element is at least 80%, at least 85%, at least 90%, at least 95%, at least 98%, at least 99%, at least 99.5%, at least 99.8%, or 100% identical to SEQ ID NO: 51.

119. The polynucleotide of aspect 118, wherein the transcription regulatory element has a sequence of SEQ ID NO: 51.

120. The polynucleotide of any one of the preceding aspects, wherein:
(i) the CFI nucleotide sequence comprises a sequence that is at least 95%, at least 98%, at least 99%, at least 99.5%, at least 99.8%, or 100% identical to SEQ ID NOs: 5 or 25; and
(ii) the polynucleotide comprises a promoter that is at least 98%, at least 99%, at least 99.5%, at least 99.8%, or 100% identical to SEQ ID NO: 47 and/or an enhancer element that is at least 98%, at least 99%, at least 99.5%, at least 99.8% or 100% identical to SEQ ID NO: 49.

121. The polynucleotide of any one of the preceding aspects, wherein:
(i) the CFI nucleotide sequence comprises a sequence that is at least 95%, at least 98%, at least 99%, at least 99.5%, at least 99.8%, or 100% identical to SEQ ID NOs: 5 or 25; and
(ii) the polynucleotide comprises a transcription regulatory element that is at least 98%, at least 99%, at least 99.5%, at least 99.8%, or 100% identical to SEQ ID NO: 51.

122. The polynucleotide of aspect 100, wherein the A1AT promoter or the fragment of an A1AT promoter is at least 200, at least 250, at least 300, 500 or fewer, between 200 and 500, between 250 and 500, between 350 and 450, or around 418 nucleotides in length.

123. The polynucleotide of aspect 122, wherein the A1AT promoter or the fragment of an A1AT promoter is between 350 and 450 nucleotides in length.

124. The polynucleotide of aspect 111, wherein the HCR enhancer or the fragment of an HCR enhancer is a fragment of at least 150, at least 190, at least 230, fewer than 400, between 150 and 400, between 190 and 370, between 230 and 340, between 250 and 340, or around 321 nucleotides in length.

125. The polynucleotide of aspect 124, wherein the HCR enhancer or the fragment of an HCR enhancer is between 250 and 340 nucleotides in length.

126. The polynucleotide of any one of aspects 98 to 101 or 104 to 125, wherein the transcription regulatory element is at least 80%, at least 85%, at least 90%, at least 95%, at least 98%, at least 99%, at least 99.5%, at least 99.8%, or 100% identical to SEQ ID NO: 52.

127. The polynucleotide of aspect 126, wherein the transcription regulatory element has a sequence of SEQ ID NO: 52.

128. The polynucleotide of any one of aspects 1 to 99 or 122 to 125, wherein:
(i) the CFI nucleotide sequence comprises a sequence that is at least 95%, at least 98%, at least 99%, at least 99.5%, at least 99.8%, or 100% identical to SEQ ID NOs: 5 or 25; and
(ii) the polynucleotide comprises a promoter that is at least 98%, at least 99%, at least 99.5%, at least 99.8%, or 100% identical to SEQ ID NO: 48 and/or an enhancer element that is at least 98%, at least 99%, at least 99.5%, at least 99.8% or 100% identical to SEQ ID NO: 50.

129. The polynucleotide of any one of aspects 1 to 99 or 122 to 128, wherein:
(i) the CFI nucleotide sequence comprises a sequence that is at least 95%, at least 98%, at least 99%, at least 99.5%, at least 99.8%, or 100% identical to SEQ ID NOs: 5 or 25; and
(ii) the polynucleotide comprises a transcription regulatory element that is at least 98%, at least 99%, at least 99.5%, at least 99.8%, or 100% identical to SEQ ID NO: 52.

130. The polynucleotide of any one of the preceding aspects, wherein the polynucleotide comprises a woodchuck hepatitis posttranscriptional regulatory element (WPRE).

131. The polynucleotide of any one of the preceding aspects, wherein the polynucleotide comprises a woodchuck hepatitis posttranscriptional regulatory element (WPRE) at least 98%, at least 99%, at least 99.5%, at least 99.8%, or 100% identical to any one of SEQ ID NOs: 62 to 64.

132. The polynucleotide of any one of the preceding aspects, wherein the polynucleotide comprises a woodchuck hepatitis posttranscriptional regulatory element (WPRE) at least 98% identical to any one of SEQ ID NOs: 62 to 64.

133. The polynucleotide of any one of the preceding aspects, wherein the polynucleotide comprises a woodchuck hepatitis posttranscriptional regulatory element (WPRE) of any one of SEQ ID NOs: 62 to 64.

134. The polynucleotide of aspect 98, wherein the transcription regulatory element (TRE):
(a) promotes greater expression than HCR-hAAT;
(b) promotes greater expression than FRE72;
(c) comprises a hAAT sequence element; and/or
(d) comprises an AMBP sequence element.

135. The polynucleotide of aspect 134, wherein the TRE is greater than 500, greater than 600, greater than 700, greater than 800, greater than 900, greater than 1000, greater than 1100, greater than 1200, greater than 1300, greater than 1400, greater than 1500, or greater than 1600 nucleotides in length.

136. The polynucleotide of aspect 134 or 135, wherein the TRE is less than 2000, less than 1800, less than 1600, less than 1500, less than 1400, less than 1300, less than 1200, less than 1100, less than 1000, less than 900, less than 800, less than 700, or less than 600 nucleotides in length.

137. The polynucleotide of any one of aspects 134-136, wherein the TRE is between 400 and 2000, between 500 and 1800, between 600 and 1400, between 800 and 1600, between 500 and 600, between 600 and 700, between 700 and 800, between 800 and 900, between 900 and 1000, between 1000 and 1100, between 1100 and 1200, between 1200 and 1300, between 1300 and 1400, between 1400 and 1500, between 1500 and 1600, or between 1600 and 1700 nucleotides in length.

138. The polynucleotide of any one of aspects 134-137, wherein the TRE comprises a hAAT sequence element.

139. The polynucleotide of any one of aspects 134-138, wherein the hAAT sequence element comprises a nucleotide sequence of SEQ ID NO: 95 or a variant of SEQ ID NO: 95 that differs by 1, 2, or 3 nucleotides.

140. The polynucleotide of any one of aspects 134-139, wherein the hAAT sequence element comprises a nucleotide sequence of SEQ ID NO: 95.

141. The polynucleotide of any one of aspects 134-140, wherein the hAAT sequence element comprises a nucleotide sequence of SEQ ID NO: 96 or a variant of SEQ ID NO: 96 that differs by 1, 2, or 3 nucleotides.

142. The polynucleotide of any one of aspects 134-141, wherein the hAAT sequence element comprises a nucleotide sequence of SEQ ID NO: 96.

143. The polynucleotide of any one of aspects 134-142, wherein the hAAT sequence element comprises a nucleotide sequence at least 80%, at least 85%, at least 90%, at least 95%, at least 98%, or at least 99% identical to SEQ ID NO: 94 or a fragment of SEQ ID NO: 94 that is at least 200, at least 300, or at least 400 nucleotides long.

144. The polynucleotide of any one of aspects 134-143, wherein the hAAT sequence element comprises a nucleotide sequence at least 98% identical to SEQ ID NO: 94.

145. The polynucleotide of any one of aspects 134-144, wherein the hAAT sequence element comprises a nucleotide sequence of SEQ ID NO: 94.

146. The polynucleotide of any one of aspects 134-145, wherein the TRE comprises a first and a second hAAT sequence element.

147. The polynucleotide of aspect 146, wherein the second hAAT sequence element is 5′ of the first hAAT sequence element and 5′ of the AMBP sequence element.

148. The polynucleotide of aspect 146 or aspect 147, wherein the second hAAT sequence element comprises a nucleotide sequence of SEQ ID NO: 95 or a variant of SEQ ID NO: 95 that differs by 1, 2, or 3 nucleotides.

149. The TRE of aspect 148, wherein the second hAAT sequence element comprises a nucleotide sequence of SEQ ID NO: 95.

150. The polynucleotide of any one of aspects 134-149, wherein the TRE comprises an AMBP sequence element.

151. The polynucleotide of any one of aspects 134-150, wherein the TRE comprising the AMBP sequence element promotes equivalent or greater expression than a corresponding TRE lacking an AMBP sequence element.

152. The polynucleotide of any one of aspects 134-151, wherein the TRE comprising the AMBP sequence element promotes greater expression than a corresponding TRE lacking an AMBP sequence element.

153. The polynucleotide of any one of aspects 134-152, wherein the AMBP sequence element comprises at least one binding site selected from the group consisting of an HNF1-1 binding site, an HNF4 binding site, an HNF3a binding site, an HNF 1-2 binding site, and an HNF3-2 binding site.

154. The polynucleotide of aspect 153, wherein the AMBP sequence element comprises an HNF1-1 binding site, an HNF4 binding site, an HNF3a binding site, an HNF1-2 binding site, and an HNF3-2 binding site.

155. The polynucleotide of any one of aspects 134-154, wherein the AMBP sequence element comprises a nucleotide sequence of SEQ ID NO: 98 or a variant of SEQ ID NO: 98 that differs by 1, 2, or 3 nucleotides.

156. The polynucleotide of any one of aspects 134-155, wherein the AMBP sequence element comprises a nucleotide sequence of SEQ ID NO: 98.

157. The polynucleotide of any one of aspects 134-156, wherein the AMBP sequence element comprises a nucleotide sequence of SEQ ID NO: 97 or a variant of SEQ ID NO: 97 that differs by 1, 2, or 3 nucleotides.

158. The polynucleotide of any one of aspects 134-157, wherein the AMBP sequence element comprises a nucleotide sequence of SEQ ID NO: 97.

159. The polynucleotide of any one of aspects 134-158, wherein the AMBP sequence element is 5′ of the hAAT sequence element.

160. The polynucleotide of any one of aspects 134-159, wherein the TRE is liver specific.

161. The polynucleotide of any one of aspects 134-160, wherein the TRE promotes greater expression than HCR-hAAT.

162. The polynucleotide of any one of aspects 134-161, wherein the TRE promotes greater expression than FRE72.

163. The polynucleotide of any one of aspects 134-162, wherein the TRE promotes expression at least 1.2 fold, at least 1.5 fold, at least 1.8 fold, or at least 2 fold higher compared to HCR-hAAT.

164. The polynucleotide of any one of aspects 134-163, wherein the TRE promotes expression at least 1.6 fold higher or at least 1.8 fold higher compared to HCR-hAAT.

165. The polynucleotide of any one of aspects 134-164, wherein the TRE promotes expression at least 1.2 fold, at least 1.5 fold, at least 1.8 fold, or at least 2 fold higher compared to FRE72.

166. The polynucleotide of any one of aspects 134-165, wherein the TRE promotes expression at least 1.6 fold or at least 1.8 fold higher compared to FRE72.

167. The polynucleotide of any one of aspects 134-166, wherein expression is measured by an expression assay comprising:
(i) transfecting human hepatocytes with a plasmid comprising an expression cassette comprising a transgene operably linked to the TRE;
(ii) incubating the transgene under conditions suitable for transgene expression to occur; and
(iii) measuring the level of the transgene by ELISA using an antibody specific for a protein encoded by the transgene.

168. The polynucleotide of aspect 167, wherein the human hepatocytes are Huh7 cells.

169. The polynucleotide of aspect 167 or 168, wherein the transgene encodes CFI (Complement Factor I).

170. The polynucleotide of any one of aspects 167-169, wherein step (i) of transfecting human hepatocytes comprises co-transfecting the human hepatocytes with a plasmid comprising an expression cassette comprising a transgene encoding luciferase operably linked to a promoter and the expression assay further comprises measuring the total luciferase expression.

171. The polynucleotide of any one of aspects 134-170, wherein the TRE comprises an ApoE-HCR1 sequence element.

172. The polynucleotide of aspect 171, wherein the TRE comprising the ApoE-HCR1 sequence element promotes equivalent or greater expression than a corresponding TRE lacking the ApoE-HCR1 sequence element.

173. The polynucleotide of aspect 171 or 172, wherein the TRE comprising the ApoE-HCR1 sequence element promotes greater expression than a corresponding TRE lacking the ApoE-HCR1 sequence element.

174. The polynucleotide of any one of aspects 171-173, wherein the ApoE-HCR1 sequence element comprises a nucleotide sequence of SEQ ID NO: 93 or a variant of SEQ ID NO: 93 that differs by 1, 2, or 3 nucleotides.

175. The polynucleotide of any one of aspects 171-174, wherein the ApoE-HCR1 sequence element comprises a nucleotide sequence of SEQ ID NO: 93.

176. The polynucleotide of any one of aspects 171-175, wherein the ApoE-HCR1 sequence element comprises a nucleotide sequence of SEQ ID NO: 92 or a variant of SEQ ID NO: 92 that differs by 1, 2, or 3 nucleotides.

177. The polynucleotide of any one of aspects 171-176, wherein the ApoE-HCR1 sequence element is 5′ of the hAAT sequence element.

178. The polynucleotide of any one of aspects 171-177, wherein the ApoE-HCR1 sequence element is 5′ of the AMBP sequence element.

179. The polynucleotide of any one of aspects 171-177, wherein the ApoE-HCR1 sequence element is 3′ of the AMBP sequence element.

180. The polynucleotide of any one of aspects 171-178, wherein the ApoE-HCR1 sequence element is 5′ of both the hAAT sequence element and the AMBP sequence element.

181. The polynucleotide of any one of aspects 171-177 or 179, wherein the ApoE-HCR1 sequence element is 5′ of the hAAT sequence element and 3′ of the AMBP sequence element.

182. The polynucleotide of any one of aspects 134-181, wherein the TRE comprises a CRM6 sequence element.

183. The polynucleotide of aspect 182, wherein the TRE comprising the CRM6 sequence element promotes equivalent or greater expression than a corresponding TRE lacking the CRM6 sequence element.

184. The polynucleotide of aspect 182 or aspect 183, wherein the TRE comprising the CRM6 sequence element promotes equivalent expression than a corresponding TRE lacking the CRM6 sequence element.

185. The polynucleotide of any one of aspects 182-184, wherein the CRM6 sequence element comprises a binding site selected from the group consisting of an Sp1 binding site, an Sp2 binding site, an HNF3a binding site, and an HNF-1 binding site.

186. The polynucleotide of aspect 185, wherein the CRM6 sequence element comprises an Sp1 binding site, an Sp2 binding site, an HNF3a binding site, and an HNF1 binding site.

187. The polynucleotide of any one of aspects 182-186, wherein the CRM6 sequence element comprises a nucleotide sequence of SEQ ID NO: 100 or a variant of SEQ ID NO: 100 that differs by 1 or 2 nucleotides.

188. The polynucleotide of any one of aspects 182-187, wherein the CRM6 sequence element comprises a nucleotide sequence of SEQ ID NO: 100.

189. The polynucleotide of any one of aspects 182-188, wherein the CRM6 sequence element comprises a nucleotide sequence of SEQ ID NO: 101 or a variant of SEQ ID NO: 101 that differs by 1, 2, or 3 nucleotides.

190. The polynucleotide of any one of aspects 182-189, wherein the CRM6 sequence element comprises a nucleotide sequence of SEQ ID NO: 101.

191. The polynucleotide of any one of aspects 182-190, wherein the CRM6 sequence element comprises a nucleotide sequence of SEQ ID NO: 100 and a nucleotide sequence of SEQ ID NO: 101.

192. The polynucleotide of any one of aspects 182-191, wherein the CRM6 sequence element comprises a nucleotide sequence of SEQ ID NO: 99 or a variant of SEQ ID NO: 99 that differs by 1, 2, or 3 nucleotides.

193. The polynucleotide of any one of aspects 182-192, wherein the CRM6 sequence element comprises a nucleotide sequence of SEQ ID NO: 99.

194. The polynucleotide of any one of aspects 182-193, wherein the CRM6 sequence element is 5′ of the hAAT sequence element.

195. The polynucleotide of any one of aspects 182-194, wherein the CRM6 sequence element is 5′ of the AMBP sequence element.

196. The polynucleotide of any one of aspects 182-194, wherein the CRM6 sequence element is 3′ of the AMBP sequence element.

197. The polynucleotide of any one of aspects 182-195, wherein the CRM6 sequence element is 5′ of both the hAAT sequence element and the AMBP sequence element.

198. The polynucleotide of any one of aspects 182-194 or 196, wherein the CRM6 sequence element is 5′ of the hAAT sequence element and 3′ of the AMBP sequence element.

199. The polynucleotide of any one of aspects 134-198, wherein the TRE comprises an ALDOB sequence element.

200. The polynucleotide of aspect 199, wherein the TRE comprising the ALDOB sequence element promotes equivalent or greater expression than a corresponding TRE lacking the ALDOB sequence element.

201. The polynucleotide of any one of aspect 199 or 200, wherein the TRE comprising the ALDOB sequence element promotes equivalent expression than a corresponding TRE lacking the ALDOB sequence element.

202. The polynucleotide of any one of aspects 199-201, wherein the ALDOB sequence element comprises at least one binding site selected from the group consisting of an SP1 binding site, an HNF1a binding site, a C/EBP binding site, a GATA2 binding site, a USF1 binding site, and a USF2 binding site.

203. The polynucleotide of aspect 202, wherein the ALDOB sequence element comprises an SP1 binding site, an HNF1a binding site, a C/EBP binding site, a GATA2 binding site, a USF1 binding site, and a USF2 binding site.

204. The polynucleotide of any one of aspects 199-203, wherein the ALDOB sequence element comprises a nucleotide sequence of SEQ ID NO: 103 or a variant of SEQ ID NO: 103 that differs by 1, 2, or 3 nucleotides.

205. The polynucleotide of any one of aspects 199-204, wherein the ALDOB sequence element comprises a nucleotide sequence of SEQ ID NO: 103.

206. The polynucleotide of any one of aspects 199-205, wherein the ALDOB sequence element comprises a nucleotide sequence of SEQ ID NO: 102 or a variant of SEQ ID NO: 102 that differs by 1, 2, or 3 nucleotides.

207. The polynucleotide of aspect 206, wherein the ALDOB sequence element comprises a nucleotide sequence of SEQ ID NO: 102.

208. The polynucleotide of any one of aspects 199-207, wherein the ALDOB sequence element is 5′ of the hAAT sequence element.

209. The polynucleotide of any one of aspects 199-208, wherein the ALDOB sequence element is 5′ of the AMBP sequence element.

210. The polynucleotide of any one of aspects 199-208, wherein the ALDOB sequence element is 3′ of the AMBP sequence element.

211. The polynucleotide of any one of aspects 199-209, wherein the ALDOB sequence element is 5′ of both the hAAT sequence element and the AMBP sequence element.

212. The polynucleotide of any one of aspects 199-208 or 210, wherein the ALDOB sequence element is 5′ of the hAAT sequence element and 3′ of the AMBP sequence element.

213. The polynucleotide of any one of aspects 134-212, wherein the TRE comprises an F2 sequence element.

214. The polynucleotide of aspect 213, wherein the TRE comprising the F2 sequence element promotes equivalent or greater expression than a corresponding TRE lacking the F2 sequence element.

215. The polynucleotide of aspect 213 or aspect 214, wherein the TRE comprising the F2 sequence element promotes greater expression than a corresponding TRE lacking the F2 sequence element.

216. The polynucleotide of any one of aspects 213-215, wherein the F2 sequence element comprises at least one binding site selected from the group consisting of an HNF4a binding site, an HNF3a binding site, an HNF1a binding site, an HNF3b binding site, an STAT3 binding site, an RBPJ binding site, and an SP3 binding site.

217. The polynucleotide of aspect 216, wherein the F2 sequence element comprises an HNF4a binding site, an HNF3a binding site, an HNF1a binding site, an HNF3b binding site, an STAT3 binding site, an RBPJ binding site, and an SP3 binding site.

218. The polynucleotide of any one of aspects 213-217, wherein the F2 sequence element comprises a nucleotide sequence of SEQ ID NO: 105 or a variant of SEQ ID NO: 105 that differs by 1, 2, or 3 nucleotides.

219. The polynucleotide of aspect 218, wherein the F2 sequence element comprises a sequence of SEQ ID NO: 105.

220. The polynucleotide of any one of aspects 213-219, wherein the F2 sequence element comprises a nucleotide sequence of SEQ ID NO: 104 or a variant of SEQ ID NO: 104 that differs by 1, 2, or 3 nucleotides.

221. The polynucleotide of aspect 220, wherein the F2 sequence element comprises a nucleotide sequence of SEQ ID NO: 104.

222. The polynucleotide of any one of aspects 213-221, wherein the F2 sequence element is 5′ of the hAAT sequence element.

223. The polynucleotide of any one of aspects 213-222, wherein the F2 sequence element is 5′ of the AMBP sequence element.

224. The polynucleotide of any one of aspects 213-222, wherein the F2 sequence element is 3′ of the AMBP sequence element.

225. The polynucleotide of any one of aspects 213-223, wherein the F2 sequence element is 5′ of both the hAAT sequence element and the AMBP sequence element.

226. The polynucleotide of any one of aspects 213-222 or 224, wherein the F2 sequence element is 5′ of the hAAT sequence element and 3′ of the AMBP sequence element.

227. The polynucleotide of any one of aspects 134-226, wherein the TRE comprises:
(a) a nucleotide sequence having at least 80%, at least 85%, at least 90%, at least 95%, at least 98%, or at least 99% sequence identity to any one of SEQ ID NO: 68-91; or
(b) a nucleotide sequence of any one of SEQ ID NO: 68-91.

228. The polynucleotide of any one of aspects 134-227, wherein the TRE comprises:
(a) a nucleotide sequence having at least 80%, at least 85%, at least 90%, at least 95%, at least 98%, or at least 99% sequence identity to SEQ ID NO: 70; or
(b) the nucleotide sequence of SEQ ID NO: 70.

229. The polynucleotide of any one of aspects 134-227, wherein the TRE comprises:
(a) a nucleotide sequence having at least 80%, at least 85%, at least 90%, at least 95%, at least 98%, or at least 99% sequence identity to SEQ ID NO: 74; or
(b) the nucleotide sequence of SEQ ID NO: 74.

230. The polynucleotide of any one of aspect 134-229, wherein the TRE further comprises:
a) a TATA box;
b) an HNF6 binding site;
c) an HNF4a binding site;
d) an HNF1 binding site; and/or
e) an HNF3 binding site.

231. The polynucleotide of aspect 230, wherein the TRE further comprises a TATA box, optionally a canonical TATA box.

232. The polynucleotide of aspect 231, wherein the TRE comprises a hAAT sequence element and the TATA box is inserted into the hAAT sequence element.

233. The polynucleotide of aspect 231 or 232, wherein the hAAT sequence element comprises a nucleotide sequence of SEQ ID NO: 95 or a variant of SEQ ID NO: 95 that differs by 1, 2, or 3 nucleotides and the TATA box is 3′ of the nucleotide sequence of SEQ ID NO: 95 or variant of SEQ ID NO: 95.

234. The polynucleotide of aspect 231 or 232, wherein the hAAT sequence element comprises a nucleotide sequence of SEQ ID NO: 95 or a variant of SEQ ID NO: 95 that differs by 1, 2, or 3 nucleotides and the TATA box is 5′ of the nucleotide sequence of SEQ ID NO: 95 or variant of SEQ ID NO: 95.

235. The polynucleotide of any one of aspects 230-234, wherein the TRE promotes equivalent or greater expression than a corresponding TRE not comprising the TATA box.

236. The polynucleotide of any one of aspects 230-235, wherein the TRE further comprises an HNF6 binding site.

237. The polynucleotide of aspect 236, wherein the TRE comprises an HNF6 binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF6 binding site is 5′of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 31 or a variant of SEQ ID NO: 96 that differs by 1, 2, or 3 nucleotides and the HNF6 binding site is 5′of the hAAT sequence element that comprises a nucleotide sequence of SEQ ID NO: 96 or a variant of SEQ ID NO: 31 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF6 binding site is 5′of the hAAT sequence element that comprises a nucleotide sequence of SEQ ID NO: 94 or a variant of SEQ ID NO: 94 that differs by 1, 2, or 3 nucleotides; and/or
d. the TRE comprises a hAAT sequence element that comprises a nucleotide sequence of SEQ ID NO: 95 or a variant of SEQ ID NO: 95 that differs by 1, 2, or 3 nucleotides and the HNF6 binding site is 3′of the hAAT sequence element that comprises a nucleotide sequence of SEQ ID NO: 95 or a variant of SEQ ID NO: 95 that differs by 1, 2, or 3 nucleotides; and/or
e. the TRE comprises a hAAT sequence element that comprises a nucleotide sequence of SEQ ID NO: 96 or a variant of SEQ ID NO: 96 that differs by 1, 2, or 3 nucleotides and the HNF6 binding site is 3′of the hAAT sequence element that comprises a nucleotide sequence of SEQ ID NO: 96 or a variant of SEQ ID NO: 96 that differs by 1, 2, or 3 nucleotides; and/or
f. the TRE comprises a hAAT sequence element that comprises a nucleotide sequence of SEQ ID NO: 94 or a variant of SEQ ID NO: 94 that differs by 1, 2, or 3 nucleotides and the HNF6 binding site is 3′of the hAAT sequence element that comprises a nucleotide sequence of SEQ ID NO: 94 or a variant of SEQ ID NO: 94 that differs by 1, 2, or 3 nucleotides; and/or
g. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF6 binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
h. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF6 binding site is 3′ of the ApoE-HCR1 sequence element that comprises a nucleotide sequence of SEQ ID NO: 92 or a variant of SEQ ID NO: 92 that differs by 1, 2, or 3 nucleotides; and/or
i. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF6 binding site is 5′ of the ApoE-HCR1 sequence element that comprises a nucleotide sequence of SEQ ID NO: 93 or a variant of SEQ ID NO: 93 that differs by 1, 2, or 3 nucleotides; and/or
j. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF6 binding site is 5′ of the ApoE-HCR1 sequence element that comprises a nucleotide sequence of SEQ ID NO: 92 or a variant of SEQ ID NO: 92 that differs by 1, 2, or 3 nucleotides; and/or
k. the TRE comprises an AMBP sequence element that comprises a nucleotide sequence of SEQ ID NO: 98 or a variant of SEQ ID NO: 98 that differs by 1, 2, or 3 nucleotides and the HNF6 binding site is 3′ of the AMBP sequence element that comprises a nucleotide sequence of SEQ ID NO: 98 or a variant of SEQ ID NO: 98 that differs by 1, 2, or 3 nucleotides; and/or
l. the TRE comprises an AMBP sequence element that comprises a nucleotide sequence of SEQ ID NO: 97 or a variant of SEQ ID NO: 97 that differs by 1, 2, or 3 nucleotides and the HNF6 binding site is 3′ of the AMBP sequence element that comprises a nucleotide sequence of SEQ ID NO: 97 or a variant of SEQ ID NO: 97 that differs by 1, 2, or 3 nucleotides; and/or
m. the TRE comprises an AMBP sequence element that comprises a nucleotide sequence of SEQ ID NO: 98 or a variant of SEQ ID NO: 98 that differs by 1, 2, or 3 nucleotides and the HNF6 binding site is 5′ of the AMBP sequence element that comprises a nucleotide sequence of SEQ ID NO: 98 or a variant of SEQ ID NO: 98 that differs by 1, 2, or 3 nucleotides; and/or
n. the TRE comprises an AMBP sequence element that comprises a nucleotide sequence of SEQ ID NO: 97 or a variant of SEQ ID NO: 97 that differs by 1, 2, or 3 nucleotides and the HNF6 binding site is 5′ of the AMBP sequence element that comprises a nucleotide sequence of SEQ ID NO: 97 or a variant of SEQ ID NO: 97 that differs by 1, 2, or 3 nucleotides.

238. The polynucleotide of any one of aspects 236-237, wherein the TRE promotes equivalent or greater expression than a corresponding TRE not comprising the HNF6 binding site.

239. The polynucleotide of any one of aspects 236-238, wherein the TRE comprises a HNF6 binding site and a TATA box and the TRE promotes equivalent or greater expression than a corresponding TRE not comprising the HNF6 binding site and the TATA box.

240. The polynucleotide of any one of aspects 230-239, wherein the TRE further comprises an HNF4a binding site.

241. The polynucleotide of aspect 240, wherein the TRE comprises an HNF4a binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF4a binding site is 5′of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 96 or a variant of SEQ ID NO: 96 that differs by 1, 2, or 3 nucleotides and the HNF4a binding site is 5′of the hAAT sequence element that comprises a nucleotide sequence of SEQ ID NO: 96 or a variant of SEQ ID NO: 96 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF4a binding site is 5′of the hAAT sequence element that comprises a nucleotide sequence of SEQ ID NO: 94 or a variant of SEQ ID NO: 94 that differs by 1, 2, or 3 nucleotides; and/or
d. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF4a binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
e. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF4a binding site is 5′ of the ApoE-HCR1 sequence element that comprises a nucleotide sequence of SEQ ID NO: 93 or a variant of SEQ ID NO: 93 that differs by 1, 2, or 3 nucleotides; and/or
f. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF4a binding site is 5′ of the ApoE-HCR1 sequence element that comprises a nucleotide sequence of SEQ ID NO: 92 or a variant of SEQ ID NO: 92 that differs by 1, 2, or 3 nucleotides; and/or
g. the TRE comprises an AMBP sequence element that comprises a nucleotide sequence of SEQ ID NO: 98 or a variant of SEQ ID NO: 98 that differs by 1, 2, or 3 nucleotides and the HNF4a binding site is 3′ of the AMBP sequence element that comprises a nucleotide sequence of SEQ ID NO: 98 or a variant of SEQ ID NO: 98 that differs by 1, 2, or 3 nucleotides; and/or
h. the TRE comprises an AMBP sequence element that comprises a nucleotide sequence of SEQ ID NO: 97 or a variant of SEQ ID NO: 97 that differs by 1, 2, or 3 nucleotides and the HNF4a binding site is 3′ of the AMBP sequence element that comprises a nucleotide sequence of SEQ ID NO: 97 or a variant of SEQ ID NO: 97 that differs by 1, 2, or 3 nucleotides; and/or
i. the TRE comprises an AMBP sequence element that comprises a nucleotide sequence of SEQ ID NO: 98 or a variant of SEQ ID NO: 98 that differs by 1, 2, or 3 nucleotides and the HNF4a binding site is 5′ of the AMBP sequence element that comprises a nucleotide sequence of SEQ ID NO: 98 or a variant of SEQ ID NO: 98 that differs by 1, 2, or 3 nucleotides; and/or
j. the TRE comprises an AMBP sequence element that comprises a nucleotide sequence of SEQ ID NO: 97 or a variant of SEQ ID NO: 97 that differs by 1, 2, or 3 nucleotides and the HNF4a binding site is 5′ of the AMBP sequence element that comprises a nucleotide sequence of SEQ ID NO: 97 or a variant of SEQ ID NO: 97 that differs by 1, 2, or 3 nucleotides.

242. The polynucleotide of any one of aspects 240-241, wherein the TRE promotes equivalent or greater expression than a corresponding TRE not comprising the HNF4a binding site.

243. The polynucleotide of any one of aspects 240-242, wherein the TRE comprises a HNF6 binding site and an HNF4a binding site and the TRE promotes equivalent or greater expression than a corresponding TRE not comprising the HNF6 binding site and the HNF4a binding site.

244. The polynucleotide of any one of aspects 240-243, wherein the TRE further comprises an HNF1 binding site.

245. The polynucleotide of aspect 244, wherein the TRE comprises an HNF1 binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF1 binding site is 5′ of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 96 or a variant of SEQ ID NO: 96 that differs by 1, 2, or 3 nucleotides and the HNF1 binding site is 5′ of the hAAT sequence element that comprises a nucleotide sequence of SEQ ID NO: 96 or a variant of SEQ ID NO: 96 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF1 binding site is 5′ of the hAAT sequence element that comprises a nucleotide sequence of SEQ ID NO: 94 or a variant of SEQ ID NO: 94 that differs by 1, 2, or 3 nucleotides; and/or
d. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF1 binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
e. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF1 binding site is 3′ of the ApoE-HCR1 sequence element that comprises a nucleotide sequence of SEQ ID NO: 92 or a variant of SEQ ID NO: 92 that differs by 1, 2, or 3 nucleotides; and/or
f. the TRE comprises an AMBP sequence element that comprises a nucleotide sequence of SEQ ID NO: 98 or a variant of SEQ ID NO: 98 that differs by 1, 2, or 3 nucleotides and the HNF1 binding site is 3′ of the AMBP sequence element that comprises a nucleotide sequence of SEQ ID NO: 98 or a variant of SEQ ID NO: 98 that differs by 1, 2, or 3 nucleotides; and/or
g. the TRE comprises an AMBP sequence element that comprises a nucleotide sequence of SEQ ID NO: 97 or a variant of SEQ ID NO: 97 that differs by 1, 2, or 3 nucleotides and the HNF1 binding site is 3′ of the AMBP sequence element that comprises a nucleotide sequence of SEQ ID NO: 97 or a variant of SEQ ID NO: 97 that differs by 1, 2, or 3 nucleotides.

246. The polynucleotide of any one of aspects 244-245, wherein the TRE promotes equivalent or greater expression than a corresponding TRE not comprising the HNF1 binding site.

247. The polynucleotide of any one of aspects 244-246, wherein the TRE comprises an HNF6 binding site, an HNF4a binding site, and a TATA box and the TRE promotes equivalent or greater expression than a corresponding TRE not comprising the HNF6 binding site, the HNF4a binding site, and the TATA box.

248. The polynucleotide of any one of aspects 230-247, wherein the TRE further comprises an HNF3 binding site.

249. The polynucleotide of aspect 248, wherein the TRE comprises an HNF3 binding site, and:
a. the TRE comprises a hAAT sequence element that comprises a nucleotide sequence of SEQ ID NO: 95 or a variant of SEQ ID NO: 95 that differs by 1, 2, or 3 nucleotides and the HNF3 binding site is 5′of the hAAT sequence element that comprises a nucleotide sequence of SEQ ID NO: 95 or a variant of SEQ ID NO: 95 that differs by 1, 2, or 3 nucleotides; and/or
b. the TRE comprises a hAAT sequence element that comprises a nucleotide sequence of SEQ ID NO: 96 or a variant of SEQ ID NO: 96 that differs by 1, 2, or 3 nucleotides and the HNF3 binding site is 5′of the hAAT sequence element that comprises a nucleotide sequence of SEQ ID NO: 96 or a variant of SEQ ID NO: 96 that differs by 1, 2, or 3 nucleotides; and/or
c. the TRE comprises a hAAT sequence element that comprises a nucleotide sequence of SEQ ID NO: 94 or a variant of SEQ ID NO: 94 that differs by 1, 2, or 3 nucleotides and the HNF3 binding site is 5′of the hAAT sequence element that comprises a nucleotide sequence of SEQ ID NO: 94 or a variant of SEQ ID NO: 94 that differs by 1, 2, or 3 nucleotides; and/or
d. the TRE comprises an ApoE-HCR1 sequence element that comprises a nucleotide sequence of SEQ ID NO: 93 or a variant of SEQ ID NO: 93 that differs by 1, 2, or 3 nucleotides and the HNF3 binding site is 3′ of the ApoE-HCR1 sequence element that comprises a nucleotide sequence of SEQ ID NO: 93 or a variant of SEQ ID NO: 93 that differs by 1, 2, or 3 nucleotides; and/or
e. the TRE comprises an ApoE-HCR1 sequence element that comprises a nucleotide sequence of SEQ ID NO: 92 or a variant of SEQ ID NO: 92 that differs by 1, 2, or 3 nucleotides and the HNF3 binding site is 3′ of the ApoE-HCR1 sequence element that comprises a nucleotide sequence of SEQ ID NO: 92 or a variant of SEQ ID NO: 92 that differs by 1, 2, or 3 nucleotides; and/or
f. the TRE comprises an AMBP sequence element that comprises a nucleotide sequence of SEQ ID NO: 98 or a variant of SEQ ID NO: 98 that differs by 1, 2, or 3 nucleotides and the HNF3 binding site is 3′ of the AMBP sequence element that comprises a nucleotide sequence of SEQ ID NO: 98 or a variant of SEQ ID NO: 98 that differs by 1, 2, or 3 nucleotides; and/or
g. the TRE comprises an AMBP sequence element that comprises a nucleotide sequence of SEQ ID NO: 97 or a variant of SEQ ID NO: 97 that differs by 1, 2, or 3 nucleotides and the HNF3 binding site is 3′ of the AMBP sequence element that comprises a nucleotide sequence of SEQ ID NO: 97 or a variant of SEQ ID NO: 97 that differs by 1, 2, or 3 nucleotides.

250. The polynucleotide of any one of aspects 248-249, wherein the TRE promotes equivalent or greater expression than a corresponding TRE not comprising the HNF3 binding site.

251. The polynucleotide of any one of aspects 248-250, wherein the TRE comprises an HNF3 binding site and a TATA box and the TRE promotes equivalent or greater expression than a corresponding TRE not comprising the HNF3 binding site and the TATA box.

252. The polynucleotide of any one of the preceding aspects, wherein the polynucleotide further comprises one or two ITR(s).

253. The polynucleotide of aspect 252, wherein the nucleotide sequence of the or each ITR is fewer than 157, fewer than 154, or around 145 nucleotides in length.

254. The polynucleotide of aspect 252 or 253, wherein the or each ITR is a wild-type ITR.

255. The polynucleotide of any one of aspects 252 to 254, wherein the or each ITR is an AAV2 ITR.

256. The polynucleotide of any one of aspects 252 to 255, wherein the nucleotide sequence of the or each ITR comprises a nucleotide sequence of SEQ ID NO: 53 or SEQ ID NO: 54.

257. The polynucleotide of any one of the preceding aspects, wherein the polynucleotide further comprises a polyA nucleotide sequence.

258. The polynucleotide of aspect 257, wherein the polyA nucleotide sequence comprises the nucleotide sequence of SEQ ID NO: 55.

259. The polynucleotide of any one of the preceding aspects, wherein the polynucleotide further comprises an intron.

260. The polynucleotide of any one of the preceding aspects, wherein the CFI polypeptide or the fragment thereof comprises a sequence:
a) at least 95%, at least 98%, at least 99%, at least 99.5%, at least 99.8%, or 100% identical to SEQ ID NO: 43 or 44; or
b) at least 95%, at least 98%, at least 99%, at least 99.5%, at least 99.8%, or 100% identical to a fragment of SEQ ID NO: 43 or 44 at least 300, at least 350, at least 400, 583 or fewer, 565 or fewer, between 300 and 583, or between 300 and 565 amino acids in length.

261. A viral particle comprising a recombinant genome comprising the polynucleotide of any one of the preceding aspects.

262. The viral particle of aspect 261, which is an AAV, adenoviral, or lentiviral viral particle.

263. The viral particle of aspect 262, which is an AAV viral particle.

264. The viral particle of any one of aspects 261 to 263, wherein the viral particle comprises a capsid.

265. The viral particle of aspect 264, wherein the viral particle comprises a liver-tropic capsid.

266. The viral particle of aspect 265, wherein the liver-tropic capsid comprises a sequence at least 98%, at least 99%, or at least 99.5% identical to a fragment of at least 600, at least 650, at least 700, between 600 and 734, between 600 and 736, between 650 and 734, between 650 and 736, between 700 and 734, between 700 and 736, around 734, or around 736 amino acids of any one of SEQ ID NOs: 56-59.

267. The viral particle of aspect 266, wherein the liver-tropic capsid comprises a sequence at least 99% identical to any one of SEQ ID NOs: 56-59.

268. The viral particle of aspect 267, wherein the liver-tropic capsid comprises a sequence at least 99% identical to SEQ ID NO: 57.

269. The viral particle of any one of aspects 261 to 268, wherein the recombinant genome is single-stranded.

270. The viral particle of any one of aspects 261 to 269, wherein the CFI polypeptide or the fragment thereof encoded by the CFI nucleotide sequence is expressed at the same or a higher level on administration of the viral particle compared to expression of a CFI polypeptide encoded by a reference CFI nucleotide sequence on administration of an equivalent viral particle comprising the reference CFI nucleotide sequence.

271. The viral particle of aspect 270, wherein the expression of the CFI polypeptide or the fragment thereof encoded by the CFI nucleotide sequence is at a higher level compared to the expression of the CFI polypeptide encoded by the reference CFI nucleotide sequence.

272. The viral particle of aspect 270 or 271, wherein the expression of the CFI polypeptide or the fragment thereof encoded by the CFI nucleotide sequence is at least 1.5x, at least 2x, at least 3x, at least 5x, at least 8x, or at least 10x higher compared to the CFI polypeptide encoded by the reference CFI nucleotide sequence.

273. The viral particle of aspect 271 or 272, wherein the reference CFI nucleotide sequence is a wild type CFI nucleotide sequence.

274. The viral particle of aspect 273, wherein the wild type CFI nucleotide sequence is the sequence of SEQ ID NO: 41 or 42.

275. The viral particle of any one of aspects 270 to 272, wherein the reference CFI nucleotide sequence is the sequence of SEQ ID NO: 1 or 21.

276. The viral particle of any one of aspects 270 to 272, wherein the reference CFI nucleotide sequence is the sequence of SEQ ID NO: 5 or 25.

277. The viral particle of any one of the preceding aspects, wherein the expression of the CFI polypeptide or the fragment thereof encoded by the CFI nucleotide sequence is at least 50%, at least 60%, at least 70%, at least 80%, at least 90%, at least 95%, or at least 100% of the expression of the CFI polypeptide encoded by the sequence of SEQ ID NO: 5 or 25.

278. A composition comprising the polynucleotide or viral particle of any one of the preceding aspects, and a pharmaceutically acceptable excipient.

279. The polynucleotide according to any one of aspects 1 to 260, the viral particle according to any one of aspects 261 to 277, or the composition according to aspect 278 for use in a method of treatment.

280. A method of treatment comprising administering an effective amount of the polynucleotide according to any one of aspects 1 to 260, the viral particle according to any one of aspects 261 to 277, or the composition according to aspect 278.

281. Use of the polynucleotide according to any one of aspects 1 to 260, the viral particle according to any one of aspects 261 to 277, or the composition according to aspect 278 in the manufacture of a medicament for use in a method of treatment.

282. The polynucleotide, viral particle or composition for use, or use of any one of aspects 279 to 281, wherein the method of treatment comprises administering an effective amount of the polynucleotide, viral particle or composition to a patient.

283. A polynucleotide comprising a Complement Factor I (CFI) nucleotide sequence, a viral particle comprising a recombinant genome comprising the polynucleotide, or a composition comprising the polynucleotide or viral particle for use in a method of treatment, wherein the method of treatment is a method of treating a complement-mediated disorder, and the CFI nucleotide sequence encodes a CFI polypeptide or a fragment thereof.

284. A method of treatment comprising administering an effective amount of a polynucleotide comprising a CFI nucleotide sequence, a viral particle comprising a recombinant genome comprising the polynucleotide, or a composition comprising the polynucleotide or viral particle, wherein the method of treatment is a method of treating a complement-mediated disorder, and the CFI nucleotide sequence encodes a CFI polypeptide or a fragment thereof.

285. Use of a polynucleotide comprising a CFI nucleotide sequence, a viral particle comprising a recombinant genome comprising the polynucleotide, or a composition comprising the polynucleotide or viral particle in the manufacture of a medicament for use in a method of treatment, wherein the method of treatment is a method of treating a complement-mediated disorder, and the CFI nucleotide sequence encodes a CFI polypeptide or a fragment thereof.

286. The polynucleotide, viral particle or composition for use, or use of any one of aspects 283 to 285, wherein the method of treatment comprises administering an effective amount of the polynucleotide, viral particle or composition to a patient.

287. The polynucleotide, viral particle or composition for use, or use of any one of aspects 279 to 282, wherein the method of treatment is a method of treating a complement-mediated disorder.

288. The polynucleotide, viral particle, or composition for use, use, or method of any one of aspects 283 to 287, wherein the disorder is a C3-mediated disorder.

289. The polynucleotide, viral particle, or composition for use, use, or method of any one of aspects 283 to 288, wherein the disorder is a kidney disorder.

290. The polynucleotide, viral particle, or composition for use, use, or method of any one of aspects 283 to 289, wherein the disorder is associated with over-activity of the complement C3b feedback cycle.

291. The polynucleotide, viral particle, or composition for use, use, or method of any one of aspects 283 to 290, wherein the disorder is selected from C3 glomerulopathy, IgA nephropathy, lupus nephritis, systemic lupus erythematosus, membranous nephropathy, membranoproliferative glomerulonephritis, paroxysmal nocturnal hemoglobinuria, atypical hemolytic uremic syndrome, autoimmune haemolytic anemia, ANCA-associated vasculitis, Gaucher disease, peritonitis, age-related macular degeneration, diabetic retinopathy, dense deposit disease, age-related inflammatory or autoinflammatory diseases, autoimmune arthritis such as rheumatoid arthritis, atherosclerosis, chronic cardiovascular disease, Alzheimer’s disease, systemic vasculitis, Guillain-Barre syndrome, and Henoch-Schonlein purpura.

292. The polynucleotide, viral particle, or composition for use, use, or method of any one of aspects 283 to 291, wherein the disorder is selected from C3 glomerulopathy, C3 glomerulonephritis and dense deposit disease.

293. The polynucleotide, viral particle, or composition for use, use, or method of any one of aspects 283 to 291, wherein the disorder is atypical hemolytic uremic syndrome with monoallelic CFH mutation.

294. The polynucleotide, viral particle, or composition for use, use, or method of any one of aspects 283 to 291, wherein the disorder is a kidney glomerular or tubular disorder.

295. The polynucleotide, viral particle, or composition for use, use, or method of any one of aspects 283 to 291 or 294, wherein the disorder is selected from C3 glomerulopathy, IgA nephropathy, lupus nephritis and membranous nephropathy.

296. The polynucleotide, viral particle, or composition for use, use, or method of any one of aspects 283 to 291 or 294 to 295, wherein the disorder is lupus nephritis.

297. The polynucleotide, viral particle, or composition for use, use, or method of any one of aspects 283 to 291, wherein the disorder is systemic lupus erythematosus.

298. The polynucleotide, viral particle, or composition for use, use, or method of any one of aspects 283 to 297, wherein the polynucleotide, viral particle, or composition is according to any one of aspects 1 to 278.

299. The polynucleotide, viral particle, or composition for use, use, or method of any one of aspects 283 to 298, wherein the administration of the polynucleotide, viral particle, or composition reduces the inflammation associated with the disorder in a subj ect.

300. The polynucleotide, viral particle or composition for use, use, or method of aspect 299, wherein administration of the polynucleotide, viral particle or composition reduces the inflammation to a level of less than or around 60%, less than or around 55%, less than or around 50%, less than or around 45%, less than or around 40%, less than or around 35%, less than or around 30%, less than or around 25%, or less than or around 20% of the level of inflammation present before the polynucleotide, viral particle or composition was administered.

301. The polynucleotide, viral particle or composition for use, use, or method of any one of aspects 279 to 300 wherein the method of treatment is a method for reducing inflammation in a patient.

302. The polynucleotide, viral particle or composition for use, use, or method of any one of aspects 283 to 301 wherein the method of treatment is a method for reducing inflammation in a patient, thereby treating the complement-mediated disorder.

303. The polynucleotide, viral particle or composition for use, use, or method of any one of aspects 283 to 302 wherein the method of treatment is a method for treating a disease by reducing inflammation in a patient.

304. The polynucleotide, viral particle or composition for use, use, or method of any one of aspects 301 to 303, wherein reducing inflammation is reducing inflammation to a level of less than or around 60%, less than or around 55%, less than or around 50%, less than or around 45%, less than or around 40%, less than or around 35%, less than or around 30%, less than or around 25%, or less than or around 20% of the level of inflammation present before the polynucleotide, viral particle or composition was administered.

305. The polynucleotide, viral particle or composition for use, use, or method of any one of aspects 282 or 286 to 304 wherein the method of treatment comprises administering an effective amount of the polynucleotide, viral particle or composition to a patient, and the effective amount is an amount sufficient to reduce inflammation in the patient.

306. The polynucleotide, viral particle or composition for use, use, or method of aspect 305, wherein an amount sufficient to reduce inflammation in the patient is an amount sufficient to reduce the inflammation to a level of less than or around 60%, less than or around 55%, less than or around 50%, less than or around 45%, less than or around 40%, less than or around 35%, less than or around 30%, less than or around 25%, or less than or around 20% of the level of inflammation present before the polynucleotide, viral particle or composition was administered.

307.The polynucleotide, viral particle, or composition for use, use, or method of any one of aspects 282 to 298, wherein the administration of the polynucleotide, viral particle, or composition reduces proteinuria in a subject.

308. The polynucleotide, viral particle, or composition for use, use, or method of aspect 307, wherein proteinuria is scored by measuring the albumin-to-creatinine ratio (ACR) in urine.

309. The polynucleotide, viral particle or composition for use, use, or method of aspect 307 or 308, wherein administration of the polynucleotide, viral particle or composition reduces proteinuria to a level of less than or around 70%, less than or around 65%, less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of proteinuria present before the polynucleotide, viral particle or composition was administered.

310. The polynucleotide, viral particle or composition for use, use, or method of any one of aspects 283 to 309 wherein the method of treatment is a method for reducing proteinuria in a patient.

311. The polynucleotide, viral particle or composition for use, use, or method of any one of aspects 283 to 310 wherein the method of treatment is a method for reducing proteinuria in a patient, thereby treating the complement-mediated disorder.

312. The polynucleotide, viral particle or composition for use, use, or method of any one of aspects 283 to 311 wherein the method of treatment is a method for treating a disease by reducing proteinuria in a patient.

313. The polynucleotide, viral particle or composition for use, use, or method of any one of aspects 310 to 312, wherein reducing proteinuria is reducing proteinuria to a level of less than or around 70%, less than or around 65%, less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of proteinuria present before the polynucleotide, viral particle or composition was administered.

314. The polynucleotide, viral particle or composition for use, use, or method of any one of aspects 282 to 305 wherein the method of treatment comprises administering an effective amount of the polynucleotide, viral particle or composition to a patient, and the effective amount is an amount sufficient to reduce proteinuria in the patient.

315. The polynucleotide, viral particle or composition for use, use, or method of aspect 314, wherein an amount sufficient to reduce proteinuria in the patient is an amount sufficient to reduce the proteinuria to a level of less than or around 70%, less than or around 65%, less than or around 60%, less than or around 55%, less than or around 50%, less than or around 45%, less than or around 40%, less than or around 35%, less than or around 30%, less than or around 25%, less than or around 20%, less than or around 15%, or less than or around 10% of the level of proteinuria present before the polynucleotide, viral particle or composition was administered.

316. The polynucleotide, viral particle, or composition for use, use, or method of any one of aspects 280 to 315, wherein the administration of the polynucleotide, viral particle, or composition reduces kidney glomeruli pathology and/or kidney tubule pathology in a subject.

317. The polynucleotide, viral particle or composition for use, use, or method of aspect 316, wherein administration of the polynucleotide, viral particle or composition reduces kidney glomeruli pathology and/or kidney tubule pathology as seen by a reduced level of pathology on a PAS stain.

318. The polynucleotide, viral particle or composition for use, use, or method of any one of aspects 283 to 317 wherein the method of treatment is a method for reducing kidney glomeruli pathology and/or kidney tubule pathology in a patient.

319. The polynucleotide, viral particle or composition for use, use, or method of any one of aspects 283 to 318 wherein the method of treatment is a method for reducing kidney glomeruli pathology and/or kidney tubule pathology in a patient, thereby treating the complement-mediated disorder.

320. The polynucleotide, viral particle or composition for use, use, or method of any one of aspects 283 to 319 wherein the method of treatment is a method for treating a disease by reducing kidney glomeruli pathology and/or kidney tubule pathology in a patient.

321. The polynucleotide, viral particle or composition for use, use, or method of any one of aspects 318 to 320, wherein reducing kidney glomeruli pathology and/or kidney tubule pathology is reducing kidney glomeruli pathology and/or kidney tubule pathology as seen by a reduced level of pathology on a PAS stain.

322. The polynucleotide, viral particle or composition for use, use, or method of any one of aspects 282 to 321 wherein the method of treatment comprises administering an effective amount of the polynucleotide, viral particle or composition to a patient, and the effective amount is an amount sufficient to reduce kidney glomeruli pathology and/or kidney tubule pathology in the patient.

323. The polynucleotide, viral particle or composition for use, use, or method of aspect 322, wherein an amount sufficient to reduce kidney glomeruli pathology and/or kidney tubule pathology in the patient is an amount sufficient to reduce the kidney glomeruli pathology and/or kidney tubule pathology as seen by a reduced level of pathology on a PAS stain.

324. The polynucleotide, viral particle, or composition for use, use, or method of any one of aspects 283 to 323, wherein the disorder is not an ocular disorder.

325.The polynucleotide, viral particle, or composition for use, use, or method of any one of aspects 283 to 324, wherein the polynucleotide, viral particle, or composition is not administered by subretinal injection, direct retinal injection, suprachoroidal injection, and/or intravitreal injection.

326. The polynucleotide, viral particle, or composition for use, use, or method of any one of aspects 279 to 325, wherein the polynucleotide, viral particle, or composition is not administered intraocularly.

327. The polynucleotide, viral particle, or composition for use, use, or method of any one of aspects 279 to 326, wherein the polynucleotide, viral particle, or composition is administered: intravenously; systemically; to the liver via peripheral vein infusion; to the liver via hepatic vessels such as hepatic vein infusion or hepatic artery infusion; or via intraparenchymal administration direct to the liver.

328. The polynucleotide, viral particle, or composition for use, use, or method of any one of aspects 279 to 327, wherein the polynucleotide, viral particle, or composition is administered intravenously.

329. The polynucleotide, viral particle, or composition for use, use, or method of any one of aspects 279 to 327, wherein the polynucleotide, viral particle, or composition is administered systemically.

330. The polynucleotide, viral particle, or composition for use, use, or method of any one of aspects 279 to 327, wherein the polynucleotide, viral particle, or composition is administered to the liver via peripheral vein infusion.

331. The polynucleotide, viral particle, or composition for use, use, or method of any one of aspects 279 to 327, wherein the polynucleotide, viral particle, or composition is administered to the liver via hepatic vessels, wherein the administration is by hepatic vein infusion or hepatic artery infusion.

332. The polynucleotide, viral particle, or composition for use, use, or method of any one of aspects 279 to 327, wherein the polynucleotide, viral particle, or composition is administered via intraparenchymal administration direct to the liver.

333. The polynucleotide, viral particle, or composition for use, use, or method of any one of aspects 279 to 327, wherein the polynucleotide, viral particle, or composition is administered by injection into the renal artery.

334. The polynucleotide, viral particle, or composition for use, use, or method of any one of aspects 279 to 327, wherein the polynucleotide, viral particle, or composition is administered by retrograde administration.

335. The polynucleotide, viral particle, or composition for use, use, or method of aspect 334, wherein the polynucleotide, viral particle, or composition is administered by retrograde administration, wherein the administration is via the ureters using a urinary catheter.

336. The polynucleotide, viral particle, or composition for use, use, or method of any one of aspects 279 to 335, wherein the CFI nucleotide sequence comprises a sequence that is at least 95%, at least 96%, at least 97%, at least 98%, at least 99%, at least 99.5%, at least 99.8%, or 100% identical to a nucleotide sequence of SEQ ID NO: 1 or 21.

337. The polynucleotide, viral particle, or composition for use, use, or method of any one of aspects 283 to 336, wherein the administration of the polynucleotide, viral particle, or composition increases the level of C3b-inactivating and/or iC3b-degradation activity in the patient.

338. The polynucleotide, viral particle or composition for use, use, or method of aspect 337, wherein administration of the polynucleotide, viral particle or composition increases the level of C3b-inactivating and/or iC3b-degradation activity above a normal level.

339. The polynucleotide, viral particle or composition for use, use, or method of aspect 337 or 338, wherein administration of the polynucleotide, viral particle or composition increases the level of C3b-inactivating and/or iC3b-degradation activity to a level 1.2-fold, 1.5-fold, 1.8-fold or 2-fold higher than the normal level.

340. The polynucleotide, viral particle or composition for use, use, or method of aspect 338 or 339, wherein a normal level is equivalent to that provided by 30-40 µg/ml of CFI polypeptide in the serum.

341. The polynucleotide, viral particle or composition for use, use, or method of any one of aspects 283 to 340 wherein the method of treatment is a method for increasing the level of C3b-inactivating and/or iC3b-degradation in a patient.

342. The polynucleotide, viral particle or composition for use, use, or method of any one of aspects 283 to 341 wherein the method of treatment is a method for increasing the level of C3b-inactivating and/or iC3b-degradation in a patient, thereby treating the complement-mediated disorder.

343. The polynucleotide, viral particle or composition for use, use, or method of any one of aspects 283 to 342 wherein the method of treatment is a method for treating a disease by increasing the level of C3b-inactivating and/or iC3b-degradation in a patient.

344. The polynucleotide, viral particle or composition for use, use, or method of aspect 337 or 338, wherein increasing the level of C3b-inactivating and/or iC3b-degradation in a patient is increasing the level of C3b-inactivating and/or iC3b-degradation activity above a normal level.

345. The polynucleotide, viral particle or composition for use, use, or method of aspect 337 or 338, wherein increasing the level of C3b-inactivating and/or iC3b-degradation in a patient is increasing the level of C3b-inactivating and/or iC3b-degradation activity to a level 1.2-fold, 1.5-fold, 1.8-fold or 2-fold higher than the normal level.

346. The polynucleotide, viral particle or composition for use, use, or method of aspect 344 or 345, wherein a normal level is equivalent to that provided by 30-40 µg/ml of CFI polypeptide in the serum.

347. The polynucleotide, viral particle, or composition for use, use, or method of any one of aspects 279 to 346, wherein the CFI nucleotide sequence is the nucleotide sequence of any one of SEQ ID NOs: 1, 2, 3 or 5 from WO2020/086735, or SEQ ID NO: 8 from WO2017/072515.

348. The polynucleotide, viral particle, or composition for use, use, or method of any one of aspects 279 to 346, wherein the CFI nucleotide sequence is the nucleotide sequence of any one of SEQ ID NOs: 18 to 26 from WO2020/128516.

1. A polynucleotide comprising a Complement Factor I (CFI) nucleotide sequence, wherein the CFI nucleotide sequence encodes a CFI polypeptide or a fragment thereof, wherein at least a portion of the CFI nucleotide sequence is not wild-type, wherein the CFI polypeptide or the fragment thereof encoded by the CFI nucleotide sequence is expressed at a higher level in a human liver cell compared to expression of a CFI polypeptide encoded by a reference CFI nucleotide sequence.

2. The polynucleotide of claim 1, wherein:
(i) the reference CFI nucleotide sequence is a wild type CFI nucleotide sequence;
(ii) the CFI polypeptide or the fragment thereof encoded by the CFI nucleotide sequence is expressed at a higher level on transfection of the polynucleotide comprising the CFI nucleotide sequence into a human liver cell compared to expression of the CFI polypeptide encoded by the reference CFI nucleotide sequence on transfection with an equivalent polynucleotide comprising the reference CFI nucleotide sequence;
(iii) the human liver cell is a Huh-7 cell; and/or
(iv) the expression of the CFI polypeptide or the fragment thereof encoded by the CFI nucleotide sequence is at least 1.5x, at least 2x, at least 3x, at least 5x, at least 8x, at least 10x, at least 20x, at least 30x, at least 40x, or at least 50x higher compared to the CFI polypeptide encoded by the reference CFI nucleotide sequence.

3. The polynucleotide of claim 1 or 2, wherein the portion of the CFI nucleotide sequence that is not wild type is codon-optimised, and wherein the portion of the CFI nucleotide sequence that is codon-optimised is codon-optimised for expression in human liver cells.

4. The polynucleotide of claim 3, wherein the portion of the CFI nucleotide sequence that is codon-optimised:
(i) is a contiguous portion;
(ii) is at least 1000, at least 1200, at least 1300, at least 1400, at least 1500, at least 1600, at least 1700, 1752 or fewer, between 1300 and 1752, between 1500 and 1752, between 1600 and 1752, or around 1752 nucleotides in length;
(iii) is at least 1000, at least 1200, at least 1300, at least 1400, at least 1500, 1698 or fewer, between 1300 and 1698, between 1500 and 1698, or around 1698 nucleotides in length; and/or
(iv) encodes a mature CFI polypeptide.

5. The polynucleotide of any one of the preceding claims, wherein the CFI nucleotide sequence is codon-optimised for expression in human liver cells.

6. The polynucleotide of any one of claims 3 to 5, wherein, in the CFI nucleotide sequence or the portion of the CFI nucleotide sequence that is codon-optimised, at least 70%, at least 75%, at least 76%, at least 77%, at least 78%, at least 79%, at least 80%, or at least 81%, at least 82%, at least 83%, or at least 84% of the codons are selected from the group consisting of: GCT, GCC, TGC, GAC, GAG, TTC, GGA, GGC, CAC, ATC, AAG, CTG, AAT, AAC, CCT, CCC, CAG, AGG, AGA, AGT, AGC, ACC, GTG and TAC.

7. The polynucleotide of any one of claims 3 to 6, wherein the CFI nucleotide sequence or the portion of the CFI nucleotide sequence that is codon-optimised:
(i) comprises a reduced number of CpGs compared to a corresponding portion of a reference CFI nucleotide sequence; and/or
(ii) comprises a reduced number of CpGs compared to a corresponding portion of a reference CFI nucleotide sequence, and:
(a) comprises 40 or fewer, 20 or fewer, 15 or fewer, 10 or fewer, or 5 or fewer CpGs;
(b) comprises 40 or fewer, 20 or fewer, 15 or fewer, 10 or fewer, or 5 or fewer CpGs, and wherein the CFI nucleotide sequence or the portion of the CFI nucleotide sequence that is codon-optimised comprises 5 or fewer, 4 or fewer, 3 or fewer, or 2 or fewer CpGs per 100 nucleotides; and/or
(c) is CpG-free.

8. The polynucleotide of claim 7, wherein the reference CFI nucleotide sequence is a wild-type CFI nucleotide sequence.

9. The polynucleotide of any one of claims 3, 4 or 6 to 8, wherein the portion of the CFI nucleotide sequence that is codon-optimised is:
(i) at least 80%, at least 85%, at least 90%, at least 95%, at least 98%, at least 99%, at least 99.5%, at least 99.8%, or 100% identical to any one of SEQ ID NOs: 5, 10, and 17;
(ii) at least 80%, at least 85%, at least 90%, at least 95%, at least 98%, at least 99%, at least 99.5%, at least 99.8%, or 100% identical to any one of SEQ ID NOs: 1-20; and/or
(iii) at least 80%, at least 85%, at least 90%, at least 95%, at least 98%, at least 99%, at least 99.5%, at least 99.8%, or 100% identical to SEQ ID NO: 5.

10. The polynucleotide of any one of the preceding claims, wherein the CFI nucleotide sequence encodes a signal peptide.

11. The polynucleotide of any one of the preceding claims, wherein the polynucleotide further comprises a transcription regulatory element.

12. The polynucleotide of claim 11, wherein the transcription regulatory element comprises a liver-specific promoter.

13. The polynucleotide of claim 11 or 12, wherein the transcription regulatory element comprises a nucleotide sequence having at least 98% identity to SEQ ID NO: 74.

14. A polynucleotide comprising a Complement Factor I (CFI) nucleotide sequence, wherein the CFI nucleotide sequence encodes a CFI polypeptide or a fragment thereof and wherein at least a portion of the CFI nucleotide sequence is not wild-type.

15. The polynucleotide of any one of the preceding claims, wherein the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1-40.

16. A polynucleotide comprising a CFI nucleotide sequence, wherein the CFI nucleotide sequence encodes a CFI polypeptide or a fragment thereof and the CFI nucleotide sequence comprises a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NOs: 1-40.

17. The polynucleotide of any one of the preceding claims, wherein the CFI nucleotide sequence comprises:
(i) a sequence that is at least 95%, at least 96%, at least 97%, at least 98%, at least 99%, at least 99.5%, at least 99.8%, or 100% identical to a fragment of at least 1200, at least 1400, at least 1500, 1698 or fewer, 1752 or fewer, between 1200 and 1698, between 1200 and 1752, between 1500 and 1698, between 1500 and 1752, around 1698, or around 1752 nucleotides of any one of SEQ ID NO: 5 or 25;
(ii) a sequence of any one of SEQ ID NOs: 1-20, or a variant thereof encoding a CFI polypeptide comprising a sequence at least 95% identical to the wild type CFI amino acid sequence of SEQ ID NO: 44; and/or
(iii) a sequence of any one of SEQ ID NOs: 1-20, or a variant thereof encoding a CFI polypeptide comprising a sequence at least 95% identical to the wild type CFI amino acid sequence of SEQ ID NO: 44, wherein the variant is a variant of SEQ ID NO: 5, and wherein the variant of SEQ ID NO: 5 is identical to SEQ ID NO: 5 except that it comprises nucleotide substitutions such that the CFI polypeptide has 1, 2 or fewer, 3 or fewer, 4 or fewer, 5 or fewer, 6 or fewer, 7 or fewer, 8 or fewer, 9 or fewer, or 10 or fewer amino acid substitutions relative to the wild type CFI amino acid sequence of SEQ ID NO:44.

18. A viral particle comprising a recombinant genome comprising the polynucleotide of any one of the preceding claims.

19. The viral particle of claim 18, which is an AAV viral particle.

20. The viral particle of claim 19, wherein the viral particle comprises:
(i) a capsid;
(ii) a liver-tropic capsid; and/or
(iii) a liver-tropic capsid, wherein the liver-tropic capsid comprises a sequence at least 98%, at least 99%, or at least 99.5% identical to a fragment of at least 600, at least 650, at least 700, between 600 and 734, between 600 and 736, between 650 and 734, between 650 and 736, between 700 and 734, between 700 and 736, around 734, or around 736 amino acids of any one of SEQ ID NOs: 56-59.

21. A composition comprising the polynucleotide or viral particle of any one of the preceding claims, and a pharmaceutically acceptable excipient.

22. The polynucleotide according to any one of claims 1 to 17, the viral particle according to any one of claims 18 to 20, or the composition according to claim 21 for use in a method of treatment.

23. A method of treatment comprising administering an effective amount of the polynucleotide according to any one of claims 1 to 17, the viral particle according to any one of claims 18 to 20, or the composition according to claim 21.

24. Use of the polynucleotide according to any one of claims 1 to 17, the viral particle according to any one of claims 18 to 20, or the composition according to claim 21 in the manufacture of a medicament for use in a method of treatment.

25. The polynucleotide, viral particle or composition for use, or use of any one of claims 22 to 24, wherein the method of treatment comprises administering an effective amount of the polynucleotide, viral particle or composition to a patient.

26. A polynucleotide comprising a Complement Factor I (CFI) nucleotide sequence, a viral particle comprising a recombinant genome comprising the polynucleotide, or a composition comprising the polynucleotide or viral particle for use in a method of treatment, wherein the method of treatment is a method of treating a complement-mediated disorder, and the CFI nucleotide sequence encodes a CFI polypeptide or a fragment thereof.

27. A method of treatment comprising administering an effective amount of a polynucleotide comprising a CFI nucleotide sequence, a viral particle comprising a recombinant genome comprising the polynucleotide, or a composition comprising the polynucleotide or viral particle, wherein the method of treatment is a method of treating a complement-mediated disorder, and the CFI nucleotide sequence encodes a CFI polypeptide or a fragment thereof.

28. Use of a polynucleotide comprising a CFI nucleotide sequence, a viral particle comprising a recombinant genome comprising the polynucleotide, or a composition comprising the polynucleotide or viral particle in the manufacture of a medicament for use in a method of treatment, wherein the method of treatment is a method of treating a complement-mediated disorder, and the CFI nucleotide sequence encodes a CFI polypeptide or a fragment thereof.

29. The polynucleotide, viral particle or composition for use, or use of any one of claims 26 to 28, wherein the method of treatment comprises administering an effective amount of the polynucleotide, viral particle or composition to a patient.

30. The polynucleotide, viral particle or composition for use, or use of any one of claims 22 to 25, wherein the method of treatment is a method of treating a complement-mediated disorder.

31. The polynucleotide, viral particle, or composition for use, use, or method of any one of claims 26 to 30, wherein the disorder:
(i) is a C3-mediated disorder;
(ii) is a kidney disorder;
(iii) is associated with over-activity of the complement C3b feedback cycle;
(iv) is selected from C3 glomerulopathy, IgA nephropathy, lupus nephritis, systemic lupus erythematosus, membranous nephropathy, membranoproliferative glomerulonephritis, paroxysmal nocturnal hemoglobinuria, atypical hemolytic uremic syndrome, autoimmune haemolytic anemia, ANCA-associated vasculitis, Gaucher disease, peritonitis, age-related macular degeneration, diabetic retinopathy, dense deposit disease, age-related inflammatory or autoinflammatory diseases, autoimmune arthritis such as rheumatoid arthritis, atherosclerosis, chronic cardiovascular disease, Alzheimer’s disease, systemic vasculitis, Guillain-Barre syndrome, and Henoch-Schonlein purpura;
(v) is selected from C3 glomerulopathy, C3 glomerulonephritis and dense deposit disease;
(vi) is atypical hemolytic uremic syndrome with monoallelic CFH mutation;
(vii) is a kidney glomerular or tubular disorder; and/or
(vii) is not an ocular disorder.

32. The polynucleotide, viral particle, or composition for use, use, or method of any one of claims 22 to 31, wherein the polynucleotide, viral particle, or composition:
(i) is not administered intraocularly;
(ii) is administered: intravenously; systemically; to the liver via peripheral vein infusion; to the liver via hepatic vessels such as hepatic vein infusion or hepatic artery infusion; or via intraparenchymal administration direct to the liver;
(iii) is administered by injection into the renal artery;
(iv) is administered by retrograde administration; and/or
(v) is administered by retrograde administration, wherein the administration is via the ureters using a urinary catheter.


SEQUENCE LISTING (202 sequences)

SEQ ID NO: 1 - DNA, 1698 bp, Artificial Sequence
  Synthetic construct
aaggtgacctatacatctcaggaggatctggtggagaagaagtgtctggccaagaagtacacacacctgagctgcgacaaggtgttctgtcagccatggcagcggtgcatcgagggaacctgcgtgtgcaagctgccttatcagtgcccaaagaacggcaccgccgtgtgcgccacaaatcggagatcttttccaacatactgccagcagaagagcctggagtgtctgcaccccggcaccaagtttctgaacaatggcacctgcacagccgagggcaagttctctgtgagcctgaagcacggcaacacagattccgagggcatcgtggaggtgaagctggtggaccaggataagaccatgttcatctgtaagagctcctggtctatgagagaggccaacgtggcctgcctggatctgggctttcagcagggcgccgacacacagaggcgcttcaagctgtccgacctgtctatcaatagcaccgagtgcctgcacgtgcactgtaggggcctggagacaagcctggccgagtgcacctttacaaagcggagaaccatgggctatcaggacttcgccgacgtggtgtgctacacccagaaggccgattctccaatggacgatttctttcagtgcgtgaacggcaagtacatcagccagatgaaggcctgcgacggcatcaatgactgtggcgatcagtccgacgagctgtgctgtaaggcctgtcagggcaagggctttcactgcaagagcggcgtgtgcatcccctcccagtatcagtgcaacggcgaggtggattgtatcaccggagaggacgaagtgggatgcgcaggcttcgcctccgtgacccaggaggagaccgagatcctgacagccgacatggatgccgagaggcgccggatcaagtctctgctgcctaagctgagctgtggcgtgaagaatagaatgcacatcagaaggaagcgcatcgtgggaggcaagagggcacagctgggcgatctgccatggcaggtggccatcaaggacgcaagcggaatcacctgcggaggcatctatatcggaggatgttggatcctgaccgcagcacactgcctgagagcatccaagacacacaggtaccagatctggaccacagtggtggattggatccaccctgacctgaagagaatcgtgatcgagtatgtggataggatcatcttccacgagaactacaatgccggcacatatcagaacgacatcgccctgatcgagatgaagaaggatggcaataagaaggactgtgagctgccacgctccatccctgcatgcgtgccctggtctccttacctgtttcagccaaacgatacctgcatcgtgagcggatggggaagggagaaggacaatgagcgggtgttctccctgcagtggggcgaggtgaagctgatctccaactgttctaagttttacggcaatcggttctatgagaaggagatggagtgcgccggcacctacgatggcagcatcgacgcctgtaagggcgattccggaggaccactggtgtgcatggacgcaaacaatgtgacatacgtgtggggagtggtgagctggggagagaactgcggcaagccagagtttcccggcgtctacaccaaggtggccaattattttgactggatctcctaccacgtgggcaggcctttcatctctcagtacaacgtgtga

SEQ ID NO: 2 - DNA, 1698 bp, Artificial Sequence
  Synthetic construct
aaagtgacctacaccagccaagaggacctggtggaaaagaagtgcctggccaagaagtacacccacctgagctgcgacaaggtgttctgccagccttggcagagatgcatcgagggcacctgtgtgtgcaagctgccctatcagtgccccaagaatggcacagccgtgtgcgccaccaacagaagaagcttccctacctactgccagcagaaaagcctggaatgtctgcaccccggcaccaagtttctgaacaacggcacctgtaccgccgagggcaagtttagcgtgtccctgaagcacggcaacaccgactctgagggcatcgtggaagtgaagctggtggaccaggacaagaccatgttcatctgcaagagcagctggtccatgcgcgaggccaatgtggcttgtctggatctgggattccagcagggcgccgacacacagagaagattcaagctgagcgacctgagcatcaacagcaccgagtgcctgcatgtgcactgtagaggcctggaaacaagcctggccgagtgcaccttcaccaagagaaggaccatgggctaccaggacttcgccgacgtcgtgtgctacacccagaaagccgactctcccatggacgatttcttccagtgcgtgaacggcaagtacatcagccagatgaaggcctgcgacggcatcaacgattgcggcgatcagagcgacgagctgtgctgcaaagcctgtcaaggcaagggcttccactgcaagtccggcgtgtgtatccctagccagtaccagtgcaatggcgaggtggactgtatcaccggcgaggatgaagtgggctgtgccggatttgccagcgtgacccaagaggaaaccgagatcctgaccgccgatatggacgccgagcggcggagaatcaaaagcctgctgcctaagctgtcctgcggcgtgaagaaccggatgcacatccggcgcaagagaatcgtcggaggcaaaagagcacagctgggcgatctgccttggcaagtggccatcaaggatgccagcggcatcacatgtggcggcatctacatcggcggctgctggattctgacagccgctcattgtctgcgggccagcaagacccaccggtatcagatctggaccaccgtggtggactggattcaccccgacctgaagcggatcgtgatcgagtacgtggaccggatcatcttccacgagaactacaacgccggcacctaccagaacgatatcgccctgatcgagatgaagaaggacgggaacaagaaggactgcgagctgcctagatctatccccgcctgtgttccttggagcccctacctgttccagcctaacgatacctgcatcgtgtccggctggggcagagagaaggataacgagagggtgttcagcctgcagtggggcgaagtgaaactgatcagcaactgcagcaagttctacggcaaccggttctacgagaaagaaatggaatgcgccggcacatacgacggctccatcgatgcctgtaaaggcgattctggcggccctctcgtgtgcatggatgccaacaatgtgacctacgtgtggggcgtcgtgtcctggggagagaattgtggcaagcctgagttccccggcgtctacaccaaggtggccaactacttcgactggatcagctaccacgtgggcagaccctttatcagccagtacaacgtgtga

SEQ ID NO: 3 - DNA, 1698 bp, Artificial Sequence
  Synthetic construct
aaggtgacctatacatctcaggaggatctggtggagaagaagtgtctggccaagaagtacacacacctgagttgtgacaaggtgttctgtcagccatggcagagatgcattgagggaacctgtgtgtgcaagctgccttatcagtgcccaaagaatggcactgctgtgtgtgccacaaataggagatcttttccaacatactgccagcagaagagcctggagtgtctgcaccctggcaccaagtttctgaacaatggcacctgcacagctgagggcaagttctctgtgagcctgaagcatggcaacacagatagtgagggcattgtggaggtgaagctggtggaccaggataagaccatgttcatctgtaagagctcctggtctatgagagaggccaatgtggcctgcctggatctgggctttcaacaaggagctgacacacagaggagattcaagctgagtgacctgtctatcaatagcacagagtgcctgcatgtgcactgtagaggcctggagacaagcctggctgagtgcacctttacaaagagaaggaccatgggctatcaggactttgctgatgtggtatgctacacccagaaggctgattctccaatggatgatttctttcaatgtgtgaatggcaagtacatcagccagatgaaggcctgtgatggcatcaatgactgtggagatcagagtgatgagctgtgttgcaaggcctgtcagggcaagggctttcactgcaagagtggagtgtgcatcccctcccaatatcagtgcaatggagaggtggattgtatcacaggagaggatgaagtgggatgtgcaggctttgcctcagtgacccaggaggagacagagatcctgacagctgacatggatgctgagaggagaaggatcaagtctctgctgcctaagctgagctgtggagtgaagaatagaatgcacatcagaaggaagagaattgtgggaggcaagagggcacagctgggagatctgccatggcaggtggccatcaaggatgcaagtggaatcacctgtggaggcatctatattggaggatgttggatcctgacagcagcacactgcctgagagcatccaagacacacagataccagatctggaccacagtggttgattggatccaccctgacctgaagagaattgtgattgagtatgtggataggatcatcttccatgagaactacaatgctggcacatatcagaatgacattgccctgattgagatgaagaaggatggcaataagaaggactgtgagctgccaagatccatccctgcatgtgtgccctggtctccttacctgtttcagccaaatgatacctgcattgtatccggatggggaagggagaaggacaatgagagagtgttctccctgcagtggggagaggtgaagctgatctccaactgttctaagttttatggcaatagattctatgagaaggagatggagtgtgcaggcacctatgatggcagcattgatgcctgtaagggagatagtggaggaccactggtgtgcatggatgcaaacaatgtgacatatgtgtggggagtggtgagctggggagagaactgtggcaagccagagtttcctggagtctacaccaaggtggccaattattttgactggatctcctaccatgtgggcaggcctttcatctctcagtacaatgtgtga

SEQ ID NO: 4 - DNA, 1698 bp, Artificial Sequence
  Synthetic construct
aaggtgacctacaccagccaagaggacctggtggaaaagaagtgcctggccaagaagtacacccacctgagctgtgacaaggtgttctgccagccttggcagagatgcattgagggcacctgtgtgtgcaagctgccctatcagtgccccaagaatggcacagctgtgtgtgccaccaacagaaggagcttccctacctactgccagcagaaaagcctggaatgtctgcaccctggcaccaagtttctgaacaatggcacctgtacagctgagggcaagtttagtgtgtccctgaagcatggcaacacagactctgagggcattgtggaagtgaagctggtggaccaggacaagaccatgttcatctgcaagagtagctggtccatgagagaggccaatgtggcttgtctggatctgggattccaacagggagctgacacacagagaagattcaagctgagtgacctgagcatcaacagcacagagtgcctgcatgtgcactgtagaggcctggaaacaagcctggctgagtgcaccttcaccaagagaaggaccatgggctaccaggactttgctgatgtggtgtgctacacccagaaagctgactctcccatggatgatttctttcaatgtgtgaatggcaagtacatcagccagatgaaggcctgtgatggcatcaatgattgtggagatcagagtgatgagctgtgctgcaaagcctgtcaaggcaagggcttccactgcaagagtggagtgtgtatccctagccagtaccagtgcaatggagaggtggactgtatcacaggagaggatgaagtgggctgtgctggatttgccagtgtgacccaagaggaaacagagatcctgacagctgatatggatgctgagagaaggagaatcaaaagcctgcttcctaagctgtcctgtggagtgaagaacagaatgcacatcagaaggaagagaattgtgggaggcaaaagagcacagctgggagatctgccttggcaagtggccatcaaggatgccagtggcatcacatgtggaggcatctacattggaggctgctggattctgacagctgctcattgtctgagagccagcaagacccacagatatcagatctggaccacagtggtagactggattcaccctgacctgaagagaattgtgattgagtatgtggacagaatcatcttccatgagaactacaatgctggcacctaccagaatgatattgccctgattgagatgaagaaggatggaaacaagaaagactgtgagctgcctagatctatccctgcctgtgttccttggagcccctacctgttccagcctaatgatacctgcatagtttctggatggggcagagagaaggataatgagagggtgttcagcctgcagtggggagaagtgaaactgatcagcaactgcagcaagttctatggcaacagattctatgagaaagaaatggaatgtgctggcacatatgatggctccattgatgcctgtaaaggagattctggaggccctcttgtgtgcatggatgccaacaatgtgacctatgtgtggggagtggtatcctggggagagaattgtggcaagcctgagttccctggagtctacaccaaggtggccaactactttgactggatcagctaccatgtgggcagaccctttatcagccagtacaatgtgtga

SEQ ID NO: 5 - DNA, 1698 bp, Artificial Sequence
  Synthetic construct
aaggtgacctacaccagccaggaggacctggtggagaagaagtgcctggccaagaagtacacccacctgagctgtgacaaggtgttctgccagccctggcagagatgcattgagggcacctgtgtgtgcaagctgccctaccagtgccccaagaatggcacagctgtgtgtgccaccaacagaaggagcttccccacctactgccagcagaagagcctggagtgcctgcaccctggcaccaagttcctgaacaatggcacctgcacagctgagggcaagttcagtgtgagcctgaagcatggcaacacagacagtgagggcattgtggaggtgaagctggtggaccaggacaagaccatgttcatctgcaagagcagttggagcatgagagaggccaatgtggcctgcctggacctgggcttccagcaaggagctgacacccagagaaggttcaagctgagtgacctgagcatcaacagcacagagtgcctgcatgtgcactgcagaggcctggagaccagcctggctgagtgcaccttcaccaagagaaggaccatgggctaccaggactttgctgatgtggtttgctacacccagaaggctgacagccccatggatgacttcttccagtgtgtgaatggcaagtacatcagccagatgaaggcctgtgatggcatcaatgactgtggagaccagagtgatgagctgtgctgcaaggcctgccagggcaagggcttccactgcaagagtggagtgtgcatccccagccagtaccagtgcaatggagaggtggactgcatcacaggagaggatgaggtgggctgtgctggctttgccagtgtgacccaggaggagacagagatcctgacagctgacatggatgctgagagaaggagaatcaagagcctgctgcccaagctgagctgtggagtgaagaacagaatgcacatcagaaggaagagaattgtgggaggcaagagagcccagctgggagacctgccctggcaggtggccatcaaggatgccagtggcatcacctgtggaggaatctacattggaggctgctggattctgacagctgcccactgcctgagagccagcaagacccacagataccagatctggaccacagtggttgactggattcaccctgacctgaagagaattgtgattgagtatgtggacagaatcattttccatgagaactacaatgctggcacctaccagaatgacattgccctgattgagatgaagaaggatggcaacaagaaagactgtgagctgcccagaagcatccctgcctgtgtgccctggagcccctacctgttccagcccaatgacacctgtattgtcagtggatggggcagagagaaggacaatgagagagtgttcagcctgcagtggggagaggtgaagctgatcagcaactgcagcaagttctatggcaacagattctatgagaaggagatggagtgtgctggcacctatgatggcagcattgatgcctgcaagggagacagtggaggccccctggtgtgcatggatgccaacaatgtgacctatgtgtggggagtggttagctggggagagaactgtggcaagcctgagttccctggagtgtacaccaaggtggccaactactttgactggatcagctaccatgtgggcagacccttcatcagccagtacaatgtgtaa

SEQ ID NO: 6 - DNA, 1698 bp, Artificial Sequence
  Synthetic construct
aaggtgacctatacatcacaggaggacctggtggagaagaagtgtctggccaagaagtatacacatctgtcatgtgacaaggtgttctgtcagccttggcagaggtgtattgagggaacatgtgtgtgtaagctgccttatcagtgtcctaagaatggaacagctgtgtgtgccacaaataggaggtcattccctacatattgtcagcagaagtcactggagtgtctgcatcctggaacaaagttcctgaataatggaacatgtacagctgagggaaagttctcagtgtcactgaagcatggaaatacagactcagagggaattgtggaggtgaagctggtggaccaggacaagacaatgttcatctgcaagtcatcatggtcaatgagggaggccaatgtggcctgtctggacctgggattccagcagggagctgacacacagaggaggttcaagctgtcagacctgtcaatcaattcaacagagtgtctgcatgtgcattgtagaggactggagacatcactggctgagtgtacattcacaaagaggagaacaatgggatatcaggactttgctgatgtggtttgttatacacagaaggctgactcacctatggatgacttctttcaatgtgtgaatggaaagtatatctcacagatgaaggcctgtgatggaatcaatgactgtggagaccagtcagatgagctgtgttgcaaggcctgtcagggaaagggattccattgcaagtcaggagtgtgtatcccttcacaatatcagtgtaatggagaggtggactgtatcacaggagaggatgaggtgggatgtgcaggatttgcctcagtgacccaggaggagacagagatcctgacagctgacatggatgctgagagaaggagaatcaagtcactgctgcctaagctgtcatgtggagtgaagaataggatgcatatcaggaggaagaggattgtgggaggaaagagggcccagctgggagacctgccttggcaagtggccatcaaggatgcctcaggaatcacatgtggaggaatctatattggaggatgttggatcctgacagctgcccattgtctgagggcctcaaagacacatagatatcagatctggacaacagtggtggactggatccatcctgacctgaagaggattgtgattgagtatgtggacaggatcatcttccatgagaattataatgctggaacatatcagaatgacattgccctgattgagatgaagaaggatggaaataagaaggactgtgagctgcctaggtcaatccctgcctgtgtgccttggtccccttatctgttccaacctaatgacacatgtattgtgtctggatggggaagggagaaggacaatgagagggtgttctcactgcagtggggagaggtgaagctgatctcaaattgttcaaagttctatggaaataggttctatgagaaggagatggagtgtgctggaacatatgatggatcaattgatgcctgtaagggagactcaggaggacctctagtatgtatggatgccaataatgtgacatatgtgtggggagtggtgtcatggggagagaattgtggaaagcctgagttccctggagtgtatacaaaggtggccaattattttgactggatctcatatcatgtgggaaggcctttcatctcacagtataatgtgtaa

SEQ ID NO: 7 - DNA, 1698 bp, Artificial Sequence
  Synthetic construct
aaggtgacctatacctctcaagaggatctggtggagaagaagtgcctggctaagaagtatactcacctgagctgtgataaggtgttctgccagccctggcagaggtgcattgagggcacctgtgtgtgtaagctgccctatcagtgccccaagaatggcactgctgtgtgtgctactaacaggaggagcttccccacctactgtcagcagaagagcctggagtgtctgcatcctgggaccaagtttctgaataatggaacctgcacagctgagggaaagtttagtgtgagcctgaagcatggaaatacagatagtgagggaattgtggaggtgaagctggtggaccaggataagaccatgttcatctgcaagagcagctggagcatgagggaggccaatgtggcctgcctggacctggggtttcagcaaggtgctgatactcagaggaggtttaagctgtctgatctgtctatcaatagcactgagtgtctgcatgtgcattgtagaggactggagaccagcctggctgagtgtacttttactaagaggaggactatgggataccaggattttgctgatgtggtgtgttatacccagaaggctgattctcccatggatgacttctttcagtgtgtgaatgggaagtacatctctcagatgaaggcctgtgatggtatcaatgattgtggagaccagagtgatgagctgtgttgtaaggcttgccaaggcaagggctttcattgcaagagtggagtgtgcatccccagccagtatcagtgcaatggagaggtggactgcatcacaggggaggatgaggtgggctgtgctggctttgcttctgtgactcaagaggagacagagatcctgactgctgacatggatgctgagaggaggaggatcaagagcctgctgcctaagctgtcttgtggagtgaagaacaggatgcacatcaggaggaagaggattgtgggaggaaagagggctcagctgggagacctgccctggcaggtggctatcaaggatgccagtggaatcacctgtgggggaatctatattggtggctgttggatcctgactgctgctcattgtctgagggccagcaagactcatagataccagatctggaccacagtggtggactggatccaccctgacctgaagaggattgtgattgagtatgtggataggatcatcttccatgagaactacaatgctggcacttaccagaatgacattgctctgattgagatgaagaaggatggaaacaagaaggattgtgagctgcctaggagcatccctgcctgtgtgccctggtctccttacctgttccaacctaatgatacctgcattgtgtctggctggggaagggagaaggataatgagagggtgtttagcctgcagtggggtgaggtgaagctgatcagcaactgctctaagttttatggaaataggttctatgagaaggagatggagtgtgctggaacctatgatggtagcattgatgcctgcaagggggactctggaggccccctggtttgtatggatgccaacaatgtgacttatgtgtggggtgtggtgagctggggggagaactgtggaaagcctgagttccctggagtgtacaccaaggtggccaattattttgactggatcagctaccatgtgggaaggccttttatctctcagtacaatgtgtaa

SEQ ID NO: 8 - DNA, 1698 bp, Artificial Sequence
  Synthetic construct
aaggtgacctatacaagccaggaggatctggtggagaagaagtgcctggcaaagaagtatacccacctgagctgtgataaggtgttttgccagccctggcagagatgcattgagggcacctgtgtttgtaagctgccctatcagtgccccaagaatggcacagcagtgtgtgcaaccaatagaaggagctttcccacatactgtcagcagaagagcctggaatgtctgcatcctgggacaaagtttctgaataatggaacatgcacagctgaaggaaagtttagtgttagcctgaagcatggaaatacagatagtgagggaatagttgaagtgaagctggtggaccaggataagacaatgtttatatgcaagagtagctggagcatgagagaagccaatgtggcctgcctggacctggggtttcagcaaggagctgatacccagagaaggtttaagctgagtgatctgagcataaatagcacagaatgtctgcatgtgcattgcagaggactggagaccagcctggctgaatgtacctttaccaagagaaggaccatgggctaccaggattttgctgatgtggtttgttatacacagaaggcagatagccccatggatgacttttttcaatgtgtgaatgggaagtacattagccagatgaaggcctgtgatggcatcaatgattgtggagaccagagtgatgaactgtgttgtaaggcatgccagggcaagggctttcattgcaagagtggagtttgcattcccagccagtatcagtgcaatggagaggtggactgcattacaggggaagatgaagttggctgtgcaggctttgcaagtgtgactcaggaagaaacagaaattctgacagctgacatggatgcagaaagaagaagaataaagagcctgctgcctaagctgagctgtggagttaagaatagaatgcacattagaaggaagagaattgtgggaggaaagagagcacagctgggagacctgccctggcaggtggcaattaaggatgccagtggaatcacctgtgggggaatttatattggaggctgttggattctgacagctgcacattgtctgagagccagcaagacccatagataccagatatggacaacagtggtggactggatacaccctgacctgaagagaatagtgattgaatatgtggatagaattatttttcatgaaaattacaatgcaggcacctaccagaatgacattgctctgattgaaatgaagaaggatggaaataagaaggattgtgagctgcctagaagcatccctgcctgtgtgccctggagcccttacctgtttcagcctaatgatacatgcattgttagtggctggggaagagaaaaggataatgaaagagtgtttagcctgcagtggggagaagttaagctgataagcaattgcagcaagttttatggaaatagattttatgaaaaggaaatggaatgtgcaggcacatatgatggcagcattgatgcctgcaagggggacagtggaggccccctagtatgtatggatgccaataatgtgacctatgtgtggggagttgtgagctggggggaaaattgtggaaagcctgagtttcctggagtttacaccaaggtggccaattattttgactggattagctaccatgtgggaagaccttttattagccagtacaatgtgtaa

SEQ ID NO: 9 - DNA, 1698 bp, Artificial Sequence
  Synthetic construct
aaggtgacctatacatctcaggaggatctggtggagaagaagtgtctggccaagaagtatacacacctctcctgtgataaggtgttctgtcagccatggcagaggtgtattgagggaacctgtgtgtgtaagctgccttatcagtgtccaaagaatggaacagctgtgtgtgccacaaacaggaggagcttcccaacatactgtcagcagaagtcactggaatgtctgcatccaggaacaaagttcctgaataatggaacatgtacagctgaaggaaagttctcagtgtccctgaagcatggaaatacagattcagagggaattgtggaagtgaagctggtggaccaggataagacaatgttcatctgtaagagcagctggagcatgagggaagccaatgtggcctgtctggacctgggattccagcagggagctgatacacagaggaggttcaagctgtctgatctctctatcaattccacagaatgtctgcatgtgcattgcagaggtctggagacctcactggctgaatgtacattcacaaagaggaggacaatgggataccaggattttgctgatgtggtgtgttatacacagaaggctgattctccaatggatgacttctttcagtgtgtgaatggaaagtacatctctcagatgaaggcctgtgatggaatcaatgattgtggagaccagtcagatgaactgtgctgcaaggcctgtcagggaaagggattccattgcaagtcaggagtgtgtatcccaagccagtatcagtgtaatggagaggtggactgtatcacaggagaagatgaagtgggatgtgcaggatttgcctctgtgactcaggaagaaacagaaatcctgacagctgacatggatgctgaaaggaggaggatcaagtcactgctgcctaagctgtcttgtggagtgaagaacaggatgcacatcaggaggaagaggattgtgggaggaaagagggcccagctgggagacctcccatggcaggtggccatcaaggatgcctcaggaatcacctgtggaggaatctatattggaggatgttggatcctgacagctgcccattgtctaagagcctcaaagacacatagataccagatctggacaacagtggtggactggatccaccctgacctgaagaggattgtgattgaatatgtggataggatcatcttccatgaaaactacaatgctggaacataccagaatgacattgctctgatagaaatgaagaaggatggaaacaagaaggattgtgagctgcctaggtccatccctgcctgtgtgccctggtctccttacctgttccaacctaatgatacatgtattgtgtctggatggggaagggaaaaggataatgaaagggtgttctcactgcagtggggagaagtgaagctgatcagcaactgttctaagttctatggaaataggttctatgaaaaggaaatggaatgtgctggaacatatgatggatccattgatgcctgtaagggagactctggaggacccctggtttgtatggatgccaacaatgtgacatatgtgtggggagtggtgtcatggggagaaaactgtggaaagccagagttcccaggagtgtacaccaaggtggccaattattttgactggatcagctaccatgtgggaaggcctttcatctctcagtacaatgtgtaa

SEQ ID NO: 10 - DNA, 1698 bp, Artificial Sequence
  Synthetic construct
aaggtgacctatacatcccaagaggatcttgtggaaaagaaatgtctggccaaaaagtacacccatctgtcttgtgacaaggtcttctgccagccttggcagagatgtattgaaggaacttgtgtctgtaaactgccctaccagtgccctaagaatggaacagctgtctgtgctactaatagaaggtcattcccaacttactgccagcagaagtctctggagtgtctccaccctggaactaaattcctgaacaatggcacatgtacagctgaaggcaagttctctgtctccctcaaacatggaaacacagactcagaagggattgtggaagtcaagctggtggatcaggataagaccatgtttatttgcaagtcatcctggtctatgagagaagcaaatgtggcctgcctggatctgggctttcaacaaggagcagacactcagaggagattcaagctctctgacctgtctattaatagcactgagtgtctgcatgtgcattgtagaggactggagacctcacttgctgaatgtacattcactaaaagaaggactatgggataccaggactttgctgatgtggtgtgctatacccagaaggcagactcccccatggatgactttttccaatgtgttaatgggaaatatattagtcagatgaaggcttgtgatggaattaatgactgtggagatcagagtgatgagctctgttgcaaagcctgtcagggaaagggtttccactgcaaaagtggggtttgtattccttctcaataccaatgcaatggggaggtggactgcattactggagaggatgaggtggggtgtgctggctttgcttctgtgacccaagaggagacagagatcctgacagctgatatggatgctgaaaggagaagaattaagtcactgctgcccaaactgtcttgtggagttaaaaacagaatgcacatcagaagaaagagaattgtgggaggaaaaagagcccagcttggagatctcccttggcaagtggccattaaggatgccagtggtatcacatgtggaggcatctatattggagggtgctggattctcacagctgcacactgtctgagagccagtaaaacccatagatatcagatctggacaacagtggttgattggattcatcctgatctcaagagaattgtgattgagtatgtggacagaattatttttcatgaaaactacaatgcaggaacctatcagaatgatattgccctcattgagatgaagaaggatgggaacaaaaaggattgtgagctgcccagaagcatcccagcttgtgtgccttggtctccatacctctttcagccaaatgatacttgcattgtcagtggatggggtagagagaaagataatgagagagtcttctccctccagtggggagaggttaagctcatcagtaattgctccaagttttatggaaacaggttctatgaaaaagagatggaatgtgcaggaacatatgatggtagtattgatgcttgcaaaggagatagtggtggacccctggtctgtatggatgccaacaatgtgacttatgtgtggggggtggtgagctggggagagaattgtgggaagcctgagtttccaggagtctacaccaaggtggctaattactttgactggatcagttaccatgtgggtaggcccttcattagccagtacaatgtctga

SEQ ID NO: 11 - DNA, 1698 bp, Artificial Sequence
  Synthetic construct
aaggttacctatacatcccaggaagacctggtggagaaaaagtgtcttgccaagaagtacacccacctcagttgtgataaggtcttttgccagccatggcagagatgtattgaaggcacttgtgtgtgcaagctgccataccagtgtcccaaaaatgggacagcagtctgtgcaactaatagaagatccttccctacatactgccagcagaagagtcttgagtgcctgcacccaggcacaaagtttctgaataatggcacatgcacagctgaaggcaaattcagtgtctccctgaaacatggaaacacagatagtgagggaattgtggaagttaagctggtggaccaggataaaaccatgttcatttgtaaatcttcatggagtatgagagaagctaatgtggcatgtctggaccttggatttcaacaaggagcagatacccagagaaggtttaagctgagtgatctctccattaactctactgagtgtctgcatgtgcattgtagaggactggagacatcactggcagaatgtacttttaccaaaaggagaaccatgggttatcaggactttgcagatgtggtctgttacacccagaaggctgactcccccatggatgacttctttcaatgtgtgaatgggaaatacatctcccagatgaaggcctgtgatggtatcaatgactgtggagaccagtctgatgagctctgctgcaaggcctgtcaggggaagggatttcactgcaaaagtggggtttgcatccccagtcagtaccagtgcaatggggaagtggactgcattacaggggaagatgaggttggttgtgctgggtttgctagtgtgacccaggaggaaacagagatcctgacagctgatatggatgcagaaagaagaagaattaagagcctcctgcctaagctcagttgtggagtcaaaaataggatgcatattaggagaaagaggattgtgggagggaaaagagcccagcttggagacctgccatggcaggttgccatcaaggatgcctcagggatcacatgtggagggatctatattggtggatgttggattctgactgctgcccattgcctgagggcctcaaagacccacagatatcagatctggaccactgtggtggactggattcatcctgacctgaagagaattgtgattgaatatgtggacaggattatctttcatgagaactataatgctggaacttatcagaatgatattgcactgattgagatgaagaaggatgggaataaaaaggactgtgaactcccaagaagcattcctgcatgtgtgccttggtctccttacctcttccaacccaatgacacttgtattgtcagtggatggggtagagaaaaagacaatgagagggttttcagtctgcagtggggggaggtcaagctcatcagcaactgctccaaattctatggaaacaggttctatgaaaaagaaatggagtgtgctgggacttatgatggtagtattgatgcctgtaaaggggattctggagggccacttgtctgtatggatgctaacaatgtgacctatgtctggggagtggtgtcatggggagaaaattgtggcaaacctgagtttcctggagtgtatacaaaggtggctaactattttgattggatcagttatcatgttggtagacccttcatcagtcagtacaatgtctag

SEQ ID NO: 12 - DNA, 1698 bp, Artificial Sequence
  Synthetic construct
aaggtgacctacaccagccaggaggacctggtggaaaagaaatgtcttgctaagaaatatacccacctgtcctgtgacaaagtcttctgtcagccttggcagagatgtattgaagggacatgtgtctgtaagctgccttaccagtgtcctaagaatgggactgcagtctgtgccactaatagaagatcattcccaacatactgccagcagaagagtctggagtgtctgcatcctggcacaaagtttctcaacaatgggacatgtacagctgaaggaaaattctctgtgagcctgaaacatggcaatacagatagtgagggaattgttgaggttaagctggtggatcaggacaaaacaatgtttatctgcaaaagcagctggtccatgagagaggctaatgtggcctgtcttgaccttggctttcaacaaggagcagatacccagagaagatttaaactgtcagatctgagcatcaattcaacagagtgtctgcatgtgcattgtagaggactggagaccagcctggctgaatgtaccttcacaaagagaagaacaatgggataccaggactttgctgatgtggtttgttacacccagaaggcagacagccccatggatgacttctttcagtgtgtgaatggaaagtatattagccagatgaaggcatgtgatggtatcaatgattgtggggaccagagtgatgagctctgttgtaaggcttgtcaaggcaaaggcttccactgcaagtcaggggtgtgcatcccctcccaataccagtgcaatggagaggtggattgcatcacaggggaggatgaagtgggttgtgctgggtttgcaagtgtgacccaggaggaaacagaaatcctgactgctgatatggatgctgagaggagaagaatcaaaagcctcctgcctaaactgtcttgtggtgttaagaatagaatgcacattagaagaaagagaattgtggggggaaagagagcacagctgggtgatctgccctggcaagtggcaattaaggatgcctcaggcatcacctgtggaggcatttacattgggggttgctggattctgacagcagcccactgtctcagagccagcaaaactcacagatatcagatttggacaacagttgtggattggattcaccctgatctgaagagaattgtcattgagtatgtggacagaattattttccatgagaactacaatgctggcacataccagaatgacattgccctcattgaaatgaagaaggatggaaataaaaaggactgtgaactcccaagatcaattcctgcctgtgtgccatggagtccttacctcttccaaccaaatgatacctgcattgtcagtggctgggggagagagaaggataatgaaagagttttctccctgcagtggggagaggtgaagctcattagtaactgttctaagttttatgggaacagattttatgagaaggagatggaatgtgcaggaacttatgatggtagcattgatgcatgcaaaggagacagtggaggtccacttgtctgcatggatgctaacaatgtgacttatgtctggggagtggtctcctggggtgaaaactgtggaaagcctgagttcccaggagtgtatacaaaagtggccaactactttgactggatttcataccatgtggggagaccttttatcagccagtacaatgtctga

SEQ ID NO: 13 - DNA, 1698 bp, Artificial Sequence
  Synthetic construct
aaggtgacctacacctctcaggaagacctggtggagaagaagtgtcttgccaagaaatatacccatctgtcatgtgataaagtgttctgtcagccttggcagagatgtattgagggaacatgtgtgtgcaagctgccctaccagtgtccaaagaatgggactgctgtttgtgccactaacagaagatcttttcctacatactgtcagcagaagagtctggagtgcctccaccctggcacaaaattcctcaataatggcacatgtacagctgagggaaagttttctgttagcctcaagcatggcaatactgattcagagggtattgttgaagtcaaactggtggatcaggataaaaccatgttcatttgtaagagtagctggtccatgagggaggctaatgtggcatgcctggacctgggctttcaacaaggggctgacactcagagaaggtttaagctgagtgacctgagcattaattcaactgagtgtctgcatgtgcattgtagagggctggagactagccttgcagagtgcacattcactaagagaagaacaatgggataccaggattttgcagatgtggtgtgctacactcagaaagctgactctcccatggatgacttcttccaatgtgtgaatgggaaatatatttcacagatgaaggcctgtgatggaattaatgattgtggtgatcagagtgatgagctctgttgtaaggcttgccaggggaagggttttcactgtaaaagtggggtttgtatcccctctcaataccagtgcaatggggaagttgattgcattacaggagaggatgaggtgggatgtgcaggctttgccagtgtgacacaggaagagacagagatcctgacagctgatatggatgcagaaagaagaagaatcaagtccctgctccccaaactgtcctgtggggtcaagaatagaatgcacatcagaaggaagagaattgtgggaggcaaaagggcacagctgggggacctcccatggcaggtggcaattaaggatgccagtggtatcacatgtggagggatttacattggaggctgctggattctcacagcagcccactgcctcagagcatctaaaactcacagataccagatttggaccactgtggtggactggattcaccctgacctgaagagaattgtgattgaatatgtggatagaatcattttccatgaaaattacaatgctggcacttatcagaatgacattgcactcattgagatgaagaaagatgggaataaaaaggactgtgaactgcccagaagcattccagcttgtgtgccatggagtccatatctgtttcagccaaatgatacatgcatagtgtctggttggggaagagagaaggacaatgaaagggtttttagcctccagtggggggaggtcaagctgattagcaactgcagtaaattctatggcaacagattttatgagaaggagatggaatgtgcaggcacttatgatggctcaattgatgcctgcaagggagattctggtggacccctggtgtgcatggatgccaataatgtcacctatgtctggggtgtggttagctggggagagaactgtgggaagcctgagtttccaggagtgtatacaaaagtggcaaattactttgactggatctcctatcatgtggggagaccctttatctctcagtacaatgtttga

SEQ ID NO: 14 - DNA, 1698 bp, Artificial Sequence
  Synthetic construct
aaggtgacctatacctctcaggaggacctggttgagaaaaagtgcctggctaaaaagtacacccacctctcttgtgataaggtgttctgccagccttggcagagatgcattgaaggcacatgtgtttgtaaactgccttaccagtgtcccaaaaatgggactgctgtgtgtgccacaaatagaaggagctttccaacctactgtcagcagaagagccttgaatgcctccatcctgggacaaaattcctgaacaatggaacctgtacagctgaaggcaaatttagtgttagcctgaaacatgggaatacagattctgagggaattgtggaggtcaaactggtggaccaggataaaaccatgtttatctgcaagagtagctggtccatgagagaggccaatgtggcatgcctggatcttgggtttcaacaaggagctgacacccagagaagattcaaactcagtgacctgagtattaactcaacagaatgtctgcatgtgcattgtagaggactggaaactagccttgcagagtgcacttttaccaagagaagaaccatgggatatcaggattttgctgatgtggtgtgctacacccagaaagcagacagtcccatggatgacttcttccagtgtgtgaatggcaagtatatttcccagatgaaagcatgtgatggcatcaatgattgtggagaccagagtgatgaactgtgctgtaaggcttgccagggaaagggtttccactgcaaaagtggagtttgcatccccagccagtaccagtgcaatggagaagtggattgcattacaggagaggatgaagtgggatgtgctggctttgcttctgttacccaagaagagacagagattctgacagctgacatggatgcagagaggagaagaattaagtccctgctgcctaaactgtcctgtggtgtcaagaatagaatgcacatcagaaggaagagaattgtggggggaaaaagagcccagcttggagacctgccttggcaagtggctattaaggatgccagtgggatcacatgtggaggaatttacattggagggtgctggattctcacagctgcccactgcctgagggccagcaaaacccacagataccagatctggacaacagttgtggattggattcatcctgatctcaagagaattgttattgagtatgtggataggatcattttccatgaaaattacaatgcaggcacataccaaaatgacattgcactcattgagatgaagaaggatgggaataaaaaggattgtgagctccccaggagcattcctgcttgtgtcccctggtccccttacctcttccaacccaatgacacctgcattgtcagtggctggggtagagaaaaagataatgagagagtcttcagcctgcagtggggggaggtgaagctgattagcaattgtagcaagttttatgggaacaggttctatgagaaggagatggagtgtgctggcacctatgatggctctattgatgcttgtaagggggacagtgggggaccactggtttgtatggatgcaaacaatgtgacctatgtttggggagtggttagctggggagaaaattgtggaaaacctgagtttcctggtgtctatacaaaagtggctaattattttgattggatcagttatcatgttgggagaccctttattagtcagtacaatgtctag

SEQ ID NO: 15 - DNA, 1698 bp, Artificial Sequence
  Synthetic construct
aaggtgacctacactagccaggaggatctggtggagaaaaagtgccttgcaaaaaagtacactcacctcagctgtgacaaggtcttctgtcagccttggcagagatgcattgaggggacatgtgtgtgcaaactcccataccagtgtcctaagaatggtacagctgtctgtgcaacaaacaggagatcctttcccacctattgtcagcagaagtccctggagtgtctgcaccctggtacaaagtttctgaataatggcacatgcactgctgagggaaagttcagtgtgtccctgaaacatggaaacactgattcagagggtattgtggaggtgaaacttgtggatcaggacaagaccatgttcatctgcaagtccagttggagcatgagagaggctaatgtggcctgtctggacctgggttttcagcaaggagctgacactcagagaaggttcaagctgtcagatctcagtatcaactccacagaatgtctgcatgtgcattgcagagggctggagactagccttgctgaatgtacttttacaaagagaagaactatgggataccaggattttgcagatgtggtctgttatactcagaaggctgattcacccatggatgacttcttccagtgtgtgaatggcaagtacatttcacaaatgaaagcatgtgatggtatcaatgattgtggagaccagagtgatgaactctgttgcaaggcatgccagggcaagggattccactgcaagagtggtgtctgtattcccagtcagtatcagtgcaatggggaggtggactgcatcactggggaagatgaagtgggctgtgctggttttgctagtgtgacacaggaggaaactgaaattctcacagcagatatggatgctgagagaaggagaatcaagtccctcctccctaaactgagttgtggagtgaaaaacagaatgcatattagaagaaaaagaattgtgggaggcaagagagctcagcttggggacctcccatggcaagtggctattaaggatgcaagtgggattacatgtggagggatctatattggggggtgctggattctgacagcagcccactgtctgagagcctctaaaacccatagatatcagatctggacaacagtggtggattggattcacccagacctcaagaggattgtgattgagtatgtggatagaattatttttcatgagaactacaatgctggaacctatcagaatgatattgctctgattgaaatgaaaaaggatgggaacaaaaaggactgtgaactgccaagatcaatccctgcatgtgtgccatggagtccctatctctttcagcccaatgatacatgcattgttagtggatgggggagggagaaagacaatgagagagtgttcagtctccagtggggtgaagtgaagctcatttctaattgtagcaagttttatgggaatagattctatgaaaaagaaatggagtgtgcaggaacttatgatggttccattgatgcctgcaagggagatagtggtgggcccctggtgtgcatggatgctaataatgtgacctatgtctggggagtggtttcatggggagagaattgtggaaagcctgagttccctggagtgtacaccaaggtggccaattactttgattggatcagctatcatgtgggaagaccatttatttcccagtacaatgtctaa

SEQ ID NO: 16 - DNA, 1698 bp, Artificial Sequence
  Synthetic construct
aaggtgacctatacaagccaggaagatcttgtggagaaaaagtgccttgctaagaagtacactcacctcagctgtgacaaagtcttttgccagccttggcagagatgtattgagggaacttgtgtctgtaagctcccatatcagtgtcccaagaatggaacagctgtgtgtgctactaacagaaggagctttcccacctactgccagcagaagagccttgaatgtctccacccagggaccaagtttctgaacaatggcacatgtacagctgaggggaaattcagtgtcagtctcaaacatgggaatactgatagtgagggcattgttgaggtgaaacttgtggaccaggataagaccatgtttatctgcaagtcaagctggtcaatgagagaagctaatgtggcttgtctggaccttgggtttcaacaaggagctgacactcagagaagatttaagctgagtgatctgagtatcaacagcactgaatgtctgcatgtgcattgcagagggctggagactagccttgctgagtgcacttttaccaagagaagaacaatgggataccaggattttgctgatgtggtctgttatactcagaaggcagattcaccaatggatgacttctttcagtgtgtgaatggaaaatacatttcccagatgaaggcttgtgatggtatcaatgactgtggggaccagagtgatgagctctgttgtaaggcatgtcagggcaaaggatttcactgcaagtctggggtctgtatccccagccagtatcagtgtaatggagaggttgattgtatcacaggggaggatgaagtgggttgtgctggctttgcatcagtgacacaggaagagacagaaattctgacagcagatatggatgctgaaagaaggagaattaaaagcctcctgccaaaactgagctgtggtgtcaagaatagaatgcacatcagaaggaagagaattgttggtggaaagagggcccagcttggggacctgccatggcaggttgcaatcaaagatgcaagtggcattacctgtggaggaatttacattggagggtgttggattctgacagcagctcattgtctaagagcctccaaaacccacagataccagatctggaccactgtggtggattggattcaccctgacctgaaaagaattgtcattgagtatgtggacaggattattttccatgagaattataatgctggcacctaccagaatgacattgccctgattgagatgaagaaagatggaaataagaaggactgtgagctcccaagatcaattcctgcttgtgtgccttggtctccatacctgtttcagcccaatgatacttgtattgtttctggatggggcagggaaaaagacaatgagagagtgttcagcctgcagtggggggaggtgaagctgatctcaaactgcagcaagttctatggaaatagattttatgagaaggaaatggagtgtgcaggcacttatgatggatcaattgatgcctgcaagggtgacagtggtggcccacttgtgtgcatggatgctaacaatgtgacctatgtttggggtgtggtgtcatggggggaaaactgtggcaaacctgagtttcctggtgtttacactaaggttgcaaactactttgactggatcagctatcatgtgggcagaccttttatttcccagtataatgtctaa

SEQ ID NO: 17 - DNA, 1698 bp, Artificial Sequence
  Synthetic construct
aaggtgacctataccagccaggaggaccttgttgagaagaaatgcctggctaagaaatacacccacctgtcctgtgacaaggtgttctgtcagccttggcagaggtgcattgaagggacctgtgtgtgtaaactcccctaccagtgccctaagaatggcactgcagtgtgtgctacaaacagaagatcattcccaacctattgccagcagaaatcactggaatgcctgcatcctggtacaaaattcctgaacaatgggacttgtactgctgaggggaaattcagtgtgtccctgaaacatgggaacacagattctgagggtattgttgaggtgaagctggtggatcaggataagactatgtttatctgcaagtcaagctggtccatgagggaagccaatgtggcatgcctggatctgggttttcaacaaggagctgatacccagagaagatttaagctgagtgacctcagcattaactctactgagtgtctgcatgtgcactgtagagggcttgaaacaagcctggcagagtgcaccttcaccaagagaaggacaatgggataccaggactttgctgatgtggtctgctacacccagaaggctgattcccccatggatgatttctttcaatgtgtgaatgggaagtacatcagccagatgaaagcatgtgatgggatcaatgactgtggagaccagagtgatgagctgtgctgcaaagcttgccaggggaaaggctttcattgcaagagtggggtctgtattccctcccaataccagtgcaatggggaggtggattgcatcacaggagaagatgaagtgggctgtgctggatttgcctcagtgactcaggaagagacagaaattctcacagctgatatggatgcagagagaaggaggatcaagtccctcctgcctaaactgagttgtggagtgaagaacaggatgcatattagaaggaagagaattgtggggggtaaaagagcccagcttggggacctgccttggcaagtggctatcaaagatgccagtggtattacctgtggtgggatctatattggtggttgctggattctcactgctgcacactgtctgagagccagcaaaacccacagatatcagatttggactacagtggttgactggattcaccctgacctgaagagaattgttattgagtatgtggatagaatcatcttccatgagaattacaatgctgggacttatcagaatgatattgccctcattgaaatgaagaaggatgggaacaaaaaggattgtgagctgcccagaagcattccagcctgtgtgccatggagcccctatctgtttcagccaaatgacacttgtattgtttctggttggggcagggaaaaagacaatgaaagagtctttagcctccagtggggagaagttaagctgatttcaaattgcagcaaattctatgggaacaggttctatgagaaggagatggagtgtgctgggacatatgatggttcaattgatgcttgcaaaggagatagtggtgggccactggtctgcatggatgccaataatgtgacatatgtctggggggtggtttcctggggagagaattgtgggaagccagagtttcctggtgtctatacaaaagtggctaactattttgactggatcagctatcatgtggggagacctttcatcagtcagtataatgtgtag

SEQ ID NO: 18 - DNA, 1698 bp, Artificial Sequence
  Synthetic construct
aaggtgacctacacctctcaggaggatctggtggagaaaaagtgcctggcaaaaaagtatactcacctcagttgtgacaaggtcttctgccagccatggcagagatgtattgagggaacctgtgtgtgcaaactcccttatcagtgtcccaagaatgggactgcagtctgtgcaaccaacagaagaagctttcccacatactgtcagcagaaatcccttgagtgcctgcaccctggaaccaaattcctgaataatggaacttgcacagcagaggggaaattcagtgtcagcctgaaacatggcaacactgatagtgaggggattgttgaagtgaagctggtggatcaggataagactatgtttatctgcaaaagttcatggagcatgagagaagcaaatgtggcatgccttgatctgggtttccaacaaggagctgacactcagagaagatttaaactcagtgatctgtcaatcaacagcacagagtgcctgcatgtgcactgtagaggacttgagacatcacttgcagaatgcacatttaccaaaagaagaaccatgggataccaggattttgcagatgtggtgtgctatactcagaaagctgatagccccatggatgacttcttccagtgtgtcaatgggaagtatatcagccagatgaaggcctgtgatggaatcaatgactgtggagatcagagtgatgaactctgttgtaaggcatgtcaggggaagggtttccattgcaagagtggagtctgcattccttcacaataccagtgcaatggagaggtggattgtattactggggaggatgaagtgggttgtgcaggatttgccagtgtgactcaggaggaaactgagattctgacagctgacatggatgctgagagaagaaggatcaagagcctgctcccaaagctgtcatgtggggtcaaaaatagaatgcacatcagaaggaagagaattgtgggagggaaaagagctcagctgggagacctcccttggcaagtggcaatcaaggatgctagtggtatcacatgtggaggaatctacattggggggtgttggattctgactgcagcccattgcctgagggcatccaaaacccacagatatcagatctggacaacagttgtggactggattcaccctgacctcaaaagaattgtcattgagtatgtggacagaatcattttccatgagaattacaatgctggcacttaccagaatgacattgccctgattgaaatgaagaaggatggaaacaagaaggattgtgaactccccaggtcaatcccagcctgtgtgccctggagcccctacctgttccagccaaatgatacatgtattgtcagtggatgggggagagagaaggacaatgagagagtgttttcactgcagtggggggaagtgaagctgatcagcaattgctccaagttctatggaaacaggttttatgagaaggagatggagtgtgctgggacttatgatggaagtattgatgcttgtaaaggggattcaggaggtcctcttgtctgcatggatgctaacaatgtgacatatgtgtggggagtagtcagttggggagaaaactgtggaaaaccagaatttcctggggtgtatactaaagtggcaaattattttgactggatcagctatcatgtgggtagacccttcattagtcagtataatgtgtga

SEQ ID NO: 19 - DNA, 1698 bp, Artificial Sequence
  Synthetic construct
aaggtgacctacacctctcaggaagaccttgttgaaaagaagtgtctggccaaaaagtatactcatctgtcttgtgacaaagtcttctgtcagccctggcagagatgcattgagggaacatgtgtctgcaagctcccttatcagtgtcccaaaaatggcacagcagtttgtgccacaaatagaaggtcttttcctacatactgccagcagaagagccttgaatgtctccacccaggcacaaagtttctgaataatgggacttgcacagcagaaggcaaatttagtgtgtccctgaaacatggaaacactgatagtgagggcattgtggaggttaagcttgtggatcaggataaaacaatgttcatctgcaagagctcatggtccatgagagaggctaatgtggcttgtctggacctgggttttcaacaaggagctgatactcagagaaggtttaagctcagtgacctgagcatcaacagtacagagtgtctgcatgtgcattgtagaggactggaaacttctctggctgagtgtacctttacaaagagaagaacaatgggataccaggattttgctgatgtggtctgctacacccagaaagctgactcccccatggatgatttctttcaatgtgttaatgggaaatacattagccagatgaaggcttgtgatggtatcaatgactgtggagaccagtctgatgaactgtgctgtaaagcctgtcaagggaaaggattccactgcaagagtggagtgtgcattccatcccagtaccagtgtaatggagaagttgattgcattacaggggaagatgaagtgggttgtgcagggtttgcttcagtaactcaggaggagacagaaatcctgacagctgacatggatgctgaaaggagaaggatcaaaagcctgctgcccaagctctcatgtggggtgaaaaacaggatgcatattagaaggaagaggattgtgggagggaaaagagcccagctgggggacctgccatggcaggttgccattaaagatgcttcagggatcacatgtgggggcatctatattggaggctgttggattctcacagcagcacactgcctgagagcttcaaagactcacagatatcagatttggacaacagtggtggattggattcaccctgatctcaaaagaattgtcattgaatatgttgacagaatcatctttcatgaaaactacaatgctggaacttatcagaatgatattgctctgattgaaatgaagaaagatggaaacaaaaaggactgtgaactgcccagaagcatccctgcttgtgttccatggtccccttacctgtttcagcctaatgacacctgcattgttagtggctgggggagagagaaggacaatgagagagtgttttccctgcagtggggagaggtgaaactcatctctaactgttcaaaattctatgggaatagattttatgagaaggagatggaatgtgctgggacctatgatgggtcaattgatgcttgtaagggtgacagtggaggacccctggtgtgcatggatgccaacaatgtgacatatgtctggggggtggttagctggggagaaaattgtgggaagcctgagttccctggggtgtacaccaaggttgcaaactattttgattggatttcttatcatgtgggtagaccatttatcagtcagtataatgtgtag

SEQ ID NO: 20 - DNA, 1698 bp, Artificial Sequence
  Synthetic construct
aaggtgacctatacttctcaagaggatctggtggagaaaaaatgcctggctaaaaaatatactcacctgtcctgtgataaagtcttctgccagccatggcagagatgcattgagggaacttgtgtgtgcaaactgccatatcagtgcccaaaaaatggaactgctgtgtgtgctactaacaggagaagcttcccaacttattgccagcagaaaagtctggaatgccttcatccaggaactaaatttctgaataatggaacttgcactgctgaaggaaaatttagtgtgtccctgaaacatggaaatactgattcagagggaattgtggaagtgaaacttgtggatcaggataaaactatgttcatttgcaaaagcagctggagcatgagggaagctaatgtggcttgccttgatcttggatttcaacaaggagctgatactcagagaaggtttaaactgtctgatctgtctattaattccactgaatgcctgcatgtgcattgcagaggactggagactagtctggctgaatgcacttttactaaaagaagaactatgggatatcaggattttgctgatgtggtgtgctatactcagaaagctgattctccaatggatgatttctttcaatgtgtgaatggaaaatatatttctcagatgaaagcttgtgatggaatcaatgattgtggagatcagagtgatgaactgtgctgcaaagcttgccagggaaaaggatttcattgcaaatctggagtgtgcattccaagccagtatcagtgcaatggagaggtggattgcattactggagaagatgaagtgggatgtgctggatttgcttctgtgactcaagaagaaactgaaattctgactgctgatatggatgctgaaagaagaagaattaaatcactgctgcctaaactgtcttgtggagtgaaaaacagaatgcacattagaaggaaaagaattgtgggaggaaaaagagctcagctgggagatctgccatggcaggtggctattaaagatgctagtggaatcacttgtggaggaatttatattggaggatgctggattctgactgctgctcattgcctgagagctagtaaaactcatagatatcagatttggactactgtggtggattggattcacccagatcttaaaagaattgtgattgaatatgtggatagaattattttccatgaaaactataatgctggaacttatcagaatgatattgctctgattgaaatgaaaaaagatggaaacaaaaaagattgtgagctgcctagatccatccctgcttgtgtcccatggtctccttatctgttccaacctaatgatacttgcattgtgtctggatggggaagagaaaaagataatgaaagagtcttttcacttcaatggggagaagtgaaactgattagcaactgctctaaattttatggaaatagattctatgaaaaagaaatggaatgtgctggaacttatgatggatccattgatgcttgcaaaggagattctggaggaccactggtctgcatggatgctaacaatgtgacttatgtctggggagtggtcagttggggagaaaactgtggaaaaccagagttcccaggagtgtatactaaagtggctaattattttgattggattagctatcatgtgggaaggccttttatttctcagtataatgtgtaa

SEQ ID NO: 21 - DNA, 1752 bp, Artificial Sequence
  Synthetic construct
atgaagctgctgcacgtgttcctgctgtttctgtgcttccacctgcgcttttgtaaggtgacctatacatctcaggaggatctggtggagaagaagtgtctggccaagaagtacacacacctgagctgcgacaaggtgttctgtcagccatggcagcggtgcatcgagggaacctgcgtgtgcaagctgccttatcagtgcccaaagaacggcaccgccgtgtgcgccacaaatcggagatcttttccaacatactgccagcagaagagcctggagtgtctgcaccccggcaccaagtttctgaacaatggcacctgcacagccgagggcaagttctctgtgagcctgaagcacggcaacacagattccgagggcatcgtggaggtgaagctggtggaccaggataagaccatgttcatctgtaagagctcctggtctatgagagaggccaacgtggcctgcctggatctgggctttcagcagggcgccgacacacagaggcgcttcaagctgtccgacctgtctatcaatagcaccgagtgcctgcacgtgcactgtaggggcctggagacaagcctggccgagtgcacctttacaaagcggagaaccatgggctatcaggacttcgccgacgtggtgtgctacacccagaaggccgattctccaatggacgatttctttcagtgcgtgaacggcaagtacatcagccagatgaaggcctgcgacggcatcaatgactgtggcgatcagtccgacgagctgtgctgtaaggcctgtcagggcaagggctttcactgcaagagcggcgtgtgcatcccctcccagtatcagtgcaacggcgaggtggattgtatcaccggagaggacgaagtgggatgcgcaggcttcgcctccgtgacccaggaggagaccgagatcctgacagccgacatggatgccgagaggcgccggatcaagtctctgctgcctaagctgagctgtggcgtgaagaatagaatgcacatcagaaggaagcgcatcgtgggaggcaagagggcacagctgggcgatctgccatggcaggtggccatcaaggacgcaagcggaatcacctgcggaggcatctatatcggaggatgttggatcctgaccgcagcacactgcctgagagcatccaagacacacaggtaccagatctggaccacagtggtggattggatccaccctgacctgaagagaatcgtgatcgagtatgtggataggatcatcttccacgagaactacaatgccggcacatatcagaacgacatcgccctgatcgagatgaagaaggatggcaataagaaggactgtgagctgccacgctccatccctgcatgcgtgccctggtctccttacctgtttcagccaaacgatacctgcatcgtgagcggatggggaagggagaaggacaatgagcgggtgttctccctgcagtggggcgaggtgaagctgatctccaactgttctaagttttacggcaatcggttctatgagaaggagatggagtgcgccggcacctacgatggcagcatcgacgcctgtaagggcgattccggaggaccactggtgtgcatggacgcaaacaatgtgacatacgtgtggggagtggtgagctggggagagaactgcggcaagccagagtttcccggcgtctacaccaaggtggccaattattttgactggatctcctaccacgtgggcaggcctttcatctctcagtacaacgtgtga

SEQ ID NO: 22 - DNA, 1752 bp, Artificial Sequence
  Synthetic construct
atgaagctgctgcatgtgtttctgctgttcctctgcttccacctgaggttctgcaaagtgacctacaccagccaagaggacctggtggaaaagaagtgcctggccaagaagtacacccacctgagctgcgacaaggtgttctgccagccttggcagagatgcatcgagggcacctgtgtgtgcaagctgccctatcagtgccccaagaatggcacagccgtgtgcgccaccaacagaagaagcttccctacctactgccagcagaaaagcctggaatgtctgcaccccggcaccaagtttctgaacaacggcacctgtaccgccgagggcaagtttagcgtgtccctgaagcacggcaacaccgactctgagggcatcgtggaagtgaagctggtggaccaggacaagaccatgttcatctgcaagagcagctggtccatgcgcgaggccaatgtggcttgtctggatctgggattccagcagggcgccgacacacagagaagattcaagctgagcgacctgagcatcaacagcaccgagtgcctgcatgtgcactgtagaggcctggaaacaagcctggccgagtgcaccttcaccaagagaaggaccatgggctaccaggacttcgccgacgtcgtgtgctacacccagaaagccgactctcccatggacgatttcttccagtgcgtgaacggcaagtacatcagccagatgaaggcctgcgacggcatcaacgattgcggcgatcagagcgacgagctgtgctgcaaagcctgtcaaggcaagggcttccactgcaagtccggcgtgtgtatccctagccagtaccagtgcaatggcgaggtggactgtatcaccggcgaggatgaagtgggctgtgccggatttgccagcgtgacccaagaggaaaccgagatcctgaccgccgatatggacgccgagcggcggagaatcaaaagcctgctgcctaagctgtcctgcggcgtgaagaaccggatgcacatccggcgcaagagaatcgtcggaggcaaaagagcacagctgggcgatctgccttggcaagtggccatcaaggatgccagcggcatcacatgtggcggcatctacatcggcggctgctggattctgacagccgctcattgtctgcgggccagcaagacccaccggtatcagatctggaccaccgtggtggactggattcaccccgacctgaagcggatcgtgatcgagtacgtggaccggatcatcttccacgagaactacaacgccggcacctaccagaacgatatcgccctgatcgagatgaagaaggacgggaacaagaaggactgcgagctgcctagatctatccccgcctgtgttccttggagcccctacctgttccagcctaacgatacctgcatcgtgtccggctggggcagagagaaggataacgagagggtgttcagcctgcagtggggcgaagtgaaactgatcagcaactgcagcaagttctacggcaaccggttctacgagaaagaaatggaatgcgccggcacatacgacggctccatcgatgcctgtaaaggcgattctggcggccctctcgtgtgcatggatgccaacaatgtgacctacgtgtggggcgtcgtgtcctggggagagaattgtggcaagcctgagttccccggcgtctacaccaaggtggccaactacttcgactggatcagctaccacgtgggcagaccctttatcagccagtacaacgtgtga

SEQ ID NO: 23 - DNA, 1752 bp, Artificial Sequence
  Synthetic construct
atgaagctgctgcatgtgttcctgctgtttctgtgcttccacctgagattttgtaaggtgacctatacatctcaggaggatctggtggagaagaagtgtctggccaagaagtacacacacctgagttgtgacaaggtgttctgtcagccatggcagagatgcattgagggaacctgtgtgtgcaagctgccttatcagtgcccaaagaatggcactgctgtgtgtgccacaaataggagatcttttccaacatactgccagcagaagagcctggagtgtctgcaccctggcaccaagtttctgaacaatggcacctgcacagctgagggcaagttctctgtgagcctgaagcatggcaacacagatagtgagggcattgtggaggtgaagctggtggaccaggataagaccatgttcatctgtaagagctcctggtctatgagagaggccaatgtggcctgcctggatctgggctttcaacaaggagctgacacacagaggagattcaagctgagtgacctgtctatcaatagcacagagtgcctgcatgtgcactgtagaggcctggagacaagcctggctgagtgcacctttacaaagagaaggaccatgggctatcaggactttgctgatgtggtatgctacacccagaaggctgattctccaatggatgatttctttcaatgtgtgaatggcaagtacatcagccagatgaaggcctgtgatggcatcaatgactgtggagatcagagtgatgagctgtgttgcaaggcctgtcagggcaagggctttcactgcaagagtggagtgtgcatcccctcccaatatcagtgcaatggagaggtggattgtatcacaggagaggatgaagtgggatgtgcaggctttgcctcagtgacccaggaggagacagagatcctgacagctgacatggatgctgagaggagaaggatcaagtctctgctgcctaagctgagctgtggagtgaagaatagaatgcacatcagaaggaagagaattgtgggaggcaagagggcacagctgggagatctgccatggcaggtggccatcaaggatgcaagtggaatcacctgtggaggcatctatattggaggatgttggatcctgacagcagcacactgcctgagagcatccaagacacacagataccagatctggaccacagtggttgattggatccaccctgacctgaagagaattgtgattgagtatgtggataggatcatcttccatgagaactacaatgctggcacatatcagaatgacattgccctgattgagatgaagaaggatggcaataagaaggactgtgagctgccaagatccatccctgcatgtgtgccctggtctccttacctgtttcagccaaatgatacctgcattgtatccggatggggaagggagaaggacaatgagagagtgttctccctgcagtggggagaggtgaagctgatctccaactgttctaagttttatggcaatagattctatgagaaggagatggagtgtgcaggcacctatgatggcagcattgatgcctgtaagggagatagtggaggaccactggtgtgcatggatgcaaacaatgtgacatatgtgtggggagtggtgagctggggagagaactgtggcaagccagagtttcctggagtctacaccaaggtggccaattattttgactggatctcctaccatgtgggcaggcctttcatctctcagtacaatgtgtga

SEQ ID NO: 24 - DNA, 1752 bp, Artificial Sequence
  Synthetic construct
atgaagctgctgcatgtgtttctgctgttcctctgcttccacctgaggttctgcaaggtgacctacaccagccaagaggacctggtggaaaagaagtgcctggccaagaagtacacccacctgagctgtgacaaggtgttctgccagccttggcagagatgcattgagggcacctgtgtgtgcaagctgccctatcagtgccccaagaatggcacagctgtgtgtgccaccaacagaaggagcttccctacctactgccagcagaaaagcctggaatgtctgcaccctggcaccaagtttctgaacaatggcacctgtacagctgagggcaagtttagtgtgtccctgaagcatggcaacacagactctgagggcattgtggaagtgaagctggtggaccaggacaagaccatgttcatctgcaagagtagctggtccatgagagaggccaatgtggcttgtctggatctgggattccaacagggagctgacacacagagaagattcaagctgagtgacctgagcatcaacagcacagagtgcctgcatgtgcactgtagaggcctggaaacaagcctggctgagtgcaccttcaccaagagaaggaccatgggctaccaggactttgctgatgtggtgtgctacacccagaaagctgactctcccatggatgatttctttcaatgtgtgaatggcaagtacatcagccagatgaaggcctgtgatggcatcaatgattgtggagatcagagtgatgagctgtgctgcaaagcctgtcaaggcaagggcttccactgcaagagtggagtgtgtatccctagccagtaccagtgcaatggagaggtggactgtatcacaggagaggatgaagtgggctgtgctggatttgccagtgtgacccaagaggaaacagagatcctgacagctgatatggatgctgagagaaggagaatcaaaagcctgcttcctaagctgtcctgtggagtgaagaacagaatgcacatcagaaggaagagaattgtgggaggcaaaagagcacagctgggagatctgccttggcaagtggccatcaaggatgccagtggcatcacatgtggaggcatctacattggaggctgctggattctgacagctgctcattgtctgagagccagcaagacccacagatatcagatctggaccacagtggtagactggattcaccctgacctgaagagaattgtgattgagtatgtggacagaatcatcttccatgagaactacaatgctggcacctaccagaatgatattgccctgattgagatgaagaaggatggaaacaagaaagactgtgagctgcctagatctatccctgcctgtgttccttggagcccctacctgttccagcctaatgatacctgcatagtttctggatggggcagagagaaggataatgagagggtgttcagcctgcagtggggagaagtgaaactgatcagcaactgcagcaagttctatggcaacagattctatgagaaagaaatggaatgtgctggcacatatgatggctccattgatgcctgtaaaggagattctggaggccctcttgtgtgcatggatgccaacaatgtgacctatgtgtggggagtggtatcctggggagagaattgtggcaagcctgagttccctggagtctacaccaaggtggccaactactttgactggatcagctaccatgtgggcagaccctttatcagccagtacaatgtgtga

SEQ ID NO: 25 - DNA, 1752 bp, Artificial Sequence
  Synthetic construct
atgaagctgctgcatgtgttcctgctgttcctgtgcttccacctgagattctgcaaggtgacctacaccagccaggaggacctggtggagaagaagtgcctggccaagaagtacacccacctgagctgtgacaaggtgttctgccagccctggcagagatgcattgagggcacctgtgtgtgcaagctgccctaccagtgccccaagaatggcacagctgtgtgtgccaccaacagaaggagcttccccacctactgccagcagaagagcctggagtgcctgcaccctggcaccaagttcctgaacaatggcacctgcacagctgagggcaagttcagtgtgagcctgaagcatggcaacacagacagtgagggcattgtggaggtgaagctggtggaccaggacaagaccatgttcatctgcaagagcagttggagcatgagagaggccaatgtggcctgcctggacctgggcttccagcaaggagctgacacccagagaaggttcaagctgagtgacctgagcatcaacagcacagagtgcctgcatgtgcactgcagaggcctggagaccagcctggctgagtgcaccttcaccaagagaaggaccatgggctaccaggactttgctgatgtggtttgctacacccagaaggctgacagccccatggatgacttcttccagtgtgtgaatggcaagtacatcagccagatgaaggcctgtgatggcatcaatgactgtggagaccagagtgatgagctgtgctgcaaggcctgccagggcaagggcttccactgcaagagtggagtgtgcatccccagccagtaccagtgcaatggagaggtggactgcatcacaggagaggatgaggtgggctgtgctggctttgccagtgtgacccaggaggagacagagatcctgacagctgacatggatgctgagagaaggagaatcaagagcctgctgcccaagctgagctgtggagtgaagaacagaatgcacatcagaaggaagagaattgtgggaggcaagagagcccagctgggagacctgccctggcaggtggccatcaaggatgccagtggcatcacctgtggaggaatctacattggaggctgctggattctgacagctgcccactgcctgagagccagcaagacccacagataccagatctggaccacagtggttgactggattcaccctgacctgaagagaattgtgattgagtatgtggacagaatcattttccatgagaactacaatgctggcacctaccagaatgacattgccctgattgagatgaagaaggatggcaacaagaaagactgtgagctgcccagaagcatccctgcctgtgtgccctggagcccctacctgttccagcccaatgacacctgtattgtcagtggatggggcagagagaaggacaatgagagagtgttcagcctgcagtggggagaggtgaagctgatcagcaactgcagcaagttctatggcaacagattctatgagaaggagatggagtgtgctggcacctatgatggcagcattgatgcctgcaagggagacagtggaggccccctggtgtgcatggatgccaacaatgtgacctatgtgtggggagtggttagctggggagagaactgtggcaagcctgagttccctggagtgtacaccaaggtggccaactactttgactggatcagctaccatgtgggcagacccttcatcagccagtacaatgtgtaa

SEQ ID NO: 26 - DNA, 1752 bp, Artificial Sequence
  Synthetic construct
atgaagctgctgcatgtgttcctgctgttcctgtgtttccatctgaggttctgtaaggtgacctatacatcacaggaggacctggtggagaagaagtgtctggccaagaagtatacacatctgtcatgtgacaaggtgttctgtcagccttggcagaggtgtattgagggaacatgtgtgtgtaagctgccttatcagtgtcctaagaatggaacagctgtgtgtgccacaaataggaggtcattccctacatattgtcagcagaagtcactggagtgtctgcatcctggaacaaagttcctgaataatggaacatgtacagctgagggaaagttctcagtgtcactgaagcatggaaatacagactcagagggaattgtggaggtgaagctggtggaccaggacaagacaatgttcatctgcaagtcatcatggtcaatgagggaggccaatgtggcctgtctggacctgggattccagcagggagctgacacacagaggaggttcaagctgtcagacctgtcaatcaattcaacagagtgtctgcatgtgcattgtagaggactggagacatcactggctgagtgtacattcacaaagaggagaacaatgggatatcaggactttgctgatgtggtttgttatacacagaaggctgactcacctatggatgacttctttcaatgtgtgaatggaaagtatatctcacagatgaaggcctgtgatggaatcaatgactgtggagaccagtcagatgagctgtgttgcaaggcctgtcagggaaagggattccattgcaagtcaggagtgtgtatcccttcacaatatcagtgtaatggagaggtggactgtatcacaggagaggatgaggtgggatgtgcaggatttgcctcagtgacccaggaggagacagagatcctgacagctgacatggatgctgagagaaggagaatcaagtcactgctgcctaagctgtcatgtggagtgaagaataggatgcatatcaggaggaagaggattgtgggaggaaagagggcccagctgggagacctgccttggcaagtggccatcaaggatgcctcaggaatcacatgtggaggaatctatattggaggatgttggatcctgacagctgcccattgtctgagggcctcaaagacacatagatatcagatctggacaacagtggtggactggatccatcctgacctgaagaggattgtgattgagtatgtggacaggatcatcttccatgagaattataatgctggaacatatcagaatgacattgccctgattgagatgaagaaggatggaaataagaaggactgtgagctgcctaggtcaatccctgcctgtgtgccttggtccccttatctgttccaacctaatgacacatgtattgtgtctggatggggaagggagaaggacaatgagagggtgttctcactgcagtggggagaggtgaagctgatctcaaattgttcaaagttctatggaaataggttctatgagaaggagatggagtgtgctggaacatatgatggatcaattgatgcctgtaagggagactcaggaggacctctagtatgtatggatgccaataatgtgacatatgtgtggggagtggtgtcatggggagagaattgtggaaagcctgagttccctggagtgtatacaaaggtggccaattattttgactggatctcatatcatgtgggaaggcctttcatctcacagtataatgtgtaa

SEQ ID NO: 27 - DNA, 1752 bp, Artificial Sequence
  Synthetic construct
atgaagctgctgcatgtgttcctgctgtttctgtgcttccacctgaggttttgcaaggtgacctatacctctcaagaggatctggtggagaagaagtgcctggctaagaagtatactcacctgagctgtgataaggtgttctgccagccctggcagaggtgcattgagggcacctgtgtgtgtaagctgccctatcagtgccccaagaatggcactgctgtgtgtgctactaacaggaggagcttccccacctactgtcagcagaagagcctggagtgtctgcatcctgggaccaagtttctgaataatggaacctgcacagctgagggaaagtttagtgtgagcctgaagcatggaaatacagatagtgagggaattgtggaggtgaagctggtggaccaggataagaccatgttcatctgcaagagcagctggagcatgagggaggccaatgtggcctgcctggacctggggtttcagcaaggtgctgatactcagaggaggtttaagctgtctgatctgtctatcaatagcactgagtgtctgcatgtgcattgtagaggactggagaccagcctggctgagtgtacttttactaagaggaggactatgggataccaggattttgctgatgtggtgtgttatacccagaaggctgattctcccatggatgacttctttcagtgtgtgaatgggaagtacatctctcagatgaaggcctgtgatggtatcaatgattgtggagaccagagtgatgagctgtgttgtaaggcttgccaaggcaagggctttcattgcaagagtggagtgtgcatccccagccagtatcagtgcaatggagaggtggactgcatcacaggggaggatgaggtgggctgtgctggctttgcttctgtgactcaagaggagacagagatcctgactgctgacatggatgctgagaggaggaggatcaagagcctgctgcctaagctgtcttgtggagtgaagaacaggatgcacatcaggaggaagaggattgtgggaggaaagagggctcagctgggagacctgccctggcaggtggctatcaaggatgccagtggaatcacctgtgggggaatctatattggtggctgttggatcctgactgctgctcattgtctgagggccagcaagactcatagataccagatctggaccacagtggtggactggatccaccctgacctgaagaggattgtgattgagtatgtggataggatcatcttccatgagaactacaatgctggcacttaccagaatgacattgctctgattgagatgaagaaggatggaaacaagaaggattgtgagctgcctaggagcatccctgcctgtgtgccctggtctccttacctgttccaacctaatgatacctgcattgtgtctggctggggaagggagaaggataatgagagggtgtttagcctgcagtggggtgaggtgaagctgatcagcaactgctctaagttttatggaaataggttctatgagaaggagatggagtgtgctggaacctatgatggtagcattgatgcctgcaagggggactctggaggccccctggtttgtatggatgccaacaatgtgacttatgtgtggggtgtggtgagctggggggagaactgtggaaagcctgagttccctggagtgtacaccaaggtggccaattattttgactggatcagctaccatgtgggaaggccttttatctctcagtacaatgtgtaa

SEQ ID NO: 28 - DNA, 1752 bp, Artificial Sequence
  Synthetic construct
atgaagctgctgcatgtttttctgctgtttctgtgctttcacctgagattttgcaaggtgacctatacaagccaggaggatctggtggagaagaagtgcctggcaaagaagtatacccacctgagctgtgataaggtgttttgccagccctggcagagatgcattgagggcacctgtgtttgtaagctgccctatcagtgccccaagaatggcacagcagtgtgtgcaaccaatagaaggagctttcccacatactgtcagcagaagagcctggaatgtctgcatcctgggacaaagtttctgaataatggaacatgcacagctgaaggaaagtttagtgttagcctgaagcatggaaatacagatagtgagggaatagttgaagtgaagctggtggaccaggataagacaatgtttatatgcaagagtagctggagcatgagagaagccaatgtggcctgcctggacctggggtttcagcaaggagctgatacccagagaaggtttaagctgagtgatctgagcataaatagcacagaatgtctgcatgtgcattgcagaggactggagaccagcctggctgaatgtacctttaccaagagaaggaccatgggctaccaggattttgctgatgtggtttgttatacacagaaggcagatagccccatggatgacttttttcaatgtgtgaatgggaagtacattagccagatgaaggcctgtgatggcatcaatgattgtggagaccagagtgatgaactgtgttgtaaggcatgccagggcaagggctttcattgcaagagtggagtttgcattcccagccagtatcagtgcaatggagaggtggactgcattacaggggaagatgaagttggctgtgcaggctttgcaagtgtgactcaggaagaaacagaaattctgacagctgacatggatgcagaaagaagaagaataaagagcctgctgcctaagctgagctgtggagttaagaatagaatgcacattagaaggaagagaattgtgggaggaaagagagcacagctgggagacctgccctggcaggtggcaattaaggatgccagtggaatcacctgtgggggaatttatattggaggctgttggattctgacagctgcacattgtctgagagccagcaagacccatagataccagatatggacaacagtggtggactggatacaccctgacctgaagagaatagtgattgaatatgtggatagaattatttttcatgaaaattacaatgcaggcacctaccagaatgacattgctctgattgaaatgaagaaggatggaaataagaaggattgtgagctgcctagaagcatccctgcctgtgtgccctggagcccttacctgtttcagcctaatgatacatgcattgttagtggctggggaagagaaaaggataatgaaagagtgtttagcctgcagtggggagaagttaagctgataagcaattgcagcaagttttatggaaatagattttatgaaaaggaaatggaatgtgcaggcacatatgatggcagcattgatgcctgcaagggggacagtggaggccccctagtatgtatggatgccaataatgtgacctatgtgtggggagttgtgagctggggggaaaattgtggaaagcctgagtttcctggagtttacaccaaggtggccaattattttgactggattagctaccatgtgggaagaccttttattagccagtacaatgtgtaa

SEQ ID NO: 29 - DNA, 1752 bp, Artificial Sequence
  Synthetic construct
atgaagctgctgcatgtgttcctgctgttcctgtgtttccacctgaggttctgtaaggtgacctatacatctcaggaggatctggtggagaagaagtgtctggccaagaagtatacacacctctcctgtgataaggtgttctgtcagccatggcagaggtgtattgagggaacctgtgtgtgtaagctgccttatcagtgtccaaagaatggaacagctgtgtgtgccacaaacaggaggagcttcccaacatactgtcagcagaagtcactggaatgtctgcatccaggaacaaagttcctgaataatggaacatgtacagctgaaggaaagttctcagtgtccctgaagcatggaaatacagattcagagggaattgtggaagtgaagctggtggaccaggataagacaatgttcatctgtaagagcagctggagcatgagggaagccaatgtggcctgtctggacctgggattccagcagggagctgatacacagaggaggttcaagctgtctgatctctctatcaattccacagaatgtctgcatgtgcattgcagaggtctggagacctcactggctgaatgtacattcacaaagaggaggacaatgggataccaggattttgctgatgtggtgtgttatacacagaaggctgattctccaatggatgacttctttcagtgtgtgaatggaaagtacatctctcagatgaaggcctgtgatggaatcaatgattgtggagaccagtcagatgaactgtgctgcaaggcctgtcagggaaagggattccattgcaagtcaggagtgtgtatcccaagccagtatcagtgtaatggagaggtggactgtatcacaggagaagatgaagtgggatgtgcaggatttgcctctgtgactcaggaagaaacagaaatcctgacagctgacatggatgctgaaaggaggaggatcaagtcactgctgcctaagctgtcttgtggagtgaagaacaggatgcacatcaggaggaagaggattgtgggaggaaagagggcccagctgggagacctcccatggcaggtggccatcaaggatgcctcaggaatcacctgtggaggaatctatattggaggatgttggatcctgacagctgcccattgtctaagagcctcaaagacacatagataccagatctggacaacagtggtggactggatccaccctgacctgaagaggattgtgattgaatatgtggataggatcatcttccatgaaaactacaatgctggaacataccagaatgacattgctctgatagaaatgaagaaggatggaaacaagaaggattgtgagctgcctaggtccatccctgcctgtgtgccctggtctccttacctgttccaacctaatgatacatgtattgtgtctggatggggaagggaaaaggataatgaaagggtgttctcactgcagtggggagaagtgaagctgatcagcaactgttctaagttctatggaaataggttctatgaaaaggaaatggaatgtgctggaacatatgatggatccattgatgcctgtaagggagactctggaggacccctggtttgtatggatgccaacaatgtgacatatgtgtggggagtggtgtcatggggagaaaactgtggaaagccagagttcccaggagtgtacaccaaggtggccaattattttgactggatcagctaccatgtgggaaggcctttcatctctcagtacaatgtgtaa

SEQ ID NO: 30 - DNA, 1752 bp, Artificial Sequence
  Synthetic construct
atgaaactgctgcatgtgtttctcctgttcctgtgctttcatctgaggttctgtaaggtgacctatacatcccaagaggatcttgtggaaaagaaatgtctggccaaaaagtacacccatctgtcttgtgacaaggtcttctgccagccttggcagagatgtattgaaggaacttgtgtctgtaaactgccctaccagtgccctaagaatggaacagctgtctgtgctactaatagaaggtcattcccaacttactgccagcagaagtctctggagtgtctccaccctggaactaaattcctgaacaatggcacatgtacagctgaaggcaagttctctgtctccctcaaacatggaaacacagactcagaagggattgtggaagtcaagctggtggatcaggataagaccatgtttatttgcaagtcatcctggtctatgagagaagcaaatgtggcctgcctggatctgggctttcaacaaggagcagacactcagaggagattcaagctctctgacctgtctattaatagcactgagtgtctgcatgtgcattgtagaggactggagacctcacttgctgaatgtacattcactaaaagaaggactatgggataccaggactttgctgatgtggtgtgctatacccagaaggcagactcccccatggatgactttttccaatgtgttaatgggaaatatattagtcagatgaaggcttgtgatggaattaatgactgtggagatcagagtgatgagctctgttgcaaagcctgtcagggaaagggtttccactgcaaaagtggggtttgtattccttctcaataccaatgcaatggggaggtggactgcattactggagaggatgaggtggggtgtgctggctttgcttctgtgacccaagaggagacagagatcctgacagctgatatggatgctgaaaggagaagaattaagtcactgctgcccaaactgtcttgtggagttaaaaacagaatgcacatcagaagaaagagaattgtgggaggaaaaagagcccagcttggagatctcccttggcaagtggccattaaggatgccagtggtatcacatgtggaggcatctatattggagggtgctggattctcacagctgcacactgtctgagagccagtaaaacccatagatatcagatctggacaacagtggttgattggattcatcctgatctcaagagaattgtgattgagtatgtggacagaattatttttcatgaaaactacaatgcaggaacctatcagaatgatattgccctcattgagatgaagaaggatgggaacaaaaaggattgtgagctgcccagaagcatcccagcttgtgtgccttggtctccatacctctttcagccaaatgatacttgcattgtcagtggatggggtagagagaaagataatgagagagtcttctccctccagtggggagaggttaagctcatcagtaattgctccaagttttatggaaacaggttctatgaaaaagagatggaatgtgcaggaacatatgatggtagtattgatgcttgcaaaggagatagtggtggacccctggtctgtatggatgccaacaatgtgacttatgtgtggggggtggtgagctggggagagaattgtgggaagcctgagtttccaggagtctacaccaaggtggctaattactttgactggatcagttaccatgtgggtaggcccttcattagccagtacaatgtctga

SEQ ID NO: 31 - DNA, 1752 bp, Artificial Sequence
  Synthetic construct
atgaagctgctgcatgtgttcctcctgtttctctgtttccatctgagattctgcaaggttacctatacatcccaggaagacctggtggagaaaaagtgtcttgccaagaagtacacccacctcagttgtgataaggtcttttgccagccatggcagagatgtattgaaggcacttgtgtgtgcaagctgccataccagtgtcccaaaaatgggacagcagtctgtgcaactaatagaagatccttccctacatactgccagcagaagagtcttgagtgcctgcacccaggcacaaagtttctgaataatggcacatgcacagctgaaggcaaattcagtgtctccctgaaacatggaaacacagatagtgagggaattgtggaagttaagctggtggaccaggataaaaccatgttcatttgtaaatcttcatggagtatgagagaagctaatgtggcatgtctggaccttggatttcaacaaggagcagatacccagagaaggtttaagctgagtgatctctccattaactctactgagtgtctgcatgtgcattgtagaggactggagacatcactggcagaatgtacttttaccaaaaggagaaccatgggttatcaggactttgcagatgtggtctgttacacccagaaggctgactcccccatggatgacttctttcaatgtgtgaatgggaaatacatctcccagatgaaggcctgtgatggtatcaatgactgtggagaccagtctgatgagctctgctgcaaggcctgtcaggggaagggatttcactgcaaaagtggggtttgcatccccagtcagtaccagtgcaatggggaagtggactgcattacaggggaagatgaggttggttgtgctgggtttgctagtgtgacccaggaggaaacagagatcctgacagctgatatggatgcagaaagaagaagaattaagagcctcctgcctaagctcagttgtggagtcaaaaataggatgcatattaggagaaagaggattgtgggagggaaaagagcccagcttggagacctgccatggcaggttgccatcaaggatgcctcagggatcacatgtggagggatctatattggtggatgttggattctgactgctgcccattgcctgagggcctcaaagacccacagatatcagatctggaccactgtggtggactggattcatcctgacctgaagagaattgtgattgaatatgtggacaggattatctttcatgagaactataatgctggaacttatcagaatgatattgcactgattgagatgaagaaggatgggaataaaaaggactgtgaactcccaagaagcattcctgcatgtgtgccttggtctccttacctcttccaacccaatgacacttgtattgtcagtggatggggtagagaaaaagacaatgagagggttttcagtctgcagtggggggaggtcaagctcatcagcaactgctccaaattctatggaaacaggttctatgaaaaagaaatggagtgtgctgggacttatgatggtagtattgatgcctgtaaaggggattctggagggccacttgtctgtatggatgctaacaatgtgacctatgtctggggagtggtgtcatggggagaaaattgtggcaaacctgagtttcctggagtgtatacaaaggtggctaactattttgattggatcagttatcatgttggtagacccttcatcagtcagtacaatgtctag

SEQ ID NO: 32 - DNA, 1752 bp, Artificial Sequence
  Synthetic construct
atgaagctgctccatgtcttcctgctgttcctgtgcttccacctgagattttgcaaggtgacctacaccagccaggaggacctggtggaaaagaaatgtcttgctaagaaatatacccacctgtcctgtgacaaagtcttctgtcagccttggcagagatgtattgaagggacatgtgtctgtaagctgccttaccagtgtcctaagaatgggactgcagtctgtgccactaatagaagatcattcccaacatactgccagcagaagagtctggagtgtctgcatcctggcacaaagtttctcaacaatgggacatgtacagctgaaggaaaattctctgtgagcctgaaacatggcaatacagatagtgagggaattgttgaggttaagctggtggatcaggacaaaacaatgtttatctgcaaaagcagctggtccatgagagaggctaatgtggcctgtcttgaccttggctttcaacaaggagcagatacccagagaagatttaaactgtcagatctgagcatcaattcaacagagtgtctgcatgtgcattgtagaggactggagaccagcctggctgaatgtaccttcacaaagagaagaacaatgggataccaggactttgctgatgtggtttgttacacccagaaggcagacagccccatggatgacttctttcagtgtgtgaatggaaagtatattagccagatgaaggcatgtgatggtatcaatgattgtggggaccagagtgatgagctctgttgtaaggcttgtcaaggcaaaggcttccactgcaagtcaggggtgtgcatcccctcccaataccagtgcaatggagaggtggattgcatcacaggggaggatgaagtgggttgtgctgggtttgcaagtgtgacccaggaggaaacagaaatcctgactgctgatatggatgctgagaggagaagaatcaaaagcctcctgcctaaactgtcttgtggtgttaagaatagaatgcacattagaagaaagagaattgtggggggaaagagagcacagctgggtgatctgccctggcaagtggcaattaaggatgcctcaggcatcacctgtggaggcatttacattgggggttgctggattctgacagcagcccactgtctcagagccagcaaaactcacagatatcagatttggacaacagttgtggattggattcaccctgatctgaagagaattgtcattgagtatgtggacagaattattttccatgagaactacaatgctggcacataccagaatgacattgccctcattgaaatgaagaaggatggaaataaaaaggactgtgaactcccaagatcaattcctgcctgtgtgccatggagtccttacctcttccaaccaaatgatacctgcattgtcagtggctgggggagagagaaggataatgaaagagttttctccctgcagtggggagaggtgaagctcattagtaactgttctaagttttatgggaacagattttatgagaaggagatggaatgtgcaggaacttatgatggtagcattgatgcatgcaaaggagacagtggaggtccacttgtctgcatggatgctaacaatgtgacttatgtctggggagtggtctcctggggtgaaaactgtggaaagcctgagttcccaggagtgtatacaaaagtggccaactactttgactggatttcataccatgtggggagaccttttatcagccagtacaatgtctga

SEQ ID NO: 33 - DNA, 1752 bp, Artificial Sequence
  Synthetic construct
atgaaactgctgcatgtgtttctgctctttctctgcttccacctgagattttgcaaggtgacctacacctctcaggaagacctggtggagaagaagtgtcttgccaagaaatatacccatctgtcatgtgataaagtgttctgtcagccttggcagagatgtattgagggaacatgtgtgtgcaagctgccctaccagtgtccaaagaatgggactgctgtttgtgccactaacagaagatcttttcctacatactgtcagcagaagagtctggagtgcctccaccctggcacaaaattcctcaataatggcacatgtacagctgagggaaagttttctgttagcctcaagcatggcaatactgattcagagggtattgttgaagtcaaactggtggatcaggataaaaccatgttcatttgtaagagtagctggtccatgagggaggctaatgtggcatgcctggacctgggctttcaacaaggggctgacactcagagaaggtttaagctgagtgacctgagcattaattcaactgagtgtctgcatgtgcattgtagagggctggagactagccttgcagagtgcacattcactaagagaagaacaatgggataccaggattttgcagatgtggtgtgctacactcagaaagctgactctcccatggatgacttcttccaatgtgtgaatgggaaatatatttcacagatgaaggcctgtgatggaattaatgattgtggtgatcagagtgatgagctctgttgtaaggcttgccaggggaagggttttcactgtaaaagtggggtttgtatcccctctcaataccagtgcaatggggaagttgattgcattacaggagaggatgaggtgggatgtgcaggctttgccagtgtgacacaggaagagacagagatcctgacagctgatatggatgcagaaagaagaagaatcaagtccctgctccccaaactgtcctgtggggtcaagaatagaatgcacatcagaaggaagagaattgtgggaggcaaaagggcacagctgggggacctcccatggcaggtggcaattaaggatgccagtggtatcacatgtggagggatttacattggaggctgctggattctcacagcagcccactgcctcagagcatctaaaactcacagataccagatttggaccactgtggtggactggattcaccctgacctgaagagaattgtgattgaatatgtggatagaatcattttccatgaaaattacaatgctggcacttatcagaatgacattgcactcattgagatgaagaaagatgggaataaaaaggactgtgaactgcccagaagcattccagcttgtgtgccatggagtccatatctgtttcagccaaatgatacatgcatagtgtctggttggggaagagagaaggacaatgaaagggtttttagcctccagtggggggaggtcaagctgattagcaactgcagtaaattctatggcaacagattttatgagaaggagatggaatgtgcaggcacttatgatggctcaattgatgcctgcaagggagattctggtggacccctggtgtgcatggatgccaataatgtcacctatgtctggggtgtggttagctggggagagaactgtgggaagcctgagtttccaggagtgtatacaaaagtggcaaattactttgactggatctcctatcatgtggggagaccctttatctctcagtacaatgtttga

SEQ ID NO: 34 - DNA, 1752 bp, Artificial Sequence
  Synthetic construct
atgaaactgctccatgtttttctcctctttctctgcttccacctgagattttgcaaggtgacctatacctctcaggaggacctggttgagaaaaagtgcctggctaaaaagtacacccacctctcttgtgataaggtgttctgccagccttggcagagatgcattgaaggcacatgtgtttgtaaactgccttaccagtgtcccaaaaatgggactgctgtgtgtgccacaaatagaaggagctttccaacctactgtcagcagaagagccttgaatgcctccatcctgggacaaaattcctgaacaatggaacctgtacagctgaaggcaaatttagtgttagcctgaaacatgggaatacagattctgagggaattgtggaggtcaaactggtggaccaggataaaaccatgtttatctgcaagagtagctggtccatgagagaggccaatgtggcatgcctggatcttgggtttcaacaaggagctgacacccagagaagattcaaactcagtgacctgagtattaactcaacagaatgtctgcatgtgcattgtagaggactggaaactagccttgcagagtgcacttttaccaagagaagaaccatgggatatcaggattttgctgatgtggtgtgctacacccagaaagcagacagtcccatggatgacttcttccagtgtgtgaatggcaagtatatttcccagatgaaagcatgtgatggcatcaatgattgtggagaccagagtgatgaactgtgctgtaaggcttgccagggaaagggtttccactgcaaaagtggagtttgcatccccagccagtaccagtgcaatggagaagtggattgcattacaggagaggatgaagtgggatgtgctggctttgcttctgttacccaagaagagacagagattctgacagctgacatggatgcagagaggagaagaattaagtccctgctgcctaaactgtcctgtggtgtcaagaatagaatgcacatcagaaggaagagaattgtggggggaaaaagagcccagcttggagacctgccttggcaagtggctattaaggatgccagtgggatcacatgtggaggaatttacattggagggtgctggattctcacagctgcccactgcctgagggccagcaaaacccacagataccagatctggacaacagttgtggattggattcatcctgatctcaagagaattgttattgagtatgtggataggatcattttccatgaaaattacaatgcaggcacataccaaaatgacattgcactcattgagatgaagaaggatgggaataaaaaggattgtgagctccccaggagcattcctgcttgtgtcccctggtccccttacctcttccaacccaatgacacctgcattgtcagtggctggggtagagaaaaagataatgagagagtcttcagcctgcagtggggggaggtgaagctgattagcaattgtagcaagttttatgggaacaggttctatgagaaggagatggagtgtgctggcacctatgatggctctattgatgcttgtaagggggacagtgggggaccactggtttgtatggatgcaaacaatgtgacctatgtttggggagtggttagctggggagaaaattgtggaaaacctgagtttcctggtgtctatacaaaagtggctaattattttgattggatcagttatcatgttgggagaccctttattagtcagtacaatgtctag

SEQ ID NO: 35 - DNA, 1752 bp, Artificial Sequence
  Synthetic construct
atgaaactcctgcatgtgtttctgctgttcctctgttttcacctgaggttttgtaaggtgacctacactagccaggaggatctggtggagaaaaagtgccttgcaaaaaagtacactcacctcagctgtgacaaggtcttctgtcagccttggcagagatgcattgaggggacatgtgtgtgcaaactcccataccagtgtcctaagaatggtacagctgtctgtgcaacaaacaggagatcctttcccacctattgtcagcagaagtccctggagtgtctgcaccctggtacaaagtttctgaataatggcacatgcactgctgagggaaagttcagtgtgtccctgaaacatggaaacactgattcagagggtattgtggaggtgaaacttgtggatcaggacaagaccatgttcatctgcaagtccagttggagcatgagagaggctaatgtggcctgtctggacctgggttttcagcaaggagctgacactcagagaaggttcaagctgtcagatctcagtatcaactccacagaatgtctgcatgtgcattgcagagggctggagactagccttgctgaatgtacttttacaaagagaagaactatgggataccaggattttgcagatgtggtctgttatactcagaaggctgattcacccatggatgacttcttccagtgtgtgaatggcaagtacatttcacaaatgaaagcatgtgatggtatcaatgattgtggagaccagagtgatgaactctgttgcaaggcatgccagggcaagggattccactgcaagagtggtgtctgtattcccagtcagtatcagtgcaatggggaggtggactgcatcactggggaagatgaagtgggctgtgctggttttgctagtgtgacacaggaggaaactgaaattctcacagcagatatggatgctgagagaaggagaatcaagtccctcctccctaaactgagttgtggagtgaaaaacagaatgcatattagaagaaaaagaattgtgggaggcaagagagctcagcttggggacctcccatggcaagtggctattaaggatgcaagtgggattacatgtggagggatctatattggggggtgctggattctgacagcagcccactgtctgagagcctctaaaacccatagatatcagatctggacaacagtggtggattggattcacccagacctcaagaggattgtgattgagtatgtggatagaattatttttcatgagaactacaatgctggaacctatcagaatgatattgctctgattgaaatgaaaaaggatgggaacaaaaaggactgtgaactgccaagatcaatccctgcatgtgtgccatggagtccctatctctttcagcccaatgatacatgcattgttagtggatgggggagggagaaagacaatgagagagtgttcagtctccagtggggtgaagtgaagctcatttctaattgtagcaagttttatgggaatagattctatgaaaaagaaatggagtgtgcaggaacttatgatggttccattgatgcctgcaagggagatagtggtgggcccctggtgtgcatggatgctaataatgtgacctatgtctggggagtggtttcatggggagagaattgtggaaagcctgagttccctggagtgtacaccaaggtggccaattactttgattggatcagctatcatgtgggaagaccatttatttcccagtacaatgtctaa

SEQ ID NO: 36 - DNA, 1752 bp, Artificial Sequence
  Synthetic construct
atgaagctcctgcatgtgtttctgctcttcctctgctttcacctgagattctgtaaggtgacctatacaagccaggaagatcttgtggagaaaaagtgccttgctaagaagtacactcacctcagctgtgacaaagtcttttgccagccttggcagagatgtattgagggaacttgtgtctgtaagctcccatatcagtgtcccaagaatggaacagctgtgtgtgctactaacagaaggagctttcccacctactgccagcagaagagccttgaatgtctccacccagggaccaagtttctgaacaatggcacatgtacagctgaggggaaattcagtgtcagtctcaaacatgggaatactgatagtgagggcattgttgaggtgaaacttgtggaccaggataagaccatgtttatctgcaagtcaagctggtcaatgagagaagctaatgtggcttgtctggaccttgggtttcaacaaggagctgacactcagagaagatttaagctgagtgatctgagtatcaacagcactgaatgtctgcatgtgcattgcagagggctggagactagccttgctgagtgcacttttaccaagagaagaacaatgggataccaggattttgctgatgtggtctgttatactcagaaggcagattcaccaatggatgacttctttcagtgtgtgaatggaaaatacatttcccagatgaaggcttgtgatggtatcaatgactgtggggaccagagtgatgagctctgttgtaaggcatgtcagggcaaaggatttcactgcaagtctggggtctgtatccccagccagtatcagtgtaatggagaggttgattgtatcacaggggaggatgaagtgggttgtgctggctttgcatcagtgacacaggaagagacagaaattctgacagcagatatggatgctgaaagaaggagaattaaaagcctcctgccaaaactgagctgtggtgtcaagaatagaatgcacatcagaaggaagagaattgttggtggaaagagggcccagcttggggacctgccatggcaggttgcaatcaaagatgcaagtggcattacctgtggaggaatttacattggagggtgttggattctgacagcagctcattgtctaagagcctccaaaacccacagataccagatctggaccactgtggtggattggattcaccctgacctgaaaagaattgtcattgagtatgtggacaggattattttccatgagaattataatgctggcacctaccagaatgacattgccctgattgagatgaagaaagatggaaataagaaggactgtgagctcccaagatcaattcctgcttgtgtgccttggtctccatacctgtttcagcccaatgatacttgtattgtttctggatggggcagggaaaaagacaatgagagagtgttcagcctgcagtggggggaggtgaagctgatctcaaactgcagcaagttctatggaaatagattttatgagaaggaaatggagtgtgcaggcacttatgatggatcaattgatgcctgcaagggtgacagtggtggcccacttgtgtgcatggatgctaacaatgtgacctatgtttggggtgtggtgtcatggggggaaaactgtggcaaacctgagtttcctggtgtttacactaaggttgcaaactactttgactggatcagctatcatgtgggcagaccttttatttcccagtataatgtctaa

SEQ ID NO: 37 - DNA, 1752 bp, Artificial Sequence
  Synthetic construct
atgaaactcctgcatgtgtttctcctgttcctctgctttcacctgagattttgtaaggtgacctataccagccaggaggaccttgttgagaagaaatgcctggctaagaaatacacccacctgtcctgtgacaaggtgttctgtcagccttggcagaggtgcattgaagggacctgtgtgtgtaaactcccctaccagtgccctaagaatggcactgcagtgtgtgctacaaacagaagatcattcccaacctattgccagcagaaatcactggaatgcctgcatcctggtacaaaattcctgaacaatgggacttgtactgctgaggggaaattcagtgtgtccctgaaacatgggaacacagattctgagggtattgttgaggtgaagctggtggatcaggataagactatgtttatctgcaagtcaagctggtccatgagggaagccaatgtggcatgcctggatctgggttttcaacaaggagctgatacccagagaagatttaagctgagtgacctcagcattaactctactgagtgtctgcatgtgcactgtagagggcttgaaacaagcctggcagagtgcaccttcaccaagagaaggacaatgggataccaggactttgctgatgtggtctgctacacccagaaggctgattcccccatggatgatttctttcaatgtgtgaatgggaagtacatcagccagatgaaagcatgtgatgggatcaatgactgtggagaccagagtgatgagctgtgctgcaaagcttgccaggggaaaggctttcattgcaagagtggggtctgtattccctcccaataccagtgcaatggggaggtggattgcatcacaggagaagatgaagtgggctgtgctggatttgcctcagtgactcaggaagagacagaaattctcacagctgatatggatgcagagagaaggaggatcaagtccctcctgcctaaactgagttgtggagtgaagaacaggatgcatattagaaggaagagaattgtggggggtaaaagagcccagcttggggacctgccttggcaagtggctatcaaagatgccagtggtattacctgtggtgggatctatattggtggttgctggattctcactgctgcacactgtctgagagccagcaaaacccacagatatcagatttggactacagtggttgactggattcaccctgacctgaagagaattgttattgagtatgtggatagaatcatcttccatgagaattacaatgctgggacttatcagaatgatattgccctcattgaaatgaagaaggatgggaacaaaaaggattgtgagctgcccagaagcattccagcctgtgtgccatggagcccctatctgtttcagccaaatgacacttgtattgtttctggttggggcagggaaaaagacaatgaaagagtctttagcctccagtggggagaagttaagctgatttcaaattgcagcaaattctatgggaacaggttctatgagaaggagatggagtgtgctgggacatatgatggttcaattgatgcttgcaaaggagatagtggtgggccactggtctgcatggatgccaataatgtgacatatgtctggggggtggtttcctggggagagaattgtgggaagccagagtttcctggtgtctatacaaaagtggctaactattttgactggatcagctatcatgtggggagacctttcatcagtcagtataatgtgtag

SEQ ID NO: 38 - DNA, 1752 bp, Artificial Sequence
  Synthetic construct
atgaagctgctgcatgtgtttctgctctttctgtgcttccacctgagattttgcaaggtgacctacacctctcaggaggatctggtggagaaaaagtgcctggcaaaaaagtatactcacctcagttgtgacaaggtcttctgccagccatggcagagatgtattgagggaacctgtgtgtgcaaactcccttatcagtgtcccaagaatgggactgcagtctgtgcaaccaacagaagaagctttcccacatactgtcagcagaaatcccttgagtgcctgcaccctggaaccaaattcctgaataatggaacttgcacagcagaggggaaattcagtgtcagcctgaaacatggcaacactgatagtgaggggattgttgaagtgaagctggtggatcaggataagactatgtttatctgcaaaagttcatggagcatgagagaagcaaatgtggcatgccttgatctgggtttccaacaaggagctgacactcagagaagatttaaactcagtgatctgtcaatcaacagcacagagtgcctgcatgtgcactgtagaggacttgagacatcacttgcagaatgcacatttaccaaaagaagaaccatgggataccaggattttgcagatgtggtgtgctatactcagaaagctgatagccccatggatgacttcttccagtgtgtcaatgggaagtatatcagccagatgaaggcctgtgatggaatcaatgactgtggagatcagagtgatgaactctgttgtaaggcatgtcaggggaagggtttccattgcaagagtggagtctgcattccttcacaataccagtgcaatggagaggtggattgtattactggggaggatgaagtgggttgtgcaggatttgccagtgtgactcaggaggaaactgagattctgacagctgacatggatgctgagagaagaaggatcaagagcctgctcccaaagctgtcatgtggggtcaaaaatagaatgcacatcagaaggaagagaattgtgggagggaaaagagctcagctgggagacctcccttggcaagtggcaatcaaggatgctagtggtatcacatgtggaggaatctacattggggggtgttggattctgactgcagcccattgcctgagggcatccaaaacccacagatatcagatctggacaacagttgtggactggattcaccctgacctcaaaagaattgtcattgagtatgtggacagaatcattttccatgagaattacaatgctggcacttaccagaatgacattgccctgattgaaatgaagaaggatggaaacaagaaggattgtgaactccccaggtcaatcccagcctgtgtgccctggagcccctacctgttccagccaaatgatacatgtattgtcagtggatgggggagagagaaggacaatgagagagtgttttcactgcagtggggggaagtgaagctgatcagcaattgctccaagttctatggaaacaggttttatgagaaggagatggagtgtgctgggacttatgatggaagtattgatgcttgtaaaggggattcaggaggtcctcttgtctgcatggatgctaacaatgtgacatatgtgtggggagtagtcagttggggagaaaactgtggaaaaccagaatttcctggggtgtatactaaagtggcaaattattttgactggatcagctatcatgtgggtagacccttcattagtcagtataatgtgtga

SEQ ID NO: 39 - DNA, 1752 bp, Artificial Sequence
  Synthetic construct
atgaagctcctgcatgtctttctgctgtttctctgcttccatctgagattttgcaaggtgacctacacctctcaggaagaccttgttgaaaagaagtgtctggccaaaaagtatactcatctgtcttgtgacaaagtcttctgtcagccctggcagagatgcattgagggaacatgtgtctgcaagctcccttatcagtgtcccaaaaatggcacagcagtttgtgccacaaatagaaggtcttttcctacatactgccagcagaagagccttgaatgtctccacccaggcacaaagtttctgaataatgggacttgcacagcagaaggcaaatttagtgtgtccctgaaacatggaaacactgatagtgagggcattgtggaggttaagcttgtggatcaggataaaacaatgttcatctgcaagagctcatggtccatgagagaggctaatgtggcttgtctggacctgggttttcaacaaggagctgatactcagagaaggtttaagctcagtgacctgagcatcaacagtacagagtgtctgcatgtgcattgtagaggactggaaacttctctggctgagtgtacctttacaaagagaagaacaatgggataccaggattttgctgatgtggtctgctacacccagaaagctgactcccccatggatgatttctttcaatgtgttaatgggaaatacattagccagatgaaggcttgtgatggtatcaatgactgtggagaccagtctgatgaactgtgctgtaaagcctgtcaagggaaaggattccactgcaagagtggagtgtgcattccatcccagtaccagtgtaatggagaagttgattgcattacaggggaagatgaagtgggttgtgcagggtttgcttcagtaactcaggaggagacagaaatcctgacagctgacatggatgctgaaaggagaaggatcaaaagcctgctgcccaagctctcatgtggggtgaaaaacaggatgcatattagaaggaagaggattgtgggagggaaaagagcccagctgggggacctgccatggcaggttgccattaaagatgcttcagggatcacatgtgggggcatctatattggaggctgttggattctcacagcagcacactgcctgagagcttcaaagactcacagatatcagatttggacaacagtggtggattggattcaccctgatctcaaaagaattgtcattgaatatgttgacagaatcatctttcatgaaaactacaatgctggaacttatcagaatgatattgctctgattgaaatgaagaaagatggaaacaaaaaggactgtgaactgcccagaagcatccctgcttgtgttccatggtccccttacctgtttcagcctaatgacacctgcattgttagtggctgggggagagagaaggacaatgagagagtgttttccctgcagtggggagaggtgaaactcatctctaactgttcaaaattctatgggaatagattttatgagaaggagatggaatgtgctgggacctatgatgggtcaattgatgcttgtaagggtgacagtggaggacccctggtgtgcatggatgccaacaatgtgacatatgtctggggggtggttagctggggagaaaattgtgggaagcctgagttccctggggtgtacaccaaggttgcaaactattttgattggatttcttatcatgtgggtagaccatttatcagtcagtataatgtgtag

SEQ ID NO: 40 - DNA, 1752 bp, Artificial Sequence
  Synthetic construct
atgaaacttcttcatgtgttcctgctgtttctgtgcttccacctgaggttttgcaaggtgacctatacttctcaagaggatctggtggagaaaaaatgcctggctaaaaaatatactcacctgtcctgtgataaagtcttctgccagccatggcagagatgcattgagggaacttgtgtgtgcaaactgccatatcagtgcccaaaaaatggaactgctgtgtgtgctactaacaggagaagcttcccaacttattgccagcagaaaagtctggaatgccttcatccaggaactaaatttctgaataatggaacttgcactgctgaaggaaaatttagtgtgtccctgaaacatggaaatactgattcagagggaattgtggaagtgaaacttgtggatcaggataaaactatgttcatttgcaaaagcagctggagcatgagggaagctaatgtggcttgccttgatcttggatttcaacaaggagctgatactcagagaaggtttaaactgtctgatctgtctattaattccactgaatgcctgcatgtgcattgcagaggactggagactagtctggctgaatgcacttttactaaaagaagaactatgggatatcaggattttgctgatgtggtgtgctatactcagaaagctgattctccaatggatgatttctttcaatgtgtgaatggaaaatatatttctcagatgaaagcttgtgatggaatcaatgattgtggagatcagagtgatgaactgtgctgcaaagcttgccagggaaaaggatttcattgcaaatctggagtgtgcattccaagccagtatcagtgcaatggagaggtggattgcattactggagaagatgaagtgggatgtgctggatttgcttctgtgactcaagaagaaactgaaattctgactgctgatatggatgctgaaagaagaagaattaaatcactgctgcctaaactgtcttgtggagtgaaaaacagaatgcacattagaaggaaaagaattgtgggaggaaaaagagctcagctgggagatctgccatggcaggtggctattaaagatgctagtggaatcacttgtggaggaatttatattggaggatgctggattctgactgctgctcattgcctgagagctagtaaaactcatagatatcagatttggactactgtggtggattggattcacccagatcttaaaagaattgtgattgaatatgtggatagaattattttccatgaaaactataatgctggaacttatcagaatgatattgctctgattgaaatgaaaaaagatggaaacaaaaaagattgtgagctgcctagatccatccctgcttgtgtcccatggtctccttatctgttccaacctaatgatacttgcattgtgtctggatggggaagagaaaaagataatgaaagagtcttttcacttcaatggggagaagtgaaactgattagcaactgctctaaattttatggaaatagattctatgaaaaagaaatggaatgtgctggaacttatgatggatccattgatgcttgcaaaggagattctggaggaccactggtctgcatggatgctaacaatgtgacttatgtctggggagtggtcagttggggagaaaactgtggaaaaccagagttcccaggagtgtatactaaagtggctaattattttgattggattagctatcatgtgggaaggccttttatttctcagtataatgtgtaa

SEQ ID NO: 41 - DNA, 1752 bp, Homo sapiens
atgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataa

SEQ ID NO: 42 - DNA, 1698 bp, Homo sapiens
aaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataa

SEQ ID NO: 43 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 44 - protein, 565 aa, Homo sapiens
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 45 - DNA, 54 bp, Homo sapiens
atgaagcttcttcatgttttcctgttatttctgtgcttccacttaaggttttgc

SEQ ID NO: 46 - protein, 18 aa, Homo sapiens
MKLLHVFLLFLCFHLRFC

SEQ ID NO: 47 - DNA, 185 bp, Artificial Sequence
  Synthetic construct
gggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 48 - DNA, 418 bp, Artificial Sequence
  Synthetic construct
ggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 49 - DNA, 117 bp, Artificial Sequence
  Synthetic construct
ccctaaaatgggcaaacattgcaagcagcaaacagcaaacacacagccctccctgcctgctgaccttggagctggggcagaggtcagacacctctctgggcccatgccacctccaac

SEQ ID NO: 50 - DNA, 321 bp, Artificial Sequence
  Synthetic construct
tggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 51 - DNA, 334 bp, Artificial Sequence
  Synthetic construct
ccctaaaatgggcaaacattgcaagcagcaaacagcaaacacacagccctccctgcctgctgaccttggagctggggcagaggtcagacacctctctgggcccatgccacctccaactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 52 - DNA, 747 bp, Artificial Sequence
  Synthetic construct
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 53 - DNA, 145 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 54 - DNA, 145 bp, adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 55 - DNA, 208 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 56 - protein, 736 aa, adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 57 - protein, 736 aa, Artificial Sequence
  Synthetic capsid protein
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 58 - protein, 736 aa, Artificial Sequence
  Synthetic capsid protein
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 59 - protein, 724 aa, adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 60 - DNA, 66 bp, Simian virus 40
gtaaatataaaatttttaagtgtataatgtgttaaactactgattctaattgtttctctcttttag

SEQ ID NO: 61 - DNA, 659 bp, Artificial Sequence
  Synthetic construct
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 62 - DNA, 588 bp, Woodchuck hepatitis virus
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - DNA, 245 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggt

SEQ ID NO: 64 - DNA, 589 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 65 - DNA, 119 bp, Artificial Sequence
  Synthetic Polynucleotide
cccagccagtggacttagcccctgtttgctcctccgataactggggtgaccttggttaatattcaccagcagcctccccctcagcttcaggcaccaccactgacctgggacagtgaatc

SEQ ID NO: 66 - DNA, 119 bp, Artificial Sequence; identical to SEQ ID NO: 65
  Synthetic Polynucleotide

SEQ ID NO: 67 - DNA, 747 bp, Artificial Sequence; identical to SEQ ID NO: 52
  Synthetic Polynucleotide

SEQ ID NO: 68 - DNA, 576 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 69 - DNA, 898 bp, Artificial Sequence
  Synthetic Polynucleotide
caggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctctacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 70 - DNA, 898 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 71 - DNA, 573 bp, Artificial Sequence
  Synthetic Polynucleotide
tgcctccccgtgttcctgctctttgtccctctgtcctacttagactaatatttgccttgggtactgcaaacaggaaatgggggagggacaggaggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatcc

SEQ ID NO: 72 - DNA, 1618 bp, Artificial Sequence
  Synthetic Polynucleotide
cacaatttttatagacttctcatcatgttttttttttccaatatttgccctcccctttctaccttgacctagtagggaggtgtttattcaacacctctgattggctcataaacaaccagtccttaaaatcatgccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtcagtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 73 - DNA, 1363 bp, Artificial Sequence
  Synthetic Polynucleotide
ggaggggtgttaattaaaagcttgatatgggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtcatgcctccccgtgttcctgctctttgtccctctgtcctacttagactaatatttgccttgggtactgcaaacaggaaatgggggagggacaggacccagccagtggacttagcccctgtttgctcctccgataactggggtgaccttggttaatattcaccagcagcctcccccggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 74 - DNA, 1484 bp, Artificial Sequence
  Synthetic Polynucleotide
gggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtcacaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 75 - DNA, 903 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggatataaacgaggacagggccctgtctcctcagcttcaggcaccaccactgacctgggacagtgaatgatccccctgatctgcggcc

SEQ ID NO: 76 - DNA, 933 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctccccctattgattgttgcccctctggatccactgcttaaatacggacgaggacagggccctgtctcctcagcttcaggcaccaccactgacctgggacagtgaatgatccccctgatctgcggcc

SEQ ID NO: 77 - DNA, 943 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggttaataatctggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctccccctattgattgttgcccctctggatccactgcttaaatacggacgaggacagggccctgtctcctcagcttcaggcaccaccactgacctgggacagtgaatgatccccctgatctgcggcc

SEQ ID NO: 78 - DNA, 913 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggctgtttgctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 79 - DNA, 915 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 80 - DNA, 909 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 81 - DNA, 907 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctctggcccttgccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 82 - DNA, 909 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggttattgatattcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 83 - DNA, 907 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 84 - DNA, 907 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacatggcccttgccagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 85 - DNA, 913 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctccccctatataaatattgattgttgcccctctggatccactgcttaaatacggacgaggacagggccctgtctcctcagcttcaggcaccaccactgacctgggacagtgaatgatccccctgatctgcggcc

SEQ ID NO: 86 - DNA, 919 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 87 - DNA, 919 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctctggcccttgccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggttattgatattcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 88 - DNA, 915 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctcccccttattgatattcgttgcccctctggatccactgcttaaatacggacgaggacagggccctgtctcctcagcttcaggcaccaccactgacctgggacagtgaatgatccccctgatctgcggcc

SEQ ID NO: 89 - DNA, 909 bp, Artificial Sequence
  Synthetic Polynucleotide
ttattgatattcggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 90 - DNA, 907 bp, Artificial Sequence
  Synthetic Polynucleotide
tggcccttgcggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 91 - DNA, 907 bp, Artificial Sequence
  Synthetic Polynucleotide
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctccccctttattgatattcgcccctctggatccactgcttaaatacggacgaggacagggccctgtctcctcagcttcaggcaccaccactgacctgggacagtgaatgatccccctgatctgcggcc

SEQ ID NO: 92 - DNA, 322 bp, Homo sapiens
caggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 93 - DNA, 117 bp, Homo sapiens; identical to SEQ ID NO: 49

SEQ ID NO: 94 - DNA, 426 bp, Homo sapiens
gtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 95 - DNA, 73 bp, Homo sapiens
agtggacttagcccctgtttgctcctccgataactggggtgaccttggttaatattcaccagcagcctccccc

SEQ ID NO: 96 - DNA, 80 bp, Homo sapiens
cccagccagtggacttagcccctgtttgctcctccgataactggggtgaccttggttaatattcaccagcagcctccccc

SEQ ID NO: 97 - DNA, 149 bp, Homo sapiens
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctc

SEQ ID NO: 98 - DNA, 93 bp, Homo sapiens
ttaatttttaaaaagcagtcaaaagtccaagtggcccttgcgagcatttactctctctgtttgctctggttaataatctcaggagcacaaaca

SEQ ID NO: 99 - DNA, 134 bp, Homo sapiens
cacaatttttatagacttctcatcatgttttttttttccaatatttgccctcccctttctaccttgacctagtagggaggtgtttattcaacacctctgattggctcataaacaaccagtccttaaaatcatgc

SEQ ID NO: 100 - DNA, 12 bp, Homo sapiens
aatttttataga

SEQ ID NO: 101 - DNA, 52 bp, Homo sapiens
gtagggaggtgtttattcaacacctctgattggctcataaacaaccagtcct

SEQ ID NO: 102 - DNA, 586 bp, Homo sapiens
gggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtca

SEQ ID NO: 103 - DNA, 113 bp, Homo sapiens
cttgcaaaattatctaggtagcatgtgatagaaccaggattcaaacccaggtgtgtctgacctcaaagtgtggggactttccactctactgctgctaatttttttctaaaaat

SEQ ID NO: 104 - DNA, 93 bp, Homo sapiens
tgcctccccgtgttcctgctctttgtccctctgtcctacttagactaatatttgccttgggtactgcaaacaggaaatgggggagggacagga

SEQ ID NO: 105 - DNA, 77 bp, Homo sapiens
tgttcctgctctttgtccctctgtcctacttagactaatatttgccttgggtactgcaaacaggaaatgggggaggg

SEQ ID NO: 106 - DNA, 14 bp, Homo sapiens
tagactaatatttg

SEQ ID NO: 107 - DNA, 9 bp, Homo sapiens
cttgcaaaa

SEQ ID NO: 108 - DNA, 10 bp, Homo sapiens
tatgtacatt

SEQ ID NO: 109 - DNA, 10 bp, Homo sapiens
cccacaacat

SEQ ID NO: 110 - DNA, 9 bp, Homo sapiens
ttgagaaat

SEQ ID NO: 111 - DNA, 10 bp, Homo sapiens
gattcatctc

SEQ ID NO: 112 - DNA, 10 bp, Homo sapiens
ttggtctaac

SEQ ID NO: 113 - DNA, 12 bp, Homo sapiens
ctttctaccttg

SEQ ID NO: 114 - DNA, 10 bp, Homo sapiens
ctgtttgctc

SEQ ID NO: 115 - DNA, 9 bp, Homo sapiens
cttgcaaaa

SEQ ID NO: 116 - DNA, 10 bp, Homo sapiens
ctgtttgctc

SEQ ID NO: 117 - DNA, 9 bp, Homo sapiens
cccaggtgt

SEQ ID NO: 118 - DNA, 8 bp, Homo sapiens
tattgatt

SEQ ID NO: 119 - DNA, 11 bp, Homo sapiens
aaaagcagtca

SEQ ID NO: 120 - DNA, 11 bp, Homo sapiens
aggcaggagaa

SEQ ID NO: 121 - DNA, 11 bp, Homo sapiens
gagaagaaatc

SEQ ID NO: 122 - DNA, 11 bp, Homo sapiens
caacatcctgg

SEQ ID NO: 123 - DNA, 11 bp, Homo sapiens
acttatcctct

SEQ ID NO: 124 - DNA, 11 bp, Homo sapiens
cgtgttcctgc

SEQ ID NO: 125 - DNA, 9 bp, Homo sapiens
gcaaacagg

SEQ ID NO: 126 - DNA, 7 bp, Homo sapiens
tcatgtt

SEQ ID NO: 127 - DNA, 8 bp, Homo sapiens
atcaacat

SEQ ID NO: 128 - DNA, 18 bp, Homo sapiens
ataatctcaggagcacaa

SEQ ID NO: 129 - DNA, 6 bp, Homo sapiens
ttatct

SEQ ID NO: 130 - DNA, 6 bp, Homo sapiens
tgatag

SEQ ID NO: 131 - DNA, 6 bp, Homo sapiens
agatag

SEQ ID NO: 132 - DNA, 11 bp, Homo sapiens
gacttatcctc

SEQ ID NO: 133 - DNA, 18 bp, Homo sapiens
tgtttattcaacacctct

SEQ ID NO: 134 - DNA, 10 bp, Homo sapiens
gttaataatc

SEQ ID NO: 135 - DNA, 13 bp, Homo sapiens
gactaatatttgc

SEQ ID NO: 136 - DNA, 13 bp, Homo sapiens
cttgcaaaattat

SEQ ID NO: 137 - DNA, 13 bp, Homo sapiens
gctgctaattttt

SEQ ID NO: 138 - DNA, 13 bp, Homo sapiens
acaaataattaac

SEQ ID NO: 139 - DNA, 13 bp, Homo sapiens
gttaatttttaaa

SEQ ID NO: 140 - DNA, 11 bp, Homo sapiens
ggttaataatc

SEQ ID NO: 141 - DNA, 12 bp, Homo sapiens
tggttaatattc

SEQ ID NO: 142 - DNA, 10 bp, Homo sapiens
tgtttgctct

SEQ ID NO: 143 - DNA, 10 bp, Homo sapiens
agcacaaaca

SEQ ID NO: 144 - DNA, 9 bp, Homo sapiens
tgtttgctc

SEQ ID NO: 145 - DNA, 7 bp, Homo sapiens
tgtttgc

SEQ ID NO: 146 - DNA, 8 bp, Homo sapiens
tgtttgct

SEQ ID NO: 147 - DNA, 11 bp, Homo sapiens
agaaatcaaca

SEQ ID NO: 148 - DNA, 15 bp, Homo sapiens
ctaatatttgccttg

SEQ ID NO: 149 - DNA, 13 bp, Homo sapiens
gactaatatttgc

SEQ ID NO: 150 - DNA, 12 bp, Homo sapiens
tctgtttgctct

SEQ ID NO: 151 - DNA, 17 bp, Homo sapiens
ttcctgctctttgtccc

SEQ ID NO: 152 - DNA, 8 bp, Homo sapiens
cagagttt

SEQ ID NO: 153 - DNA, 12 bp, Homo sapiens
ccctgtttgctc

SEQ ID NO: 154 - DNA, 10 bp, Homo sapiens
tggcccttgc

SEQ ID NO: 155 - DNA, 15 bp, Homo sapiens
agcagtcaaaagtcc

SEQ ID NO: 156 - DNA, 12 bp, Homo sapiens
ttattgatattc

SEQ ID NO: 157 - DNA, 8 bp, Homo sapiens
tattgatt

SEQ ID NO: 158 - DNA, 15 bp, Homo sapiens
ttttttttccaatat

SEQ ID NO: 159 - DNA, 21 bp, Homo sapiens
atttactctctctgtttgctc

SEQ ID NO: 160 - DNA, 21 bp, Homo sapiens
ttactctctctgtttgctctg

SEQ ID NO: 161 - DNA, 13 bp, Homo sapiens
ttaataatctcag

SEQ ID NO: 162 - DNA, 17 bp, Homo sapiens
tggttaatattcaccag

SEQ ID NO: 163 - DNA, 11 bp, Homo sapiens
caacatcctgg

SEQ ID NO: 164 - DNA, 13 bp, Homo sapiens
agtccaagtggcc

SEQ ID NO: 165 - DNA, 12 bp, Homo sapiens
gtccaagtggcc

SEQ ID NO: 166 - DNA, 10 bp, Homo sapiens
ctctggttaa

SEQ ID NO: 167 - DNA, 10 bp, Homo sapiens
aaatggggga

SEQ ID NO: 168 - DNA, 10 bp, Homo sapiens
tcaaaagtcc

SEQ ID NO: 169 - DNA, 14 bp, Homo sapiens
tagactaatatttg

SEQ ID NO: 170 - DNA, 15 bp, Homo sapiens
caggagaagaaatca

SEQ ID NO: 171 - DNA, 14 bp, Homo sapiens
tgttaattaaaagc

SEQ ID NO: 172 - DNA, 10 bp, Homo sapiens
gttaattttt

SEQ ID NO: 173 - DNA, 10 bp, Homo sapiens
ttactctctc

SEQ ID NO: 174 - DNA, 10 bp, Homo sapiens
cttgggtact

SEQ ID NO: 175 - DNA, 9 bp, Homo sapiens
gcaaacagg

SEQ ID NO: 176 - DNA, 8 bp, Homo sapiens
ttaatttt

SEQ ID NO: 177 - DNA, 8 bp, Homo sapiens
gggattca

SEQ ID NO: 178 - DNA, 8 bp, Homo sapiens
ggtactgt

SEQ ID NO: 179 - DNA, 8 bp, Homo sapiens
ttaatccc

SEQ ID NO: 180 - DNA, 8 bp, Homo sapiens
ggtattga

SEQ ID NO: 181 - DNA, 8 bp, Homo sapiens
ggctctaa

SEQ ID NO: 182 - DNA, 8 bp, Homo sapiens
ggcatgat

SEQ ID NO: 183 - DNA, 8 bp, Homo sapiens
ttcaaacc

SEQ ID NO: 184 - DNA, 8 bp, Homo sapiens
atagaacc

SEQ ID NO: 185 - DNA, 10 bp, Homo sapiens
tggaggcagg

SEQ ID NO: 186 - DNA, 7 bp, Homo sapiens
acaggaa

SEQ ID NO: 187 - DNA, 13 bp, Homo sapiens
atgggggagggac

SEQ ID NO: 188 - DNA, 9 bp, Homo sapiens
gcagtcaaa

SEQ ID NO: 189 - DNA, 9 bp, Homo sapiens
gagcacaaa

SEQ ID NO: 190 - DNA, 11 bp, Homo sapiens
attcctggagg

SEQ ID NO: 191 - DNA, 11 bp, Homo sapiens
caaacaggaaa

SEQ ID NO: 192 - DNA, 6 bp, Homo sapiens
tataaa

SEQ ID NO: 193 - DNA, 8 bp, Homo sapiens
tatataaa

SEQ ID NO: 194 - DNA, 13 bp, Homo sapiens
aaacattcctgga

SEQ ID NO: 195 - DNA, 7 bp, Homo sapiens
acgtgtg

SEQ ID NO: 196 - DNA, 9 bp, Homo sapiens
caggtgtca

SEQ ID NO: 197 - DNA, 9 bp, Homo sapiens
cagatgtca

SEQ ID NO: 198 - DNA, 11 bp, Homo sapiens
tccaagtggcc

SEQ ID NO: 199 - DNA, 11 bp, Homo sapiens
agcatgtgata

SEQ ID NO: 200 - DNA, 7 bp, Homo sapiens
tataaaa

SEQ ID NO: 201 - DNA, 7 bp, Homo sapiens
ttaaata

SEQ ID NO: 202 - DNA, 335 bp, Artificial Sequence
  Synthetic Polynucleotide
ccctaaaatgggcaaacattgcaagcagcaaacagcaaacacacagccctccctgcctgctgaccttggagctggggcagaggtcagacacctctctgggcccatgccacctccaactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc